(19) United States
(12) Patent Application Publication
(10) Pub. No.: US 2008/0275032 A1
(43) Pub. Date: Nov. 6, 2008

(54) Pyridazinone compounds

(76) Inventors: Yuefen Zhou, San Diego, CA (US); Liansheng Li, San Diego, CA (US); Stephen E. Webber, San Diego, CA (US); Peter Dragovich, San Diego, CA (US); Douglas E. Murphy, San Diego, CA (US); Chinh V. Tran, San Diego, CA (US); Jingjing Zhao, San Diego, CA (US); Frank Ruebsam, San Diego, CA (US)

Correspondence Address: CONNOLLY BOVE LODGE & HUTZ LLP, 1875 EYE STREET, N.W., SUITE 1100, WASHINGTON, DC, 20036, US

(21) Appl. No.: 11/898,334

(22) Filed: Sep. 11, 2007

Related U.S. Application Data
(60) Provisional application No. 60/877,629, filed on Dec. 29, 2006.

(51) Int. Cl. A61K 31/549; C07D 401/04; C07D 285/26; A61K 31/497; A61P 31/14

(52) U.S. Cl. 514/223.5; 544/238; 544/13; 514/252.01

(57) ABSTRACT

The invention is directed to pyridazinone compounds and pharmaceutical compositions containing such compounds that are useful in treating infections by hepatitis C virus.

This application claims the benefit of U.S. provisional application No. 60/876,171, which was filed on Dec. 21, 2006.

### FIELD OF THE INVENTION

The invention is directed to pyridazinone compounds and pharmaceutical compositions containing such compounds that are useful in treating infections by hepatitis C virus.

### BACKGROUND OF THE INVENTION

Hepatitis C is a major health problem world-wide. The World Health Organization estimates that 170 million people are chronic carriers of the hepatitis C virus (HCV), with 4 million carriers in the United States alone. In the United States, HCV infection accounts for 40% of chronic liver disease and HCV disease is the most common cause for liver transplantation. HCV infection leads to a chronic infection and about 70% of persons infected will develop chronic histological changes in the liver (chronic hepatitis) with a 10-40% risk of cirrhosis and an estimated 4% lifetime risk of hepatocellular carcinoma. The CDC estimates that each year in the United States there are 35,000 new cases of HCV infection and approximately ten thousand deaths attributed to HCV disease.

The current standard of care is a pegylated interferon/ribavirin combination at a cost of approximately $31,000/year. These drugs have difficult dosing problems and side-effects that preclude their use in almost half of diagnosed patients. Pegylated interferon treatment is associated with menacing flu-like symptoms, irritability, inability to concentrate, suicidal ideation, and leukocytopenia. Ribavirin is associated with hemolytic anemia and birth defects.

The overall response to this standard therapy is low; approximately one third of patients do not respond. Of those who do respond, a large fraction relapses within six months of completing 6-12 months of therapy. As a consequence, the long-term response rate for all patients entering treatment is only about 50%. The relatively low response rate and the significant side-effects of current therapy anti-HCV drug treatments, coupled with the negative long term effects of chronic HCV infection, result in a continuing medical need for improved therapy. Antiviral pharmaceuticals to treat RNA virus diseases like HCV are few, and as described above are often associated with multiple adverse effects. While there are, in some cases, medicines available to reduce disease symptoms, there are few drugs to effectively inhibit replication of the underlying virus. The significance and prevalence of RNA virus diseases, including but not limited to chronic infection by the hepatitis C virus, and coupled with the limited availability and effectiveness of current antiviral pharmaceuticals, have created a compelling and continuing need for new pharmaceuticals to treat these diseases.

### SUMMARY OF THE INVENTION

The present invention describes novel pyridazinone compounds, pharmaceutically acceptable salts, and pharmaceutically acceptable solvates thereof, which are useful in treating or preventing a hepatitis C virus infection in a patient in need thereof comprising administering to the patient a therapeutically or prophylactically effective amount of a pyridazinone compound.

In a general aspect, the invention relates to compounds of Formula I

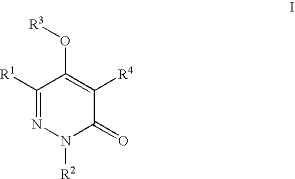

wherein
R¹ is hydrogen, cyano, halo, C₁-C₆ alkyl, C₁-C₆ haloalkyl, C₁-C₆ hydroxyalkyl, C₁-C₆ alkoxy, C₁-C₆ alkenyl, C₁-C₆ alkynyl, —CO₂R⁷, —C(O)NR⁷R⁸, C₃-C₈ cycloalkyl, aryl, or heterocyclyl having 1, 2, or 3 N, O, or S atoms, wherein R⁷ and R⁸ are independently hydrogen, C₁-C₆ alkyl, aryl, or heterocyclyl,
R² is hydrogen, C₁-C₆ alkyl, C₁-C₆ alkenyl, C₁-C₆ alkynyl, C₁-C₆ haloalkyl, C₁-C₆ hydroxyalkyl, C₁-C₆ alkoxy, C₃-C₈ cycloalkyl, aryl, or heterocyclyl having 1, 2, or 3 N, O, or S atoms, —C₁-C₆ alkylene(C₃-C₈ cycloalkyl), —C₁-C₆ alkylene(aryl), or —C₁-C₆ alkylene (heterocyclyl),
R³ is hydrogen, C₁-C₆ alkyl, or —(C₁-C₆ alkylene)ₙ-(O)ₜC(O)R⁹, wherein n and t are independently 0 or 1, wherein R⁹ is C₁-C₆ alkyl, aryl, or heterocyclyl,
R⁴ is selected from

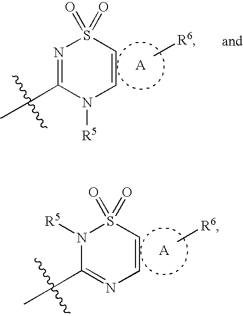

wherein R⁵ is hydrogen or C₁-C₆ alkyl, and
Ring A is a 6-membered aryl or heterocyclyl, substituted by 1-3
R⁶ moieties, wherein R⁶ is —NR¹⁰CO₂R¹¹, —NR¹⁰SO₂R¹¹, —NR¹⁰SO₂NR¹²R¹³, —NR¹⁰ (C₁-C₆ alkylene)SO₂NR²R³, —(C₁-C₆ alkylene)-SO₂R¹¹, —(C₁-C₆ alkylene)-CHR¹⁴SO₂R¹¹ or —(C₁-C₆ alkenyl)-SO₂R¹¹, wherein R¹⁰ is hydrogen, C₁-C₆ alkyl, —CO₂R¹¹, or —(C₁-C₆ alkylene)-OC(O)(C₁-C₆ alkyl), R¹¹ is C₁-C₆ alkyl, C₁-C₆ alkenyl, —(C₁-C₆ alkylene)NH₂, aryl, C₃-C₈ cycloalkyl, or heterocyclyl, R¹² and R¹³ are independently hydrogen, C₁-C₆ alkyl, or —CO₂R⁹, R¹⁴ is H or C₁-C₆ alkyl, or R¹⁰ and R¹¹ or R¹¹ and R¹⁴ combine with the hetero atom(s) to which they are attached to form a 5- or 6-membered heterocyclyl ring, and
wherein the above alkyl, alkylene, alkenyl, alkynyl, aryl, cycloalkyl, or heterocyclyl moieties are each optionally and independently substituted by 1-3 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents,
or a pharmaceutically acceptable salt, hydrate, solvate, tautomer or stereoisomer thereof.

In one embodiment, the invention relates to compounds of Formula I wherein R¹ is hydrogen, cyano, halo, C₁-C₆ alkyl, C₁-C₆ haloalkyl, C₁-C₆ hydroxyalkyl, C₁-C₆ alkoxy, C₁-C₆ alkenyl, C₁-C₆ alkynyl, —CO₂R⁷, —C(O)NR⁷R⁸, aryl, or heterocyclyl having 1, 2, or 3 N, O, or S atoms, wherein R⁷ and R⁸ are independently hydrogen, C₁-C₆ alkyl, aryl or heterocyclyl having 1 or 2 N, O, or S atoms.

In another embodiment, R¹ is heterocyclyl having 1 N, O, or S atom. In another embodiment, the invention relates to compounds of Formula I wherein R¹ is selected from

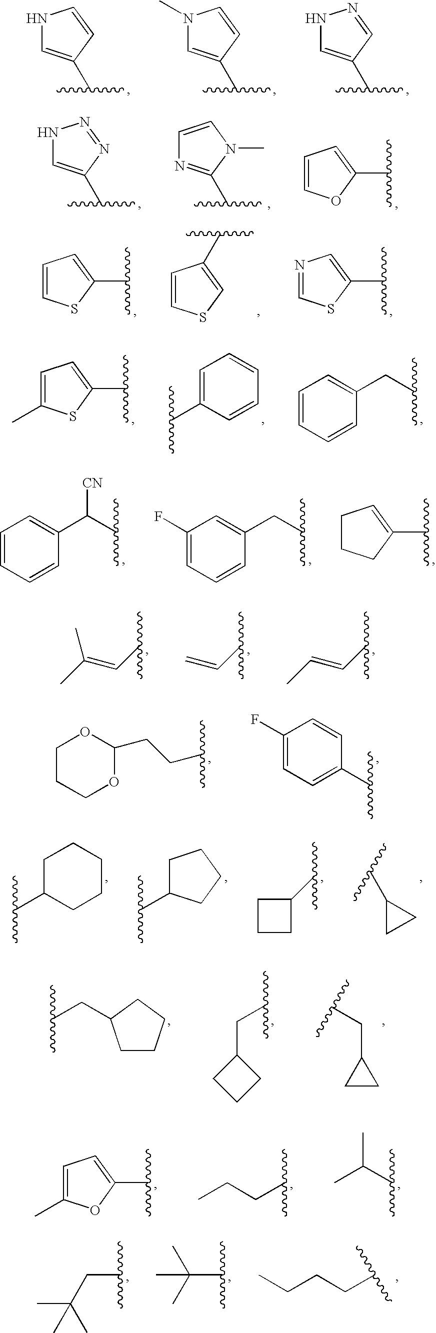

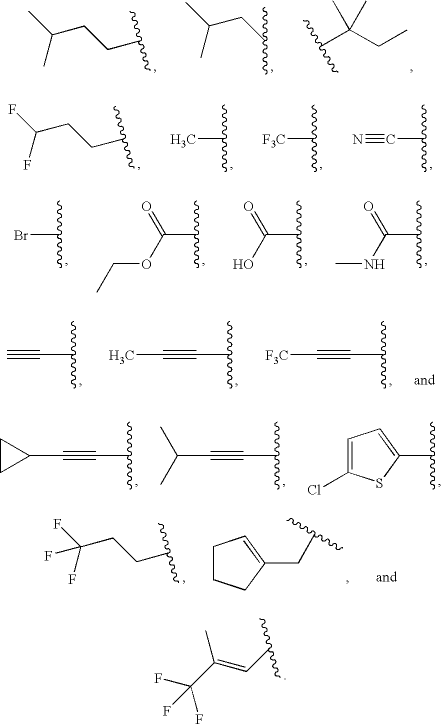

In a further embodiment, the invention relates to compounds of Formula I wherein R¹ is selected from

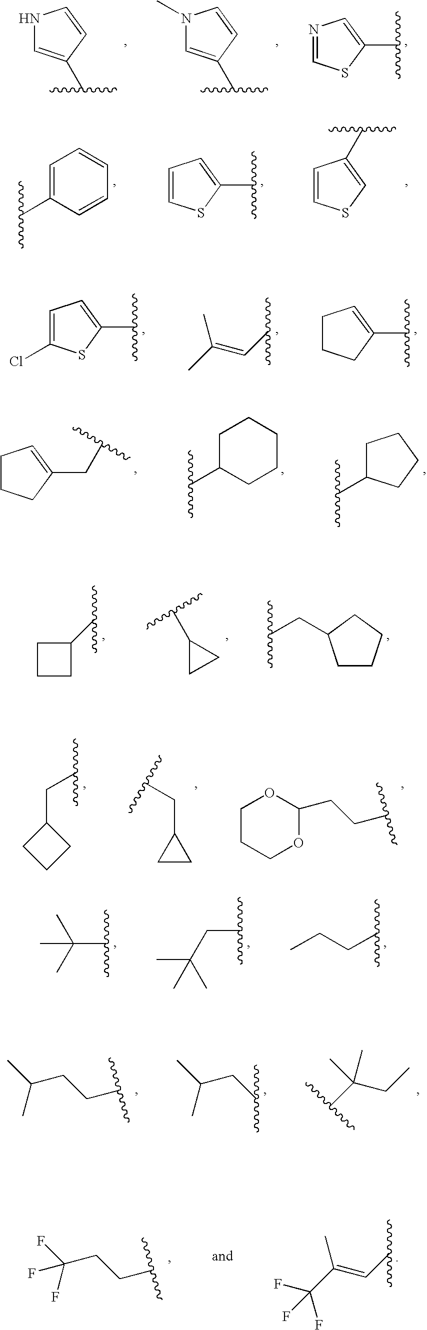

In one embodiment, the invention relates to compounds of Formula I wherein R² is selected from C₁-C₆ alkyl, alkenyl, alkynyl, C₁-C₆ haloalkyl, C₃-C₈ cycloalkyl, aryl, and heterocyclyl having 1, 2, or 3 N, O, or S atoms.

In another embodiment, the invention relates to compounds of Formula I wherein R² is selected from C₁-C₆ alkyl, C₃-C₈ cycloalkyl, aryl, and heterocyclyl. having 1, 2, or 3 N, O, or S atoms.

In another embodiment, the invention relates to compounds of Formula I wherein R² is selected from

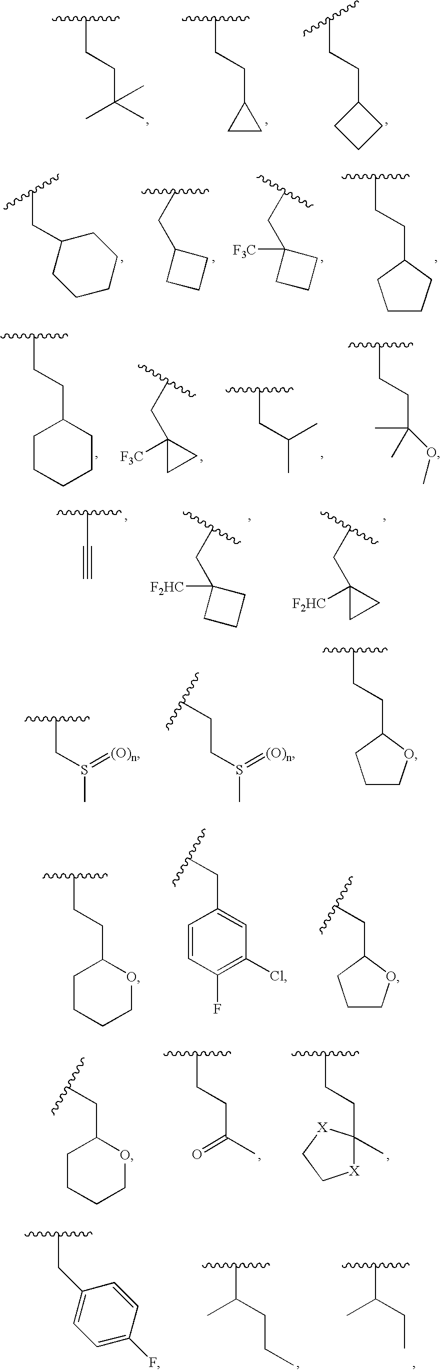

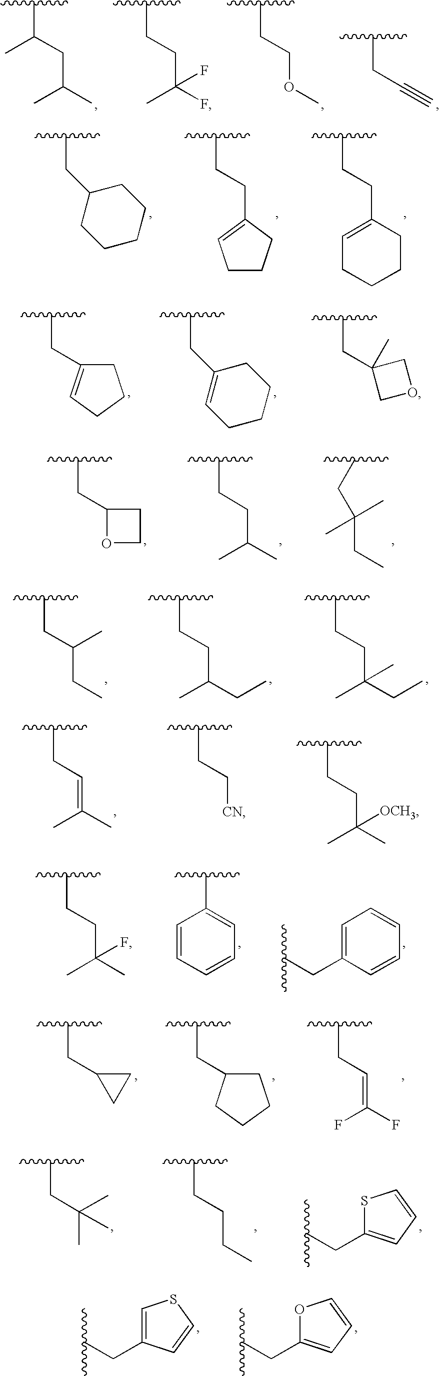

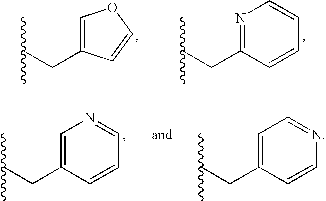

wherein X is O or S and n=0, 1, or 2.

In a further embodiment, R² is selected from

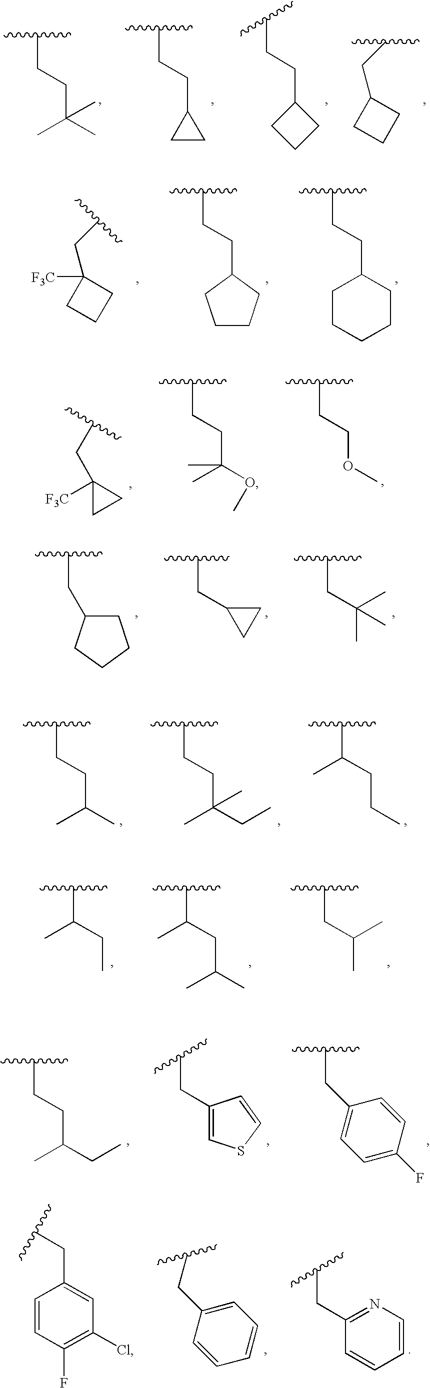

In one embodiment, the invention relates to compounds of Formula I wherein R³ is hydrogen, methyl, or —(CH₂)ₙ—(O)ₜC(O)R⁹, wherein n and t are independently 0 or 1.

In one embodiment, the invention relates to compounds of Formula I wherein R⁵ is hydrogen or methyl.

In one embodiment, the invention related to compounds of Formula I wherein Ring A is selected from

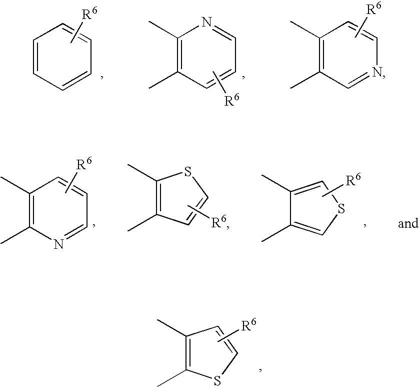

wherein R⁶ is defined as above.

In a further embodiment, Ring A is selected from

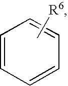

wherein R⁶ is defined as above. In a particular embodiment, R⁶ is —NR¹⁰SO₂R¹¹ or —NR¹⁰SO₂NR¹²R¹³, wherein R¹⁰ is hydrogen or C₁-C₆ alkyl R¹¹ is C₁-C₆ alkyl and R¹² and R¹³ are independently hydrogen or C₁-C₆ alkyl.

In another embodiment, the invention relates to compounds selected from
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(4-Fluoro-benzyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
Ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-sec-Butyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[5-Hydroxy-2-(3-methoxy-3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Ethanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
2-Methyl-propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
N-{3-[2-(3,3-Dimethyl-pentyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(1-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(1,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclohexyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
Ethanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[2-(4-Fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-3-oxo-6-thiophen-2-yl-2-(1-trifluoromethyl-cyclobutylmethyl)-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclobutyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopentyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-pentyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-methanesulfonamide;
N-{3-[2-(3-Chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Cyclopropanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
N-[3-(5-Hydroxy-2-isobutyl-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(2-Cyclopropylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(2-Cyclopentylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[2-(2,2-Dimethyl-propyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
2,2-Dimethyl-prop ionic acid ({3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methyl ester;
N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-methanesulfonamide;
N-[3-(2-Benzyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(5-Hydroxy-3-oxo-2-pyridin-2-ylmethyl-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[6-(5-Chloro-thiophen-2-yl)-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[2-(3-chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
Propane-1-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
Propane-2-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Propane-1-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[2-(4-Fluoro-benzyl)-5-hydroxy-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide;
2,2-Dimethyl-propionic acid ({3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methyl ester;
N-{3-[6-Cyclohexyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-propyl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-(1H-pyrrol-3-yl)-2,3-dihydro-pyridazin-4-yl]-1,1-dioxo-1,2-dihydro-1λ⁶-benzo[1,2,4]thiadiazin-7-yl}-methanesulfonamide;
2,2-Dimethyl-propionic acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yloxymethyl ester;
N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[2-(2-Cyclopropyl-ethyl)-5-hydroxy-3-oxo-6-thiophen-3-yl-2,3-dihydro-pyridazin-4-yl]-1,1-dioxo-1,2-dihydro-1λ⁶-benzo[1,2,4]thiadiazin-7-yl}-methanesulfonamide;
Propane-2-sulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-6-(1-methyl-1H-pyrrol-3-yl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-isopropyl-methanesulfonamide;
N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[6-(1,1-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopentyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
5-Hydroxy-4-(7-methanesulfonylmethyl-1,1-dioxo-1,2-dihydro-1λ⁶-benzo[1,2,4]thiadiazin-3-yl)-2-(3-methyl-butyl)-6-thiophen-2-yl-2H-pyridazin-3-one;
4-[7-(1,1-Dioxo-1l6-isothiazolidin-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one;
5-Hydroxy-4-[7-(2-methanesulfonyl-vinyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one;
5-Hydroxy-4-[7-(2-methanesulfonyl-ethyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one;
N-[3-(2-Cyclopropylmethyl-5-hydroxy-3-oxo-6-phenyl-2,3-dihydro-pyridazin-4-yl)-1,1-dioxo-1,2-dihydro-1λ⁶-benzo[1,2,4]thiadiazin-7-yl]-methanesulfonamide;
Isobutyric acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yl ester;
N-{3-[6-tert-Butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-(2-[1,3]Dioxan-2-yl-ethyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-6-isobutyl-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methoxymethyl-methanesulfonamide;
N-Benzyloxymethyl-N-{3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-3-carbamic-acid-benzyl-ester-sulfamide;
N-{3-[5-Hydroxy-2-(2-methoxy-ethyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[6-(2,2-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide;
N-{3-[5-Methoxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide;
N-{3-[6-Cyclopropylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclobutylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[6-tert-butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-3-oxo-6-(3,3,3-trifluoro-propyl)-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-Cyclobutylmethyl-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[5-Hydroxy-2-(3-methyl-butyl)-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
2-Amino-ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
2-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-ylamino}-ethanesulfonic acid amide;
N-{3-[2-(3,3-Dimethyl-butyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid methyl ester;
{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid isopropyl ester;
N-{3-[6-Cyclobutyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopentylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-sulfamide;
N-{3-[6-Cyclopentylmethyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2,6-bis-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-[3-(5-Hydroxy-3-oxo-6-thiophen-2-yl-2-thiophen-3-ylmethyl-2,3-dihydro-pyridazin-4-yl)-1,1-dioxo-1,2-dihydro-1λ⁶-benzo[1,2,4]thiadiazin-7-yl]-methanesulfonamide;
N-{3-[6-tert-Butyl-2-(3-chloro-4-fluoro-benzyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopropyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-(2-Hydroxy-ethyl)-N-{3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-(3,3,3-trifluoro-2-methyl-propenyl)-2,3-dihydro-pyridazin-4-yl]-1,1-dioxo-1,2-dihydro-1λ⁶-benzo[1,2,4]thiadiazin-7-yl}-methanesulfonamide;
Ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methyl-amide;
4-[7-(1,1-Dioxo-tetrahydro-1,6-thiophen-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[2-(2-Cyclopropyl-ethyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
2-Amino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[6-Cyclopent-1-enylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide, sodium salt; and
2-Diethylamino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1×6 benzo[1,2,4]thiadiazin-7-yl}-amide.

The invention is also directed to pharmaceutically acceptable salts, hydrates, and solvates of the compounds of Formula I. Advantageous methods of making the compounds of Formula I are also described.

In one aspect, the invention encompasses a method for treating or preventing hepatitis C virus infection in a mammal in need thereof, preferably in a human in need thereof, comprising administering to the patient a therapeutically or prophylactically effective amount of a Formula I compound. In one embodiment, the invention encompasses a method for treating or preventing hepatitis C virus infection by administering to a patient in need thereof a therapeutically or prophylactically effective amount of a Formula I compound that is an inhibitor of HCV NS5B polymerase.

In another aspect, the invention encompasses a method for treating or preventing hepatitis C virus infection in a patient in need thereof, comprising administering to the patient a therapeutically or prophylactically effective amount of a compound of Formula I and a pharmaceutically acceptable excipient, carrier, or vehicle.

In another aspect, the invention encompasses a method for treating or preventing hepatitis C virus infection in a patient in need thereof, comprising administering to the patient a therapeutically or prophylactically effective amount of a compound of Formula I and an additional therapeutic agent, preferably an additional antiviral agent or an immunomodulatory agent.

### DETAILED DESCRIPTION OF THE INVENTION

Where the following terms are used in this specification, they are used as defined below:

The terms “comprising,” “having” and “including” are used herein in their open, non-limiting sense.

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkylene”, as used herein, unless otherwise indicated, includes a divalent radical derived from alkyl, as exemplified by —CH₂CH₂CH₂CH₂—.

The term “alkoxy”, as used herein, unless otherwise indicated, includes O-alkyl groups wherein alkyl is as defined above.

The term “Me” means methyl, “Et” means ethyl, and “Ac” means acetyl.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

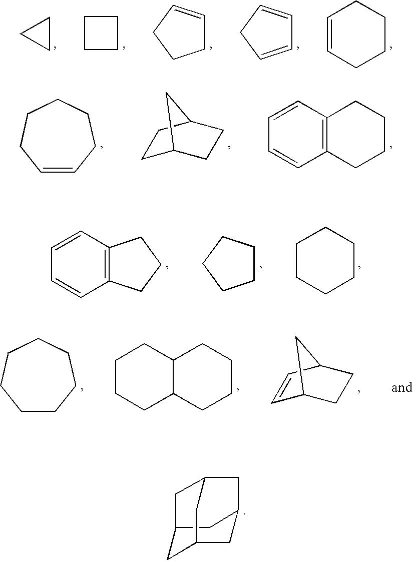

The term “aryl”, as used herein, unless otherwise indicated, includes an organic radical derived from an aromatic hydrocarbon by removal of one hydrogen, such as phenyl or naphthyl.

The term “heterocyclic” or “heterocyclyl”, as used herein, unless otherwise indicated, includes aromatic (e.g., heteroaryls) and non-aromatic heterocyclic groups containing one to four heteroatoms each selected from O, S and N, wherein each heterocyclic group has from 4-10 atoms in its ring system, and with the proviso that the ring of said group does not contain two adjacent O atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other illustrative examples of 4-10 membered heterocyclic are derived from, but not limited to, the following:

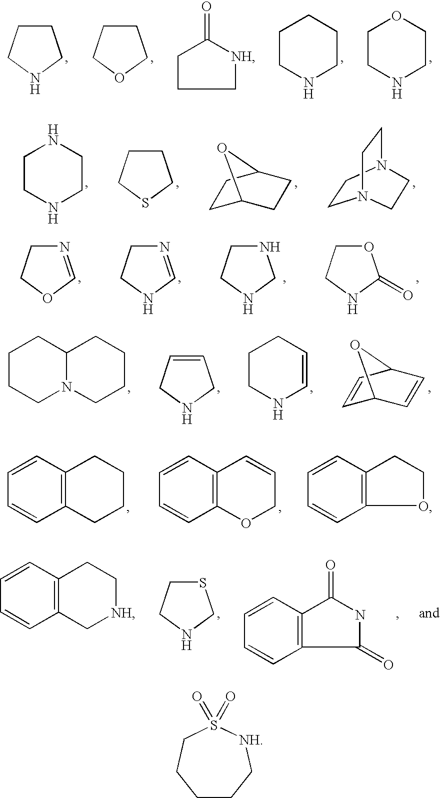

Unless defined otherwise, “alkyl,” “alkylene,” “alkenyl,” “alkynyl,” “aryl,” “cycloalkyl,” and “heterocyclyl” are each optionally and independently substituted by 1-3 substituents selected from alkanoyl, alkylamine, amino, aryl, cycloalkyl, heterocyclyl, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms, carboxyl, cyano, halo, hydroxy, nitro, —C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkylene), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)cycloalkyl, wherein each of these optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents, e.g., CF₃.

The term “immunomodulator” refers to natural or synthetic products capable of modifying the normal or aberrant immune system through stimulation or suppression.

The term “preventing” refers to the ability of a compound or composition of the invention to prevent a disease identified herein in patients diagnosed as having the disease or who are at risk of developing such disease. The term also encompasses preventing further progression of the disease in patients who are already suffering from or have symptoms of such disease.

The term “patient” or “subject” means an animal (e.g., cow, horse, sheep, pig, chicken, turkey, quail, cat, dog, mouse, rat, rabbit, guinea pig, etc.) or a mammal, including chimeric and transgenic animals and mammals. In the treatment or prevention of HCV infection, the term “patient” or “subject” preferably means a monkey or a human, most preferably a human. In a specific embodiment the patient or subject is infected by or exposed to the hepatitis C virus. In certain embodiments, the patient is a human infant (age 0-2), child (age 2-17), adolescent (age 12-17), adult (age 18 and up) or geriatric (age 70 and up) patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

The term a “therapeutically effective amount” refers to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

The term a “prophylactically effective amount” refers to an amount of a compound of the invention or other active ingredient sufficient to result in the prevention of infection, recurrence or spread of viral infection. A prophylactically effective amount may refer to an amount sufficient to prevent initial infection or the recurrence or spread of the infection or a disease associated with the infection. Used in connection with an amount of a compound of the invention, the term preferably encompasses a non-toxic amount that improves overall prophylaxis or enhances the prophylactic efficacy of or synergies with another prophylactic or therapeutic agent.

The term “in combination” refers to the use of more than one prophylactic and/or therapeutic agents simultaneously or sequentially and in a manner that their respective effects are additive or synergistic.

The term “treating” refers to:

(i) preventing a disease, disorder, or condition from occurring in an animal that may be predisposed to the disease, disorder and/or condition, but has not yet been diagnosed as having it;

(ii) inhibiting the disease, disorder, or condition, i.e., arresting its development; and

(iii) relieving the disease, disorder, or condition, i.e., causing regression of the disease, disorder, and/or condition.

The terms “α” and “β” indicate the specific stereochemical configuration of a substituent at an asymmetric carbon atom in a chemical structure as drawn.

The compounds of the invention may exhibit the phenomenon of tautomerism. While Formula I cannot expressly depict all possible tautomeric forms, it is to be understood that Formula I is intended to represent any tautomeric form of the depicted compound and is not to be limited merely to a specific compound form depicted by the formula drawings. For illustration, and in no way limiting the range of tautomers, the compounds of Formula I may exist as the following:

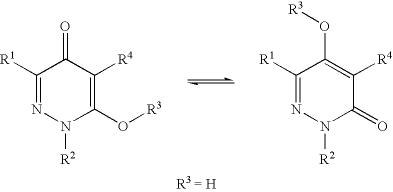

In another example, R⁴ may exist as the following when R⁵ is hydrogen:

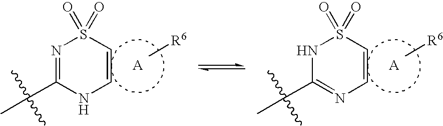

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the Formula I is intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the compounds of Formula I using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the Formula I compounds, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites,” are more polar than the parent drugs, although a polar drug does sometime yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilide is given to a person, the successive metabolites peak and decay in the plasma sequentially. During the first hour, acetanilide is the principal plasma component. In the second hour, as the acetanilide level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

### Methods of Treatment and Prevention of Hepatitis C Viral Infections

The present invention provides methods for treating or preventing a hepatitis C virus infection in a patient in need thereof.

The present invention further provides methods for introducing a therapeutically effective amount of the Formula I compound or combination of such compounds into the blood stream of a patient in the treatment and/or prevention of hepatitis C viral infections.

The magnitude of a prophylactic or therapeutic dose of a Formula I compound of the invention or a pharmaceutically acceptable salt, solvate, or hydrate, thereof in the acute or chronic treatment or prevention of an infection will vary, however, with the nature and severity of the infection, and the route by which the active ingredient is administered. The dose, and in some cases the dose frequency, will also vary according to the infection to be treated, the age, body weight, and response of the individual patient. Suitable dosing regimens can be readily selected by those skilled in the art with due consideration of such factors.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to treatment such as resistance to reverse transcriptase inhibitors, protease inhibitors, etc.

### Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lie preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture; alternatively, the dose of the Formula I compound may be formulated in animal models to achieve a circulating plasma concentration range of the compound that corresponds to the concentration required to achieve a fixed magnitude of response. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are responsive to the effects of the Formula I compounds are exposed to the ligand and the magnitude of response is measured by an appropriate technique. The assessment of the Formula I compound is then evaluated with respect to the Formula I compound potency, and the degree of conversion of the Formula I compound prodrug. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a prodrug of a Formula I compound of the invention or a pharmaceutically acceptable salt, solvate, or hydrate thereof in the acute or chronic treatment or prevention of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. Also, the dose may differ for various particular Formula I compounds; suitable doses can be predicted on the basis of the aforementioned in vitro measurements and on the basis of animal studies, such that smaller doses will be suitable for those Formula I compounds that show effectiveness at lower concentrations than other Formula I compounds when measured in the systems described or referenced herein. In general, the dose per day is in the range of from about 0.001 to 100 mg/kg, preferably about 1 to 25 mg/kg, more preferably about 5 to 15 mg/kg. For treatment of humans infected by hepatitis C viruses, about 0.1 mg to about 15 g per day is administered in about one to four divisions a day, preferably 100 mg to 12 g per day, more preferably from 100 mg to 8000 mg per day.

Additionally, the recommended daily dose ran can be administered in cycles as single agents or in combination with other therapeutic agents. In one embodiment, the daily dose is administered in a single dose or in equally divided doses. In a related embodiment, the recommended daily dose can be administered once time per week, two times per week, three times per week, four times per week or five times per week.

In one embodiment, the compounds of the invention are administered to provide systemic distribution of the compound within the patient. In a related embodiment, the compounds of the invention are administered to produce a systemic effect in the body.

In another embodiment the compounds of the invention are administered via oral, mucosal (including sublingual, buccal, rectal, nasal, or vaginal), parenteral (including subcutaneous, intramuscular, bolus injection, intraarterial, or intravenous), transdermal, or topical administration. In a specific embodiment the compounds of the invention are administered via mucosal (including sublingual, buccal, rectal, nasal, or vaginal), parenteral (including subcutaneous, intramuscular, bolus injection, intraarterial, or intravenous), transdermal, or topical administration. In a further specific embodiment, the compounds of the invention are administered via oral administration. In a further specific embodiment, the compounds of the invention are not administered via oral administration.

Different therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

### Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, α-interferons, β-interferons, ribavirin, alkylating agents, hormones, cytokines, or toll receptor-like modulators. In one embodiment the invention encompasses the administration of an additional therapeutic agent that is HCV specific or demonstrates anti-HCV activity.

The Formula I compounds of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The Formula I compounds of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The Formula I compounds of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The Formula I compounds of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofulvin.

The Formula I compounds of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The Formula I compounds of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, levovirin, viramidine and ribavirin, as well as foscamet, amantadine, rimantadine, saquinavir, indinavir, amprenavir, lopinavir, ritonavir, the α-interferons; β-interferons; adefovir, clevadine, entecavir, pleconaril.

The Formula I compound of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boehringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), anti-B7 antibodies (e.g., IDEC-114 (IDEC)), CTLA4-immunoglobulin, and toll receptor-like (TLR) modulators. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The Formula I compounds of the invention can be administered or formulated in combination with an agent which inhibits viral enzymes, including but not limited to inhibitors of HCV protease, such as BILN 2061 and inhibitors of NS5b polymerase such as NM107 and its prodrug NM283 (Idenix Pharmaceuticals, Inc., Cambridge, Mass.).

The Formula I compounds of the invention can be administered or formulated in combination with an agent which inhibits HCV polymerase such as those described in Wu, Curr Drug Targets Infect Disord. 2003; 3(3):207-19 or in combination with compounds that inhibit the helicase function of the virus such as those described in Bretner M, et al Nucleosides Nucleotides Nucleic Acids. 2003; 22(5-8):1531, or with inhibitors of other HCV specific targets such as those described in Zhang X., IDrugs, 5(2), 154-8 (2002).

The Formula I compounds of the invention can be administered or formulated in combination with an agent which inhibits viral replication.

The Formula I compounds of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-α, and IFN-γ).

The Formula I compounds of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The Formula I compounds of the invention can be administered or formulated in combination with β-interferons which include, but are not limited to, interferon β-1a, interferon β-1b.

The Formula I compounds of the invention can be administered or formulated in combination with α-interferons which include, but are not limited to, interferon α-1, interferon α-2a (roferon), interferon α-2b, intron, Peg-Intron, Pegasys, consensus interferon (infergen) and albuferon.

The Formula I compounds of the invention can be administered or formulated in combination with an absorption enhancer, particularly those which target the lymphatic system, including, but not limited to sodium glycocholate; sodium caprate; N-lauryl-β-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more Formula I compounds of the invention and one or more absorption enhancers.

The Formula I compounds of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other therapeutics agent can act additively or, more preferably, synergistically. In one embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more Formula I compounds of the invention without an additional therapeutic agent.

### Pharmaceutical Compositions and Dosage Forms

Pharmaceutical compositions and single unit dosage forms comprising a Formula I compound of the invention, or a pharmaceutically acceptable salt, or hydrate thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients. Sterile dosage forms are also contemplated.

In an alternative embodiment, pharmaceutical composition encompassed by this embodiment includes a Formula I compound of the invention, or a pharmaceutically acceptable salt, or hydrate thereof, and at least one additional therapeutic agent. Examples of additional therapeutic agents include, but are not limited to, those listed above.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise Formula I compounds of the invention, or a pharmaceutically acceptable salt or hydrate thereof comprise 0.1 mg to 1500 mg per unit to provide doses of about 0.01 to 200 mg/kg per day.

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985).

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The Formula I compounds of the invention may also be administered directly to the lung by inhalation. For administration by inhalation, a Formula I compound can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a Formula I compound directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a Formula I compound to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a Formula I compound to the lung is a liquid spray device supplied, for example, by Aradigm Corporation. Liquid spray systems use extremely small nozzle holes to aerosolize liquid drug formulations that can then be directly inhaled into the lung.

In one embodiment, a nebulizer device is used to deliver a Formula I compound to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In one embodiment, an electrohydrodynamic (“EID”) aerosol device is used to deliver Formula I compounds to the lung. EHD aerosol devices use electrical energy to aerosolize liquid drug solutions or suspensions (see, e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the Formula I compounds formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of Formula I compounds will be known to the skilled artisan and are within the scope of the invention.

Liquid drug formulations suitable for use with nebulizers and liquid spray devices and EHD aerosol devices will typically include a Formula I compound with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of the Formula I compound. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. Nos. 5,112,598; Biesalski, 5,556,611, which are herein incorporated by reference) A Formula I compound can also be formulated in rectal or vaginal compositions such as suppositories or retention enemas, e.g., containing conventional suppository bases such as cocoa butter or other glycerides.

In addition to the formulations described previously, a Formula I compound can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver Formula I compounds. Certain organic solvents such as dimethylsulfoxide can also be employed, although usually at the cost of greater toxicity. A Formula I compound can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989, 25, 351; Howard et al., J. Neurosurg., 71, 105 (1989). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

The invention provides a pharmaceutical pack or kit comprising one or more containers comprising a Formula I compound useful for the treatment or prevention of a Hepatitis C virus infection. In other embodiments, the invention provides a pharmaceutical pack or kit comprising one or more containers comprising a Formula I compound useful for the treatment or prevention of a Hepatitis C virus infection and one or more containers comprising an additional therapeutic agent, including but not limited to those listed above, in particular an antiviral agent, an interferon, an agent which inhibits viral enzymes, or an agent which inhibits viral replication, preferably the additional therapeutic agent is HCV specific or demonstrates anti-HCV activity.

The invention also provides a pharmaceutical pack or kit comprising one or more containers comprising one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The inventive agents may be prepared using the reaction routes and synthesis schemes as described below, employing the general techniques known in the art using starting materials that are readily available. The synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### Preparation of Compounds

In the synthetic schemes described below, unless otherwise indicated all temperatures are set forth in degrees Celsius and all parts and percentages are by weight.

Reagents were purchased from commercial suppliers such as Aldrich Chemical Company or Lancaster Synthesis Ltd. and were used without further purification unless otherwise indicated. All solvents were purchased from commercial suppliers such as Aldrich, EMD Chemicals or Fisher and used as received.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried.

The reactions were assayed by TLC and/or analyzed by LC-MS and terminated as judged by the consumption of starting material. Analytical thin layer chromatography (TLC) was performed on glass-plates precoated with silica gel 60 F₂₅₄ 0.25 mm plates (EME Chemicals), and visualized with UV light (254 nm) and/or iodine on silica gel and/or heating with TLC stains such as ethanolic phosphomolybdic acid, ninhydrin solution, potassium permanganate solution or ceric sulfate solution. Preparative thin layer chromatography (prepTLC) was performed on glass-plates precoated with silica gel 60 F₂₅₄ 0.5 mm plates (20×20 cm, from Thomson Instrument Company) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Column chromatography was completed under positive pressure using Merck silica gel 60, 230-400 mesh or 50-200 mesh neutral alumina, ISCO Flash-chromatography using prepacked RediSep silica gel columns, or Analogix flash column chromatography using prepacked SuperFlash silica gel columns. Hydrogenolysis was done at the pressure indicated in the examples or at ambient pressure.

¹H-NMR spectra and ¹³C-NMR were recorded on a Varian Mercury-VX400 instrument operating at 400 MHz. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), bs (broad singlet), dd (doublet of doublets), dt (doublet of triplets). Coupling constants, when given, are reported in Hertz (Hz).

Infrared (IR) spectra were recorded on an ATR FT-IR Spectrometer as neat oils or solids, and when given are reported in wave numbers (cm⁻¹). Mass spectra reported are (+)-ES or APCI (+) LC/MS conducted by the Analytical Chemistry Department of Anadys Pharmaceuticals, Inc. Elemental analyses were conducted by the Atlantic Microlab, Inc. in Norcross, Ga. Melting points (mp) were determined on an open capillary apparatus, and are uncorrected.

The described synthetic pathways and experimental procedures utilize many common chemical abbreviations, 2,2-DMP (2,2-dimethoxypropane), Ac (acetyl), ACN (acetonitrile), Bn (benzyl), BOC (tert-butoxycarbonyl), Bz (benzoyl), DBU (1,8-diazabicyclo[5,4,0]undec-7-ene, DCC(N,N′-dicyclohexylcarbodiimide), DCE (1,2-dichloroethane), DCM (dichloromethane), DEAD (diethylazodicarboxylate), DIEA (diisopropylethylamine), DMA (N,N-dimethylacetamide), DMAP (4-(N,N-dimethylamino)pyridine), DMF (N,N-dimethylformamide), DMSO (dimethyl sulfoxide), EDC (1-(3-dimethylaminopropyl)-3-ethylcarbodiimide hydrochloride), Et (ethyl), EtOAc (ethyl acetate), EtOH (ethanol), HATU (O-(7-azabenzotriazol-1-yl)-1,1,3,3-tetramethyluronium hexafluorophosphate), HBTU (O-benzotriazol-1-yl-N,N,N′,N′-tetramethyluronium hexafluorophosphate), HF (hydrogen fluoride), HOBT (1-hydroxybenzotriazole hydrate), HPLC (high pressure liquid chromatography), IPA (isopropyl alcohol), KO^{t}Bu (potassium tert-butoxide), LDA (lithium diisopropylamine), MCPBA (3-chloroperbenzoic acid), Me (methyl), MeCN (acetonitrile), MeOH (methanol), NaH (sodium hydride), NaOAc (sodium acetate), NaOEt (sodium ethoxide), Phe (phenylalanine), PPTS (pyridinium p-toluenesulfonate), PS (polymer supported), Py (pyridine), pyBOP (benzotriazol-1-yloxy)tripyrrolidinophosphonium hexafluorophosphate), TEA (triethylamine), TFA (trifluoroacetic acid), TFAA (trifluoroacetic anhydride), THF (tetrahydrofuran), TLC (thin layer chromatography), Tol (toluoyl), Val (valine), and the like.

Methods 1-15 provide general procedures that may be used to prepare compounds of Formula I.

Method 1: Scheme 1 provides a general procedure that was used to prepare compounds of Formula I.

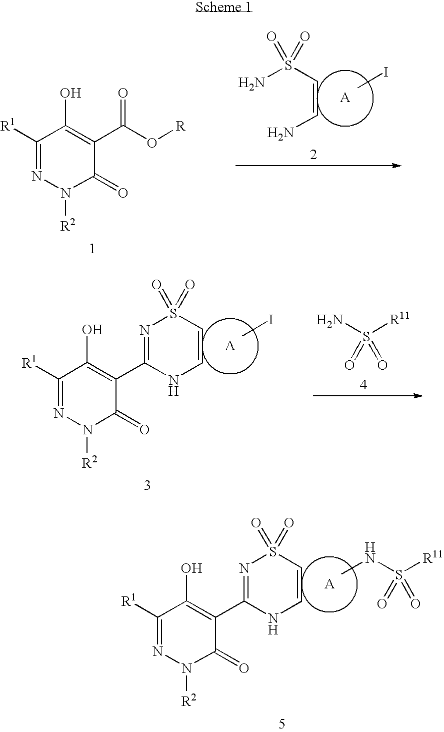

In a typical synthetic route, the preparation of cyclized pyridazinone intermediate 1 was described in WO 2006/066079 (method 4, scheme 4), which is hereby incorporated by reference in its entirety.

The pyridazinone intermediate 1 can be condensed with intermediate 2 with or without solvent under a heated condition to give the iodide compounds 3. The aryl iodide 3 can undergo CuI/amino acid-catalyzed cross-coupling reaction with alkanesulfonamide 4 to give title compound 5. See, e.g. W. Deng et al., Tetrahedron Letters, 46, 7295-7298 (2005).

### EXAMPLE 1-1

Scheme 1a describes the synthesis of compound 5a.

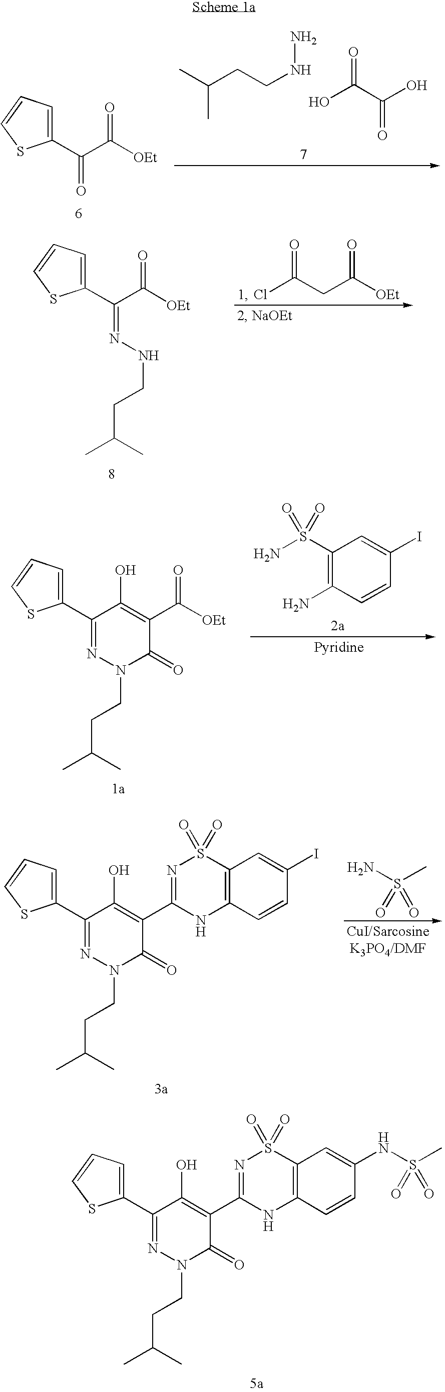

### [(3-Methyl-butyl)-hydrazono]-thiophen-2-yl-acetic acid ethyl ester (8)

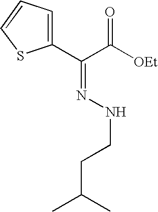

To a solution of oxo-thiophen-2-yl-acetic acid ethyl ester (6) (3.81 g, 20.7 mmol) in absolute ethanol (100 mL), (3-Methyl-butyl)-hydrazine oxalate (7) (3.97 g, 20.7 mmol) was added. The mixture was stirred at 80° C. under N₂ atmosphere for 24 hours. The reaction mixture was concentrated under reduced pressure and the residue was purified by flash chromatography on silica gel to give the desired product (8) (2.08 g, 37%) that was directly used in the next step. LC-MS (ESI⁺): m/e 269.2 [M+1]⁺, 537.4 [2M+1]⁺, 559.0 [2M+Na]⁺ (exact ms: 268.12).

### 5-Hydroxy-2-(3-methyl-butyl)-3-oxo-6-thiophen-2-yl-2,3-dihydro-pyridazine-4-carboxylic acid ethyl ester (1a)

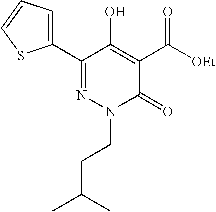

To a solution of [(3-Methyl-butyl)-hydrazono]-thiophen-2-yl-acetic acid ethyl ester (8) (2.08 g, 7.76 mmol) in anhydrous dioxane under N₂ atmosphere, ethyl malonyl chloride (90%, Alfa Aesar) (1.32 mL, 9.31 mmol) was added. The reaction mixture was stirred at 100° C. for 20 min, cooled to rt, diluted with EtOAc and washed with aqueous NaHCO₃ and brine, dried over Na₂SO₄. Solvent was removed under reduced pressure, and the residue was dissolved in EtOH (25 mL) at room temperature, sodium ethoxide solution (Aldrich) (21 wt % in ethanol, 3.50 mL, 9.3 mmol) was added, and the resulting mixture was stirred for 30 min. Aqueous HCl (5%, 6.3 mL) was added to the reaction mixture in a period of 10 min, followed by liquid-liquid extraction with H₂O/EtOAc. The combined organic layer was washed with brine, dried over Na₂SO₄. Solvent was removed under reduced pressure and the residue was purified by flash chromatography on silica gel to give the desired product (1a) (1.67 g, 64%) as yellow solid. ¹H NMR (400 MHz, CDCl₃): δ 7.89 (dd, 1H, J=3.6, 1.2 Hz), 7.39 (dd, 1H, J=5.2, 1.2 Hz), 7.10 (dd, 1H, J=5.2, 3.6 Hz), 4.53 (q, 2H, J=7.2 Hz), 4.22 (m, 2H), 1.73 (m, 2H), 1.68 (m, 1H), 1.50 (t, 3H, J=7.2 Hz), 0.99 (d, 6H, J=6.4 Hz); LC-MS (ESI): m/e=337.30 [M+1]⁺ (exact ms: 336.11).

### 5-Hydroxy-4-(7-iodo-1,1-dioxo-1,4-dihydro-1l6-benzo[1,2,4]thiadiazin-3-yl)-2-(3-methyl-butyl)-6-thiophen-2-yl-2H-pyridazin-3-one (3a)

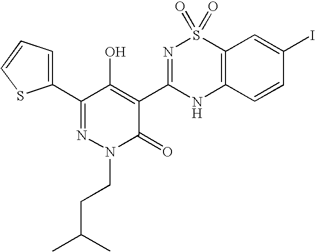

Compound 1a (1.67 g, 4.96 mmol) and intermediate 2a (Scheme 16) (1.48 g, 4.96 mmol) were dissolved in anhydrous pyridine (12 mL) and stirred at 120° C. for 24 hours under N₂ atmosphere. LC-MS indicated completion of the reaction. The reaction mixture was cooled to rt, and concentrated under reduced pressure. The residue was taken in MeOH, and the solid crashed out was collected and further washed with MeOH (6 times) to give pure desired product (3a) (1.21 g, 43%). ¹H NMR (400 MHz, CDCl₃): δ 8.29 (d, 1H, J=1.6 Hz), 7.99 (dd, 1H, J=4.0, 1.2 Hz), 7.94 (dd, 1H, J=8.8, 2.0 Hz), 7.47 (dd, 1H, J=5.2 Hz), 7.15 (dd, 1H, J=5.2, 3.6 Hz), 7.09 (d, 1H, J=8.8 Hz), 4.31 (t, 2H, J=7.2 Hz), 1.78 (m, 2H), 1.70 (m, 1H), 1.03 (d, 6H, J=6.4 Hz); LC-MS (ESI⁺): m/e=570.1 [M]⁺ (exact ms: 569.99).

### N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide (5a)

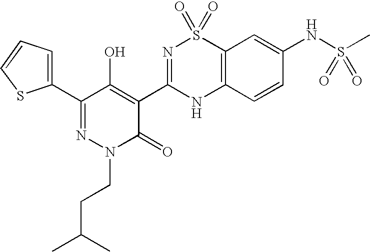

A reaction flask was charged with CuI (20 mg, 0.11 mmol), sarcosine (N-methyl glycine) (23.4 mg, 0.26 mmol), methanesulfonamide (4a) (75 mg, 0.79 mmol), and potassium phosphate (334 mg, 1.58 mmol). The flask was evacuated and back-filled with Nitrogen twice, and then a suspension of intermediate 3a (300 mg, 0.53 mmol) in anhydrous DMF (4 mL) was added under nitrogen atmosphere. The resulting mixture was stirred at 100° C. for 24 hours, cooled to rt, and concentrated under reduced pressure. The crude compound was purified by prep-TLC plate, followed by trituration with methanol twice to give desired product (5a) as yellow solid (90 mg, 32%). ¹H NMR (400 MHz, DMSO-d₆): δ 10.22 (s, 1H), 7.90 (dd, 1H, J=3.6, 1.2 Hz), 7.67 (m, 1H), 7.64 (d, 1H, J=8.8 Hz), 7.60 (d, 1H, J=2.4 Hz), 7.54 (dd, 1H, J=8.8, 2.8 Hz), 7.16 (dd, 1H, J=4.8, 3.6 Hz), 4.15 (t, 2H, J=7.2 Hz), 3.07 (s, 3H), 1.67 (m, 3H), 0.95 (d, 6H, J=6.8 Hz), LC-MS (EST): m/e=538.26 [M+H]⁺ (exact ms: 537.08).

The following compounds of formula I were also made in an analogous manner to the procedure described in Method 1.

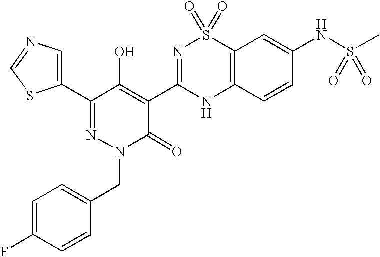

### N-{3-[2-(4-Fluoro-benzyl)-5-hydroxy-3-oxo-6-thiazol-5-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.10 (s, 1H), 7.56 (s, H), 7.48 (m, 2H), 7.40 (t, 2H, J=8.0 Hz), 7.15 (t, 2H, J=8.4 Hz), 5.26 (s, 2H), 3.04 (s, 3H); LC-MS (ESI⁺): m/e=577.1 [M+H]⁺ (exact ms: 576.04).

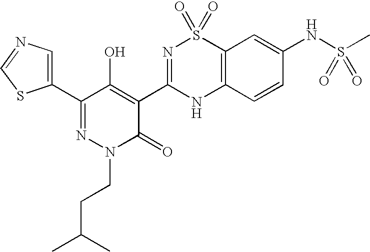

### N-{3-[5-Hydroxy-2-(3-methyl-butyl)-3-oxo-6-thiazol-5-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 9.92 (s, 1H), 8.99 (s, 1H), 8.57 (s, 1H), 7.48 (d, 1H, J=2.4 Hz), 7.41 (dd, 1H, J=8.4, 2.4 Hz), 7.33 (d, 1H, J=9.2 Hz), 4.00 (t, 2H, J=7.2 Hz), 3.00 (s, 3H), 1.59 (m, 3H), 0.93 (d, 6H, J=6.8 Hz); LC-MS (ESI⁺): m/e=539.2 [M+H]⁺ (exact ms: 538.08).

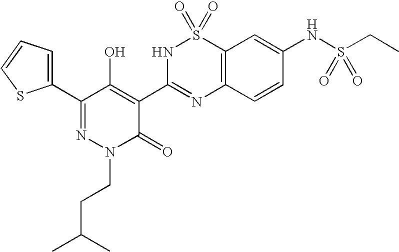

### Ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 7.96 (d, 1H, J=4.0 Hz), 7.69 (dd, 1H, J=9.2, 2.8 Hz), 7.62 (d, 1H, J=2.4 Hz), 7.43 (d, 1H, J=4.8 Hz), 7.29 (d, 1H J=8.8 Hz), 7.11 (dd, 1H, J=4.8, 3.6 Hz), 4.27 (t, 2H, J=7.2 Hz), 3.12 (q, 2H, J=7.6 Hz), 1.74 (m, 2H), 1.67 (m, 1H), 1.37 (t, 3H, J=7.6 Hz), 0.99 (d, 6H, J=6.4 Hz); LC-MS (ESI): m/e 552.2 [M+H]⁺ (exact ms: 551.10).

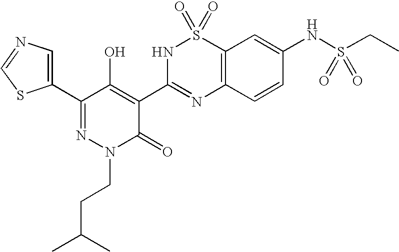

### Ethanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.02 (s, 1H), 9.00 (s, 1H), 8.57 (s, 1H), 7.49 (d, 1H, J=2.0 Hz), 7.43 (dd, 1H, J=9.2, 2.4 Hz), 7.34 (d, 1H, J=8.8 Hz), 4.01 (t, 2H, J=6.8 Hz), 3.10 (q, 2H, J=7.6 Hz), 1.60 (m, 3H), 1.21 (t, 3H, J=7.6 Hz), 0.93 (d, 6H, J=6.4 Hz); LC-MS (ESI⁺): m/e 553.2 [M+H]⁺ (exact ms: 552.10).

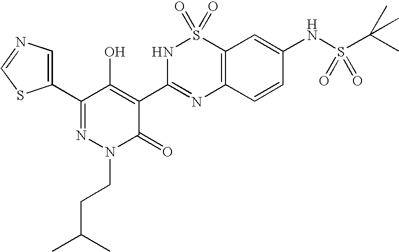

### 2-Methyl-propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 9.89 (s, 1H), 9.01 (s, 1H), 8.58 (s, 1H), 7.56 (d, 1H, J=2.0 Hz), 7.48 (dd, 1H, J=8.8, 2.4 Hz), 7.30 (d, 1H, J=8.8 Hz), 4.01 (t, 2H, J=6.8 Hz), 1.60 (m, 3H), 1.29 (s, 9H), 0.93 (d, 6H, J=6.4 Hz); LC-MS (ESI): m/e 581.2 [M+H]⁺ (exact ms: 580.12).

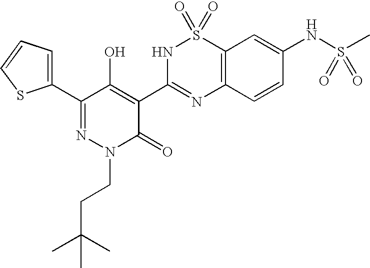

### N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.22 (s, 1H), 7.90 (dd, 1H, J=4.0, 1.2 Hz), 7.67 (m, 1H), 7.64 (d, 1H, J=9.2 Hz), 7.60 (d, 1H, J=2.4 Hz), 7.54 (dd, 1H, J=8.8, 2.0 Hz), 7.16 (dd, 1H, J=5.2, 3.6 Hz), 4.16 (m, 2H), 3.07 (s, 3H), 1.68 (m, 2H), 0.98 (s, 9H); LC-MS (ESI⁺): m/e 552.24 [M+H]⁺ (exact ms: 551.10).

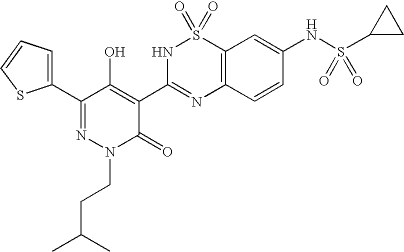

### Cyclopropanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.22 (s, 1H), 7.90 (dd, 1H, J=3.6, 1.2 Hz), 7.67 (m, 1H), 7.63 (m, 2H), 7.56 (dd, 1H, J=8.8, 2.0 Hz), 7.16 (dd, 1H, J=4.8, 3.2 Hz), 4.17 (t, 2H, J=6.8 Hz), 2.70 (m, 1H), 1.67 (m, 3H), 0.96 (m, 10H); LC-MS (ESI⁺): m/e 564.46 [M+H]⁺ (exact ms: 563.10).

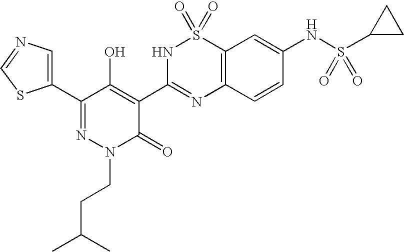

### Cyclopropanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 9.99 (s, 1H), 9.04 (s, 1H), 8.58 (s, 1H), 7.54 (d, 1H, J=2.8 Hz), 7.47 (dd, 1H, J=8.8, 2.4 Hz), 7.39 (d, 1H, J=8.4 Hz), 4.04 (t, 2H, J=6.8 Hz), 2.64 (m, 1H), 1.60 (m, 3H), 0.94 (m, 10H); LC-MS (ESI⁺): m/e 565.1 [M+H]⁺ (exact ms: 564.09).

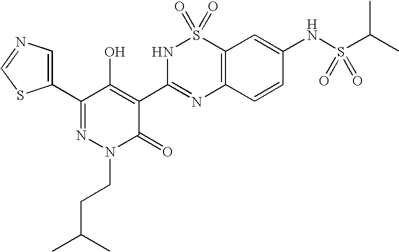

### Propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.21 (s, 1H), 9.16 (s, 1H), 8.60 (s, 1H), 7.59-7.52 (m, 3H), 4.12 (t, 2H, J=6.4 Hz), 3.29 (m, 1H), 1.65 (m, 3H), 1.26 (d, 6H, J=6.4 Hz), 0.94 (t, 6H, J=6.0 Hz); LC-MS (ESI): m/e 567.3 [M+H]⁺ (exact ms: 566.11).

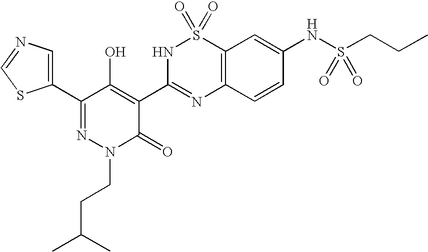

### Propane-1-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.04 (s, 1H), 9.03 (s, 1H), 8.57 (s, 1H), 7.50 (d, 1H, J=2.4 Hz), 7.43 (dd, 1H, J=8.4, 2.4 Hz), 7.38 (d, 1H, J=9.2 Hz), 4.03 (t, 2H, J=6.4 Hz), 3.08 (m, 2H), 1.69 (m, 2H), 1.60 (m, 3H), 0.93 (m, 9H); LC-MS (ESI⁺): m/e 567.3 [M+H]⁺ (exact ms: 566.11).

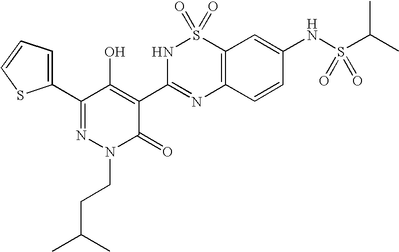

### Propane-2-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.26 (s, 1H), 7.89 (dd, 1H, J=4.0, 1.2 Hz), 7.67 (dd, 1H, J=4.8, 1.2 Hz), 7.62 (m, 2H), 7.55 (dd, 1H, J=8.8, 2.4 Hz), 7.16 (dd, 1H, J=4.8, 3.6 Hz), 4.16 (t, 2H, J=6.8 Hz), 3.30 (m, 1H), 1.66 (m, 3H), 1.26 (d, 6H, J=6.8 Hz), 0.95 (d, 6H, J=6.0 Hz); LC-MS (ESI⁺): m/e 566.2 [M+H]⁺ (exact ms: 565.11).

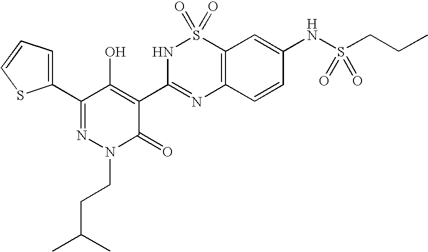

### Propane-1-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆): δ 10.20 (s, 1H), 7.89 (dd, J=4.0, 1.2 Hz, 1H), 7.62 (dd, J=5.2, 0.8 Hz, 1H), 7.56 (d, J=2.4 Hz, 1H), 7.52 (d, J=8.4 Hz, 1H), 7.50 (dd, J=9.2, 2.4 Hz, 1H), 7.13 (dd, J=5.2, 4.0 Hz, 1H), 4.12 (t, J=7.2 Hz, 2H), 3.12 (m, 2H), 1.69 (m, 2H), 1.64 (m, 3H), 0.95 (m, 9H); LC-MS (ESI⁺): m/e 566.2 [M+H]⁺ (exact ms: 565.11).

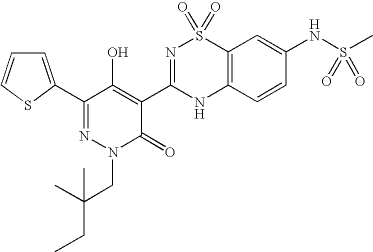

### N-{3-[2-(2,2-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.10 (s, 1H), 7.92 (d, 1H, J=5.6 Hz), 7.65 (d, 1H, J=5.6 Hz), 7.58 (m, 2H), 7.51 (d, 1H J=7.2 Hz), 7.15 (dd, 1H, J=4.8, 3.6 Hz), 4.01 (m, 3H), 3.06 (m, 2H), 1.36 (m, 2H), 0.94 (m, 9H); LC-MS (ESI⁺): m/e 552.30 [M+H]⁺ (exact ms: 551.10).

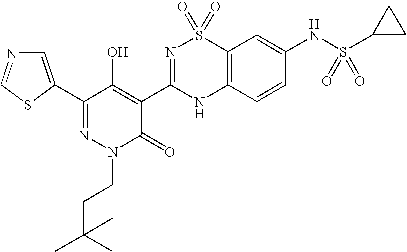

### N-{3-[2-(2,2-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.10 (s, 1H), 9.07 (s, 1H), 8.60 (s, 1H), 7.89 (d, 1H, J=2.4 Hz), 7.80 (dd, 1H, J=4.8, 1.6 Hz), 7.73 (d, 1H, J=9.2 Hz), 4.32 (t, 2H, J=7.2 Hz), 3.32 (m, 2H), 1.82 (m, 2H), 1.34 (m, 1H), 0.98 (m, 13H); LC-MS (ESI): m/e 579.2 [M+H]⁺ (exact ms: 578.11).

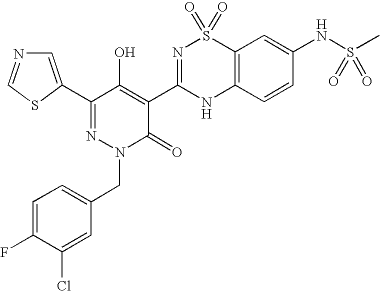

### N-{3-[2-(3-Chloro-4-fluoro-benzyl)-5-hydroxy-3-oxo-6-thiazol-5-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.03 (s, 1H), 7.56 (d, 2H, J=9.6 Hz), 7.45 (dd, 2H, J=8.8, 9.2 Hz), 7.37 (d, 2H, J=8.8 Hz), 5.22 (s, 2H), 3.03 (s, 3H); LC-MS (ESI): m/e 612.0 [M+H]⁺ (exact ms: 610.00).

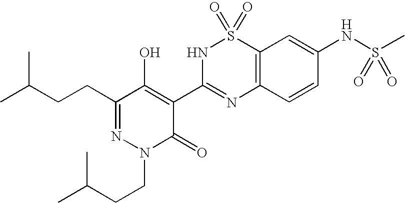

### N-{3-[5-Hydroxy-2,6-bis-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, CDCl₃) δ: 7.72 (s, 1H), 7.69 (d, 1H, J=9.2 Hz), 7.33 (d, 1H, J=8.7 Hz), 7.18 (bs, 1H), 4.20 (t, 2H, J=7.0 Hz), 3.09 (s, 3H), 2.76 (t, 2H, J=7.9 Hz), 1.55-1.73 (m, 7H), 1.27 (s, 1H), 1.00 (dd, 12H, J₁=14.7 Hz, J₂=6.3 Hz); LC-MS (ESI): m/e=526.29 [M+1]⁺ (Exact Mass: 525.17).

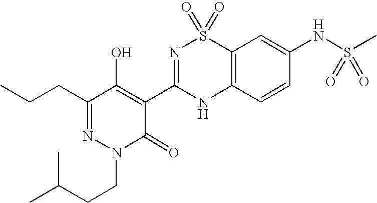

### N-{3-[5-Hydroxy-2-(3-methyl-butyl)-3-oxo-6-propyl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 10.23 (s, 1H), 7.66 (d, 1H, J=8.7 Hz), 7.58 (d, 1H, J=15.7 Hz), 7.55 (dd, 1H, J₁=11.4 Hz, J₂=2.4 Hz), 4.11 (t, 2H, J=7.2 Hz), 3.08 (s, 3H), 2.64 (t, 2H, J=7.4 Hz), 1.60-1.69 (m, 5H), 0.94 (d, 3H, J=9.6 Hz), 0.95 (t, 3H, J=10.2 Hz); LC-MS (ESI): m/e=499.1 [M+2]⁺ (Exact Mass: 497.14).

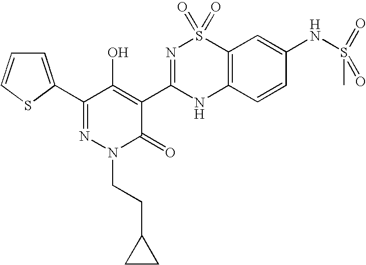

### N-{3-[2-(2-Cyclopropyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 10.23 (s, 1H), 7.91 (d, 1H, J=3.3 Hz), 7.61-7.68 (m, 3H), 7.55 (d, 1H, J=8.4 Hz), 7.16 (t, 1H, J=4.3 Hz), 4.22 (t, 2H, J=7.2 Hz), 3.08 (s, 3H), 1.69 (q, 2H, J=6.8 Hz), 0.71-0.79 (m, 1H), 0.39-0.44 (m, 2H), 0.05 (q, 2H, J=4.9 Hz); LC-MS (ESI⁺): m/e=536.40 [M+1]⁺ (exact mass: 535.07). The product is a yellow solid with 53% yield.

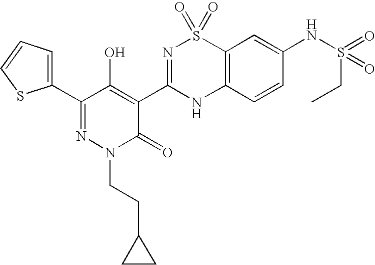

### Ethanenesulfonic acid {3-[2-(2-Cyclopropyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide

¹H NMR (400 MHz, CDCl₃) δ: 7.98 (s, 1H), 7.71 (s, 1H), 7.68 (d, 1H, J=9.2 Hz), 7.45 (d, 1H, J=5.6 Hz), 7.32 (d, 1H, J=8.8 Hz), 7.14 (t, 1H, J=4.6 Hz), 7.04 (s, 1H), 4.37 (t, 2H, J=6.7 Hz), 3.20 (q, 2H, J=7.0 Hz), 1.80 (q, 2H, J=7.0 Hz), 1.43 (t, 3H, J=7.4 Hz), 0.72-0.79 (m, 1H), 0.50 (q, 2H, J=6.0 Hz), 0.10 (q, 2H, J=5.0 Hz); LC-MS (ESI⁺): m/e=550.30 [M+1]⁺ (exact mass: 549.08). The product is a yellow solid with 7% yield.

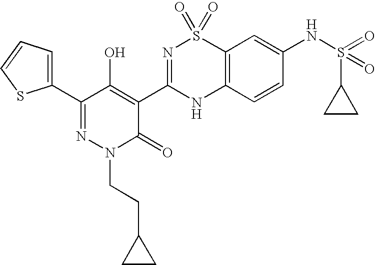

### Cyclopropanesulfonic acid {3-[2-(2-Cyclopropyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide

¹H NMR (400 MHz, DMSO-d₆) δ: 10.13 (s, 1H), 7.90 (bs, 1H), 7.61 (d, 2H, J=5.6 Hz), 7.52 (s, 2H), 7.13 (t, 1H, J=4.2 Hz), 4.17 (t, 2H, J=6.7 Hz), 2.67-2.70 (m, 1H), 1.67 (q, 2H, J=6.8 Hz), 0.95-0.98 (m, 4H), 0.72-0.76 (m, 1H), 0.41 (q, 2H, J=5.9 Hz), 0.04 (q, 2H, J=4.7 Hz); LC-MS (ESI⁺): m/e=562.30 [M+1]⁺ (exact mass: 561.08). The product is a yellow solid with 26% yield.

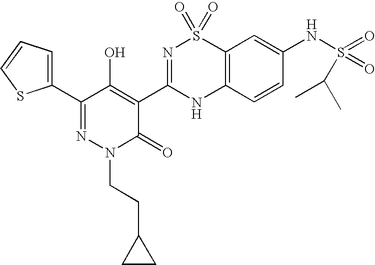

### Propane-2-sulfonic acid {3-[2-(2-cyclopropyl-ethyl)-5-hydroxy-3-oxo-6-thiophen-2-yl-2,3-dihydro-pyridazin-4-yl]-1,1-dioxo-1,4-dihydro-1l6-benzo[1,2,4]thiadiazin-7-yl}-amide

¹H NMR (400 MHz, DMSO-d₆) δ: 10.15 (s, 1H), 7.90 (d, 1H, J=2.3 Hz), 7.60 (d, 1H, J=6.3 Hz), 7.58 (bs, 1H), 7.51 (bs, 2H), 7.12 (t, 1H, J=4.3 Hz), 4.15 (t, 2H, J=7.0 Hz), 3.25-3.32 (m, 1H), 1.66 (q, 2H, J=7.0 Hz), 1.28 (s, 3H), 1.26 (s, 3H), 0.70-0.76 (m, 1H), 0.41 (q, 2H, J=6.0 Hz), 0.04 (q, 2H, J=4.9 Hz)); LC-MS (ES⁺): m/e=564.30 [M+1]⁺ (exact mass: 563.10). The product is a yellow solid with 2% yield.

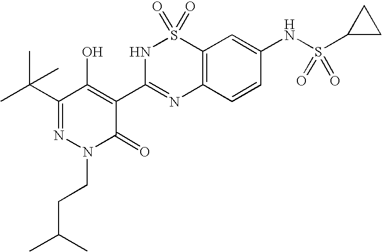

### N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}cyclopropanesulfonamide

Yield: 19% (last step). ¹H NMR (400 MHz, DMSO-d₆): δ 10.26 (s, 1H), 7.66 (m, 2H), 7.56 (m, 1H), 4.13 (t, 2H, J=6.8 Hz), 2.72 (m, 2H), 1.67 (m, 3H), 1.42 (d, 6H, J=6.8 Hz), 0.92 (m, 1H). LC-MS (ESI⁺): m/e=538.3 [M+H]⁺ (exact ms: 537.08).

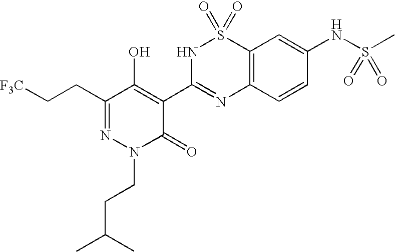

### N-{3-[5-Hydroxy-2-(3-methyl-butyl)-3-oxo-6-(3,3,3-trifluoro-propyl)-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.10 (s, 1H), 7.56 (m, 2H), 7.48 (m, 1H), 4.04 (m, 2H), 3.04 (s, 3H), 2.88 (M, 2H), 2.60 (m, 2H), 1.68 (m, 3H), 0.99 (d, 6H, J=6.8 Hz), LC-MS (ESI): m/e=552.3 [M+H]⁺ (exact ms: 551.11).

In another example, the starting material (5,5,5-Trifluoro-2-oxo-pentanoic acid ethyl ester) was made by the method as shown in Scheme 1b.

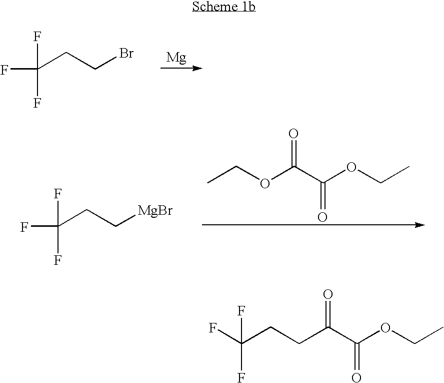

In this specific method, Mg (0.17 g, 6.4 mmol) and small amount of 12 were added into dry THF (5 mL), heated to 65° C. followed by the addition of trifluoropropane bromide (1 g, 5.85 mmol) dropwise. The mixture was cooled to room temperature after 15 minutes and then transferred to a solution of diethyl oxalate (0.75 ml, 6.4 mmol) in 10 mL of toluene at −78° C. After the reaction was complete (based on TLC result), the reaction was quenched by 50 mL of brine and H₂O. The product was extracted out by ethyl acetate. The organic layer was dried over Na₂SO₄. The solvent was removed under reduced pressure and the residue was purified by flash chromatography on silica gel to give the desired α-ketoester (1.2 g) in 98% yield as pale-yellow liquid.

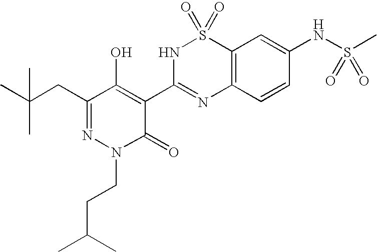

### N-{3-[6-(2,2-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.24 (s, 1H), 7.56 (d, 1H, J=8.8 Hz), 7.60 (d, 1H, J=2.4 Hz), 7.54 (dd, 1H, J=8.8, 6.4 Hz), 4.12 (d, 2H, J=8.4, 7.6 Hz), 3.07 (s, 3H), 2.51 (m, 2H), 1.68 (m, 2H), 0.99 (m, 18H); LC-MS (ESI): m/e=540.5 [M+H]⁺ (exact ms: 539.19).

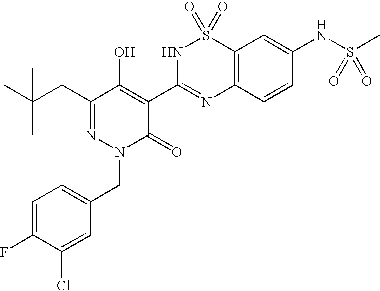

### N-{3-[2-(3-chloro-4-fluoro-benzyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.26 (s, 1H), 7.56 (m, 4H), 7.37 (m, 2H), 5.26 (m, 2H), 3.08 (s, 3H), 1.38 (m, 9H); LC-MS (ESI⁺): m/e=584.5 [M+H]⁺ (exact ms: 583.08).

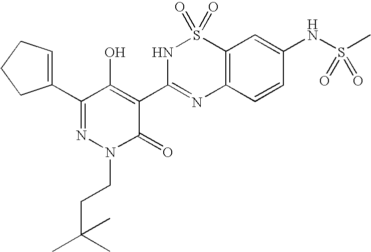

### N-{3-[6-Cyclopent-1-enyl-2-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.26 (s, 1H), 7.70 (d, 1H, J=8.8 Hz), 7.61 (m, 1H), 7.56 (dd, 1H, J=9.2, 3.2 Hz), 6.93 (m, 1H), 4.13 (m, 2H), 3.08 (s, 3H), 2.72 (m, 2H), 2.58 (m, 2H), 1.87 (m, 2H), 1.65 (m, 2H), 0.96 (m, 9H); LC-MS (ESI⁺): m/e=536.3 [M+H]⁺ (exact ms: 535.16).

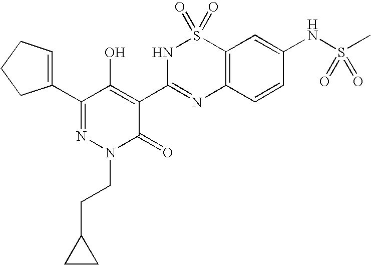

### N-{3-[6-Cyclopent-1-enyl-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆): δ 10.25 (s, 1H), 7.67 (d, 1H, J=8.8 Hz), 7.61 (d, 1H, J=2.4 Hz), 7.56 (dd, 1H, J=8.8, 2.4 Hz), 6.95 (m, 1H), 4.21 (m, 2H), 3.08 (s, 3H), 2.72 (m, 2H), 2.58 (m, 2H), 1.86 (m, 2H), 1.67 (m, 2H), 0.71 (m, 1H), 0.40 (m, 2H), 0.02 (m, 2H); LC-MS (ESI⁺): m/e=520.2 [M+H]⁺ (exact ms: 519.12).

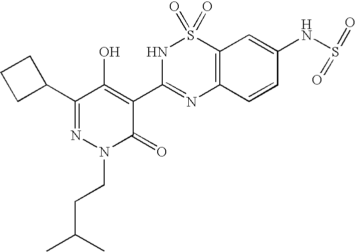

### N-{3-[6-Cyclobutyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, CDCl₃) δ: 1.02 (d, 6H, J=7.1 Hz), 1.68-1.76 (m, 3H), 2.06-2.14 (m, H), 2.29-2.36 (m, H), 3.08 (s, H), 3.75-3.79 (m, 1H), 4.23 (t, 2H, J=7.0 Hz), 6.79 (s, 1H), 7.32 (d, 1H, J=8.3 Hz), 7.64 (dd, 1H, J₁=8.5 Hz, J₂=2.3 Hz), 7.70 (d, 1H, J=2.3 Hz), 13.81 (s, 1H). LC-MS (ESI⁺): m/e 510.5 [M+1]⁺ (exact ms: 509.14).

### EXAMPLE 1-2

Scheme 1c describes a method for preparation of compound 5b of Formula I.

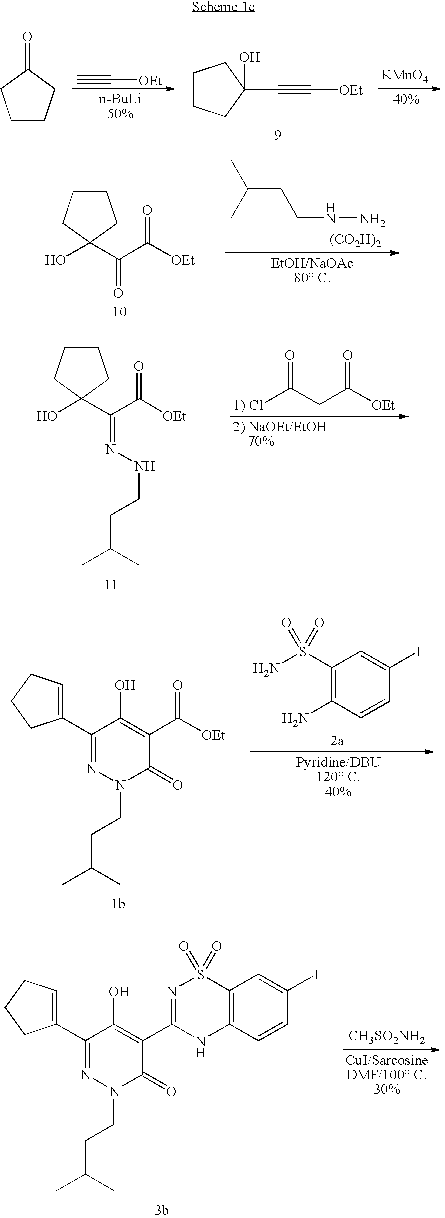

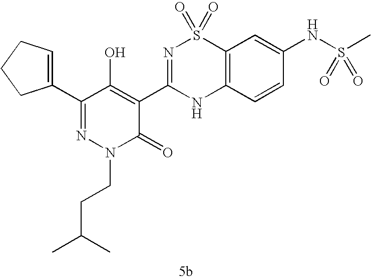

In a typical synthetic route, α-keto-ester 10 was made from cyclopentanone. See J. H. Tatlock, J. Org. Chem. 60, 6221-6223 (1995).

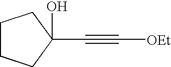

### 1-Ethoxyethynyl-cyclopentanol (9)

To a solution of ethyl ethynyl ether (5 g, mmol, 50 wt % solution in hexanes, 71.3 mmol) in anhydrous THF at −78° C. under N₂, n-BuLi (1.6 M in Hexanes, 44.5 mL, 71.3 mmol) was added slowly over 20 min. The reaction mixture was stirred at −78° C. for 2 h. A solution of cyclopentanone (4.52 mL, 50.9 mmol) in dry THF (50 mL) was added slowly over 15 min. The resulting mixture was stirred for 2 h until TLC showed completion of the reaction. The mixture was poured into aq. NH₄Cl solution, extracted with EtOAc. The combined organic layers were washed brine and dried over Na₂SO₄. The solvents were removed under reduced pressure and the residue was purified by flash chromatography on silica gel to give the desired product 1-Ethoxyethynyl-cyclopentanol (9) (3.90 g, 50%). ¹H NMR (400 MHz, CDCl₃): δ 4.09 (q, 2H, J=7.2 Hz), 1.91 (2H, m), 1.82 (m, 2H), 1.60 (m, 4H), 1.39 (t, 3H, J=7.2 Hz).

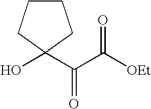

### (1-Hydroxy-cyclopentyl)-oxo-acetic acid ethyl ester (10)

To a solution of 1-Ethoxyethynyl-cyclopentanol (9) (770 mg, 5 mmol) in acetone (50 mL), a solution of NaHCO₃ (252 mg, 3 mmol) and MgSO₄ (1.20 g, 10 mmol) in H₂O (50 mL) was added, followed by KMnO₄ (2.37 g, 15 mmol). The reaction mixture was stirred at rt for 20 min, and then poured into H₂O, extracted with EtOAc (3×). The combined organic layers were washed with H₂O (3×) and brine until colorless, and dried over Na₂SO₄. The solvents were removed under reduced pressure and the residue was purified by flash chromatography on silica gel to give the desired product (1-Hydroxy-cyclopentyl)-oxo-acetic acid ethyl ester (10) (380 mg, 40%). ¹H NMR (400 MHz, CDCl₃): δ 4.37 (q, 2H, J=7.2 Hz), 2.22 (2H, m), 1.95 (m, 2H), 1.85 (m, 4H), 1.40 (t, 3H, J=7.2 Hz).

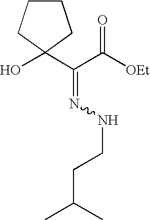

### (1-Hydroxy-cyclopentyl)-[(3-methyl-butyl)-hydrazono]-acetic acid ethyl ester (11)

To a solution of (1-Hydroxy-cyclopentyl)-oxo-acetic acid ethyl ester (10) (560 mg g, 3.0 mmol) in absolute ethanol (25 mL), (3-Methyl-butyl)-hydrazine oxalate (2a) (576 mg, 3.0 mmol) and NaOAc (295 mg, 3.6 mmol) were added. The mixture was stirred at rt under N₂ atmosphere for 3 h. The reaction mixture was diluted with EtOAc, washed with H₂O and brine, dried over Na₂SO₄. The solvents were removed under reduced pressure and the residue was purified by flash chromatography on silica gel to give the desired product (1-Hydroxy-cyclopentyl)-[(3-methyl-butyl)-hydrazono]-acetic acid ethyl ester (11) (405 mg, 50%). LC-MS (ESI⁺): m/e 269.2 [M+1]⁺, 537.4 [2M+1]⁺, 271.30 [2M+Na]⁺ (exact ms: 270.19).

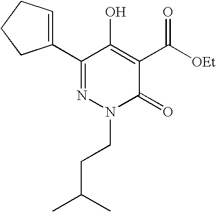

### 6-Cyclopent-1-enyl-5-hydroxy-2-(3-methyl-butyl)-3-oxo-2,3-dihydro-pyridazine-4-carboxylic acid ethyl ester (1b)

To a solution of (1-Hydroxy-cyclopentyl)-[(3-methyl-butyl)-hydrazono]-acetic acid ethyl ester (11) (350 mg, 1.3 mmol) in anhydrous dioxane under N₂ atmosphere, ethyl malonyl chloride (90%, Alfa Aesar) (0.2 mL, 1.56 mmol) was added. The reaction mixture was stirred at 100° C. for 40 minutes, cooled to rt, diluted with EtOAc and washed with aqueous NaHCO₃ and brine, dried over Na₂SO₄. Solvent was removed under reduced pressure, and the residue was dissolved in EtOH (5 mL) at room temperature, sodium ethoxide solution (Aldrich) (21 wt % in ethanol, 0.58 mL, 1.56 mmol) was added, and the resulting mixture was stirred for 20 min. Aqueous HCl (5%, 1.1 mL) was added to the reaction mixture slowly, followed by liquid-liquid extraction with H₂O/EtOAc. The combined organic layer was washed with brine, dried over Na₂SO₄. Solvent was removed under reduced pressure and the residue was purified by flash chromatography on silica gel to give the desired product 6-Cyclopent-1-enyl-5-hydroxy-2-(3-methyl-butyl)-3-oxo-2,3-dihydro-pyridazine-4-carboxylic acid ethyl ester (1b) (290 mg, 70%) as yellow solid. ¹H NMR (400 MHz, CDCl₃): δ 13.75 (s, 1H), 6.88 (m, 1H), 4.52 (q, 2H, J=7.2 Hz), 4.16 (m, 2H), 2.77 (m, 2H), 2.63 (m, 2H), 1.94 (m. 2H), 1.69 (m, 3H), 1.49 (t, 3H, J=7.2 Hz), 0.97 (d, 6H, J=6.0 Hz); LC-MS (ESI⁺): m/e=321.27 [M]⁺ (exact ms: 320.17).

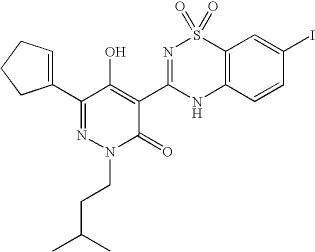

### 6-Cyclopent-1-enyl-5-hydroxy-4-(7-iodo-1,1-dioxo-1,4-dihydro-1λ⁶-benzo[1,2,4]thiadiazin-3-yl)-2-(3-methyl-butyl)-2H-pyridazin-3-one (3b)

6-Cyclopent-1-enyl-5-hydroxy-2-(3-methyl-butyl)-3-oxo-2,3-dihydro-pyridazine-4-carboxylic acid ethyl ester (1b) (370 mg, 1.16 mmol) and 4-Amino-1-iodo-penta-1,3-diene-3-sulfonic acid amide (2a) (378 mg, 1.26 mmol) were dissolved in anhydrous pyridine (5 mL) and stirred at 120° C. for 16 hours under N₂ atmosphere. DBU (0.17 mL, 1.16 mmol) was added and the resulting mixture was stirred at 120° C. for 4 hrs. LC-MS indicated completion of the reaction. The reaction mixture was cooled to rt, and concentrated under reduced pressure. The residue was purified by flash chromatography on silica gel to give the desired product 6-Cyclopent-1-enyl-5-hydroxy-4-(7-iodo-1,1-dioxo-1,4-dihydro-1λ⁶-benzo[1,2,4]thiadiazin-3-yl)-2-(3-methyl-butyl)-2H-pyridazin-3-one (3b) (130 mg, 20.4%) as yellow solid. ¹H NMR (400 MHz, DMSO-d₆): δ 8.12 (d, 1H, J=2.0 Hz), 8.03 (dd, 1H, J=8.8, 2.0 Hz), 7.43 (d, 1H, J=8.4 Hz), 6.94 (m, 1H), 4.12 (t, 2H, J=6.8 Hz), 2.70 (m, 2H), 2.56 (m, 2H), 1.85 (m, 2H), 1.62 (m, 3H), 0.92 (d, 6H, J=6.4 Hz); LC-MS (ESI⁺): m/e=554.97 [M]⁺ (exact ms: 554.05).

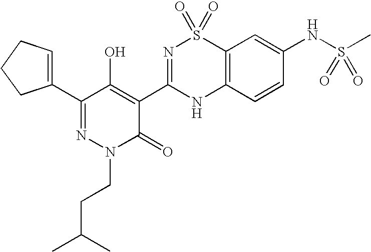

### N-{3-[6-Cyclopent-1-enyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide (5b)

6-Cyclopent-1-enyl-5-hydroxy-4-(7-iodo-1,1-dioxo-1,4-dihydro-1λ⁶-benzo[1,2,4]thiadiazin-3-yl)-2-(3-methyl-butyl)-2H-pyridazin-3-one (3b) (105 mg, 0.19 mmol), CuI (14.5 mg, 0.076 mmol), sarcosine (N-methyl glycine) (10.0 mg, 0.11 mmol), methanesulfonamide (72.2 mg, 0.76 mmol), and potassium phosphate (121.3 mg, 0.57 mmol). Anhydrous DMF (3 mL) was added into the flask. The flask was evacuated and back-filled with Nitrogen twice, and then stirred at 100° C. for 16 hours under nitrogen atmosphere. The reaction mixture was cooled to rt, and concentrated under reduced pressure. The crude compound was purified by prep-HPLC, followed by trituration with methanol twice to give desired product N-{3-[6-Cyclopent-1-enyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide (5b) as yellow solid (29 mg, 30%). ¹H NMR (400 MHz, DMSO-d₆): δ 10.30 (s, 1H), 7.68 (d, 1H, J=8.8 Hz), 7.61 (d, 1H, J=2.4 Hz), 7.55 (dd, 1H, J=8.8, 2.4 Hz), 6.94 (m, 1H), 4.14 (t, 2H, J=6.8 Hz), 3.08 (s, 3H), 2.71 (m, 1H), 2.59 (m, 1H), 1.87 (m, 2H), 1.62 (m, 3H), 0.92 (d, 6H, J=6.8 Hz), LC-MS (ESI⁺): m/e=522.32 [M+H]⁺ (exact ms: 521.14).

### EXAMPLE 1-3

Scheme 1d describes another procedure for making compound 5c of Formula I.

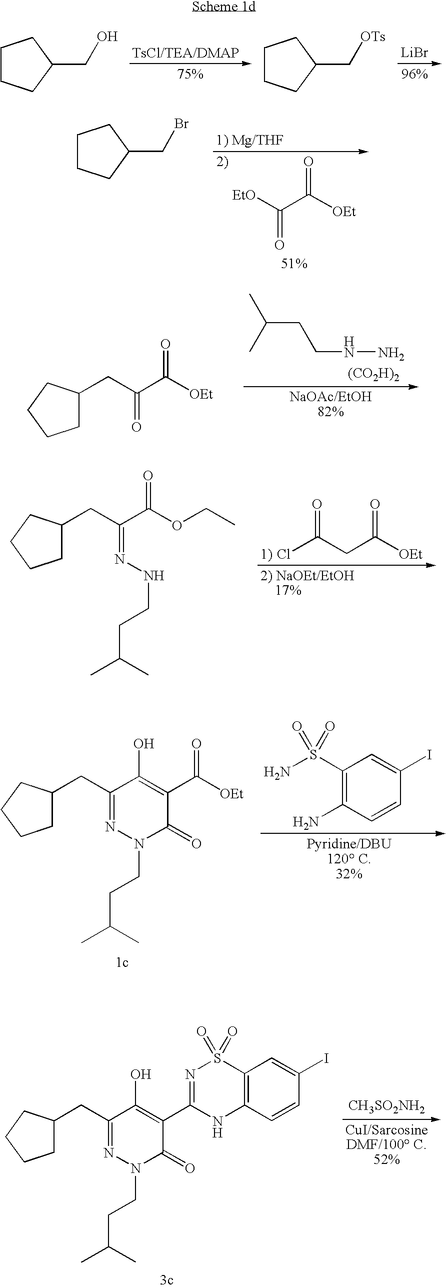

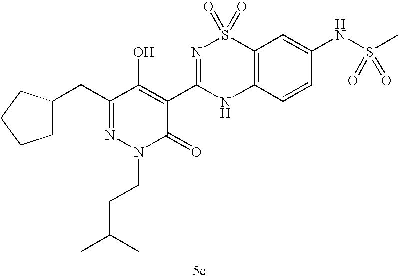

### Toluene-4-sulfonic acid cyclopentylmethyl ester

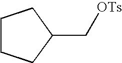

Cyclopentanemethanol (5.0 g, 50 mmol) was dissolved in anhydrous DCM (150 mL), and cooled to 0° C. under N₂ atmosphere. Triethylamine (10.4 mL, 75 mmol) was added, followed by toluenesulfonyl chloride (11.40 g, 60 mmol) in several portions. DMAP (305 mg, 2.5 mmol) was added to the resulting mixture and stirred for 48 h. TLC showed the completion of the reaction. Methanol (10 mL) was added to quench the excess amount of toluenesulfonyl chloride and stirred for 10 min. The mixture was then poured into saturated aq. NH₄Cl solution, and extracted with DCM. The combined organic layers washed with brine and dried over Na₂SO₄. The solid was removed by filter paper and the filtrate was concentrated by rotary evaporator. The crude residue was then purified by flash chromatography on silica gel to give the desired product, toluene-4-sulfonic acid cyclopentylmethyl ester (9.8 g, 77%). ¹H NMR (400 MHz, CDCl₃): δ 7.89 (m, 2H), 7.34 (m, 2H), 3.91 (d, 2H, J=6.8 Hz), 2.47 (s, 3H), 2.22 (m, 1H), 1.74 (m, 2H), 1.56 (m, 4H), 1.21 (m, 2H).

### Bromomethyl-cyclopentane

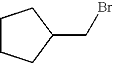

Toluene-4-sulfonic acid cyclopentylmethyl ester (9.8 g, 38.6 mmol) was dissolved in dry acetone (dried over K₂CO₃ overnight), and LiBr (5.03 g, 57.9 mmol) was added to the solution. The resulting mixture was stirred at 65° C. for 36 h, and ¹H NMR showed completion of the reaction. The suspension was filtered through center funnel and concentrated by rotary evaporator (temperature of water bath was less then 10° C.). The residue was dissolved in Et₂O and washed with H₂O and brine, dried over Na₂SO₄. The solid was removed by filter paper and the filtrate was concentrated by rotary evaporator (temperature of water bath was less then 10° C.) to give the desired product, bromomethyl-cyclopentane (6.0 g, 96%). ¹H NMR (400 MHz, CDCl₃): δ 3.41 (d, 2H, J=6.8 Hz), 2.32 (m, 1H), 1.88 (m, 2H), 1.68 (m, 2H), 1.61 (m, 2H), 1.30 (m, 2H).

### 3-Cyclopentyl-2-oxo-propionic acid ethyl ester

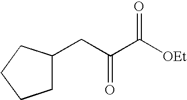

Bromomethyl-cyclopentane (4.9 g, 30 mmol) was dissolved in anhydrous THF (30 mL), Mg (800 mg, 33 mmol) and small amount of 12 solid were added to the solution. The mixture was heated to reflux until the brown color disappeared, and stirring was continued for 30 min. The mixture was cooled to room temperature and used to the next step directly.

Diethyl oxalate (3.54 mL, 26 mmol) was dissolved in 150 mL of dry toluene, and cooled to −78° C. under N₂ atmosphere. The Grignard reagent made above was added slowly into the solution via syringe over a period of 15 min. The reaction mixture was stirred at −78° C. for 1 h, quenched with aq. NH₄Cl solution. The two layers were separated by separatory funnel, and the organic layer was extracted with EtOAc. The combined organic layers washed with brine and dried over Na₂SO₄. The solid was removed by filter paper and the filtrate was concentrated by rotary evaporator (temperature of water bath was less then 20° C.). The crude residue was then purified by flash chromatography on silica gel to give the desired product, 3-Cyclopentyl-2-oxo-propionic acid ethyl ester (3.5 g, 51%). ¹H NMR (400 MHz, CDCl₃): δ 4.33 (q, 2H, J=7.2 Hz), 2.87 (d, 2H, J=7.2 Hz), 2.30 (m, 1H), 1.88 (m, 2H), 1.65 (m, 2H), 1.58 (m, 2H), 1.40 (t, 3H, J=7.2 Hz), 1.15 (m, 2H).

Once the above α-keto-ester was made, the rest of the synthetic procedure for making 1c, 3c and 5c was substantially similar as that described in Scheme 1a.

### N-{3-[6-Cyclopentylmethyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide (5c)

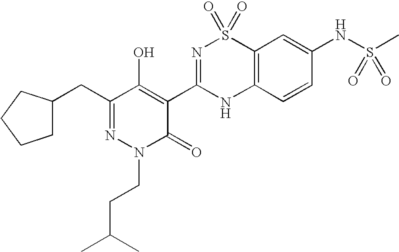

¹H NMR (400 MHz, DMSO-d₆): δ 10.25 (s, 1H), 7.67 (d, 1H, J=8.8 Hz), 7.60 (d, 1H, J=2.8 Hz), 7.55 (dd, 1H, J=9.2, 2.8 Hz), 4.12 (t, 2H, J=7.2 Hz), 3.08 (s, 3H), 2.66 (d, 2H, J=7.2 Hz), 2.26 (m, 1H), 1.78-1.46 (m, 9H), 1.21 (m, 2H), 0.92 (d, 6H, 6.0 Hz); LC-MS (ESI): m/e=538.3 [M+1]⁺ (exact ms: 537.17).

The below compounds were synthesized in a manner similar to that described in scheme 1d.

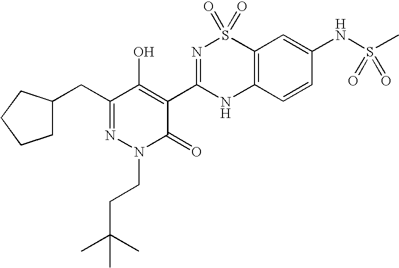

### N-{3-[6-Cyclopentylmethyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.25 (s, 1H), 7.68 (d, 1H, J=8.4 Hz), 7.60 (d, 1H, J=2.4 Hz), 7.54 (dd, 1H, J=9.2, 2.4 Hz), 4.12 (m, 2H), 3.08 (s, 3H), 2.66 (d, 2H, J=7.2 Hz), 2.25 (m, 1H), 1.72 (m, 2H), 1.61 (m, 4H), 1.50 (m, 2H), 1.22 (m, 2H), 0.95 (s, 9H); LC-MS (ESI⁺): m/e=552.5 [M+1]⁺ (exact ms: 551.19).

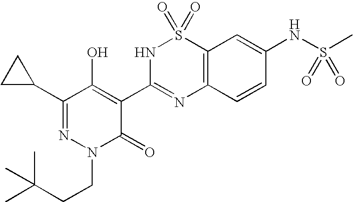

### N-{3-[6-Cyclopropyl-2-(3,3-dimethyl-butyl)-5-hydroxy-3-oxo-2,3-dihydro-pyridazin-4-yl]-1,1-dioxo-1,2-dihydro-1l6-benzo[1,2,4]thiadiazin-7-yl}-methanesulfonamide

The cyclopropanylmaganesiumbromide is commercially available. The rest of the synthesis is substantially similar to that provided in Scheme 1d.

¹H NMR (400 MHz, CDCl₃) δ: 1.01 (s, 9H), 1.01-1.07 (m, 2H), 1.27 (s, 2H), 1.63-1.67 (m, 2H), 2.28-2.35 (m, 1H), 3.09 (s, 3H), 4.13-4.17 (m, 2H), 6.60 (s, 1H), 7.34 (d, 1H, J=8.5 Hz), 7.65 (dd, 1H, J=8.5 Hz, J₂=5.3 Hz), 7.70 (d, 1H, J=2.4 Hz). LC-MS (ESI⁺): m/e=510.4 [M+1]⁺ (Exact Mass: 509.14).

### EXAMPLE 1-4

Scheme 1e describes the synthesis of compound 5d of Formula I.

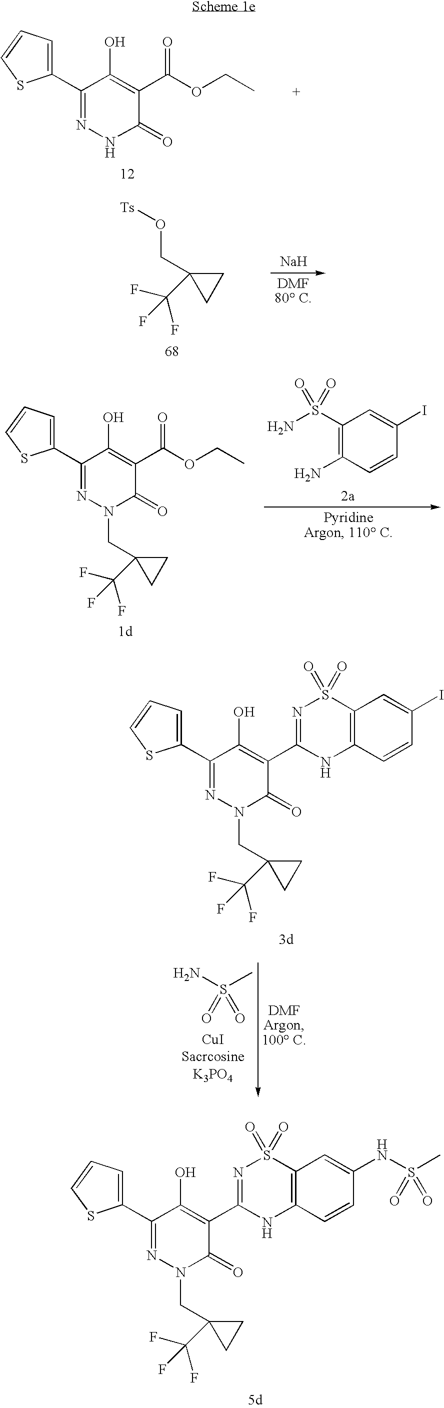

### 5-Hydroxy-3-oxo-6-thiophen-2-yl-2-(1-trifluoromethyl-cyclopropylmethyl)-2,3-dihydro-pyridazine-4-carboxylic acid ethyl ester (1d)

5-hydroxy-3-oxo-6-thiophen-2-yl-2,3-dihydro-pyridazine-4-carboxylic acid ethyl ester (12, see Schemes 1g and 1h) (0.5 g, 1.88 mmol) was suspended in anhydrous DMF (9.4 mL). A 60% suspension of NaH in mineral oil (0.166 g, 4.14 mmol) was added. The mixture stirred in a sealed vial for 10 minutes with occasional venting. Toluene-4-sulfonic acid 1-trifluoromethyl-cyclopropylmethyl ester (68, see Scheme 13e) (0.609 g, 2.07 mmol) was added and the mixture stirred at 80° C. for 7 hours. Upon cooling, the mixture was diluted with EtOAc (300 mL), washed with 1M HCl (2×100 mL), water (50 mL), brine (50 mL), dried over MgSO₄ filtered and concentrated in vacuo. Purification by flash column chromatography (2% MeOH in CH₂Cl₂, Merck silica gel 60, 40-63 μm) afforded the desired product, 5-hydroxy-3-oxo-6-thiophen-2-yl-2-(1-trifluoromethyl-cyclopropylmethyl)-2,3-dihydro-pyridazine-4-carboxylic acid ethyl ester (1d) (0.252 g, 0.649 mmol, 35% yield), as a yellow, waxy solid. ¹H NMR (400 MHz, CDCl₃) δ: 1.07-1.14 (m, 4H), 1.49 (t, 3H, J=7.0 Hz), 4.43 (s, 2H), 4.54 (quartet, 2H, J=7.3 Hz), 7.11 (dd, 1H, J=5.3 Hz, J₂=3.5 Hz), 7.41 (d, 1H, J=5.5 Hz), 7.91 (d, 1H, J=3.8 Hz). LC-MS (ESI⁺): m/e=389.23 [M+H⁺] (100%) (exact mass: 388.07).

### 5-Hydroxy-4-(7-iodo-1,1-dioxo-1,4-dihydro-1λ⁶-benzo[1,2,4]thiadiazin-3-yl)-6-thiophen-2-yl-2-(1-trifluoromethyl-cyclopropylmethyl)-2H-pyridazin-3-one (3d)

5-Hydroxy-3-oxo-6-thiophen-2-yl-2-(1-trifluoromethyl-cyclopropylmethyl)-2,3-dihydro-pyridazine-4-carboxylic acid ethyl ester (1d) (0.2 g, 0.515 mmol) and 2-amino-5-iodo-benzenesulfonamide (0.168 g, 0.566 mmol) were combined and anhydrous pyridine (3.25 mL) was added. The solution was degassed while stirring under vacuum and the flask charged with argon. The mixture stirred at 110° C. for 16 h. Upon cooling, the solution was concentrated in vacuo to a thick brown oil. Addition of MeOH (5 mL) caused the desired product to precipitate. Collection by filtration and rinsing with MeOH (5 mL) followed by drying in vacuo for 16 h afforded the desired product, 5-hydroxy-4-(7-iodo-1,1-dioxo-1,4-dihydro-1λ⁶-benzo[1,2,4]thiadiazin-3-yl)-6-thiophen-2-yl-2-(1-trifluoromethyl-cyclopropylmethyl)-2H-pyridazin-3-one (3d) (0.16 g, 0.257 mmol, 50% yield), as a beige/yellow powder. LC-MS (ESI⁺): m/e=623.08 [M+H⁺] (100%) (exact mass: 621.95).

### N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,4-dihydro-1λ⁶-benzo[1,2,4]thiadiazin-7-yl}-methanesulfonamide (5d)

5-Hydroxy-4-(7-iodo-1,1-dioxo-1,4-dihydro-1λ⁶-benzo[1,2,4]thiadiazin-3-yl)-6-thiophen-2-yl-2-(1-trifluoromethyl-cyclopropylmethyl)-2H-pyridazin-3-one (3d) (0.1 g, 0.161 mmol), potassium triphosphate (0.171 g, 0.803 mmol), sarcosine (0.009 g, 0.096 mmol), and copper (1) iodide (0.008 g, 0.04 mmol) were combined. Anhydrous DMF (1 mL) was added followed by N-methyl-methanesulfonamide (0.152 g, 1.61 mmol). The solution was degassed while stirring under vacuum and the flask charged with argon. The mixture stirred at 100° C. for 4 h. Upon cooling, the mixture was diluted with EtOAc (50 mL), washed with 1M HCl (15 mL), brine (15 mL), dried over MgSO₄ and concentrated in vacuo. Purification by reverse phase HPLC (50%-100% acetonitrile in water, 20 min), followed by trituration from a minimal amount of methanol followed by filtration afforded the desired product, N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,4-dihydro-1λ⁶-benzo[1,2,4]thiadiazin-7-yl}-methanesulfonamide (5d) (0.0156 g, 0.0265 mmol, 17% yield), as a yellow powder. ¹H NMR (400 MHz, DMSO-d₆) δ: 1.02-1.08 (m, 2H), 1.17-1.19 (m, 2H), 3.05 (s, 3H), 4.37 (s, 2H), 7.14 (dd, 1H, J₁=5.4 Hz, J₂=3.9 Hz), 7.50 (s, 2H), 7.56 (s, 1H), 7.62 (d, 1H, J=3.8 Hz), 7.91 (d, 1H, J=4.7 Hz), 10.10 (s, 1H). LC-MS (ESI⁺): m/e=590.46 [M+W] (100%) (exact mass: 589.04).

The following compounds of Formula I were also made in an analogous manner to the procedure described in Scheme 1e.

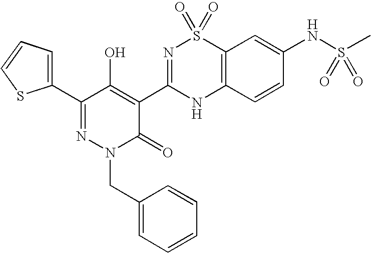

### N-[3-(2-Benzyl-5-hydroxy-3-oxo-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 3.06 (s, 3H), 5.30 (s, 2H), 7.14 (dd, 1H, J₁=5.0 Hz, J₂=3.5 Hz), 7.25-7.37 (m, 5H), 7.51 (dd, 1H, J₁=9.3 Hz, J₂=2.3 Hz), 7.55 (s, 1H), 7.57-7.58 (m, 1H), 7.65 (d, 1H, J=6.3 Hz), 7.90 (dd, 1H, J₁=3.1 Hz, J₂=0.8 Hz), 10.15 (s, 1H). LC-MS (ESI): m/e=558.06 [M+H⁺] (100%) (exact mass: 557.05).

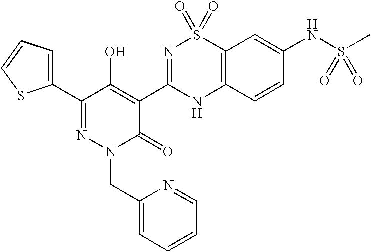

### N-[3-(5-Hydroxy-3-oxo-2-pyridin-2-ylmethyl-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 3.04 (s, 3H), 5.44 (s, 2H), 7.12 (dd, 1H, J=5.5 Hz, J₂=3.9 Hz), 7.47-7.50 (m, 4H), 7.55 (s, 1H), 7.61 (d, 1H, J=3.8 Hz), 7.89 (d, 1H, J=3.9 Hz), 7.99 (t, 1H, J=7.5 Hz), 8.60 (d, 1H, J=4.7 Hz), 10.09 (s, 1H). LC-MS (ESI⁺): m/e=559.13 [M+H⁺] (100%) (exact mass: 558.04).

### EXAMPLE 1-5

Scheme 1f describes the procedure for making compound 5e of Formula I.

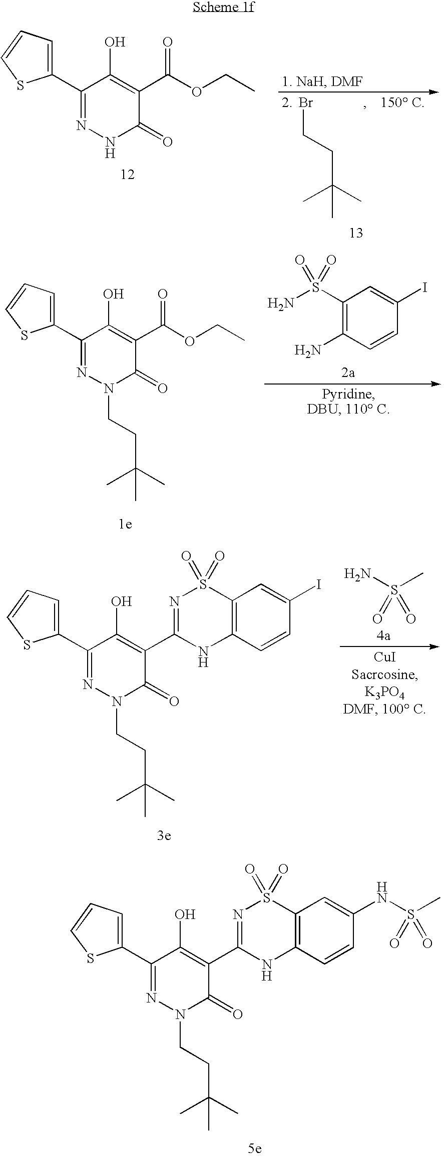

### 2-(3,3-Dimethyl-butyl)-5-hydroxy-3-oxo-6-thiophen-2-yl-2,3-dihydro-pyridazine-4-carboxylic acid ethyl ester (1e)

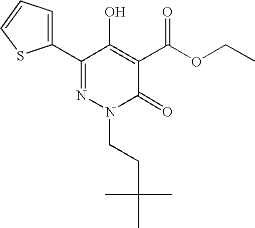

5-hydroxy-3-oxo-6-thiophen-2-yl-2,3-dihydro-pyridazine-4-carboxylic acid ethyl ester (12) (1.75 g, 6.57 mmol) was suspended in dimethylformamide (32 mL) followed by addition of sodium hydride (578 mg, 14.4 mmol) at 25° C. Mixture was allowed to stir for 20 min after which time 1-bromo-3,3-dimethyl-butane (13) was added and mixture heated to 50° C. for 3 h. Cooled mixture was diluted with ethyl acetate (250 mL) and washed twice with 1N HCl (2×250 mL). The organic layer was then concentrated to dryness under reduced pressure to afford 2-(3,3-dimethyl-butyl)-5-hydroxy-3-oxo-6-thiophen-2-yl-2,3-dihydro-pyridazine-4-carboxylic acid ethyl ester (1e) (2.27 g, 98%) as a solid.

### 2-(3,3-Dimethyl-butyl)-5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (3 e)

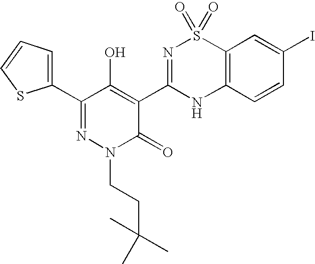

To a solution of 2-(3,3-dimethyl-butyl)-5-hydroxy-3-oxo-6-thiophen-2-yl-2,3-dihydro-pyridazine-4-carboxylic acid ethyl ester (1e) (2.2 g, 6.0 mmol) in pyridine (30 mL), 2-amino-5-iodo-benzenesulfonamide (2a) (1.96 g, 6.6 mmol) was added and the mixture was heated to 110° C. for 23 h. At this time, DBU (0.9 mL, 6.0 mmol) was added to the mixture and stirring continued for another 2 h. Cooled mixture was concentrated to a thick slurry under reduced pressure, dissolved in methanol (40 mL) and then washed in a sepparatory funnel with 1N HCl (40 mL), resulting in precipitation of a solid. Product was filtered, resuspended in methanol (12 mL) and slurried for 2 h before filtering. Isolated cake was dried under high vacuum to afford 2-(3,3-Dimethyl-butyl)-5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (3e) (1.5 g, 43%) as a solid.

### N-{3-[2-(3,3-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide (5e)

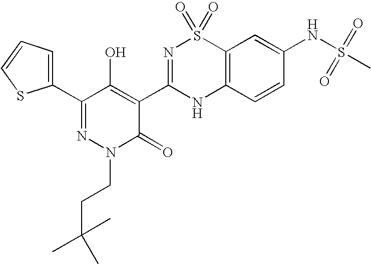

2-(3,3-Dimethyl-butyl)-5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (3e) (1.53 g, 2.61 mmol) was charged into the reaction vessel along with methane sulfonamide (2.48 g, 26.1 mmol), potassium phosphate (2.78 g, 13.0 mmol), copper iodide (0.124 g, 0.65 mmol), sarcosine (0.14 g, 1.5 mmol), followed by addition of DMF (26 mL). Resulting mixture was vacuum purged and back-filled with nitrogen gas three times and then heated to 100° C. for 19 h. Cooled mixture was dissolved in ethyl acetate (400 mL) and washed in a separatory funnel twice with 1N HCl (2×400 mL). The resulting organic layer was concentrated under reduced pressure to a volume of approximately 100 mL to give a suspension of solid, which was then filtered to provide desired product. Solids were then slurried in methanol (100 mL) for an additional 2 h before filtering and drying under high vacuum to afford N-{3-[2-(3,3-Dimethyl-butyl)-5-hydroxy-3-oxo-6-thiophen-2-yl-2,3-dihydro-pyridazin-4-yl]-1,1-dioxo-1,4-dihydro-1l6-benzo[1,2,4]thiadiazin-7-yl}-methanesulfonamide (5e) (970 mg, 67%). ¹H NMR (400 MHz, DMSO-d₆) δ: 0.98 (s, 9H), 1.66-1.70 (m, 2H), 3.07 (s, 3H), 4.13-4.18 (m, 2H), 7.15-7.17 (m, 1H), 7.54 (dd, 1H, J=8.8 Hz, J₂=2.4 Hz), 7.60 (d, 1H, J=2.4 Hz), 7.63 (d, 1H, J=8.8 Hz), 7.66-7.67 (m, 1H), 7.90 (d, 1H, J=3.7 Hz), 10.20 (s, 1H), ), LC-MS (ESI⁺): m/e=552.2 [M+1]⁺ (exact mass: 551.10).

Compound 5e can be coverted to the corresponding sodium salt as N-{3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide, sodium salt (102).

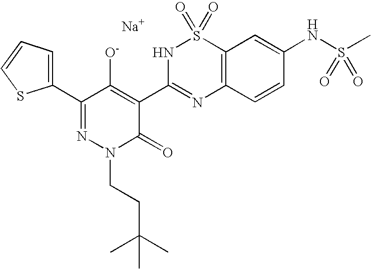

N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide (5e) (250 mg, 0.453 mmol) was dissolved in the minimum amount of 1,4-dioxane at 73° C. Once a clear solution was obtained, a 1.0 M solution of sodium ethoxide in ethanol (498 μL, 0.498 mmol) was added dropwise. The reaction mixture was stirred at 73° C. for 5 minutes and then was concentrated in vacuo. The residue was treated with diethyl ether, sonicated, and concentrated and dried in vacuo at 60° C. for 16 h to afford the desired product, N-{3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide, sodium salt (102) (230 mg, 0.401 mmol, 88.6% yield), as a solid. ¹H NMR (400 MHz, DMSO-d₆) δ: 0.96 (9H, s), 1.59-1.63 (2H, m), 2.95 (3H, s), 3.98-4.02 (2H, m), 7.05-7.07 (1H, m), 7.26-7.28 (1H, m), 7.35-7.38 (1H, m), 7.43-7.45 (1H, m), 7.46-7.48 (1H, m), 7.47 (1H, d, J=5.6 Hz), 7.87 (1H, d, J=3.8 Hz), 9.92 (1H, bs). LC-MS (ESI⁺): m/e=552.3 [M+1]⁺ (exact mass for neutral compound: 551.10).

The compound 12 was made in two different methods as shown in Scheme 1g and Scheme 1h.

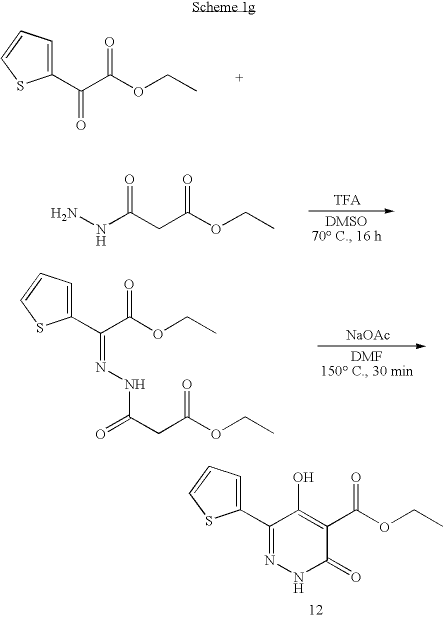

### [(2-Ethoxycarbonyl-acetyl)-hydrazono]-thiophen-2-yl-acetic acid ethyl ester

Oxo-thiophen-2-yl-acetic acid ethyl ester (2 g, 10.86 mmol) was dissolved in anhydrous DMSO (54.3 mL). Hydrazinocarbonyl-acetic acid ethyl ester (1.75 g, 11.95 mmol) was added followed by TFA (0.2 mL). The flask was evacuated and filled with N₂. The mixture was heated at 70° C. for 16 h. Upon cooling to 25° C., the mixture was diluted with EtOAc and washed with 0.1 M HCl (3 times). The organic phase was further washed with brine, dried over MgSO₄ and concentrated in vacuo. Purification of the residue by flash column chromatography (10-15% EtOAc/Hexanes) afforded ([(2-ethoxycarbonyl-acetyl)-hydrazono]-thiophen-2-yl-acetic acid ethyl ester (2.89 g, 9.25 mmol, 85% yield) as a faintly yellow oil that crystallized to a beige, waxy, solid upon standing. ¹H NMR (400 MHz, CDCl₃, 10:1 mixture of isomers observed, data for major isomer reported): δ=1.28 (t, 3H, J=6.9 Hz), 1.47 (t, 3H, J=7.1 Hz), 3.80 (s, 2H), 4.22 (q, 2H, J=7.1 Hz), 4.47 (q, 2H, J=7.1 Hz), 7.03 (t, 1H, J=4.2 Hz), 7.33 (d, 1H, J=4.4 Hz), 7.60 (d, 1H, J=3.7 Hz), 11.80 (br s, 1H). ¹³C NMR (100 MHz, CDCl₃): δ=14.2, 14.3, 40.7, 61.5, 62.9, 127.5, 128.1, 129.0, 130.9, 138.2, 160.5, 166.9, 168.5. LC-MS (ESI): m/e=313.1 [M+H⁺] (100%).

### 5-Hydroxy-3-oxo-6-thiophen-2-yl-2,3-dihydro-pyridazine-4-carboxylic acid ethyl ester (12)

[(2-Ethoxycarbonyl-acetyl)-hydrazono]-thiophen-2-yl-acetic acid ethyl ester (1 g, 3.2 mmol) was dissolved in DMF (16 mL) and NaOAc (0.525 g, 2.55 mmol) was added. The flask was evacuated and filled with N₂. The mixture was heated at 150° C. for 30 min. Upon cooling to 25° C., 1 M HCl (32 mL) was added and the product precipitated. After stirring for 5 min, the solid was collected by filtration, washed with 1 M HCl and dried in vacuo for 16 h to afford 5-hydroxy-3-oxo-6-thiophen-2-yl-2,3-dihydro-pyridazine-4-carboxylic acid ethyl ester (12) as a light beige powder (0.68 g, 2.55 mmol, 80% yield). ¹H NMR (400 MHz, DMSO-d₆): δ=1.29 (t, 3H, J=7.3 Hz), 4.30 (q, 2H, J=7.3 Hz), 7.12 (dd, 1H, J=5.4, 3.8 Hz), 7.62 (d, 1H, J=3.8 Hz), 7.80 (d, 1H, J=4.6 Hz), 13.00 (br s, 1H). ¹³C NMR (100 MHz, DMSO-d₆): δ=14.0, 61.6, 107.6, 127.5, 127.8, 127.8, 135.7, 137.1, 158.3, 158.5, 166.2. LC-MS (ESI⁺): m/z=267.1 [M+H⁺] (100%), 533.2 [2M+H⁺] (25%).

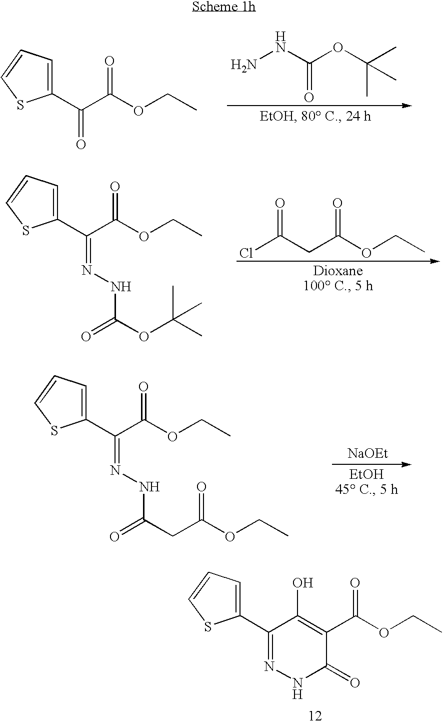

### (tert-Butoxycarbonyl-hydrazono)-thiophen-2-yl-acetic acid ethyl ester

Oxo-thiophen-2-yl-acetic acid ethyl ester (12.2 g, 66.23 mmol, Alfa Aesar) was dissolved in ethyl alcohol (120 mL). Hydrazinecarboxylic acid tert-butyl ester (15.75, 119.21 mmol, Aldrich) was added. The mixture was heated at 90° C. while stirring for 24 hours. The mixture was concentrated in vacuo to a yellow oil. Purification by flash column chromatography (10% EtOAc in hexanes, Merck silica gel 60, 40-63 μm) afforded the desired product, (tert-butoxycarbonyl-hydrazono)-thiophen-2-yl-acetic acid ethyl ester (13.66 g, 45.79 mmol, 69% yield), as a yellow oil. ¹H NMR (400 MHz, DMSO-d₆) δ: 1.33 (t, 3H, J=7.0 Hz), 1.48 (s, 9H), 4.37 (quartet, 2H, J=7.2 Hz), 7.06 (dd, 1H, J₁=5.5 Hz, J₂=4.0 Hz), 7.37 (dd, 1H, J₁=4.0 Hz, J₂=1.6 Hz), 7.58 (d, 1H, J=6.3 Hz), 11.01 (s, 1H).

### [(2-Ethoxycarbonyl-acetyl)-hydrazono]-thiophen-2-yl-acetic acid ethyl ester

(tert-Butoxycarbonyl-hydrazono)-thiophen-2-yl-acetic acid ethyl ester (13.66 g, 45.79 mmol) was dissolved in anhydrous 1-5-dioxane (230 mL). Chlorocarbonyl-acetic acid ethyl ester (10.34 g, 68.7 mmol) was added. While stirring, the mixture was degassed under vacuum and the flask charged with N₂. The mixture was heated at 100° C. while stirring for 5 hours. The mixture was poured into ½ saturated aqueous NaHCO₃ (250 mL). The product was extracted into EtOAc (2×200 mL). The organic phase was dried over MgSO₄ and concentrated in vacuo to afford the crude [(2-ethoxycarbonyl-acetyl)-hydrazono]-thiophen-2-yl-acetic acid ethyl ester as an orange oil.

### 5-Hydroxy-3-oxo-6-thiophen-2-yl-2,3-dihydro-pyridazine-4-carboxylic acid ethyl ester (12)

The crude [(2-ethoxycarbonyl-acetyl)-hydrazono]-thiophen-2-yl-acetic acid ethyl ester (−45 mmol) was dissolved in ethyl alcohol. A 21% solution of sodium ethoxide in ethyl alcohol (28 mL) was added. The mixture stirred at 45° C. for 5 hours. Upon cooling, the mixture was poured into 1M HCl (250 mL). A beige solid formed. The solid was collected by vacuum filtration and rinsed with ethyl alcohol (10 mL) and EtOAc (10 mL) to afford the desired product as a beige solid. The filtrate was concentrated in vacuo to a volume of 250 mL and diluted with water (100 mL). Additional product was extracted into EtOAc (2×250 mL), dried over MgSO₄ and concentrated to give a brownish orange solid. The solid was triturated with methanol (2×10 mL) and filtered to give the desired product as a beige solid. The solids were combined to afford the desired product, 5-hydroxy-3-oxo-6-thiophen-2-yl-2,3-dihydro-pyridazine-4-carboxylic acid ethyl ester (12) (5.4 g, 20.28 mmol, 44.3% yield).

The following compounds were synthesized in an analogous manner to that described in Scheme 1f.

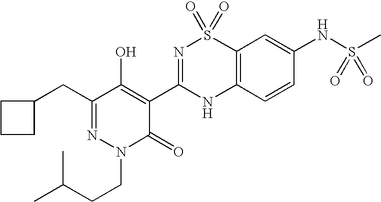

### N-{3-[6-Cyclobutylmethyl-5-hydroxy-2-(3-methyl-butyl)-3-oxo-2,3-dihydro-pyridazin-4-yl]-1,1-dioxo-1,4-dihydro-1l6-benzo[1,2,4]thiadiazin-7-yl}-methanesulfonamide

¹H NMR (400 MHz, CDCl₃) δ: 1.00 (6H, d, J=6.2 Hz), 1.60-1.83 (6H, m), 1.86-1.95 (2H, m), 2.06-2.14 (2H, m), 2.76 (1H, apparent sextet, J=7.6 Hz), 2.84 (2H, d, J=7.6 Hz), 3.09 (3H, s), 4.20 (2H, t, J=7.4 Hz), 6.57 (1H, s), 7.33 (1H, d, J=8.8 Hz), 7.65 (1H, dd, J=8.8 Hz, J₂=2.6 Hz), 7.69 (1H, d, J=2.4 Hz), 13.89 (1H, s); LC-MS (ESI⁺): m/e=524.41 [M+1]⁺ (Exact Mass: 523.16).

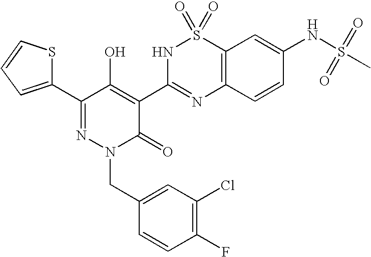

### N-{3-[2-(3-Chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, DMSO-d₆) δ: 3.01 (s, 3H), 5.15 (s, 2H), 7.06-7.08 (m, 1H), 7.33-7.36 (m, 3H), 7.42-7.45 (m, 1H), 7.49-7.53 (m, 3H), 7.88 (d, 1H, J=3.8 Hz), 9.95 (s, 1H). LC-MS (ESI): m/e 610.1 [M+1]⁺ (exact ms: 609.0).

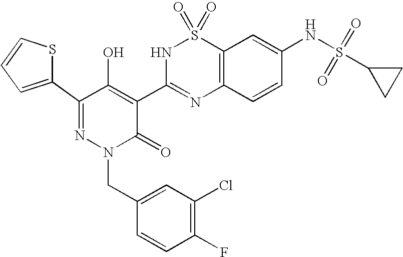

### Cyclopropanesulfonic acid {3-[2-(3-chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide

¹H NMR (400 MHz, DMSO-d₆) δ: 0.93-0.96 (m, 4H), 2.64-2.69 (m, 1H), 5.21 (s, 2H), 7.11 (t, 1H, J=4.3 Hz), 7.38-7.40 (m, 2H), 7.42-7.44 (m, 1H), 7.49-7.54 (m, 2H), 7.56 (bs, 1H), 7.58 (d, 1H, J=5.4 Hz), 7.89 (d, 1H, J=3.9 Hz), 10.05 (s, 1H). LC-MS (ESI⁺): m/e 636.3 [M+1]⁺ (exact ms: 635.02).

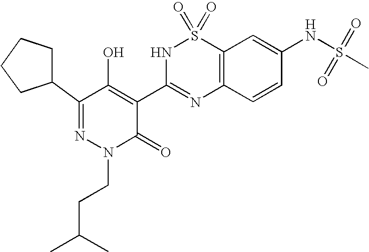

### N-{3-[6-Cyclopentyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, CDCl₃) δ: 1.00 (d, 6H, J=6.3 Hz), 1.60-1.65 (m, 1H), 1.68-1.73 (m, 4H), 1.77-1.82 (m, 4H), 2.00-2.06 (m, 2H), 3.09 (s, 3H), 3.40 (t, 1H, J=7.5 Hz), 4.20 (t, 2H, J=7.5 Hz), 6.67 (s, 1H), 7.33 (d, 1H, J=8.4 Hz), 7.66 (dd, 1H, J=8.7 Hz, J₂=2.4 Hz), 7.69 (d, 1H, J=2.3 Hz). LC-MS (ESI⁺): m/e 524.3 [M+1]⁺ (exact ms: 523.16).

### EXAMPLE 1-6

Scheme 11 describes another procedure for making compound (5f) of Formula I.

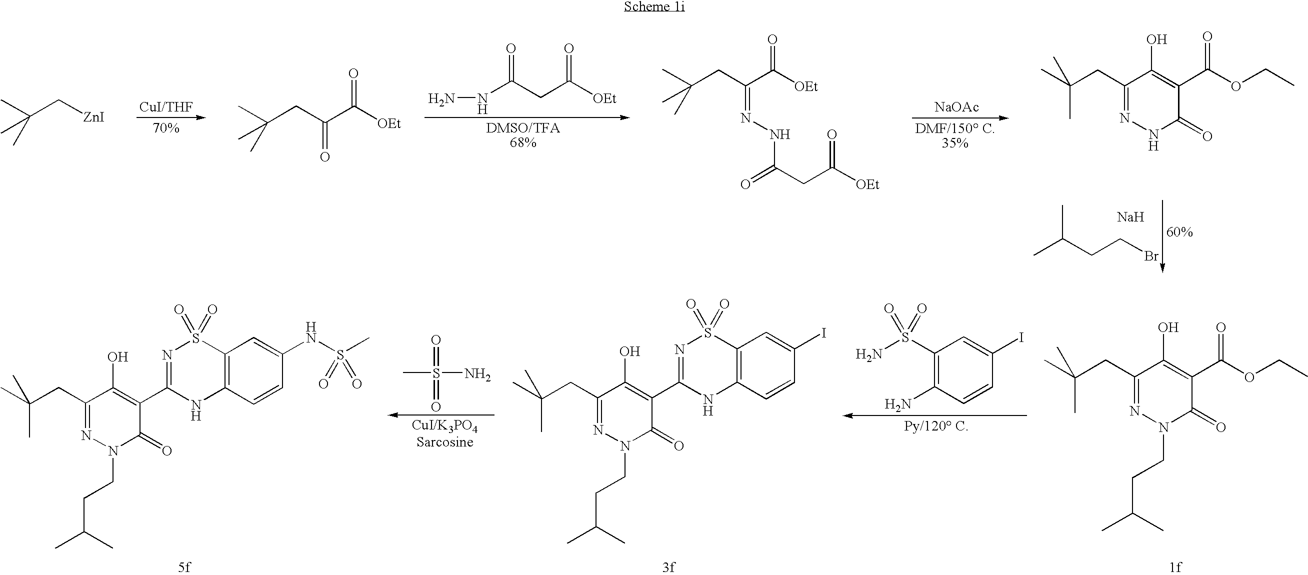

### 4,4-Dimethyl-2-oxo-pentanoic acid ethyl ester

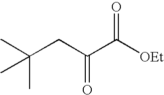

To a suspension of CuI (2.29 g, 12 mmol) in THF (15 mL) at −20° C. under N2 atmosphere, 2,2-dimethylpropylzinc iodide (neopentylzinc iodide) (0.5 M in THF, 24 mL, 12 mmol) was added slowly. The mixture was stirred at −20° C. for 10 min and at 0° C. for 20 min. The resulting mixture was then cooled down to −20° C. and ethylchlorooxoacetate (1.12 mL, 10 mmol) was added. The reaction mixture was warmed to 25° C. over 1 h. The reaction was quenched by the addition of saturated NH₄Cl and the product was extracted with Et₂O. The combined organic layers were washed with brine and dried over Na₂SO₄. The solid was removed by filter paper and the filtrate was concentrated by rotary evaporator (Temperature of water bath was less then 20° C.). The crude residue was then purified by flash chromatography on silicon gel to give the desired product, 4,4-Dimethyl-2-oxo-pentanoic acid ethyl ester, as a colorless oil (1.35 g, 78%). ¹H NMR (400 MHz, CDCl₃): δ 4.33 (q, 2H, J=7.2 Hz), 2.76 (s, 2H), 1.39 (t, 3H, J=7.2 Hz), 1.07 (s, 9H).

### 2-[(2-Ethoxycarbonyl-acetyl)-hydrazono]-4,4-dimethyl-pentanoic acid ethyl ester

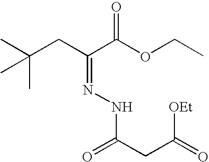

Ethyl 3-hydrazino-3-oxopropionate (1.26 g, 8.62 mmol) was added to a solution of 4,4-Dimethyl-2-oxo-pentanoic acid ethyl ester (1.35 g, 7.84 mmol) in 40 mL DMSO at room temperature. Trifluoroacetic acid (8 drops) was then added and the reaction mixture was stirred at room temperature for 1.5 h and at 60° C. for 4 h. TLC showed completion of the reaction. After cooling to room temperature, the mixture was partitioned between 0.5 M HCl and EtOAc. The combined organic layers were washed with brine, dried over Na₂SO₄ and concentrated. The residue was purified by column chromatography on silica gel to afford product, 2-[(2-Ethoxycarbonyl-acetyl)-hydrazono]-4,4-dimethyl-pentanoic acid ethyl ester, as a white solid (1.60 g, 68%). ¹H NMR (400 MHz, CDCl₃) δ: 8.95 (s, 1H), 4.29 (q, 2H, J=7.2 Hz), 4.21 (q, 2H, J=7.2 Hz), 3.79 (s, 2H), 2.54 (s, 2H), 1.36 (t, 3H, J=7.2 Hz), 1.31 (t, 2H, J=7.2 Hz), 1.01 (s, 9H).

### 6-(2,2-Dimethyl-propyl)-5-hydroxy-3-oxo-2,3-dihydro-pyridazine-4-carboxylic acid ethyl ester

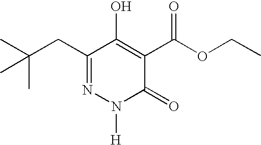

Sodium acetate (0.87 g, 10.66 mmol) was added to a solution of 2-[(2-Ethoxycarbonyl-acetyl)-hydrazono]-4,4-dimethyl-pentanoic acid ethyl ester (1.60 g, 5.33 mmol) in 25 mL DMF at room temperature. The resulting suspension was stirred at 150° C. for 2 h. The reaction mixture was then cooled to room temperature and 1.0 M aqueous HCl (40 mL) was added. The resulting beige precipitate was collected by filtration, washed with water (2×20 mL) and dried with high vacuum pump to afford compound 6-(2,2-Dimethyl-propyl)-5-hydroxy-3-oxo-2,3-dihydro-pyridazine-4-carboxylic acid ethyl ester as a tan solid (0.51 g, 37%). ¹H NMR (400 MHz, DMSO-d₆) δ: 12.65 (s, 1H), 4.27 (q, 2H, J=7.2 Hz), 2.50 (s, 2H), 1.26 (t, 3H, J=7.2 Hz), 0.91 (s, 9H); LC-MS (ESI): m/e=255.2 [M+H]⁺ (exact ms: 254.13).

### 6-(2,2-Dimethyl-propyl)-5-hydroxy-2-(3-methyl-butyl)-3-oxo-2,3-dihydro-pyridazine-4-carboxylic acid ethyl ester (1f)

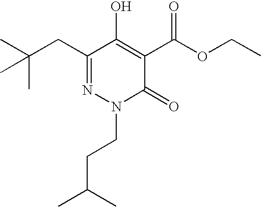

Sodium hydride (0.112 g of a 60% suspension in mineral oil, 2.80 mmol) was added to a solution of compound 6-(2,2-Dimethyl-propyl)-5-hydroxy-3-oxo-2,3-dihydro-pyridazine-4-carboxylic acid ethyl ester (0.355 g, 1.40 mmol) in DMF (3 mL) at room temperature. The resulting suspension was stirred for 10 min, and then 1-bromo-3-methylbutane (0.22 mL, 1.68 mmol) was added. The reaction mixture was stirred at room temperature for 1 h, and then was poured into 40 mL of 1.0 M HCl, extracted with EtOAc (2×). The combined organic layers were washed with brine, dried over Na₂SO₄ and concentrated. The residue was purified by column chromatography on silica gel to afford product 6-(2,2-Dimethyl-propyl)-5-hydroxy-2-(3-methyl-butyl)-3-oxo-2,3-dihydro-pyridazine-4-carboxylic acid ethyl ester (1f) (0.24 g, 53%). ¹H NMR (400 MHz, CDCl₃) δ: 13.21 (s, 1H), 4.50 (q, 2H, J=7.2 Hz), 4.14 (t, 2H, J=7.2 Hz), 2.61 (s, 2H), 1.67 (m, 3H), 1.49 (t, 3H, J=7.2 Hz), 0.99 (s, 9H), 0.97 (d, 6H, J=6.8 Hz); LC-MS (ESI⁺): m/e=325.20 [M+H]⁺ (exact ms: 324.20).

The procedure for making compounds 3f and 5f was the same as that described in Scheme 1g to give the final product (5f) of Formula I.

### N-{3-[6-(2,2-Dimethyl-propyl)-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide (5f)

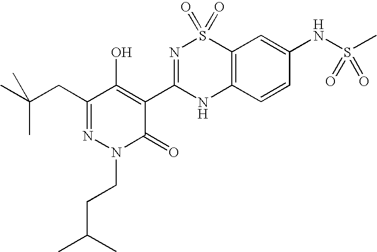

¹H NMR (400 MHz, DMSO-d₆): δ 10.26 (s, 1H), 7.68 (d, 1H, J=8.8 Hz), 7.60 (d, 1H, J=2.4 Hz), 7.55 (dd, 1H, J=9.2, 2.4 Hz), 4.14 (T, 2H, J=7.2 Hz), 3.08 (s, 3H), 2.60 (s, 2H), 1.63 (m, 3H), 0.96 (s, 9H), 0.91 (d, 6H, J=6.4 Hz); LC-MS (ES⁺): m/e=526.5 [M+1]⁺ (exact ms: 525.17).

### EXAMPLE 1-7

Scheme 1j describes another procedure for making compound (3g) of formula I.

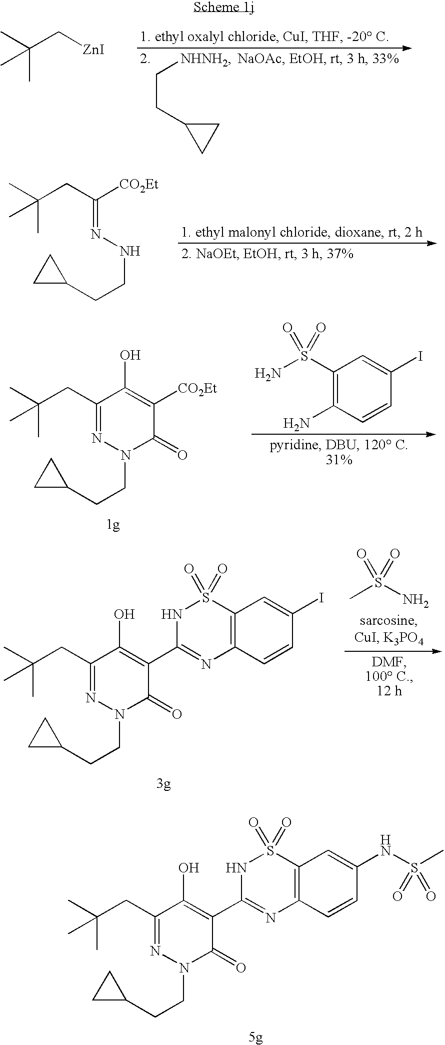

The α-ketoester was made via Zinc reagent and ethyl oxalyl chloride similar to the method described in Scheme 1i. The rest of the synthesis for making 1g, 3g and 5g was analogous to that described in Scheme 1a.

### N-{3-[2-(2-Cyclopropyl-ethyl)-6-(2,2-dimethyl-propyl)-5-hydroxy-3-oxo-2,3-dihydro-pyridazin-4-yl]-1,1-dioxo-1,2-dihydro-1l6-benzo[1,2,4]thiadiazin-7-yl}-methanesulfonamide (5g)

¹H NMR (400 MHz, CDCl₃) δ: 7.71 (d, 1H, J=2.3 Hz), 7.68 (dd, 1H, J₁=9.4 Hz, J₂=3.1 Hz), 7.33 (d, 1H, J=8.5 Hz), 6.89 (s, 1H), 4.30 (t, 2H, J=7.4 Hz), 3.09 (s, 3H), 2.68 (s, 2H), 1.74 (q, 2H, J=7.3 Hz), 1.01 (s, 9H), 0.71-0.77 (m, 1H), 0.45-0.51 (m, 2H), 0.08 (q, 2H, J=5.0 Hz); LC-MS (ESI⁺): m/e=524.5 [M+1]⁺ (Exact Mass: 523.2).

### EXAMPLE 1-8

Scheme 1k describes another procedure for making compound of Formula I. In this procedure, the synthesis of the corresponding α-keto-ester was followed a procedure described by E. L. Eliel and A. A. Hartmann, J. Org. Chem., 37, 505-06 (1972).

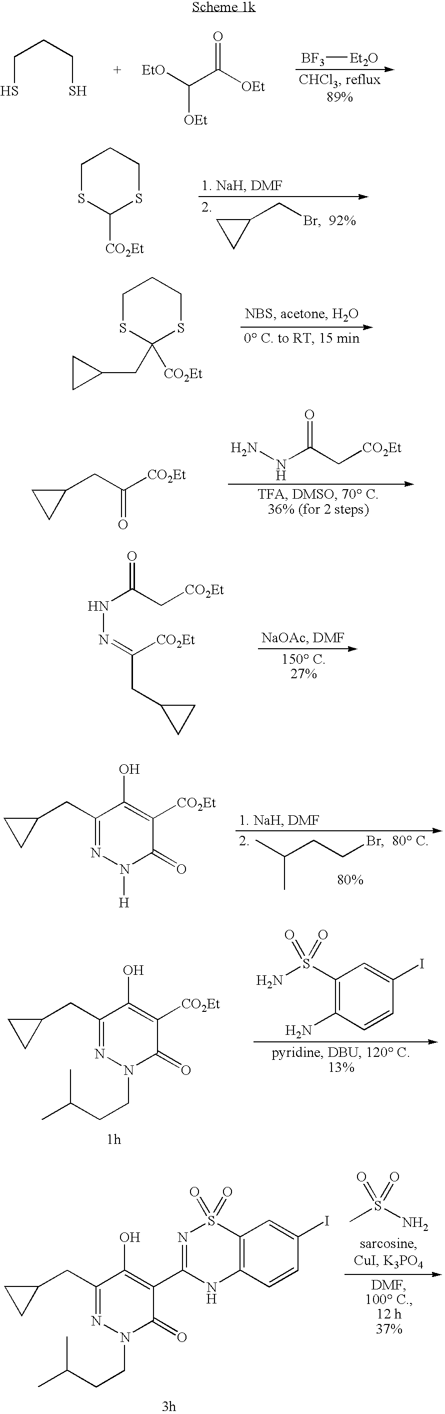

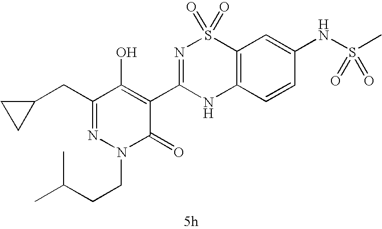

### [1,3]Dithiane-2-carboxylic acid ethyl ester

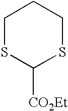

Diethoxy-acetic acid ethyl ester (3.22 g, 18.3 mmol), and propane-1,3-dithiol (1.8 mL, 18.3 mmol) dissolved in dry chloroform (2 mL), was added dropwise to a refluxing solution of BF₃.Et₂O (5.19 g, 36.3 mmol) in dry chloroform (6 mL) via dropping funnel. The resulting pale yellow solution was refluxed for 0.5 h, cooled to room temperature, washed with H₂O, 20% aqueous potassium carbonate, and H₂O. The aqueous layer was extracted with chloroform, and the combined organic layers were dried over MgSO₄. The solution was filtered and concentrated in vacuo. The desired product was distilled, bp 76.5° C. (0.2 mmHg), yield 1.29 g (37%).

### 2-Cyclopropylmethyl-[1,3]dithiane-2-carboxylic acid ethyl ester

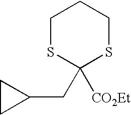

Dithiane-2-carboxylic acid ethyl ester (1.29 g, 6.71 mmol) and bromomethyl-cyclopropane (0.99 g, 7.38 mmol) dissolved in dry DMF (1 mL), was added slowly to a stirred suspension of NaH (60% in mineral oil, 268 mg, 6.71 mmol) in dry toluene (2 mL). The mixture was stirred at room temperature overnight and LC-MS showed completion of the reaction. The reaction was quenched with H₂O, diluted with EtOAc. The organic layers were washed with H₂O and dried with MgSO₄. The solution was filtered and concentrated in vacuo to give desired product, 2-cyclopropylmethyl-[1,3]dithiane-2-carboxylic acid ethyl ester, as yellow oil (1.49 g, 91%). LC-MS (ESI⁺): m/e=247.0 [M+1]⁺ (exact ms: 246.07).

### 3-Cyclopropyl-2-oxo-propionic acid ethyl ester

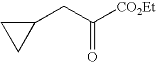

2-Cyclopropylmethyl-[1,3]dithiane-2-carboxylic acid ethyl ester (5.86 g, 23.6 mmol) dissolved in acetone (100 mL), was added to a solution of NBS (25.2 g, 99 mmol) in a 4:1 mixture of acetone and water (250 mL) at 0° C. The mixture was stirred from 0° C. to room temperature in a period of 15 min. TLC showed the completion of the reaction. The reaction was quenched with saturated aqueous NaHSO₃, extracted with Et₂O. The combined organic layer was washed with brine, dried over MgSO₄ and concentrated. The residue was purified by column chromatography on silica gel to give desired product, 3-cyclopropyl-2-oxo-propionic acid ethyl ester. The crude product was used directly to next step without purification. The remainder of the synthesis for making compounds of 1h, 3h and 5h was done in an analogous manner to that described in Scheme 1i.

### N-{3-[6-Cyclopropylmethyl-5-hydroxy-2-(3-methyl-butyl)-3-oxo-2,3-dihydro-pyridazin-4-yl]-1,1-dioxo-1,4-dihydro-1l6-benzo[1,2,4]thiadiazin-7-yl}-methanesulfonamide (5h)

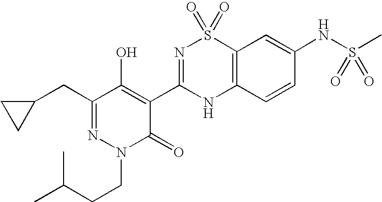

¹H NMR (400 MHz, CDCl₃) δ: 7.73 (d, 1H, J=2.4 Hz), 7.68 (dd, 1H, J₁=9.4 Hz, J₂=2.2 Hz), 7.33 (d, 1H, J=8.5 Hz), 7.22 (s, 1H), 4.22 (t, 2H, J=7.3 Hz), 3.09 (s, 3H), 3.00 (s, 1H), 2.65 (d, 2H, J=7.0 Hz), 1.61-1.74 (m, 3H), 1.27 (s, 1H), 1.09-1.19 (m, 1H), 1.01 (d, 6H, J=6.2 Hz), 0.53-0.57 (m, 2H), 0.27 (q, 2H, J=5.1 Hz); LC-MS (ESI⁺): m/e=510.46 [M+1]⁺ (Exact Mass: 509.14).

### EXAMPLE 1-9

Scheme 1L describes another procedure to make compound 5i of Formula I.

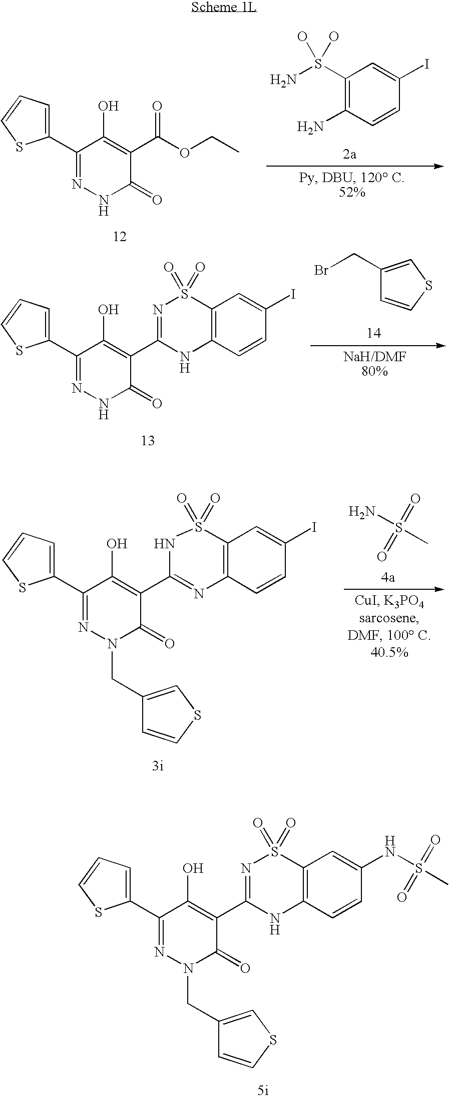

### 5-Hydroxy-4-(7-iodo-1,1-dioxo-1,4-dihydro-1λ⁶-benzo[1,2,4]thiadiazin-3-yl)-6-thiophen-2-yl-2H-pyridazin-3-one (13)

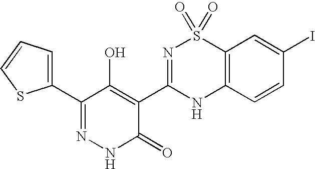

To a solution of 5-Hydroxy-3-oxo-6-thiophen-2-yl-2,3-dihydro-pyridazine-4-carboxylic acid ethyl ester (12) (0.556 g, 2.08 mmol) in pyridine (10.4 mL), 2-amino-5-iodo-benzenesulfonamide (2a) (0.684 g, 2.3 mmol) was added and the mixture was heated to 120° C. for 16 h. Cooled mixture was concentrated to a thick slurry under reduced pressure, suspended in methanol (40 mL) and filtered after brief agitation. Process repeated two more time with methanol (2×25 mL) before filtering the solids to afford desired 5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (13) (0.542 g, 52%) as a solid. LC-MS (ESI⁺): m/e=500.90 [M+1]⁺ (exact mass: 499.91).

### 5-Hydroxy-4-(7-iodo-1,1-dioxo-1,2-dihydro-1λ⁶-benzo[1,2,4]thiadiazin-3-yl)-6-thiophen-2-yl-2-thiophen-3-ylmethyl-2H-pyridazin-3-one (3i)

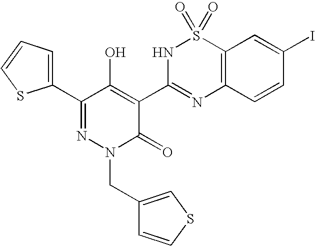

5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (13) (0.137 g, 0.27 mmol) was suspended in dimethylformamide (1.36 mL) followed by addition of sodium hydride (0.035 mg, 0.87 mmol) at 25° C. Mixture was allowed to stir for 20 min at 25° C. after which time 3-Bromomethyl-thiophene (14) (0.05 g, 0.28 mmol) was added and mixture stirred for 2.5 h. Resulting mixture was diluted with ethyl acetate (50 mL) and washed with 1N HCl (50 mL) and brine (50 mL). The organic layer was then concentrated to dryness under reduced pressure, resuspended in methanol (20 mL) to give a suspension of solids, and finally filtered to afford 5-Hydroxy-4-(7-iodo-1,1-dioxo-1,2-dihydro-1λ⁶-benzo[1,2,4]thiadiazin-3-yl)-6-thiophen-2-yl-2-thiophen-3-ylmethyl-2H-pyridazin-3-one (3i) (0.084 g, 41%) as a solid. LC-MS (ESI⁺): m/e=597.0 [M+1]⁺ (exact mass: 595.91).

### N-[3-(5-Hydroxy-3-oxo-6-thiophen-2-yl-2-thiophen-3-ylmethyl-2,3-dihydro-pyridazin-4-yl)-1,1-dioxo-1,4-dihydro-1λ⁶-benzo[1,2,4]thiadiazin-7-yl]-methanesulfonamide (5i)

The synthesis of compound 5i from compound 3i is similar to that described in Scheme 1a in the last step. ¹H NMR (400 MHz, DMSO-d₆) δ: 3.06 (s, 3H), 5.29 (s, 2H), 7.13-7.16 (m, 2H), 7.44 (bs, 1H), 7.49-7.51 (m, 1H), 7.53 (d, 1H, J=2.3 Hz), 7.55 (s, 1H), 7.58 (d, 1H, J=2.4 Hz), 7.64-7.66 (m, 1H), 7.91 (d, 1H, J=4.0 Hz), 10.16 (s, 1H). LC-MS (ESI⁺): m/e 564.3 [M+1]⁺ (exact ms: 563.01).

### EXAMPLE 1-10

Scheme 1m describes another procedure to make compounds of 5j and 15 of Formula I.

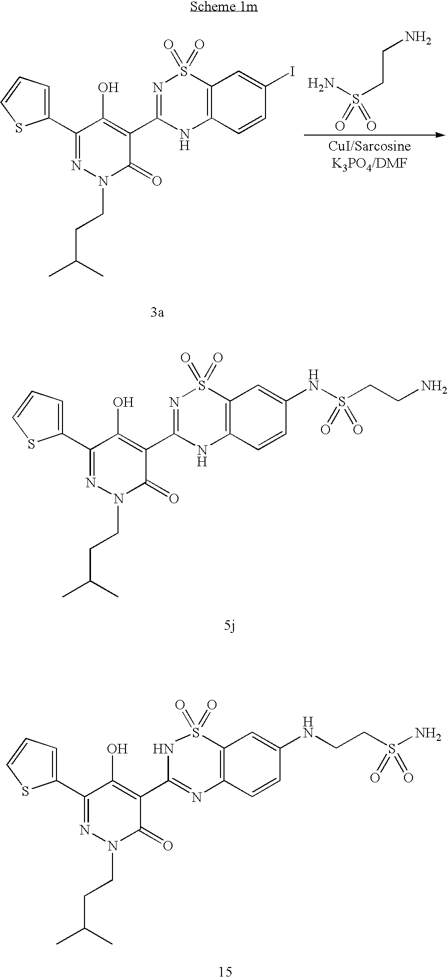

### 2-Amino-ethanesulfonic acid {3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5i)

A reaction flask was charged with 5-hydroxy-4-(7-iodo-1,1-dioxo-1,4-dihydro-1l6-benzo[1,2,4]thiadiazin-3-yl)-2-(3-methyl-butyl)-6-thiophen-2-yl-2H-pyridazin-3-one (3a) (347 mg, 0.61 mmol), CuI (46 mg, 0.24 mmol), sarcosine (N-methyl glycine) (33.0 mg, 0.37 mmol), 2-amino-ethanesulfonamide (293 mg, 1.83 mmol), and potassium phosphate (649 mg, 3.05 mmol). Anhydrous DMF (5 mL) was added into the flask. The flask was evacuated and back-filled with nitrogen twice, and then stirred at 100° C. for 3 h under nitrogen atmosphere. LC-MS showed the formation of two desired products and some starting material left. CuI (23 mg, 0.12 mmol) and sarcosine (16 mg, 0.18 mmol) were added to the reaction mixture and stirring was continued for 3 h. The reaction mixture was cooled to room temperature, and concentrated under reduced pressure. The crude compound was purified by prep-HPLC, followed by trituration with methanol to give two desired products:

### Product 1 (5j):

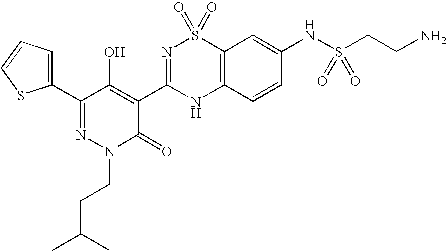

2-Amino-ethanesulfonic acid {3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5j) as white solid (30 mg, 8.7%); ¹H NMR (400 MHz, DMSO-d₆): δ 10.41 (s, 1H), 7.87 (dd, 1H, J=3.6, 0.8 Hz), 7.80 (broad s, 2H), 7.56 (m, 2H), 7.48 (m, 2H), 7.09 (dd, 1H, J=5.2, 3.6 Hz), 4.06 (t, 2H, J=6.8 Hz), 3.44 (m, 2H), 3.16 (m, 2H), 1.62 (m, 3H), 0.94 (d, 6H, J=6.4 Hz); LC-MS (ESI): m/e=567.4 [M+1]⁺ (exact ms: 566.11).

### Product 2 (15):

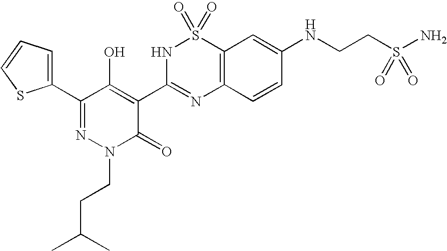

2-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-ylamino}-ethanesulfonic acid amide (15) as a brown solid (35 mg, 9%); ¹H NMR (400 MHz, DMSO-d₆): δ 13.70 (s, 1H), 7.91 (dd, 1H, J=4.0, 1.2 Hz), 7.70 (m, 1H), 7.48 (d, 1H, J=8.8 Hz), 7.17 (dd, 1H, J=5.2, 4.0 Hz), 7.00 (dd, 1H, J=8.8, 2.8 Hz), 6.93 (s, 2H), 6.90 (d, 1H, J=2.8 Hz), 4.19 (t, 2H, J=7.2 Hz), 3.53 (t, 2H, J=8.0 Hz), 3.24 (m, 2H), 1.68 (m, 3H), 0.94 (d, 6H, J=6.4 Hz); LC-MS (ESI): m/e=567.5 [M+1]⁺ (exact ms: 566.11).

The following compound was made using an analogous method as that described in Scheme 1m.

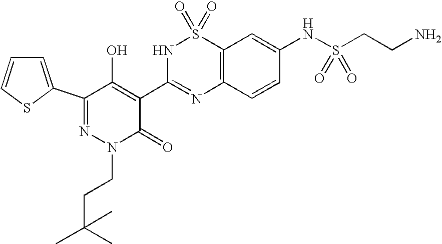

### N-{3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-2-amino-ethanesulfonamide

¹H NMR (400 MHz, DMSO-d₆): δ 10.42 (s, 1H), 7.88 (d, 1H, J=3.6 Hz), 7.54 (m, 2H), ), 7.47 (m, 2H), 7.10 (d, 1H, J=3.6 Hz), 4.07 (m, 2H), 3.46 (m, 2H), 3.17 (m, 2H), 1.63 (m, 2H), 0.97 (m, 9H); LC-MS (ESI⁺): m/e=581.2 [M+H]⁺ (exact ms: 580.12).

Method 2: Scheme 2 provides a general procedure that was used to prepare compound (5) of Formula I.

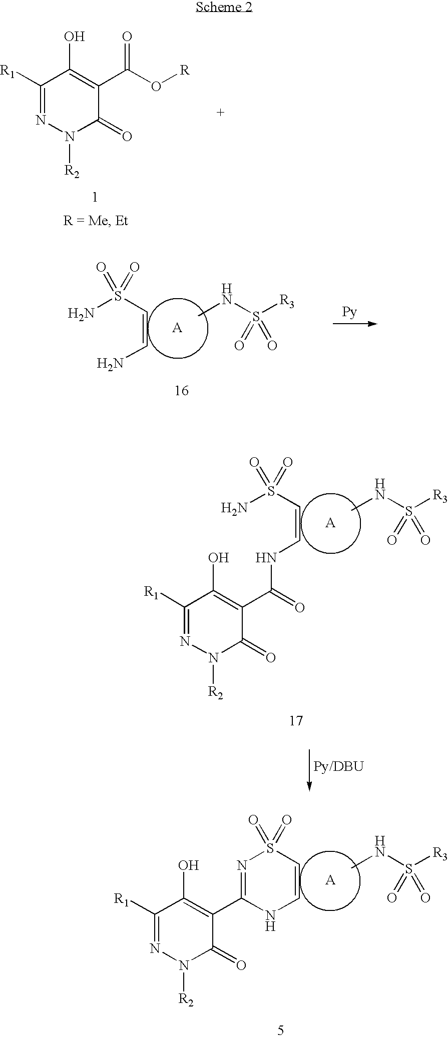

In the general procedure, intermediates 1 and 16 can be mixed in pyridine in the presence or absence of molecular sieves and heated at temperature between 100-120° C. for 3-24 hours, followed by treatment with 1,8-Diazabicyclo[5,4,0]undec-7-ene (DBU) to give desired cyclized product 5. The reaction is continued until completion, which typically occurs from about 16 to 48 hours.

### EXAMPLE 2-1

Scheme 2a describes the synthesis of compound 5k.

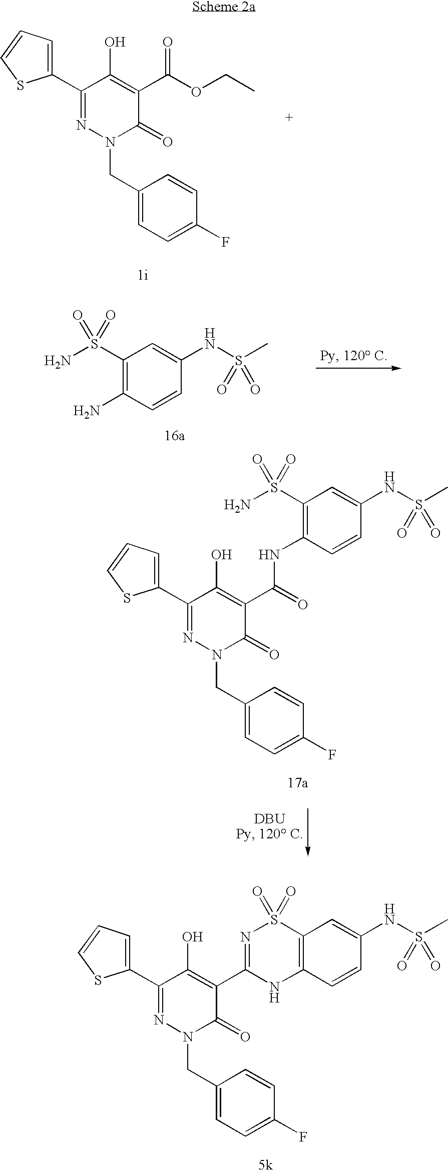

In this example, compound 1i was made in a similar fashion as that described in method 1 (Scheme 1a). In this specific example, compound 1i (109.3 mg, 0.29 mmol) was mixed with compound 16a (see Scheme 18a) (84.2 mg, 0.32 mmol), 106 mg of molecular sieves and 1.5 mL of pyridine. The resulted mixture was stirred under N₂ atmosphere at 120° C. (oil bath temperature) for 21 hours. LC-MS spectrum confirmed the disappearance of the starting material and the existence of both the uncyclized intermediated 17a and the final product 5k. Additional molecular sieves (100 mg) were added and the reaction mixture was stirred for additional 18 hours. No improvement was observed based on the LC-MS spectrum. 1,8-Diazabicyclo[5,4,0]undec-7-ene (DBU) (210 μL, 4.4 equivalents) was added and the resulted mixture was stirred under N₂ atmosphere at 120° C. (oil bath temperature) for 24 hours. LC-MS spectrum showed that only 50% of the intermediate 17a converted to the cyclized final product 5k. Additional DBU (400 μL) was added and the reaction mixture was continued to stir at 120° C. for 2 days. The LC-MS spectrum showed that the reaction was completed and the reaction mixture was concentrated under reduced pressure to remove the pyridine solvent. Saturated NH₄Cl (2 mL), H₂O (2 mL) and brine (2 mL) were added. The aqueous layer was extracted with ethyl acetate (10 mL×2) and CHCl₃ (10 mL×3). The combined organic layer was dried over anhydrous MgSO₄, filtered and concentrated to give a crude product (352.4 mg) which was further purified by flash column chromatography using silica gel eluted with 0-10% MeOH/CH₂Cl₂ to give 45.4 mg of the pure desired product (5k) with 27% isolated yield. ¹H NMR (DMSO-d₆): δ 13.9 (s, 1H), 10.18 (s, 1H), 7.90 (dd, 1H, J₁=3.6 Hz, J₂=1.2 Hz), 7.66 (dd, 1H, J₁=4 Hz, J₂=1.2 Hz), 7.51-7.59 (m, 3H), 7.42 (dd, 2H, J=8.8 Hz, J₂=5.6 Hz), 7.14-7.19 (m, 3H), 5.30 (s, 2H), 3.06 (s, 3H); LC-MS (ESI⁺): m/e 576.3 [M+1]⁺ (exact MS: 575.04).

The following compounds of formula I were also made in an analogous manner to the procedure described in Scheme 2a.

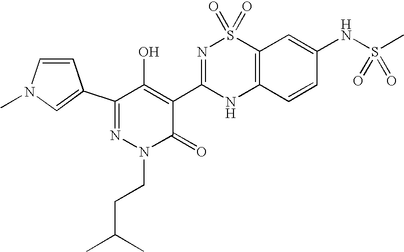

### N-{3-[5-Hydroxy-2-(3-methyl-butyl)-6-(1-methyl-1H-pyrrol-3-yl)-3-oxo-2,3-dihydro-pyridazin-4-yl]-1,1-dioxo-1,4-dihydro-1λ⁶-benzo[1,2,4]thiadiazin-7-yl}-methanesulfonamide

Yield: 12% (last step). ¹H NMR (400 MHz, CDCl₃): δ 10.14 (s, 1H), 7.72 (d, 1H, J=2.4 Hz), 7.62 (d, 1H, J=1.2 Hz), 7.55 (dd, 1H, J=7.8, 2.4 Hz), 6.80 (d, 1H, J=1.6 Hz), 6.55 (dd, 1H, J=2.0, 4.4 Hz), 4.17 (t, 2H, J=7.8 Hz), 3.67 (s, 3H), 3.08 (s, 1H), 1.67 (m, 3H), 1.03 (d, 6H, J=6.0 Hz); LC-MS (ESI): m/e=535.4 [M+1]⁺ (exact ms: 534.14).

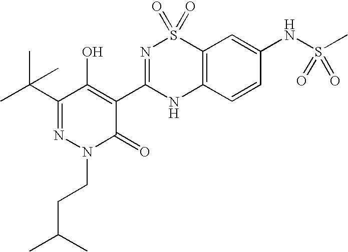

### N-{3-[6-tert-Butyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide

Yield: 15% (last step). ¹H NMR (400 MHz, CDCl₃): δ 10.14 (s, 1H), 7.72 (d, 1H, J=2.6 Hz), 7.60 (d, 1H, J=1.4 Hz), 7.55 (dd, 1H, J=7.8, 2.6 Hz), 4.06 (t, 2H, J=7.8 Hz), 3.08 (s, 1H), 1.67 (m, 1H), 1.36 (s, 9H), 0.99 (d, 6H, J=6.8 Hz); LC-MS (ESI⁺): m/e=512.3 [M+1]⁺ (exact ms: 511.16).

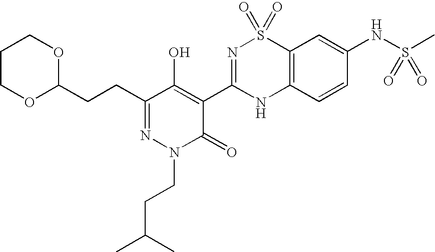

¹H NMR (400 MHz, CDCl₃): δ 10.35 (s, 1H), 7.68 (d, 1H, J=5.4 Hz), 7.60 (d, 1H, J=1.6 Hz), 7.55 (dd, 1H, J=7.8, 2.6 Hz), 4.80 (d, 1H, J=5.4 Hz), 4.08 (m, 2H), 4.00 (dd, 2H, J=5.4, 2.8 Hz), 3.86 (d, 2H, J=4.8, 2.6 Hz), 3.06 (s, 1H), 2.86 (m, 2H), 1.96 (m, 2H), 1.80 (m, 2H), 1.4 (d, 1H, J=3.6 Hz), 0.99 (d, 6H, J=6.4 Hz); LC-MS (ESI⁺): m/e=571.2 [M+1]⁺ (exact ms: 569.16).

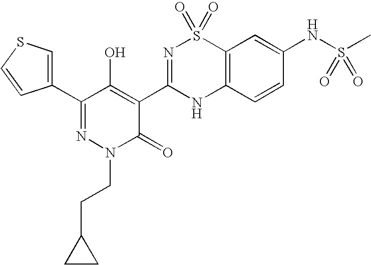

### N-{3-[2-(2-Cyclopropyl-ethyl)-5-hydroxy-3-oxo-6-thiophen-3-yl-2,3-dihydro-pyridazin-4-yl]-1,1-dioxo-1,4-dihydro-1-benzo[1,2,4]thiadiazin-7-yl}-methanesulfonamide

Yield (last step): 29.2%. ¹H NMR (400 MHz, ppm, DMSO-d₆): δ 10.26 (s, 1H), 8.27 (dd, 1H, J1=2.6 Hz, J2=1.4 Hz), 7.69 (d, 1H J=8.8 Hz), 7.61-7.64 (m, 3H), 7.56 (dd, 1H, J1=8.8 Hz, J2=2.4 Hz), 4.26 (t, 2H, J=7.0 Hz), 3.08 (s, 3H), 1.71 (q, 2H, J=7.0 Hz), 0.70-0.80 (m, 1H), 0.38-0.43 (m, 2H), 0.02-0.07 (m, 2H); LC-MS (ESI⁺): m/e=536.2 [M+1]⁺ (exact ms: 535.07).

Oxo-thiophen-3-yl-acetic acid ethyl ester was made from the corresponding 3-thiophenylzinc iodine in a similar fashion to the method described in Scheme 1i.

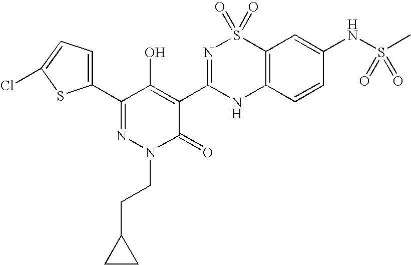

### N-{3-[6-(5-Chloro-thiophen-2-yl)-2-(2-cyclopropyl-ethyl)-5-hydroxy-3-oxo-2,3-dihydro-pyridazin-4-yl]-1,1-dioxo-1,4-dihydro-1l6-benzo[1,2,4]thiadiazin-7-yl}-methanesulfonamide

Yield (last step): 15.2%. ¹H NMR (400 MHz, ppm, DMSO-d₆): δ 13.93 (s, br, <1H), 10.17 (s, 1H), 7.70 (d, 1H, J=4.4 Hz), 7.50-7.60 (m, 3H), 7.15 (d, 1H, 4.4 Hz), 4.18 (t, 2H, J=6.8 Hz), 3.08 (s, 3H), 1.66 (q, 2H, J=7.2 Hz), 0.68-0.78 (m, 1H), 0.38-0.43 (m, 2H), 0.01-0.07 (m, 2H). LC-MS (ESI⁺): m/e=570 [M+1]⁺ (exact ms: 569). 5-chloro-thiophen-2-yl)-oxo-acetic acid ethyl ester was made from the corresponding 3-chloro-2-thiophenylzinc bromide in a similar fashion as that described in Scheme 1i.

### EXAMPLE 2-2

Scheme 2b describes the synthesis of compound 5L.

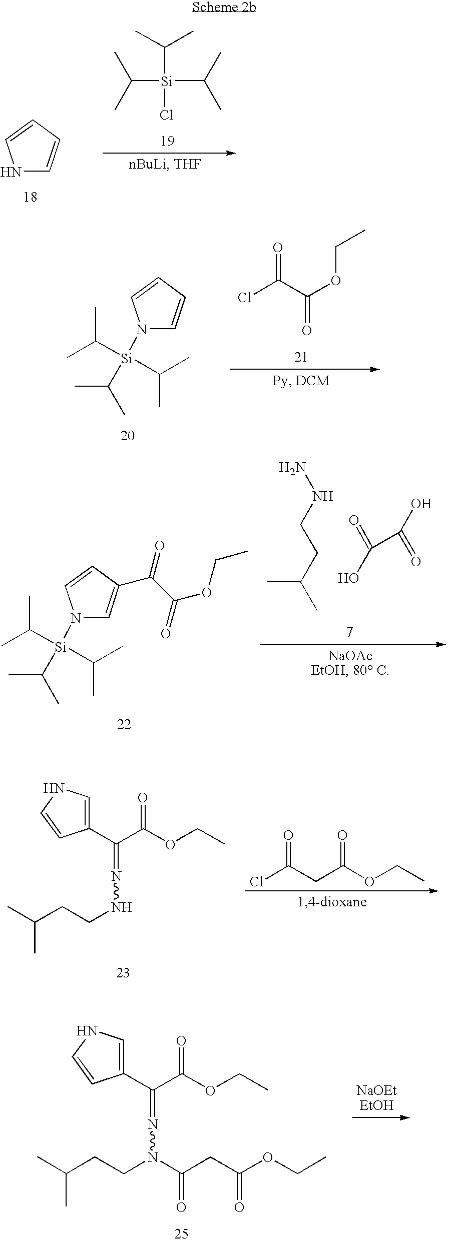

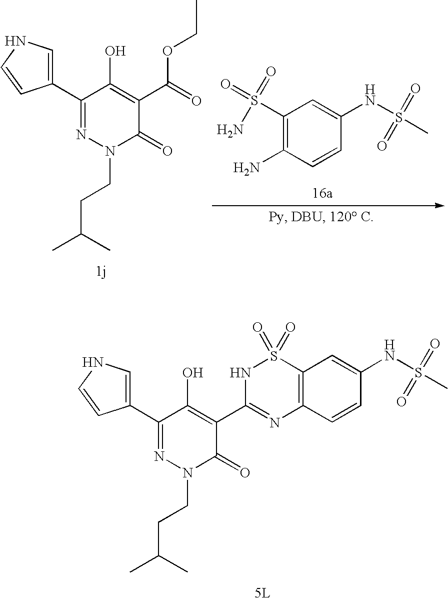

### 1-triisopropylsilanyl-1H-pyrrole (20)

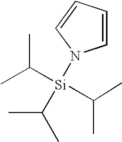

To a solution of 1H-pyrrole (18) (5 g, 74.5 mmol) in tetrahydrofurane (125 mL) cooled to −78° C. was added n-butyl lithium (51 mL, 1.6M in hexanes), dropwise. The mixture was stirred at −78° C. for 10 min, warmed and kept at 25° C. for an additional 10 min. After cooling back down to −78° C., chloro-triisopropyl-silane (19) (16 mL, 74.5 mmol) was added and the mixture was allowed to slowly warm up to 25° C. over a period of 1 h. The reaction mixture was concentrated and the residue dissolved in diethyl ether (250 mL) and washed with water (250 mL). The aqueous layer extracted three more times with diethyl ether (200 mL), with the organic layers combined, dried over Na₂SO₄ and concentrated. The residue was distilled at 220° C. isolating the desired product 1-triisopropylsilanyl-1H-pyrrole (20) (10.7 g, 64%) as a clear, yellow liquid.

### Oxo-(1-triisopropylsilanyl-1H-pyrrol-3-yl)-acetic acid ethyl ester (22)

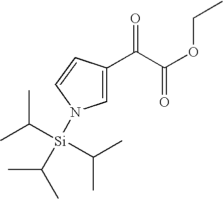

To a solution of chloro-oxo-acetic acid ethyl ester (21) (3.3 mL, 29.6 mmol) in anhydrous dichloromethane (20 mL) cooled to −20° C. was added a mixture of pyridine (2.4 mL, 29.6 mmol) in anhydrous dichloromethane (20 mL). A solution of 1-triisopropylsilanyl-1H-pyrrole (20) and anhydrous dichloromethane (30 mL) was then added to the above mixture dropwise. The reaction was allowed to warm up to 25° C. and stirred for 48 h. The reaction mixture was poured into a separatory funnel containing cooled solution of saturated NaHCO₃ (aq) (200 mL), wherein the organic layer was removed and the aqueous layer was extracted two more times with diethyl ether (2×200 mL). The combined organic layers were dried over MgSO₄ and concentrated. The residue was purified by column chromatography on silica gel to give desired product oxo-(1-triisopropylsilanyl-1H-pyrrol-3-yl)-acetic acid ethyl ester (22) (1.682 g, 58%) as a clear oil.

### [(3-methyl-butyl)-hydrazono]-(1H-pyrrol-3-yl)-acetic acid ethyl ester (23)

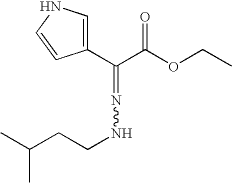

To a mixture of oxo-(1-triisopropylsilanyl-1H-pyrrol-3-yl)-acetic acid ethyl ester (22) in ethanol (160 mL) was added sodium acetate (2.28 g, 27.8 mmol) and (3-methyl-butyl)-hydrazine oxalic acid salt (7) (2.14 g, 11.1 mmol), which was heated to 80° C. for 2 h. The cooled mixture was concentrated under reduced pressure, resuspended in ethyl acetate (200 mL) and transferred to a separatory funnel. The mixture was washed twice with ½ saturated NaHCO₃ (aq) solution (2×200 mL), dried over MgSO₄ and concentrated. The residue was purified by column chromatography on silica gel to give desired product, [(3-methyl-butyl)-hydrazono]-(1H-pyrrol-3-yl)-acetic acid ethyl ester (23) (0.443 g, 11.7%) as an oil. LC-MS (ESI): m/e=252.2 [M+1]⁺ (exact mass: 251.16).

The compounds of 25 and 1j were made in an analogous manner as that described in Scheme 1a, wherein the last step of synthesis 5L was in a similar fashion to that described in Scheme 2a.

### N-{3-[5-Hydroxy-2-(3-methyl-butyl)-3-oxo-6-(1H-pyrrol-3-yl)-2,3-dihydro-pyridazin-4-yl]-1,1-dioxo-1,2-dihydro-1λ⁶-benzo[1,2,4]thiadiazin-7-yl}-methanesulfonamide (5L)

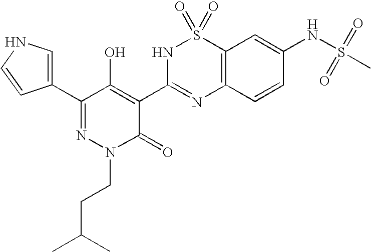

¹H NMR (400 MHz, DMSO-d₆) δ: 0.95 (d, 6H, J=6.3 Hz), 1.62-1.71 (m, 3H), 3.09 (s, 3H), 4.19 (t, 2H, J=6.9 Hz), 6.60 (bs, 1H), 6.84 (bs, 1H), 7.55-7.58 (m, 2H), 7.63 (d, 1H, J=2.1 Hz), 7.71 (d, 1H, J=8.4 Hz), 10.28 (s, H), 11.18 (bs, H).

LC-MS (ESI⁺): m/e 521.9 [M+1]⁺ (exact ms: 520.12).

### EXAMPLE 2-3

Scheme 2c describes the synthesis of compound 8c.

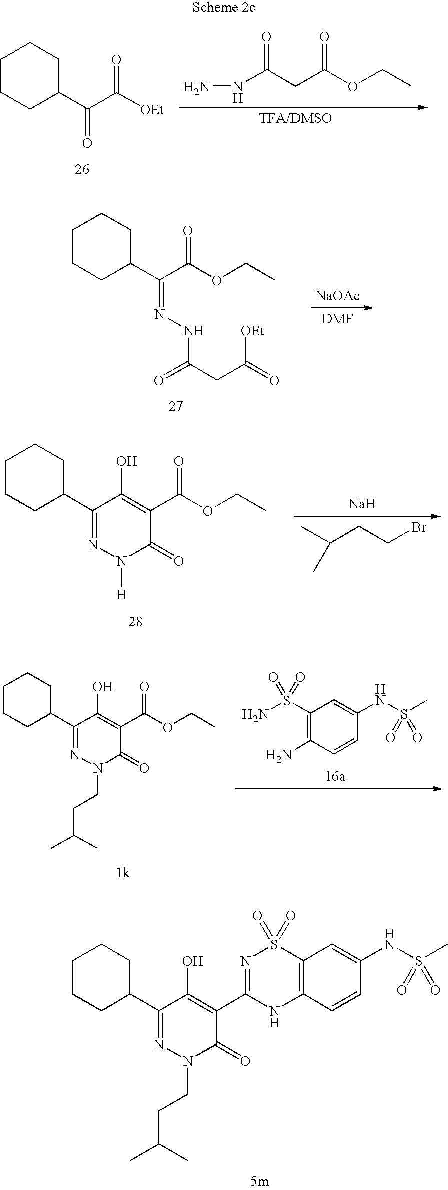

### Cyclohexyl-[(2-ethoxycarbonyl-acetyl)-hydrazono]-acetic acid ethyl ester (27)

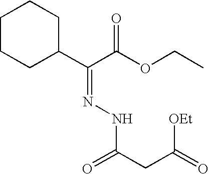

Ethyl 3-hydrazino-3-oxopropionate (3.79 g, 25.9 mmol) was added to a solution of compound 26 (4.34 g, 23.6 mmol) in 100 mL DMSO at rt. Trifluoroacetic acid (10 drops) was then added and the reaction mixture was heated at 70° C. for 20 h. After cooling to rt, the mixture was partitioned between 0.5 M HCl (400 mL) and EtOAc (2×350 mL). The combined organic layers were dried over Na₂SO₄ and were concentrated. The residue was purified via ISCO column chromatography (220 g column, 0→70% EtOAc in hexanes over 40 min, flow=50 mL/min) to afford product 27 {Cyclohexyl-[(2-ethoxycarbonyl-acetyl)-hydrazono]-acetic acid ethyl ester, 10:1 mixture of isomers, 5.40 g, 73%} as a white solid. Major isomer: ¹H NMR (400 MHz, CDCl₃) δ: 1.16-1.41 (m, 6H), 1.28 (t, 3H, J=7.1 Hz), 1.36 (t, 3H, J=6.9 Hz), 1.70-1.74 (m, 1H), 1.79-1.87 (m, 3H), 2.62-2.69 (m, 1H), 3.69 (s, 2H), 4.19 (q, 2H, J=7.3 Hz), 4.31 (q, 2H, J=7.2 Hz), 11.87 (s, 1H).

### 6-Cyclohexyl-5-hydroxy-3-oxo-2,3-dihydro-pyridazine-4-carboxylic acid ethyl ester (28)

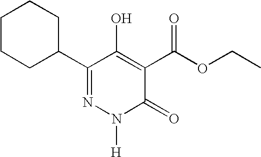

Sodium acetate (2.78 g, 33.9 mmol) was added to a solution of compound 27 (5.3 g, 17.0 mmol) in 60 mL DMF at rt. The resulting suspension was heated to 150° C. and maintained at that temperature for 2.5 hours. The reaction mixture was then cooled to rt and 0.5 M HCl (100 mL) was added. The resulting beige precipitate was collected by filtration, washed with water (2×50 mL) and air-dried overnight. The dried solid was triturated with a 1:1 mixture of Et₂O and hexanes (50 mL) then was collected by filtration, washed with a 1:1 mixture of Et₂O and hexanes (50 mL), and air-dried overnight to afford compound 28 (6-Cyclohexyl-5-hydroxy-3-oxo-2,3-dihydro-pyridazine-4-carboxylic acid ethyl ester, 2.30 g, 51%) as a tan solid. ¹H NMR (400 MHz, DMSO-d₆) δ: 1.13-1.38 (m, 6H), 1.26 (t, 3H, J=7.0 Hz), 1.66-1.69 (m, 1H), 1.75-1.80 (m, 3H), 2.72-2.88 (m, 1H), 4.26 (q, 2H, J=7.0 Hz), 12.54 (s, 1H).

### 6-Cyclohexyl-5-hydroxy-2-(3-methyl-butyl)-3-oxo-2,3-dihydro-pyridazine-4-carboxylic acid ethyl ester (1k)

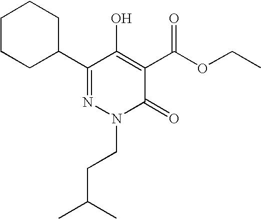

Sodium hydride (0.115 g of a 60% suspension in mineral oil, 2.88 mmol) was added to a solution of compound 28 (0.305 g, 1.15 mmol) in DMF (5 mL) at rt. The resulting suspension was stirred at rt for 15 min, and then 1-bromo-3-methylbutane (0.178 mL, 1.49 mmol) was added. The reaction mixture was heated at 80° C. for 1.5 hours, then was cooled to rt and concentrated under reduced pressure. The residue was partitioned between 0.5 M HCl (100 mL) and EtOAc (2×100 mL). The combined organic layers were dried over Na₂SO₄ and were concentrated. The residue was purified via ISCO column chromatography (80 g column, 0-50% EtOAc in hexanes over 40 min, flow=40 mL/min) to afford product 1k [6-Cyclohexyl-5-hydroxy-2-(3-methyl-butyl)-3-oxo-2,3-dihydro-pyridazine-4-carboxylic acid ethyl ester, 0.297 g, 77%] as a tan solid. ¹H NMR (400 MHz, CDCl₃) δ: 0.96 (d, 7H, J=6.3 Hz), 1.22-1.51 (m, 5H), 1.46 (t, 3H, J=7.1 Hz), 1.58-1.70 (m, 2H), 1.73-1.76 (m, 1H), 1.83-1.96 (m, 2H), 2.85-2.94 (m, 1H), 4.09-4.14 (m, 2H), 4.48 (q, 2H, J=6.9 Hz), 13.19 (s, 1H).

### N-{3-[6-Cyclohexyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide (5m)

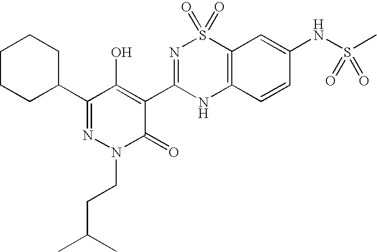

Compound 1k (0.190 g, 0.565 mmol) and compound 16a (0.150 g, 0.565 mmol) were dissolved in 4 mL of dry pyridine. The reaction mixture was then heated to 110° C. and was maintained at that temperature for 22 hours. 1,8-Diazabicyclo[5.4.0]undec-7-ene (0.170 mL, 1.13 mmol) was then added and the mixture heated at 110° C. for an additional 4 hours. After cooling to rt, the volatiles were removed under reduced pressure and the residue was partitioned between 0.5 M HCl (100 mL) and EtOAc (2×100 mL). The combined organic layers were dried over Na₂SO₄ and were concentrated. The residue was purified via ISCO column chromatography (80 g column, 0→100% EtOAc in hexanes over 45 min, flow=40 mL/min) to afford product (5m) {N-{3-[6-Cyclohexyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide} as a yellow solid (contaminated with several impurities). Trituration of this material with Et₂O (4×3 mL) provided 0.029 g (9.5%) of clean 5m as a white solid. ¹H NMR (400 MHz, DMSO-d₆) δ: 0.93 (d, 8H, J=6.2 Hz), 1.18-1.27 (m, 1H), 1.32-1.47 (m, 4H), 1.54-1.65 (m, 4H), 1.68-1.71 (m, 1H), 1.78-1.90 (m, 2H), 2.89-2.94 (m, 1H), 3.08 (s, 3H), 4.12 (t, 2H, J=7.0 Hz), 7.55 (dd, 1H, J=8.3 Hz, J₂=2.3 Hz), 7.61 (d, 1H, J=2.3 Hz), 7.68 (d, 1H, J=8.3 Hz), 10.26 (s, 1H), 13.88 (s, 1H); LCMS (ESI⁺): m/e=538.6 [+H]⁺.

The following compounds were made in the analogous manner as that described in Scheme 2c.

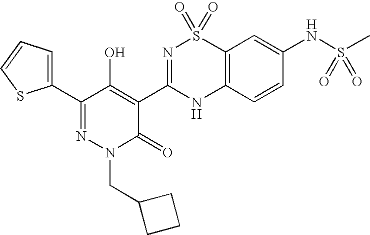

### N-[3-(2-Cyclobutylmethyl-5-hydroxy-3-oxo-6-thiophen-2-yl-2,3-dihydro-pyridazin-4-yl)-1,1-dioxo-1,4-dihydro-1l6-benzo[1,2,4]thiadiazin-7-yl]-methanesulfonamide

Yield (last step): 24% yield. ¹H NMR (400 MHz, DMSO-d₆) δ: 1.83-1.91 (m, 4H), 1.99-2.08 (m, 2H), 2.75-2.85 (m, 1H), 3.07 (s, 3H), 4.16 (d, 2H, J=7.0 Hz), 7.15 (dd, 1H, J₁=5.3 Hz, J₂=3.5 Hz), 7.53 (dd, 1H, J₁=9.2 Hz, J₂=1.9 Hz), 7.58-7.61 (m, 2H), 7.66 (d, 1H, J=4.8 Hz), 7.90 (d, 1H, J=3.8 Hz), 10.19 (s, 1H). LC-MS (ESI⁺): m/e=536.27 [M+H⁺] (100%) (exact mass: 535.07).

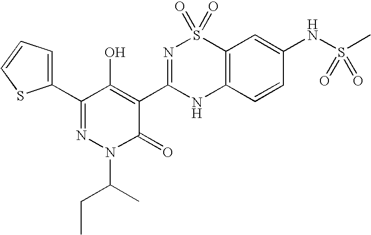

### N-[3-(2-sec-Butyl-5-hydroxy-3-oxo-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 0.83 (t, 3H, J=7.5 Hz), 1.35 (d, 3H, J=6.3 Hz), 1.68-1.78 (m, 1H), 1.79-1.90 (m, 1H), 3.08 (s, 3H), 5.04 (sextet, 1H, J=6.9 Hz), 7.17 (dd, 1H, J₁=5.4 Hz, J₂=3.8 Hz), 7.56 (dd, 1H, J=8.8 Hz, J₂=2.0 Hz), 7.61-7.69 (m, 3H), 7.92 (d, 1H, J=3.1 Hz), 10.24 (s, 1H), 13.92 (s, 1H). LC-MS (EST): m/e=524.25 [M+H]⁺ (100%) (exact mass: 523.07).

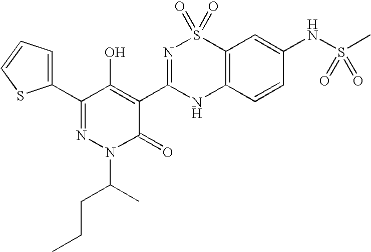

### N-{3-[5-Hydroxy-2-(1-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 0.88 (t, 3H, J=7.4 Hz), 1.18-1.28 (m, 2H), 1.34 (d, 3H, J=6.1 Hz), 1.62-1.70 (m, 1H), 1.80-1.89 (m, 1H), 3.08 (s, 3H), 5.10-5.16 (m, 1H), 7.17 (dd, 1H, J=5.0 Hz, J₂=3.5 Hz), 7.55 (dd, 1H, J=8.5 Hz, J₂=2.3 Hz), 7.61-7.68 (m, 3H), 7.92 (d, 1H, J=3.8 Hz), 10.22 (s, 1H), 13.98 (s, 1H). LC-MS (ESI⁺): m/e=538.44 [M+H⁺] (100%) (exact mass: 537.08).

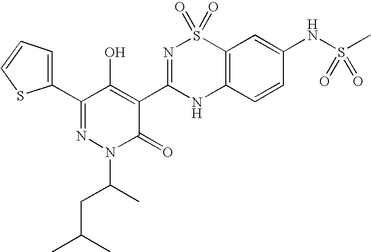

### N-{3-[2-(1,3-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 0.86 (d, 3H, J=6.2 Hz), 0.91 (d, 3H, J=6.2 Hz), 1.32 (d, 3H, J=6.4 Hz), 1.41-1.53 (m, 2H), 1.84-1.91 (m, 1H), 3.08 (s, 3H), 5.17-5.25 (m, 1H), 7.17 (t, 1H, J=4.3 Hz), 7.55 (dd, 1H, J₁=9.4 Hz, J₂=2.3 Hz), 7.61-7.69 (m, 3H), 7.92 (dd, 1H, J₁=4.0 Hz, J₂=1.6 Hz), 10.23 (s, 1H), 13.92 (s, 1H).

LC-MS (ESI): m/e=552.29 [M+H⁺] (100%) (exact mass: 551.10).

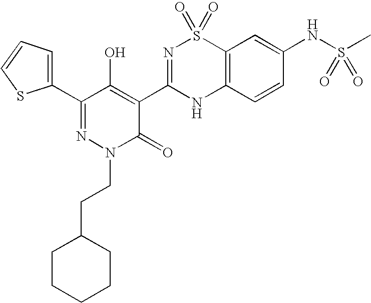

### N-{3-[2-(2-Cyclohexyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 0.90-1.00 (m, 2H), 1.12-1.26 (m, 3H), 1.29-1.36 (m, 1H), 1.59-1.69 (m, 5H), 1.76-1.80 (m, 2H), 3.08 (s, 3H), 4.17 (t, 2H, J=7.1 Hz), 7.16 (dd, 1H, J₁=5.5 Hz, J₂=3.9 Hz), 7.54 (dd, 1H, J₁=8.5 Hz, J₂=2.3 Hz), 7.60-7.68 (m, 3H), 7.91 (d, 1H, J=4.6 Hz), 10.21 (s, 1H). LC-MS (ESI): m/e=578.30 [M+H] (100%) (exact mass: 577.11).

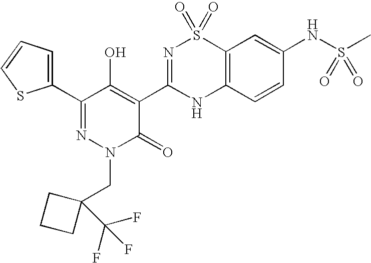

### N-{3-[5-Hydroxy-3-oxo-6-thiophen-2-yl-2-(1-trifluoromethyl-cyclobutylmethyl)-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 1.82-1.94 (m, 2H), 2.22-2.29 (m, 2H), 2.37-2.45 (m, 2H), 3.06 (s, 3H), 4.44 (s, 2H), 7.14-7.16 (m, 1H), 7.49-7.58 (m, 3H), 7.64-7.66 (m, 1H), 7.92 (dd, 1H, J₁=3.8 Hz, J₂=1.5 Hz), 10.14 (s, 1H). LC-MS (ESI⁺): m/e=604.1 [M+H⁺] (100%) (exact mass: 603.05).

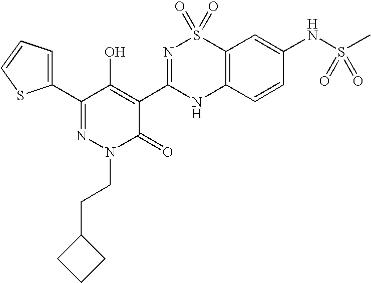

### N-{3-[2-(2-Cyclobutyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 1.58-1.68 (m, 2H), 1.74-1.90 (m, 4H), 1.98-2.07 (m, 2H), 2.32 (quintet, 1H, J=7.9 Hz), 3.08 (s, 3H), 4.07 (t, 2H, J=6.6 Hz), 7.16 (dd, 1H, J=5.4 Hz, J₂=3.8 Hz), 7.54 (dd, 1H, J=8.7 Hz, J₂=2.7 Hz), 7.60-7.68 (m, 3H), 7.91 (d, 1H, J=4.6 Hz), 10.21 (s, 1H). LC-MS (ESI): m/e=550.2 [M+H⁺] (100%) (exact mass: 549.08).

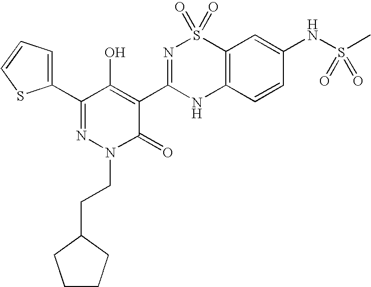

### N-{3-[2-(2-Cyclopentyl-ethyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 1.13-1.19 (m, 2H), 1.44-1.64 (m, 4H), 1.75-1.86 (m, 5H), 3.08 (s, 3H), 4.16 (t, 2H, J=6.6 Hz), 7.17 (dd, 1H, J₁=5.2 Hz, J₂=3.5 Hz), 7.55 (dd, 1H, J=8.4 Hz, J₂=2.3 Hz), 7.61-7.69 (m, 3H), 7.91 (d, 1H, J=4.0 Hz), 10.23 (s, 1H), 13.91 (s, 1H). LC-MS (ESI⁺): m/e=564.2 [M+H⁺] (100%) (exact mass: 563.10).

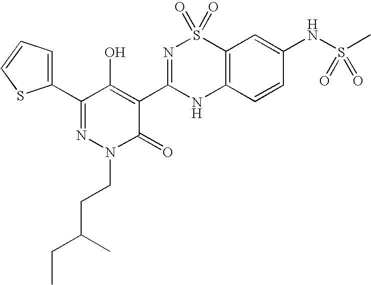

### N-{3-[5-Hydroxy-2-(3-methyl-pentyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 0.86 (t, 3H, J=7.0 Hz), 0.94 (d, 3H, J=6.3 Hz), 1.18-1.25 (m, 1H), 1.35-1.49 (m, 2H), 1.54-1.63 (m, 1H), 1.76-1.85 (m, 1H), 3.08 (s, 3H), 4.17 (t, 2H, J=7.1 Hz), 7.16 (t, 1H, J=4.3 Hz), 7.54 (dd, 1H, J=9.5 Hz, J₂=2.3 Hz), 7.60-7.69 (m, 3H), 7.91 (d, 1H, J=4.0 Hz), 10.22 (s, 1H), 13.97 (s, 1H). LC-MS (ESI⁺): m/e=552.2 [M+H⁺] (100%) (exact mass: 551.10).

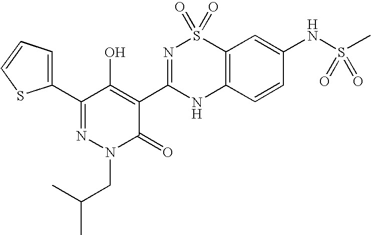

### N-[3-(5-Hydroxy-2-isobutyl-3-oxo-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 0.94 (d, 6H, J=6.8 Hz), 2.22 (septet, 1H, J=6.8 Hz), 3.08 (s, 3H), 3.97 (d, 2H, J=6.8 Hz), 7.15-7.17 (m, 1H), 7.54 (dd, 1H, J=8.5 Hz, J₂=2.3 Hz), 7.60-7.68 (m, 3H), 7.90-7.92 (m, 1H), 10.21 (s, 1H). LC-MS (ESI): m/e=524.26 [M+H] (100%) (exact mass: 523.61).

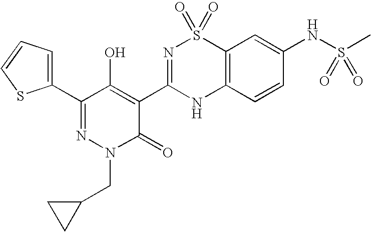

### N-[3-(2-Cyclopropylmethyl-5-hydroxy-3-oxo-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 0.43-0.49 (m, 2H), 0.50-0.56 (m, 2H), 1.25-1.35 (m, 1H), 3.07 (s, 3H), 4.01 (d, 2H, J=7.9 Hz), 7.15-7.17 (m, 1H), 7.54 (dd, 1H, J=8.6 Hz, J₂=2.3 Hz), 7.60-7.62 (m, 2H), 7.68 (d, 1H, J=3.8 Hz), 7.91-7.92 (m, 1H), 10.21 (s, 1H), 13.97 (s, 1H). LC-MS (ESI⁺): m/e=522.33 [M+H⁺] (100%) (exact mass: 521.05).

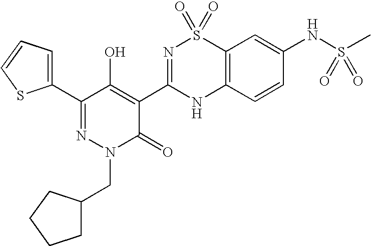

### N-[3-(2-Cyclopentylmethyl-5-hydroxy-3-oxo-6-thiophen-2-yl-2,3-dihydro-pyridazin-4-yl)-1,1-dioxo-1,4-dihydro-1λ⁶-benzo[1,2,4]thiadiazin-7-yl]-methanesulfonamide

¹H NMR (400 MHz, DMSO-d₆) δ: 1.31-1.39 (m, 2H), 1.48-1.57 (m, 2H), 1.58-1.75 (m, 4H), 2.44 (quintet, 1H, J=9.0 Hz), 3.08 (s, 3H), 4.09 (d, 2H, J=6.9 Hz), 7.16-7.18 (m, 1H), 7.55 (dd, 1H, J=8.7 Hz, J₂=2.3 Hz), 7.61-7.69 (m, 3H), 7.91 (d, 1H, J=4.0 Hz), 10.23 (s, 1H), 13.96 (s, 1H). LC-MS (ESI⁺): m/e=550.43 [M+H⁺] (100%) (exact mass: 549.08).

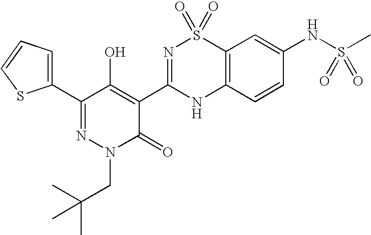

### N-{3-[2-(2,2-Dimethyl-propyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 1.00 (s, 9H), 3.08 (s, 3H), 4.02 (s, 2H), 7.15-7.18 (m, 1H), 7.55 (dd, 1H, J=8.8 Hz, J₂=2.4 Hz), 7.60-7.68 (m, 3H), 7.91 (d, 1H, J=3.9 Hz), 10.22 (s, 1H). LC-MS (ESI⁺): m/e=538.29 [M+H⁺] (100%) (exact mass: 537.08).

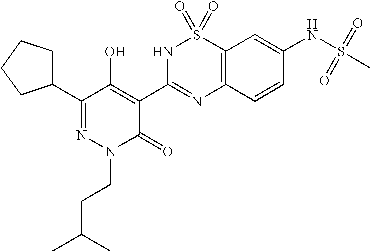

### N-{3-[6-Cyclopentyl-5-hydroxy-2-(3-methyl-butyl)-3-oxo-2,3-dihydro-pyridazin-4-yl]-1,1-dioxo-1,2-dihydro-1-benzo[1,2,4]thiadiazin-7-yl}-methanesulfonamide

¹H NMR (400 MHz, CDCl₃) δ: 1.00 (d, 6H, J=6.3 Hz), 1.60-1.65 (m, 1H), 1.68-1.73 (m, 4H), 1.77-1.82 (m, 4H), 2.00-2.06 (m, 2H), 3.09 (s, 3H), 3.40 (t, 1H, J=7.5 Hz), 4.20 (t, 2H, J=7.5 Hz), 6.67 (s, 1H), 7.33 (d, 1H, J=8.4 Hz), 7.66 (dd, 1H, J₁=8.7 Hz, J₂=2.4 Hz), 7.69 (d, 1H, J=2.3 Hz). LC-MS (ESI⁺): m/e 524.3 [M+1]⁺ (exact ms: 523.16).

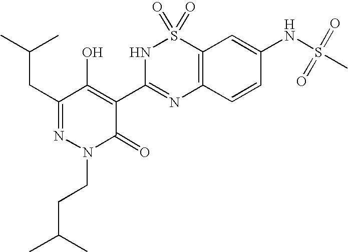

### N-{3-[5-Hydroxy-6-isobutyl-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide

¹H NMR (400 MHz, CDCl₃) δ: 0.98-1.01 (m, 12H), 1.63-1.73 (m, 3H), 2.11-2.18 (m, 1H), 2.63 (d, 2H, J=7.1 Hz), 3.09 (s, 3H), 4.21 (t, 2H, J=7.3 Hz), 6.63 (s, 1H), 7.33 (d, 1H, J=8.7 Hz), 7.66 (dd, 1H, J=8.9 Hz, J₂=2.8 Hz), 7.69 (d, 1H, J=2.3 Hz). LC-MS (ESI⁺): m/e 512.3 [M+1]⁺ (exact ms: 511.16).

### EXAMPLE 2-4

Scheme 2d describes the synthesis of compound 5n of Formula I.

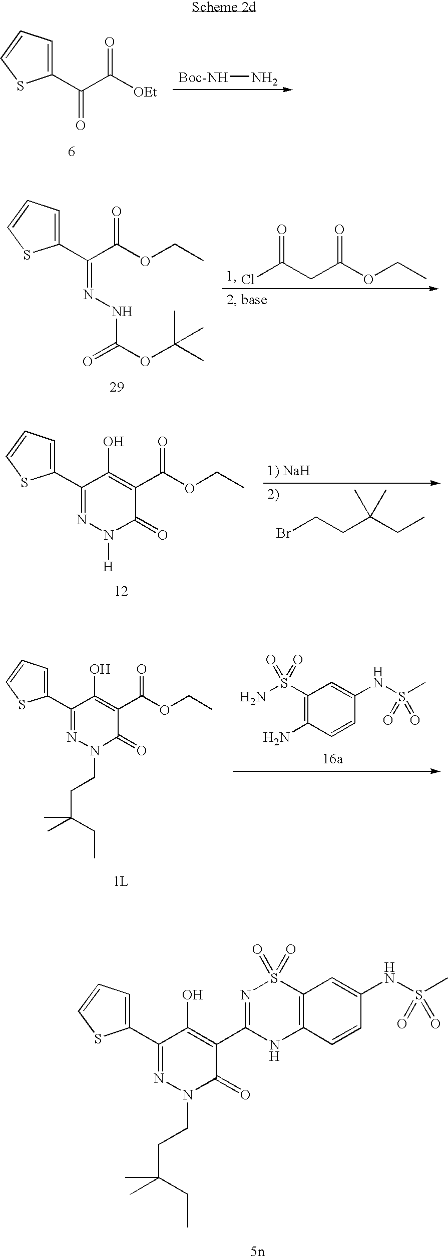

### (tert-Butoxycarbonyl-hydrazono)-thiophen-2-yl-acetic acid ethyl ester (29)

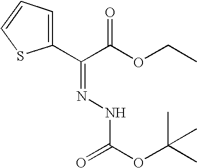

Oxo-thiophen-2-yl-acetic acid ethyl ester (6) (12.2 g, 66.23 mmol, Alfa Aesar) was dissolved in ethyl alcohol (120 mL). Hydrazinecarboxylic acid tert-butyl ester (15.75, 119.21 mmol, Aldrich) was added. The mixture was heated at 90° C. while stirring for 24 hours. The mixture was concentrated in vacuo to a yellow oil. Purification by flash column chromatography (10% ethyl acetate in hexanes, Merck silica gel 60, 40-63 μm) afforded the desired product 29, (tert-butoxycarbonyl-hydrazono)-thiophen-2-yl-acetic acid ethyl ester (13.66 g, 69% yield), as a yellow oil. ¹H NMR (400 MHz, DMSO-d₆) δ: 1.33 (t, 3H, J=7.0 Hz), 1.48 (s, 9H), 4.37 (q, 2H, J=7.2 Hz), 7.06 (dd, 1H, J₁=5.5 Hz, J₂=4.0 Hz), 7.37 (dd, 1H, J₁=4.0 Hz, J₂=1.6 Hz), 7.58 (d, 1H, J=6.3 Hz), 11.01 (s. 1H).

### 5-Hydroxy-3-oxo-6-thiophen-2-yl-2,3-dihydro-pyridazine-4-carboxylic acid ethyl ester (12)

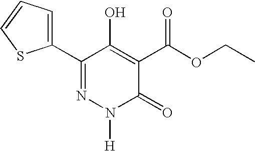

(tert-Butoxycarbonyl-hydrazono)-thiophen-2-yl-acetic acid ethyl ester (29) (13.66 g, 45.79 mmol) was dissolved in anhydrous 1-5-dioxane (230 mL). Chlorocarbonyl-acetic acid ethyl ester (10.34 g, 68.7 mmol) was added. While stirring, the mixture was degassed under vacuum and the flask charged with nitrogen gas. The mixture was heated at 100° C. while stirring for 5 hours. The mixture was poured into ½ saturated aqueous sodium bicarbonate solution (250 mL). The product was extracted into ethyl acetate (2×200 mL). The organic phase was dried over magnesium sulfate and concentrated in vacuo to afford the crude [(2-ethoxycarbonyl-acetyl)-hydrazono]-thiophen-2-yl-acetic acid ethyl ester as an orange oil.

The crude [(2-ethoxycarbonyl-acetyl)-hydrazono]-thiophen-2-yl-acetic acid ethyl ester (˜45 mmol) was dissolved in ethyl alcohol. A 21% solution of sodium ethoxide in ethyl alcohol (28 mL) was added. The mixture stirred at 45° C. for 5 hours. Upon cooling, the mixture was poured into 1M aqueous hydrochloric acid (250 mL). A beige solid formed. The solid was collected by vacuum filtration and rinsed with ethyl alcohol (10 mL) and ethyl acetate (10 mL) to afford the desired product as a beige solid. The filtrate was concentrated in vacuo to a volume of ˜250 mL and diluted with water (100 mL). Additional product was extracted into ethyl acetate (2×250 mL), dried over magnesium sulfate and concentrated to give a brownish orange solid. The solid was triturated with methanol (2×10 mL) and filtered to give the desired product as a beige solid. The solids were combined to afford the desired product 12, 5-hydroxy-3-oxo-6-thiophen-2-yl-2,3-dihydro-pyridazine-4-carboxylic acid ethyl ester (5.4 g, 44.3% yield). ¹H NMR (400 MHz, DMSO-d₆) δ: 1.29 (t, 3H, J=6.9 Hz), 4.30 (q, 2H, J=7.3 Hz), 7.12 (dd, 1H, J=5.2 Hz, J₂=3.5 Hz), 7.62 (d, 1H, J=6.3 Hz), 7.79 (d, 1H, J=3.9 Hz), 13.00 (s, 1H).

### 2-(3,3-Dimethyl-pentyl)-5-hydroxy-3-oxo-6-thiophen-2-yl-2,3-dihydro-pyridazine-4-carboxylic acid ethyl ester (1L)

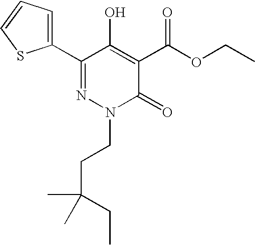

5-hydroxy-3-oxo-6-thiophen-2-yl-2,3-dihydro-pyridazine-4-carboxylic acid ethyl ester (12) (0.266 g, 1 mmol) was suspended in anhydrous N,N-dimethylformamide. A 60% suspension of sodium hydride in mineral oil (0.088 g, 2.2 mmol) was added. The mixture stirred in a sealed vial for 10 minutes with occasional venting. 1-Bromo-3,3-dimethyl-pentane (0.197 g, 1.1 mmol) was added and the mixture stirred at 80° C. for 5 hours. Upon cooling, the mixture was poured into 1 M aqueous hydrochloric acid (50 mL) and the product extracted into ethyl acetate (2×50 mL). The organic phase was dried over magnesium sulfate and concentrated in vacuo to an orange oil. Purification by flash column chromatography (100% methylene chloride, Merck silica gel 60, 40-63 μm) afforded the desired product, 2-(3,3-dimethyl-pentyl)-5-hydroxy-3-oxo-6-thiophen-2-yl-2,3-dihydro-pyridazine-4-carboxylic acid ethyl ester (1L) (0.162 g, 44.4% yield), as a waxy yellow solid. ¹H NMR (400 MHz, CDCl₃) δ: 0.90 (t, 3H, J=7.3 Hz), 0.97 (s, 6H), 1.34 (q, 2H, J=7.5 Hz), 1.50 (t, 3H, J=7.3 Hz), 1.70-1.74 (m, 2H), 4.17-4.21 (m, 2H), 4.54 (q, 2H, J=6.9 Hz), 7.09-7.11 (m, 1H), 7.39 (d, 1H, J=5.4 Hz), 7.89 (d, 1H, J=3.7 Hz), 13.87 (s, 1H).

### N-{3-[2-(3,3-Dimethyl-pentyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide (5n)

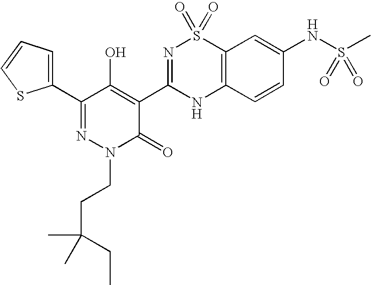

2-(3,3-Dimethyl-pentyl)-5-hydroxy-3-oxo-6-thiophen-2-yl-2,3-dihydro-pyridazine-4-carboxylic acid ethyl ester (1L) (0.11 g, 0.302 mmol) and 2-amino-5-methanesulfonylamino-benzenesulfonamide (16a) (0.08 g, 0.302 mmol) were combined and anhydrous pyridine (1.5 mL) was added. The solution was degassed while stirring under vacuum and the flask charged with argon. The mixture stirred at 110° C. for 16 hours. Upon cooling, the solution was diluted with methanol (8 mL). Upon standing, a yellow precipitate formed. The solid was collected by filtration and rinsed with additional methanol (2 mL). The solid was dried in vacuo to afford the desired product, N-{3-[2-(3,3-dimethyl-pentyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide (5n) (0.0274 g, 16% yield). ¹H NMR (400 MHz, DMSO-d₆) δ: 0.85 (t, 3H, J=7.4 Hz), 0.91 (s, 6H), 1.29 (q, 2H, J=7.4 Hz), 1.56-1.60 (m, 2H), 3.01 (s, 3H), 3.95-3.99 (m, 2H), 7.06 (dd, 1H, J₁=5.5 Hz, J₂=3.9 Hz), 7.33 (d, 1H, J=9.6 Hz), 7.42 (dd, 1H, J₁=9.2 Hz, J₂=2.8 Hz), 7.47-7.49 (m, 2H), 7.87-7.88 (m, 1H), 9.93 (s, 1H); LC-MS (ESI): (exact mass: 565.11) m/z=566.48 [M+H⁺] (100%).

The following compounds of Formula I were also made in an analogous manner to the procedure described in Scheme 2.

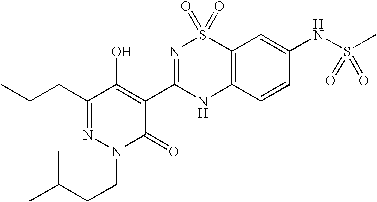

### N-{3-[5-Hydroxy-2-(3-methyl-butyl)-3-oxo-6-propyl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 10.22 (s, 1H), 7.65 (d, 1H, J=9.0 Hz), 7.60 (d, 1H, J=2.3 Hz), 7.55 (dd, 1H, J=9.0, 2.3 Hz), 4.10 (t, 2H, J=7.0 Hz), 3.07 (s, 3H), 2.63 (t, 2H, J=7.4 Hz), 1.70-1.57 (m, 5H), 0.94 (t, 3H, J=7.4 Hz), 0.92 (d, 6H, J=6.2 Hz); LC-MS (ESI⁺): m/e 498.16 [M+H]⁺ (exact ms: 497.14).

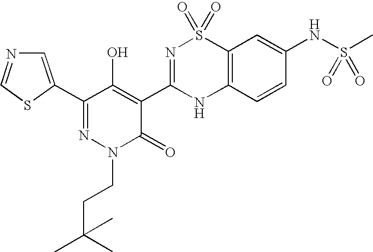

### N-{3-[2-(3,3-Dimethyl-butyl)-5-hydroxy-3-oxo-6-thiazol-5-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.10 (s, 1H), 9.12 (s, 1H), 8.60 (s, 1H), 7.56 (d, 1H, J=2.0 Hz), 7.52 (d, 1H, J=0.8 Hz), 7.50 (d, 1H, J=3.2 Hz), 4.27 (t, 1H, J=8.4 Hz), 3.06 (s, 3H), 1.64 (m, 2H), 0.97 (m, 9H); LC-MS (ESI): m/e 553.30 [M+H]⁺ (exact ms: 552.09).

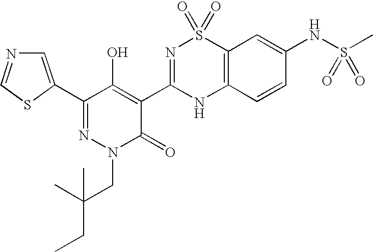

### N-{3-[2-(2,2-Dimethyl-butyl)-5-hydroxy-3-oxo-6-thiazol-5-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 10.10 (s, 1H), 9.10 (s, 1H), 8.59 (s, 1H), 7.56 (d, 1H, J=2.4 Hz), 7.50 (m, 2H), 3.97 (t, 2H, J=6.4 Hz), 3.05 (s, 3H), 1.33 (m, 2H), 0.92 (m, 9H); LC-MS (ESI⁺): m/e 553.30 [M+H]⁺ (exact ms: 552.09).

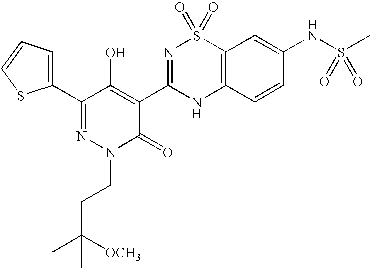

### N-{3-[5-Hydroxy-2-(3-methoxy-3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 1.17 (bs, 6H), 1.23 (bs, 1H), 1.84-1.88 (m, 2H), 1.98 (bs, 1H), 3.01 (bs 3H), 3.16 (bs, 3H), 3.99-4.03 (m, 2H), 7.06-7.07 (m, 1H), 7.34 (d, 1H, J=8.4 Hz), 7.43 (d, 1H, J=8.6 Hz), 7.49 (bs, 2H), 7.88 (bs, 1H), 9.93 (s, 1H); LCMS (ESI): m/e=536.3 [M−OCH₃]⁺.

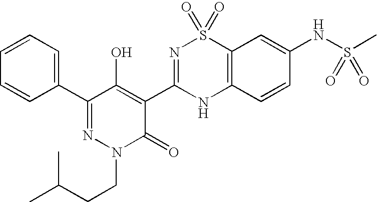

### N-{3-[5-Hydroxy-2-(3-methyl-butyl)-3-oxo-6-phenyl-2,3-dihydro-pyridazin-4-yl]-1,1-dioxo-1,4-dihydro-1λ⁶-benzo[1,2,4]thiadiazin-7-yl}-methanesulfonamide

Yield: 26% (last step). ¹H NMR (400 MHz, CDCl₃) δ: 7.79-7.82 (m, 2H), 7.67-7.71 (m, 2H), 7.48-7.51 (m, 2H), 7.37 (d, 1H, J=8.5 Hz), 6.72 (s, 1H), 4.33 (t, 2H, J=7.4 Hz), 3.10 (s, 3H), 1.68-1.82 (m, 3H), 1.03 (d, 6H, J=7.0 Hz); LC-MS (ESI): m/e=532.27 [M]⁺ (exact ms: 531.12).

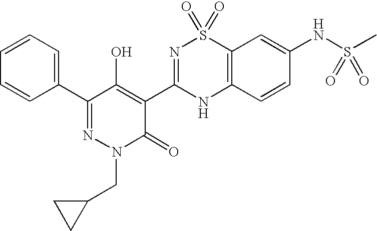

### N-[3-(2-Cyclopropylmethyl-5-hydroxy-3-oxo-6-phenyl-2,3-dihydro-pyridazin-4-yl)-1,1-dioxo-1,4-dihydro-1λ⁶-benzo[1,2,4]thiadiazin-7-yl]-methanesulfonamide

Yield: (last step): 3.3%. ¹H NMR (400 MHz, CDCl₃) δ: 7.80-7.83 (m, 2H), 7.66-7.70 (m, 2H), 7.49-7.50 (m, 2H), 7.36 (d, 1H, J=8.7 Hz), 6.62 (s, 1H), 4.17 (d, 2H, J=7.5 Hz), 3.10 (s, 3H), 1.40-1.47 (m, 1H), 0.64 (quartet, 2H, J=6.2 Hz), 0.52 (quartet, 2H, J=5.3 Hz); LC-MS (ESI⁺): m/e=516.3 [M+H]⁺ (exact ms: 515.09).

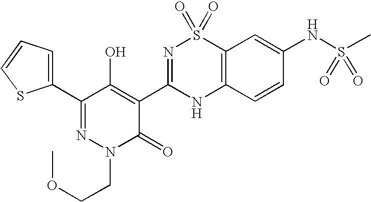

### N-{3-[5-Hydroxy-2-(2-methoxy-ethyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

Yield (last step): 23%. ¹H NMR (400 MHz, DMSO-d₆) δ: 10.15 (s, 1H), 7.91 (d, 1H, J=3.9 Hz), 7.65 (d, 1H, J=5.4 Hz), 7.57-7.58 (m, 1H), 7.53-7.54 (m, 1H), 7.15 (t, 1H, J=4.3 Hz), 4.28 (t, 2H, J=5.3 Hz), 3.74 (t, 2H, J=5.6 Hz), 3.28 (s, 3H), 3.06 (s, 3H); LC-MS (ESI): m/e=526.11 [M+H]⁺ (exact ms: 525.04).

Method 3-1: Scheme 3a provides a general procedure that can be used to prepare compound (5o) of Formula I.

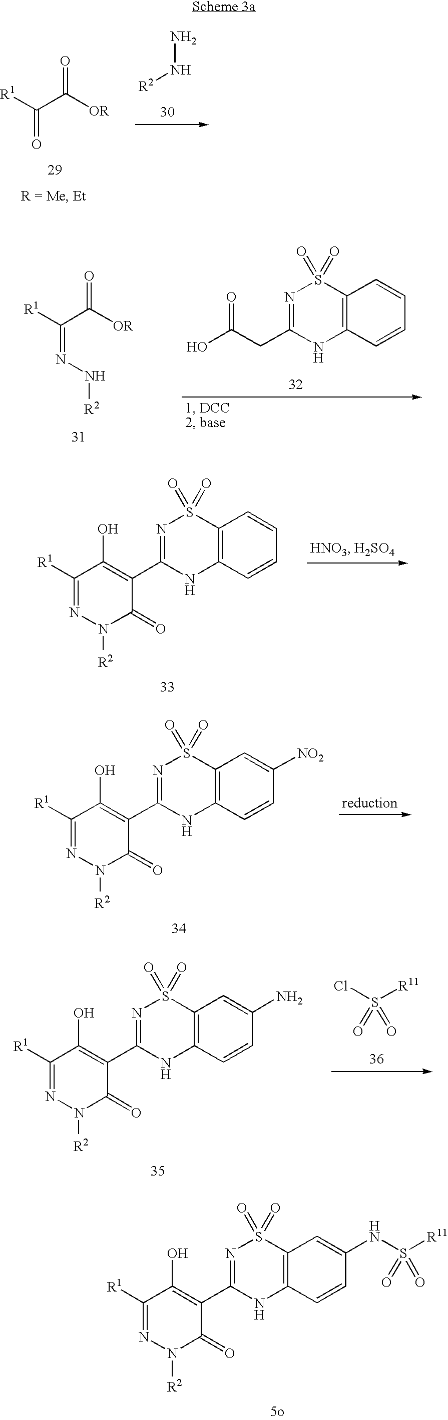

In this general method, compound 3i was made in the same way as that described in Scheme 1a, which can couple with compound 32 (for synthesis see International Publication No. 2006/066079) to make compound 33. Nitration followed by the reduction of the nitro-group affords compound 35, which can be further acylated by alkylsulfonyl chloride (36) to give desired the product (5o).

One example of the synthesis of aniline intermediate 35, wherein R¹═(CH₃)₂CH and R²═(CH₃)₂CHCH₂CH₂— was described International Publication No. 2006/066079 (see example 3-2, Scheme 3b).

The aniline intermediate 35 can be treated with alkanesulfonayl chloride (36) under basic conditions such as pyridine to afford the desired compounds 5o of Formular I.

Method 3-2: Scheme 3b provides a general procedure that can be used to prepare compound (5o) of Formula I.

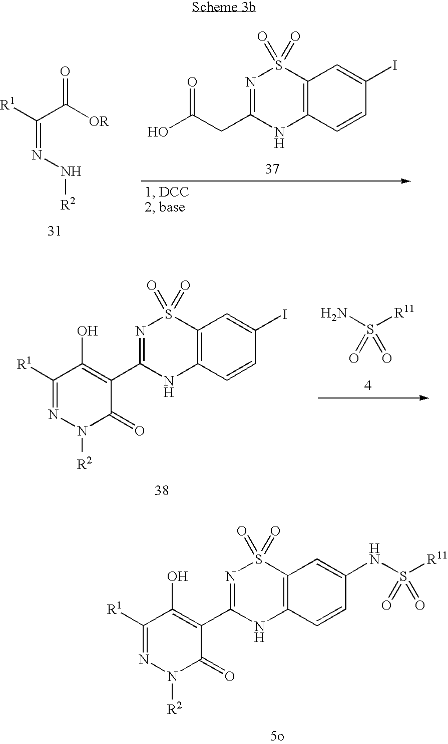

In the general method according to scheme 3b, compound 3i can be coupled with compound 37 using DCC as amide coupling reagent to give compound 38, which can react with alkylsulfonamide in the presence of catalyst to form the final product 5o in a similar manner as that described in Scheme 1a. Compound 37 can be made in a similar fashion as that described in Scheme 19 (steps 3, 5, and 6) when compound 2a is used instead of compound 42a as the starting material.

### EXAMPLE 3-2

Scheme 3c describes a specific method of making compound 5p of Formula I using Method 3-2.

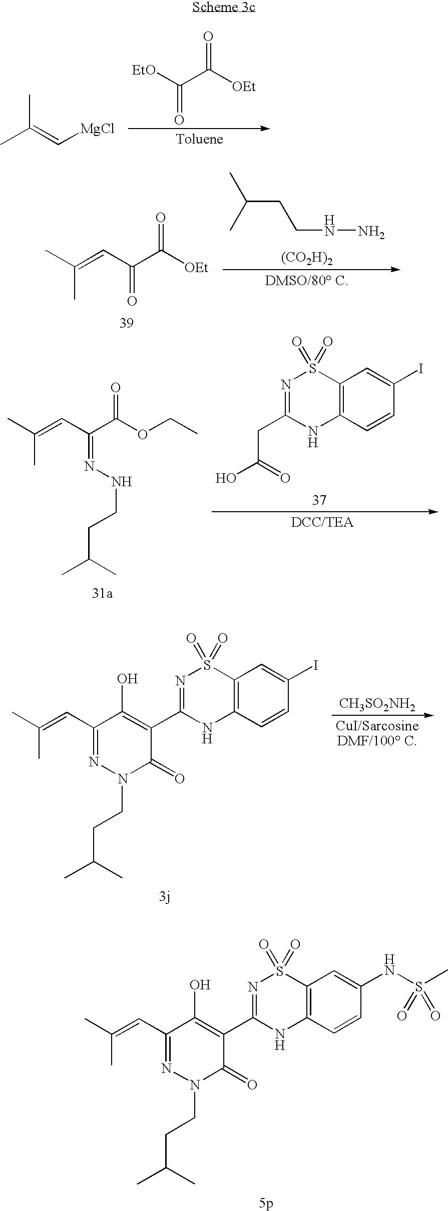

### 4-Methyl-2-oxo-pent-3-enoic acid ethyl ester (39)

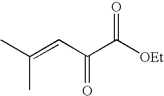

To a stirred solution of diethyl oxalate (3.40 mL, 25 mmol) in 120 mL of dry toluene at −78° C. under N₂ atmosphere, 2-Methyl-1-propenyl magnesium bromide (0.5 M in THF, 50 mL, 50 mmol) was added slowly via addition funnel (15 min). The reaction mixture was stirred at same temperature for 2 h and quenched with aq. NH₄Cl solution. The two layers were separated by separatory funnel, and the organic layer was extracted with EtOAc. The combined organic layers washed with brine and dried over Na₂SO₄. The solid was removed by filter paper and the filtrate was concentrated by rotary evaporator (temperature of water bath was less then 20° C.). The crude residue was then purified by flash chromatography on silica gel to give the desired product 4-Methyl-2-oxo-pent-3-enoic acid ethyl ester (39) (1.55 g, 40%). ¹H NMR (400 MHz, CDCl₃): δ 6.78 (m, 1H), 4.39 (q, 2H, J=7.2 Hz), 2.28 (d, 3H, J=0.8 Hz), 2.05 (d, 3H, J=0.8 Hz), 1.42 (t, 3H, J=7.2 Hz).

### 4-Methyl-2-[(3-methyl-butyl)-hydrazono]-pent-3-enoic acid ethyl ester (31a)

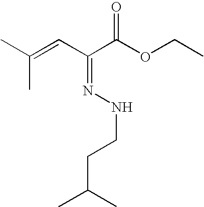

To a stirred solution of 4-Methyl-2-oxo-pent-3-enoic acid ethyl ester (39) (1.40 g, 8.9 mmol) in 25 mL of anhydrous DMSO under N₂ atmosphere, (3-Methyl-butyl)-hydrazine oxalate (1.70 g, 8.9 mmol) was added. The resulting suspension was stirred at 80° C. for 1.5 h. After cooling to rt, the reaction mixture was diluted with EtOAc, washed with H₂O and brine, and dried over Na₂SO₄. The solid was removed by filter paper and the filtrate was concentrated by rotary evaporator. The crude residue was then purified by flash chromatography on silica gel to give one of the two isomers of desired product 4-Methyl-2-[(3-methyl-butyl)-hydrazono]-pent-3-enoic acid ethyl ester (31a) (230 mg, 10%). ¹H NMR (400 MHz, CDCl₃): δ 5.99 (m, 1H), 4.22 (q, 2H, J=7.2 Hz), 3.49 (m, 2H), 1.86 (s, 3H), 1.68 (m, 3H), 1.34 (t, 3H, J=7.2 Hz), 0.94 (d, 6H, J=6.8 Hz).

### 5-Hydroxy-4-(7-iodo-1,1-dioxo-1,4-dihydro-1λ⁶-benzo[1,2,4]thiadiazin-3-yl)-2-(3-methyl-butyl)-6-(2-methyl-propenyl)-2H-pyridazin-3-one (3j)

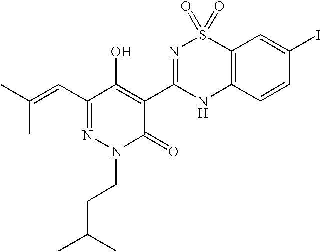

To a solution of 4-Methyl-2-[(3-methyl-butyl)-hydrazono]-pent-3-enoic acid ethyl ester (31a) (79 mg, 0.33 mmol) and (7-Iodo-1,1-dioxo-1,4-dihydro-1l6-benzo[1,2,4]thiadiazin-3-yl)-acetic acid (37) (100 mg, 0.27 mmol) in 0.75 mL of anhydrous DMF and 2.25 mL of anhydrous methylene chloride, 1,3-dicyclohexyl-carbodiimide (DCC) (1.0 M in DCM, 0.33 mL, 0.33 mmol) was added and the resulting mixture was stirred at room temperature for 3 h. Triethylamine (TEA) (0.1 mL, 2 eq.) was added to the reaction mixture and stirred at rt for 1.5 h. The solid was filtered off through a centered funnel and the filtrate was concentrated under reduced pressure. The crude product was then purified by flash chromatography on silica gel to give the desired product 5-Hydroxy-4-(7-iodo-1,1-dioxo-1,4-dihydro-1λ⁶-benzo[1,2,4]thiadiazin-3-yl)-2-(3-methyl-butyl)-6-(2-methyl-propenyl)-2H-pyridazin-3-one (3j) (120 mg, 80%). ¹H NMR (400 MHz, CDCl₃): δ 8.26 (d, 1H, J=1.6 Hz), 7.92 (dd, 1H, J=8.4, 1.6 Hz), 7.06 (d, 1H, J=8.4 Hz), 7.47 (dd, 1H, J=5.2 Hz), 6.32 (m, 1H), 4.24 (t, 2H, J=7.2 Hz), 2.06 (d, 3H, J=1.2 Hz), 2.04 (d, 3H, J=1.2 Hz), 10.73 (m, 3H), 1.00 (d, 6H, J=6.4 Hz); LC-MS (ESI⁺): m/e 543.5 [M+H]⁺ (exact ms: 542.05).

### N-{3-[5-Hydroxy-2-(3-methyl-butyl)-6-(2-methyl-propenyl)-3-oxo-2,3-dihydro-pyridazin-4-yl]-1,1-dioxo-1,4-dihydro-1λ⁶-benzo[1,2,4]thiadiazin-7-yl}-methanesulfonamide (5p)

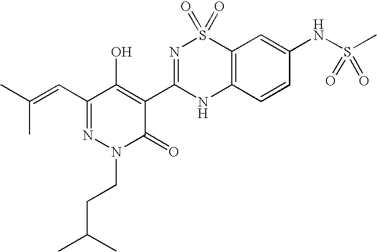

A reaction flask was charged with 5-Hydroxy-4-(7-iodo-1,1-dioxo-1,4-dihydro-1l6-benzo[1,2,4]thiadiazin-3-yl)-2-(3-methyl-butyl)-6-(2-methyl-propenyl)-2H-pyridazin-3-one (3j) (150 mg, 0.277 mmol), CuI (21 mg, 0.11 mmol), sarcosine (N-methyl glycine) (14.8 mg, 0.17 mmol), methanesulfonamide (263 mg, 2.77 mmol), and potassium phosphate (236 mg, 1.11 mmol). Anhydrous DMF (4 mL) was added into the flask. The flask was evacuated and back-filled with nitrogen twice, and then stirred at 100° C. for 6 hours under nitrogen atmosphere. The reaction mixture was cooled to rt, and concentrated under reduced pressure. The crude compound was purified by prep-TLC plate, followed by trituration with methanol twice to give desired product N-{3-[5-Hydroxy-2-(3-methyl-butyl)-6-(2-methyl-propenyl)-3-oxo-2,3-dihydro-pyridazin-4-yl]-1,1-dioxo-1,4-dihydro-1λ⁶-benzo[1,2,4]thiadiazin-7-yl}-methanesulfonamide (5p) as yellow solid (48 mg, 35%). ¹H NMR (400 MHz, DMSO-d₆): δ 10.24 (s, 1H), 7.66 (d, 1H, J=9.2 Hz), 7.60 (d, 1H, J=2.8 Hz), 7.54 (dd, 1H, J=9.2, 2.8 Hz), 6.24 (m, 1H), 4.14 (t, 2H, J=6.8 Hz), 3.08 (s, 3H), 1.98 (d, 3H, J=1.2 Hz), 1.95 (d, 3H, J=1.2 Hz), 1.64 (m, 3H), 0.92 (d, 6H, J=6.8 Hz), LC-MS (ESI⁺): m/e=510.50 [M+H]⁺ (exact ms: 509.14).

The following compounds were made using an analogous method as that described in Scheme 3c.

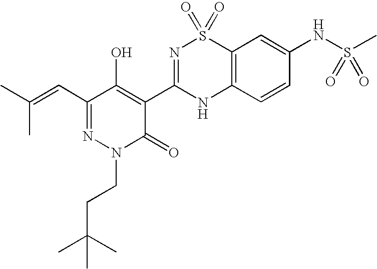

### N-{3-[2-(3,3-Dimethyl-butyl)-5-hydroxy-6-(2-methyl-propenyl)-3-oxo-2,3-dihydro-pyridazin-4-yl]-1-dioxo-1,4-dihydro-1λ⁶-benzo[1,2,4]thiadiazin-7-yl}-methanesulfonamide

¹H NMR (400 MHz, DMSO-d₆): δ 10.24 (s, 1H), 7.67 (d, 1H, J=8.8 Hz), 7.60 (d, 1H, J=2.4 Hz), 7.54 (dd, 1H, J=9.2, 2.4 Hz), 6.24 (m, 1H), 4.14 (m, 2H), 3.08 (s, 3H), 1.98 (d, 3H, J=0.8 Hz), 1.96 (d, 3H, J=0.8 Hz), 1.64 (m, 2H), 0.96 (s, 9H); LC-MS (ESI⁺): m/e=524.3 [M+H]⁺ (exact ms: 523.16).

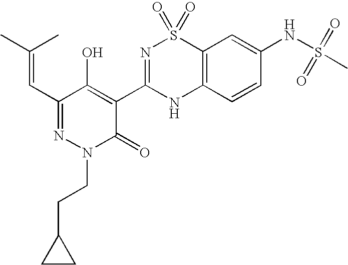

### N-{3-[2-(2-Cyclopropyl-ethyl)-5-hydroxy-6-(2-methyl-propenyl)-3-oxo-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆) δ: 0.00-0.03 (m, 2H), 0.36-0.40 (m, 2H), 0.67-0.72 (m, 1H), 1.65 (quartet, 2H, J=7.0 Hz), 1.94 (bs, 6H), 4.19 (t, 2H, J=7.0 Hz), 6.23 (s, 1H), 7.53 (dd, 1H, J₁=8.7 Hz, J₂=2.4 Hz), 7.59 (d, 1H, J=2.3 Hz), 7.64 (d, 1H, J=9.4 Hz), 10.22 (s, 1H). LC-MS (ESI⁺): m/e 508.3 [M+1]⁺ (exact ms: 507.12).

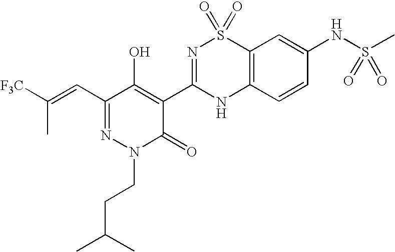

### N-{3-[5-Hydroxy-2-(3-methyl-butyl)-3-oxo-6-(3,3,3-trifluoro-2-methyl-propenyl)-2,3-dihydro-pyridazin-4-yl]-1,1-dioxo-1,4-dihydro-1λ6-benzo[1,2,4]thiadiazin-7-yl}-methanesulfonamide

¹H NMR (400 MHz, DMSO-d₆): δ 10.18 (s, 1H), 7.58 (m, 2H), 7.52 (dd, 1H, J=8.4, 2.4 Hz), 5.86 (s, 1H), 5.54 (s, 1H), 4.07 (t, 2H, J=6.0 Hz), 3.60 (s, 3H), 3.06 (s, 3H), 1.59 (m, 3H), 0.91 (d, 6H, J=6.4 Hz); LC-MS (ESI⁺): m/e=564.5 [M+H]⁺ (exact ms: 563.11).

### N-{3-[6-Cyclopent-1-enylmethyl-5-hydroxy-2-(3-methyl-butyl)-3-oxo-2,3-dihydro-pyridazin-4-yl]-1,1-dioxo-1,4-dihydro-1λ⁶-benzo[1,2,4]thiadiazin-7-yl}-methanesulfonamide

This compound was made from the corresponding iodine intermediate (6-Cyclopent-1-enylmethyl-5-hydroxy-4-(7-iodo-1,1-dioxo-1,4-dihydro-1l6-benzo[1,2,4]thiadiazin-3-yl)-2-(3-methyl-butyl)-2H-pyridazin-3-one) using the same reaction condition as that described in Scheme 1a in the last step.

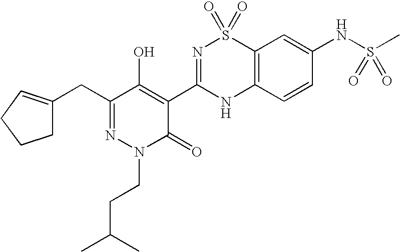

¹H NMR (400 MHz, DMSO-d₆): δ 10.25 (s, 1H), 7.67 (d, 1H, J=8.8 Hz), 7.60 (d, 1H, J=2.0 Hz), 7.55 (dd, 1H, J=8.8, 2.0 Hz), 5.36 (s, 1H), 4.10 (t, 2H, J=6.8 Hz), 3.43 (s, 2H), 3.08 (s, 3H), 2.66 (t, 4H, J=7.2 Hz), 1.82 (m, 2H), 1.61 (m, 3H), 0.91 (d, 6H, J=6.0 Hz); LC-MS (ESI): m/e=535.16 [M+H]⁺ (exact ms: 536.4).

Method 3-3: Scheme 3d provides a general procedure that can be used to prepare compound (5o) of Formula I.

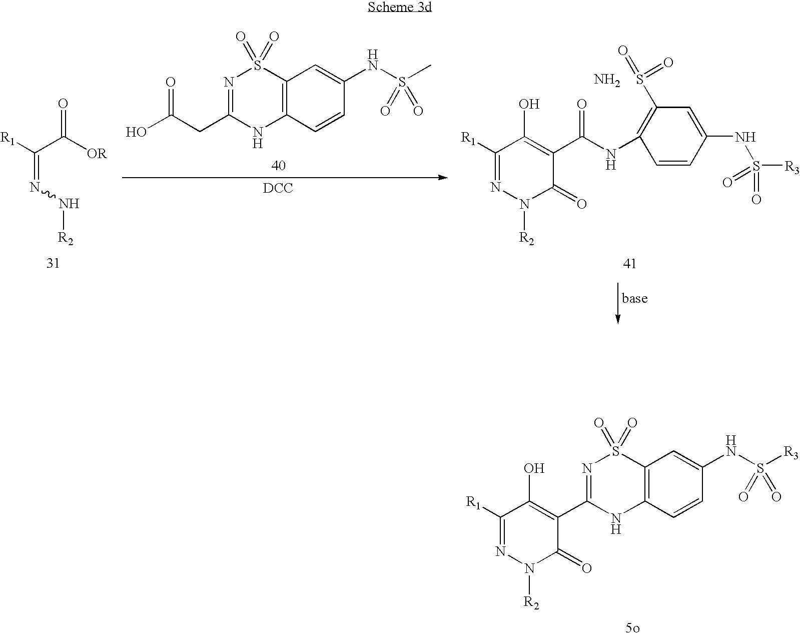

In this general method, compound 31 can be coupled with compound 40 (see Scheme 19) to form the amide intermediate 41 in the presence of DCC as coupling reagent. Compound 41 can be cyclized in the presence of a base to form the desired product (5o) of Formula I.

Method 4: Scheme 4 provides a general procedure that can be used to prepare compound 5 of Formula I.

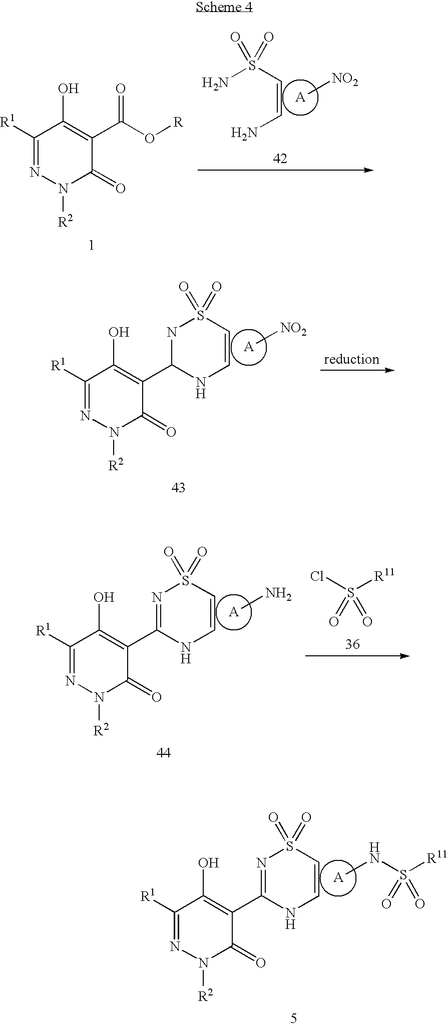

In this general procedure, the compound 1 was prepared according to the procedure described in International Publication No. WO 2006/066079 (see Scheme 4). The compound 1 was condensed with nitro-substituted 2-amino-sulfonamide (42) in the presence of a base such as pyridine at 120° C. to form compound 43 which was subsequently reduced by the Raney Nickel in the presence of hydrazine to the corresponding aniline (44). Compound 44 can be easily converted to the compounds 5 of Formula I by acylation using alkylsulfonyl chloride (36) and this transformation is carried out in a similar manner as that described in Scheme 3a.

### EXAMPLE 4-1

Scheme 4a describes the synthesis of compound 5q.

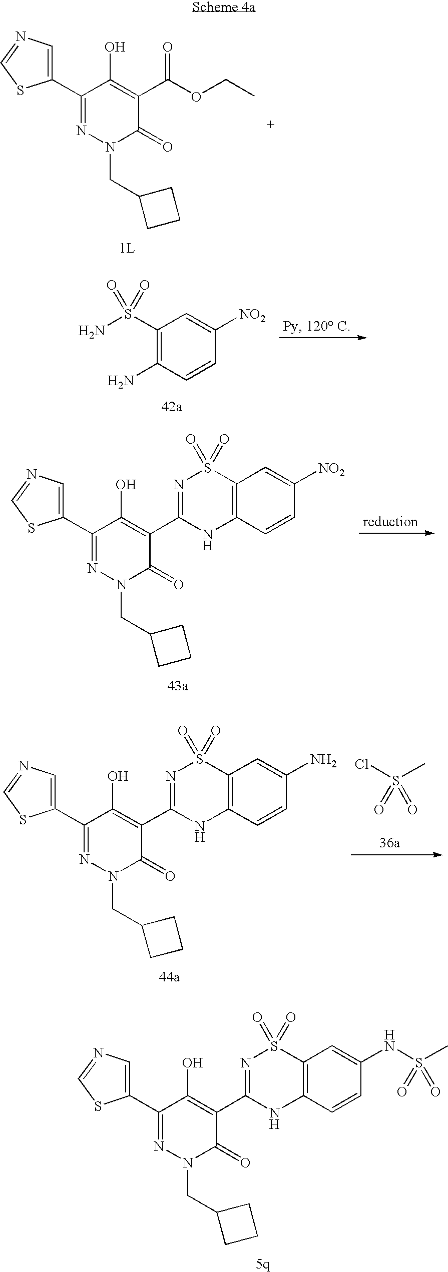

In this specific example, compound 1L (313.6 mg, 0.936 mmol) was mixed with compound 42a (224 mg, 1.03 mmol) (see Scheme 6a) and pyridine (2.8 mL) and the mixture was stirred at 120° C. (oil bath temperature) under N₂ atmosphere for 48 hours. LC-MS spectrum confirmed the existence of the desired product (43a) and the disappearance of the starting material (1L). The reaction mixture was concentrated under reduced pressure to remove the pyridine solvent. The crude compound was purified by flash column chromatography using silica gel eluted with 0-100% ethyl acetate/hexane to give 77.5 mg pure desired product (43a) as yellow solid with 17% isolated yield with 100% of HPLC purity. LC-MS (ESI⁺): m/e 489.8 [M+1]⁺.

The intermediate 43a can be reduced to aniline intermediate 44a based on known methods in the art such as Raney Nickel and hydrazine, and then intermediate 44a can be easily converted to the desired compound 5q by reacting with methylsulfonyl chloride in the presence of a base such as pyridine.

### EXAMPLE 4-2

Scheme 4b describes the synthesis of 2-diethylamino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5t).

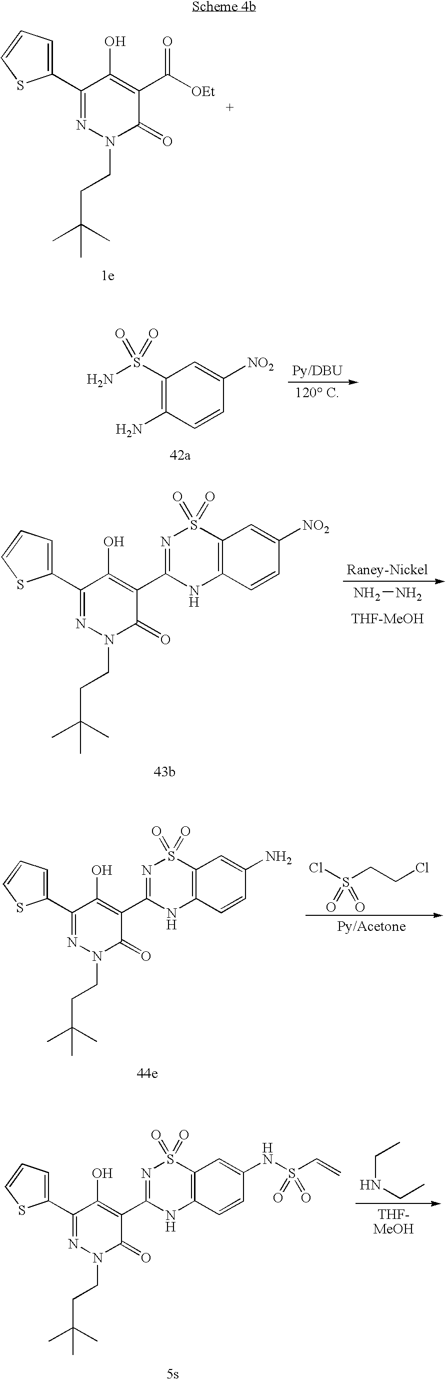

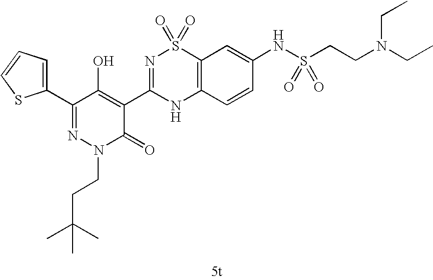

### 2-(3,3-Dimethyl-butyl)-5-hydroxy-4-(7-nitro-1,1-dioxo-1,4-dihydro-1λ⁶-benzo[1,2,4]thiadiazin-3-yl)-6-thiophen-2-yl-2H-pyridazin-3-one (43 b)

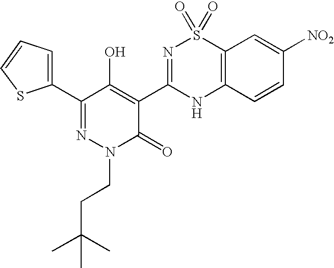

2-(3,3-Dimethyl-butyl)-5-hydroxy-3-oxo-6-thiophen-2-yl-2,3-dihydro-pyridazine-4-carboxylic acid ethyl ester (1e) (1.46 g, 4.17 mmol) and 2-amino-5-nitro-benzenesulfonamide (42a) (1.0 g, 4.61 mmol) were dissolved in anhydrous pyridine (10 mL). DBU (2-4 equivalents) was added and the resulted reaction mixture was stirred at 120° C. for 48 h under a nitrogen atmosphere. The reaction mixture was cooled to 25° C., and concentrated in vacuo. The residue was purified by prep-HPLC to afford the desired product, 2-(3,3-dimethyl-butyl)-5-hydroxy-4-(7-nitro-1,1-dioxo-1,4-dihydro-1λ⁶-benzo[1,2,4]thiadiazin-3-yl)-6-thiophen-2-yl-2H-pyridazin-3-one (400 mg, 20%) as a yellow solid (43b). ¹H NMR (400 MHz, CDCl₃) δ: 8.91 (d, 1H, J=2.4 Hz), 8.51 (dd, 1H, J=9.2, 2.8 Hz), 8.00 (dd, 1H, J=3.6, 1.2 Hz), 7.51 (m, 2H), 7.17 (dd, 1H, J=4.4, 3.6 Hz), 4.32 (m, 2H), 1.79 (m, 2H), 1.07 (s, 9H); LC-MS (ESI⁺: m/e=504.3 [M+H]⁺ (exact ms: 503.20).

### 4-(7-Amino-1,1-dioxo-1,4-dihydro-1λ⁶-benzo[1,2,4]thiadiazin-3-yl)-2-(3,3-dimethyl-butyl)-5-hydroxy-6-thiophen-2-yl-2H-pyridazin-3-one (44e)

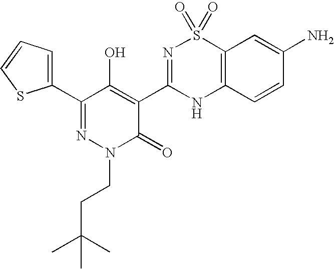

To a stirred solution of 2-(3,3-dimethyl-butyl)-5-hydroxy-4-(7-nitro-1,1-dioxo-1,4-dihydro-1λ⁶-benzo[1,2,4]thiadiazin-3-yl)-6-thiophen-2-yl-2H-pyridazin-3-one (43b) (400 mg, 0.79 mmol) in anhydrous THF-MeOH (1:1, 34 mL), Raney-Nickle catalyst (50% slurry in H₂O, 2.15 mL) and anhydrous hydrazine (1.07 mL) were added sequentially. The resulting mixture was stirred at room temperature for 1 h, passed through a plug of celite and rinsed with 10% MeOH in DCM. The filtrate was concentrated in vacuo to afford the desired product, 4-(7-amino-1,1-dioxo-1,4-dihydro-1λ⁶-benzo[1,2,4]thiadiazin-3-yl)-2-(3,3-dimethyl-butyl)-5-hydroxy-6-thiophen-2-yl-2H-pyridazin-3-one (44e), which was used directly in the next step without purification. LC-MS (ESI⁺): m/e=474.3 [M+H]⁺ (exact ms: 473.20).

### Ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5s)

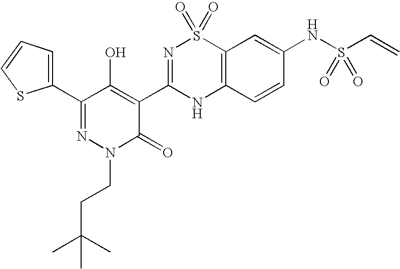

To a stirred solution of 4-(7-amino-1,1-dioxo-1,4-dihydro-1λ⁶-benzo[1,2,4]thiadiazin-3-yl)-2-(3,3-dimethyl-butyl)-5-hydroxy-6-thiophen-2-yl-2H-pyridazin-3-one (44e) (50 mg, 0.11 mmol) in anhydrous acetone (2.5 mL) at 0° C. under a nitrogen atmosphere, pyridine (0.09 mL, 1.1 mmol) and 2-chloro-ethanesulfonyl chloride (90 mg, 0.55 mmol) were added sequentially. The resulting mixture was allowed to warm to 25° C. and stirred for 15 minutes, LC-MS analysis confirmed the completion of the reaction. The mixture was concentrated in vacuo to afford the desired product, ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5s) as a yellow solid, which was used directly in the next step without purification. LC-MS (ESI⁺): m/e=564.6 [M+H]⁺ (exact ms: 563.10).

### 2-Diethylamino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5t)

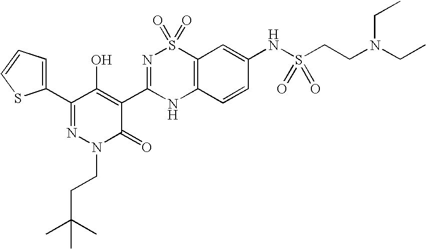

To a stirred solution of the crude compound ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5s) in anhydrous THF/MeOH (1:1, 4 mL) at nitrogen atmosphere, diethyl-amine (0.1 mL, 1.0 mmol) was added slowly. The resulting mixture was stirred at 25° C. for 2 h, LC-MS analysis confirmed the completion of the reaction. The mixture was concentrated in vacuo and the residue was purified by prep-HPLC to afford the desired product, 2-diethylamino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide (5t) (40 mg, 57% yield for two steps) as a yellow solid. ¹H NMR (400 MHz, DMSO-d₆) δ: 10.44 (s, 1H), 9.27 (s, 1H), 7.88 (dd, 1H, J=4.0, 1.2 Hz), 7.58 (s, 1H), 7.55 (d, 1H, J=4.0 Hz), 7.49 (s, 2H), 7.09 (dd, 1H, J=5.2, 4.0 Hz), 4.06 (m, 2H), 3.65 (m, 2H), 3.42 (m, 2H), 3.15 (m, 4H), 1.64 (m, 2H), 1.15 (t, 6H, J=6.8 Hz), 0.96 (s, 9H); LC-MS (ESI): m/e=637.4 [M+H]⁺ (exact ms: 636.19).

Method 5: Scheme 5 provides a general procedure that can be used to prepare compound 5 of Formula I.

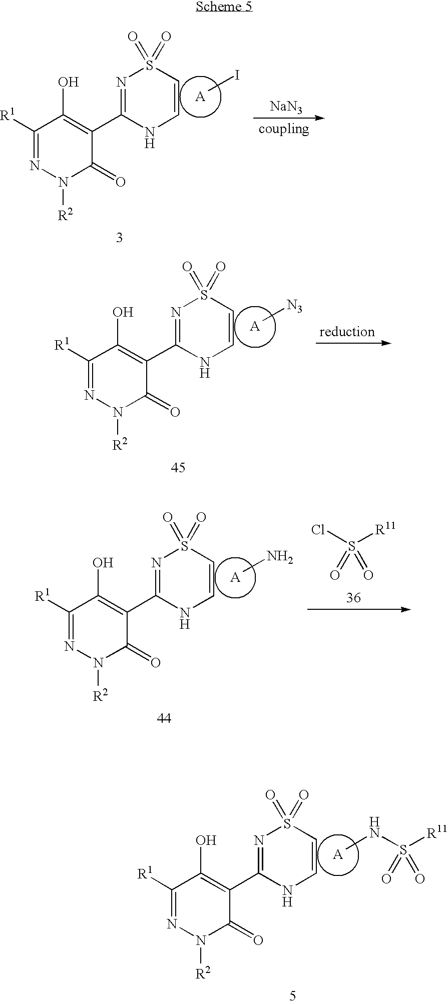

In a general procedure, the iodide-intermediate 3 can be treated with NaN₃ under CuI/amino acid catalyzed coupling conditions to give azide intermediate 45. See, e.g., W. Zhu, et. al., Chem. Commun., 888-889 (2004). Compound 45 can be converted to aniline compound 44 by reduction of the azide functionality based on the known method in the art. Compound 44 can be transformed into the compound 5 using the same method as that described in Scheme 3a.

### EXAMPLE 5-1

Scheme 5a describes the synthesis of compound 5r of Formula I.

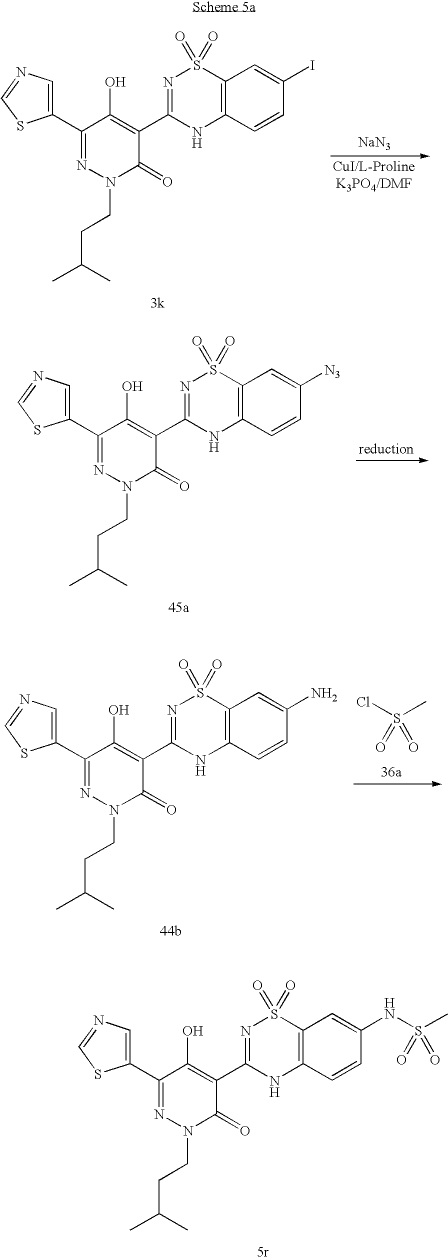

In this specific example, compound 3k (50.0 mg, 0.09 mmol) was mixed with NaN₃ (59 mg, 0.9 mmol), L-proline (4.14 mg, 0.036 mmol), CuI (3.44 mg, 0.018 mmol) and K₃PO₄ (57.3 mg, 0.27 mmol) in anhydrous DMF (2 mL) in a reaction vial, and the mixture was purged with N₂, and then stirred at 60-80° C. (oil bath temperature) for 48 hours. The reaction mixture was cooled to rt and filtered to remove the inorganic salts. The solvent was removed under reduced pressure, and the residue was purified by Prep-HPLC to give pure desired product (45a) (18 mg, 41%) as yellow solid with 100% of HPLC purity. LC-LC-MS (ESI): (exact mass: 486.09) m/z=487.4 [M+H⁺].

The intermediate 45a can be reduced to aniline intermediate 44b based on known methods in the art, and then 44b can be easily converted to the desired compound 5r using a similar transformation as that described in Scheme 3a.

Method 6: Scheme 6 provides a general procedure that can be used to prepare compound 47 of Formula I.

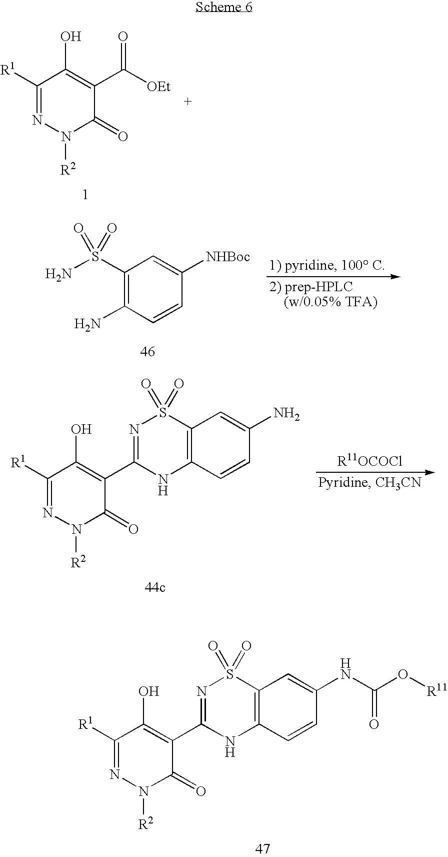

### EXAMPLE 6-1

Scheme 6a describes a specific procedure that was used to synthesize compound 48 and 49 of Formula I.

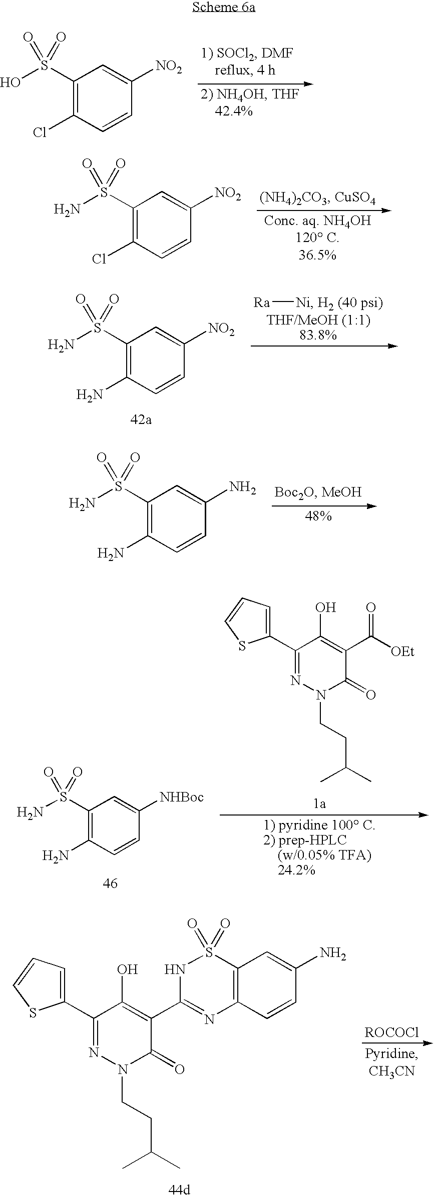

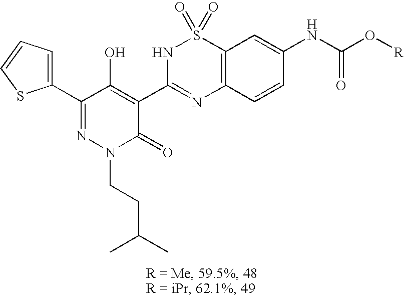

### 2-Chloro-5-nitrobenzenesulfonamide

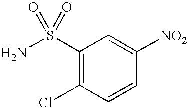

To a solution of thionyl chloride (11 mL) and 2-chloro-5-nitro-benzenesulfonic acid (4.78 g, 20.1 mmol) was added N,N-dimethylformamide (0.92 mL) and the reaction mixture was heated to reflux for 4 h. The reaction mixture was then carefully quenched by pouring it into water and the product was isolated by vacuum filtration. The sulfonyl chloride was dissolved in a minimal amount of toluene and the added to a mixture of concentrated aqueous ammonium hydroxide solution (25 mL) and tetrahydrofuran (25 mL) at −10° C. After stirring for 2 h the reaction was quenched by adding a 6.0 M aqueous hydrochloric acid solution until pH 4 was reached. The layers were separated and the organic layer was concentrated in vacuo to a slurry. Pentane was added and the product was isolated by vacuum filtration to afford 2-chloro-5-nitrobenzenesulfonamide (2.0 g, 42.4%) as a solid.

### 2-Amino-5-nitrobenzenesulfonamide (42a)

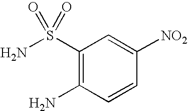

A mixture of 2-chloro-5-nitrobenzenesulfonamide (0.88 g, 3.72 mmol), ammonium carbonate (0.88 g, 9.16 mmol), and copper(II) sulfate (0.175 g, 1.10 mmol) in concentrated aqueous ammonium hydroxide solution (4.4 mL) was heated for 4 h at 120° C. in a pressure reaction vessel. The mixture was allowed to cool to 25° C. and the resulting solid was collected by vacuum filtration, washed with water and dried to afford 2-amino-5-nitrobenzenesulfonamide (42a) (0.295 g, 36.5%) as a tan solid.

### 2,5-Diaminobenzenesulfonamide

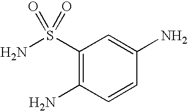

A mixture of 2-amino-5-nitrobenzenesulfonamide (10 g, 46.04 mmol), Raney-Nickel (10 g washed 3 times with 20 mL of tetrahydrofuran) in tetrahydrofuran (150 mL) and methanol (150 mL) was hydrogenated in a Parr shaker for 4 h at 25° C. under H₂ pressure (40 psi). The mixture was then filtered through celite and the solvent removed in vacuo. The solid was washed with methyl tert-butyl ether (40 mL) and dried under high vacuum to afford 2,5-diaminobenzenesulfonamide (7.22 g, 83.8%) as a reddish-brown solid.

### tert-Butyl 4-amino-3-(aminosulfonyl)phenyl carbamate (46)

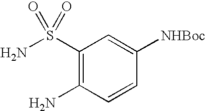

To a solution of 2,5-diaminobenzenesulfonamide (7.22 g, 38.56 mmol) in methanol (50 mL) at 15° C. was added to Boc-anhydride (8.92 g, 40.88 mmol) in methanol (20 mL). The solution was then warmed to 25° C. and mixed for 4 h then the reaction was quenched by addition of N,N-dimethylenediamine (0.63 mL). The solvent was removed under reduced pressure, the solid was redissolved in ethyl acetate and filtered through a fritted funnel and the ethyl acetate was removed under reduced pressure. The crude product recrystallized from ethyl acetate/heptane (1:1) to give tert-butyl 4-amino-3-(aminosulfonyl)phenyl carbamate (46) (5.32 g, 48%).

### 4-(7-Amino-1,1-dioxo-1,2-dihydro-1λ⁶-benzo[1,2,4]thiadiazin-3-yl)-5-hydroxy-2-(3-methyl-butyl)-6-thiophen-2-yl-2H-pyridazin-3-one (44d)

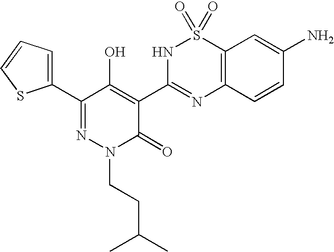

5-Hydroxy-2-(3-methyl-butyl)-3-oxo-6-thiophen-2-yl-2,3-dihydro-pyridazine-4-carboxylic acid ethyl ester (1.0 g, 2.975 mmol) and (4-amino-3-sulfamoyl-phenyl)-carbamic acid tert-butyl ester (854 mg, 2.975 mmol) were dissolved in pyridine (20 mL) and heated to 100° C. for 16 h. The solvent was removed in vacuo and the crude product, {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid tert-butyl ester, was purified by prep-HPLC (containing 0.05% TFA) at which step the Boc protecting group was removed to afford the desired product, 4-(7-amino-1,1-dioxo-1,2-dihydro-1λ⁶-benzo[1,2,4]thiadiazin-3-yl)-5-hydroxy-2-(3-methyl-butyl)-6-thiophen-2-yl-2H-pyridazin-3-one (44d) (330 mg, 24.2%). LC-MS (ESI): m/e=460.2 [M+H]⁺ (exact ms: 459.10)

### {3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid methyl ester (48)

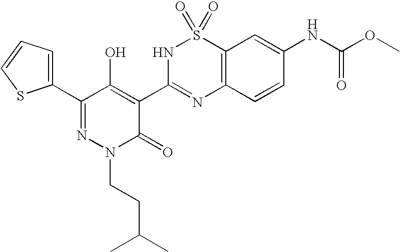

4-(7-Amino-1,1-dioxo-1,2-dihydro-1λ⁶-benzo[1,2,4]thiadiazin-3-yl)-5-hydroxy-2-(3-methyl-butyl)-6-thiophen-2-yl-2H-pyridazin-3-one (44d) (30 mg, 0.065 mmol) was dissolved in anhydrous acetonitrile (5 mL) and N,N-diisopropylethylamine (37.5 μL, 0.215 mmol) followed by pyridine (17.4 μL, 0.215 mmol) were added. Methyl chloroformate (5.04 μL, 0.0715 mmol) was added and the reaction mixture was stirred at 25° C. for 16 h. The reaction mixture was evaporated in vacuo and purified by prep-HPLC to afford the desired product, {3-[5-hydroxy-2-(3-methyl-butyl)-3-oxo-6-thiophen-2-yl-2,3-dihydro-pyridazin-4-yl]-1,1-dioxo-1,2-dihydro-1×6 benzo[1,2,4]thiadiazin-7-yl}-carbamic acid methyl ester (48) (20 mg, 59.5%). ¹H NMR (400 MHz, DMSO-d₆) δ: 0.96 (d, 6H, J=6.2 Hz), 1.60-1.70 (m, 3H), 3.71 (s, 3H), 4.16 (t, 2H, J=6.9 Hz), 7.15-7.17 (m, 1H), 7.56 (d, 11, J=8.5 Hz), 7.66-7.71 (m, 2H), 7.91 (d, 1H, J=4.9 Hz), 8.03 (bs, 1H), 10.10 (s, 1H). LC-MS (EST): m/e=518.28 [M+H]⁺ (exact ms: 517.11)

### {3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid isopropyl ester (49)

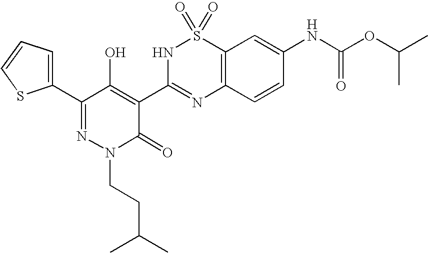

4-(7-Amino-1,1-dioxo-1,2-dihydro-1λ⁶-benzo[1,2,4]thiadiazin-3-yl)-5-hydroxy-2-(3-methyl-butyl)-6-thiophen-2-yl-2H-pyridazin-3-one (44d) (30 mg, 0.065 mmol) was dissolved in anhydrous acetonitrile (5 mL) and N,N-diisopropylethylamine (37.5 μL, 0.215 mmol) followed by pyridine (17.4 μL, 0.215 mmol) were added. A 1.0 M solution of isopropyl chloroformate in toluene (65.3 μL, 0.0715 mmol) was added and the reaction mixture was stirred at 25° C. for 16 h.

The reaction mixture was evaporated in vacuo and purified by prep-HPLC to afford the desired product, {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid isopropyl ester (49) (22 mg, 62.1%). ¹H NMR (400 MHz, DMSO-d₆) δ: 0.96 (6H, d, J=6.2 Hz), 1.29 (6H, d, J=6.2 Hz), 1.60-1.72 (3H, m), 4.17 (2H, t, J=6.5 Hz), 4.89-4.96 (1H, m), 7.17 (1H, t, J=4.6 Hz), 7.58 (1H, d, J=8.6 Hz), 7.68-7.69 (3H, m), 7.91 (1H, d, J=4.0 Hz), 8.09 (1H, bs), 10.05 (1H, s). LC-MS (ESI⁺): m/e=546.47 [M+H]⁺ (exact ms: 545.14)

Method 7: Scheme 7 describes a general procedure that was used for the synthesis of compound 53 and 54 of the Formula I.

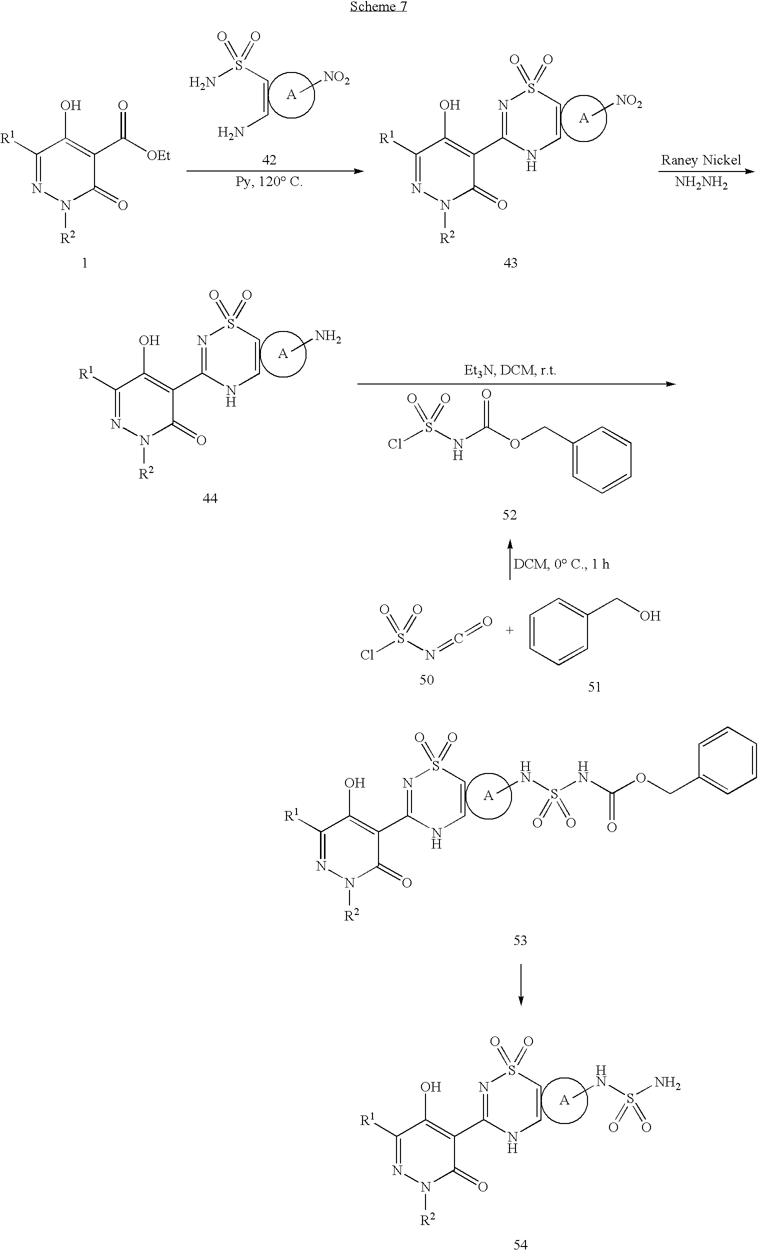

The synthesis of the compound 44 was described in Scheme 4. Chloro-sulfonamide isocyanate (50) was treated with benzyl alcohol (51) in DCM at 0° C. to give compound 52. Aniline 44 was treated with compound 52 in the presence of tri-ethyl amine in DCM at room temperature to give compound 53 that was further reduced by 10% of Pd/C to form compound 54.

### EXAMPLE 7-1

Scheme 7a describes the synthesis of compound 53a and compound 54a of Formula I.

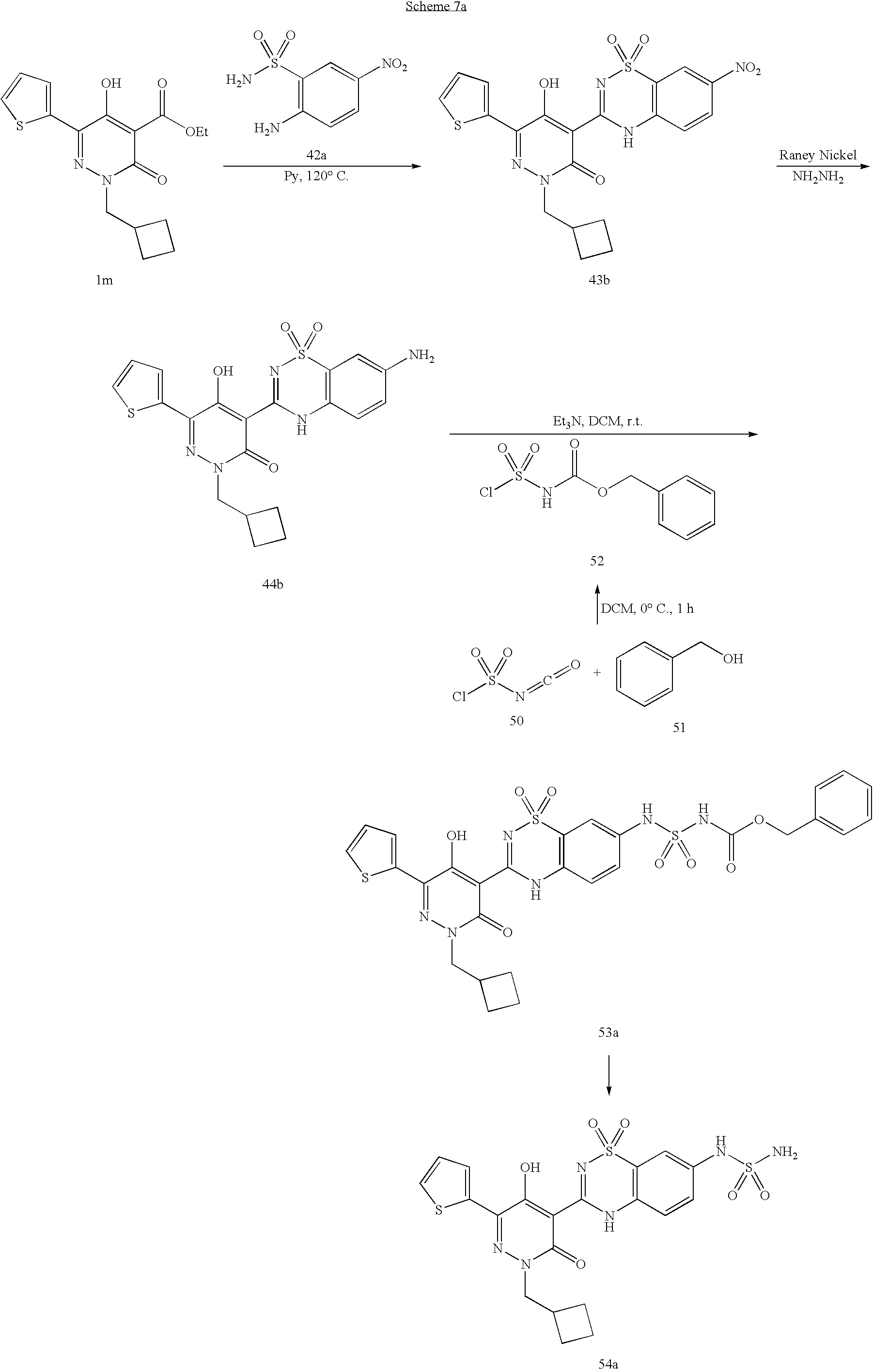

### 2-Cyclobutylmethyl-5-hydroxy-4-(7-nitro-1,1-dioxo-1,4-dihydro-1λ⁶-benzo[1,2,4]thiadiazin-3-yl)-6-thiophen-2-yl-2H-pyridazin-3-one (43b)

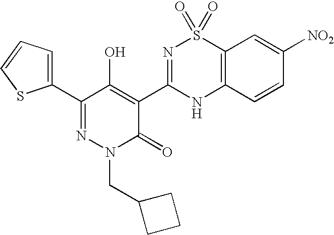

To a solution of compound 1m (313.6 mg) (that was prepared according to the procedure described in International Publication No. WO 2006/066079, Scheme 4) in 2.8 mL of pyridine, aniline (42a) (224 mg) was added. The reaction mixture was heated at 120° C. for 48 hours. The reaction mixture was concentrated under reduced vacuum to remove pyridine solvent at 70° C. The residue was purified by flash chromatography on silica gel (EtOAc and hexane) to give the desired product (43b) (77.5 mg) as yellow solid in 17% isolated yield. LC-MS (ES): m/e=489.8 [M+1]⁺ (exact ms: 488.06).

### 4-(7-Amino-1,1-dioxo-1,4-dihydro-1λ⁶-benzo[1,2,4]thiadiazin-3-yl)-2-cyclobutylmethyl-5-hydroxy-6-thiophen-2-yl-2H-pyridazin-3-one (44b)

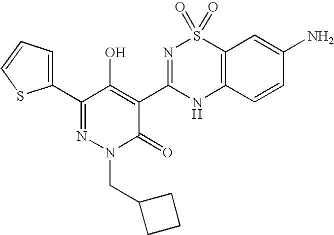

THF (3.5 mL) and MeOH (3.5 mL) were added into compound 43b (77.5 mg) to form a slurry. Raney Nickel (50% slurry in H₂O, 433 uL) was added followed by NH₂NH₂ (213 uL) at room temperature with stirring. The reaction mixture was stirred at room temperature for 4 hours. The completion of the reaction was confirmed by LC-MS spectrum. The reaction mixture was filtered through a plug of Celite, washed with a mixture of MeOH and THF (1:1), concentrated, dried over oil pump overnight to give the desired product (44b) (68.2 mg) as a yellow solid in 93.8% crude yield. LC-MS (ES⁺): m/e=459.8 [M+1]⁺ (exact ms: 458.08).

### Synthesis of the sulfonyl chloride intermediate 52

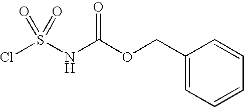

Chloro-sulfonyl isocyanate (50) (19.4 μL) was dissolved in 2 mL of DCM and the resulted solution was cooled down to 0° C. using an ice-water bath. Benzyl alcohol (51) (23 uL) was added and the reaction mixture was stirred at 0° C. for 1 hour to form compound 52.

### Synthesis of the sulfamide carbamet analog (53a)

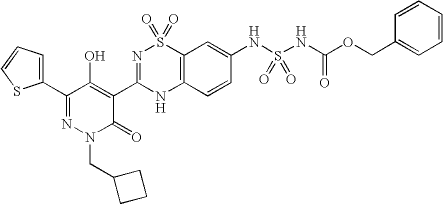

Triethyl amine (neat 31 uL) was added into the above solution of compound 5 at 0° C. followed by a solution of aniline (44b) (68.2 mg) in DCM (1.5 mL) at 0° C. The reaction mixture was then stirred at room temperature for 2 days. LC-MS spectrum confirmed the completion of the reaction. The reaction mixture was centrifuged and then the top clear solution was removed. DCM (2 mL) was added and the mixture was sonicated, centrifuged again and the top clear solution was removed. The remaining solid was dried over the high vacuum overnight to give the desired product (53a) (72 mg) in 72% yield. LC-MS (ESI⁺): m/e=672.38 [M+1]⁺ (exact ms: 671.09). ¹H NMR (DMSO-d₆): δ 11.99 (s, br, 1H), 10.88 (s, br, 1H), 7.54 (s, br, 1H), 7.36-7.43 (m, 2H), 7.24-7.32 (m, 7H), 5.10 (s, 2H), 4.07-4.13 (m, 2H), 2.88 (m, 1H), 1.95-2.05 (m, 2H), 1.80-1.91 (m, 4H).

### Synthesis of the sulfamide analog (54a)

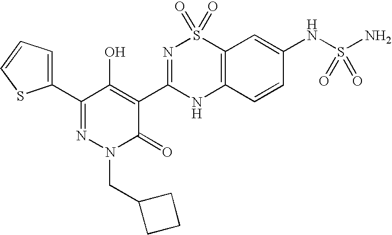

Compound 53a (27.7 mg) was dissolved in a mixture of MeOH (0.2 mL), EtOAc (0.2 mL), DMF (3 mL) and acetic acid (1 mL) to form close to a clear solution. The solution was degassed and Pd/C (10%, 50% wet with water) (20 mg) was added under N₂ atmosphere. A hydrogen balloon was then attached to the reaction vial. The reaction mixture was stirred at room temperature for 24 hours. LC-MS spectrum showed that there was only trace amount of the desired product formed and the majority of the starting material was remained. Additional of Pd/C (10%, dry, 50 mg) was added and the reaction mixture was stirred at room temperature for 24 hours. LC-MS spectrum indicated only about 40% of the desired product formed with 60% of the starting material remaining. Additional Pd/C (10%, dry, 50 mg) was added and the reaction mixture was stirred at room temperature for 3 days. LC-MS spectrum indicated about 60% of the desired product formed with 40% of the starting material still remaining.

The reaction was stopped at this point and the reaction mixture was filtered through a small plug of the Celite, washed with DMF (2 mL×2) and concentrated. The crude was purified by reverse phase HPLC (ACN and H₂O) to give 0.77 mg of the desired product (54a) and 0.65 mg of the recovered starting material. LC-MS (ESI⁺): m/e=538.0 [M+1]⁺ (exact ms: 537.06).

The following compound was made in an analogous manner to the procedure described in Scheme 7a except for the use of an appropriate starting material.

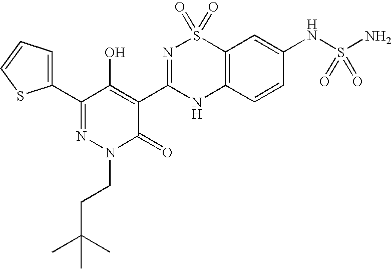

LC-MS (ESI⁺): m/e=553.5 [M+1]⁺ (exact ms: 552.09). ¹H NMR (DMSO-d₆): δ 9.94 (s, 1H), 7.90 (1H, dd, J₁=4 Hz, J₂=1.2 Hz), 7.64 (d, 1H, J=4.4 Hz), 7.54-7.58 (m, 2H), 7.45 (dd, 1H, J₁=9.2 Hz, J₂=2 Hz), 7.32 (s, br, 2H), 7.15 (dd, 1H, J₁=5.2 Hz, J₂=4 Hz), 4.12-4.16 (m, 2H), 1.65-1.69 (m, 2H), 0.97 (s, 9H).

Method 8: Scheme 8 describes a general procedure that can be used to prepare the alkylated sulfamide 56 of the Formula I.

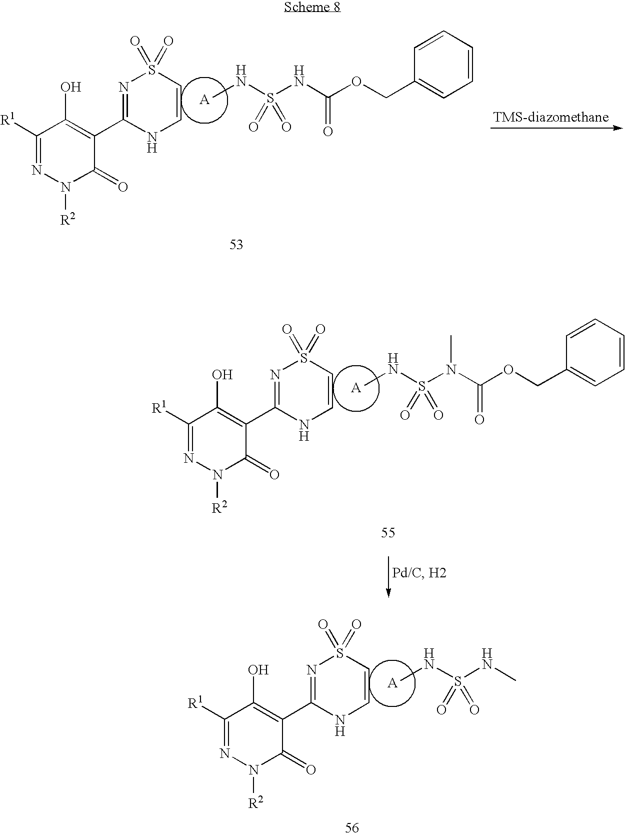

In this general procedure, compound 53 can be methylated by treating with TMS-diazomethane to form compound 55 which can be reduced by 10% of Pd/C in the presence of H₂ to give compound 56 of Formula I. The synthesis for both steps can be referred to the procedure described in a publication by A. Chris Krueger et al., Bioorg. Med. Chem. Lett., 16, 3367-70 (2006).

Method 9: Scheme 9 describes a general procedure that can be used to prepare compound 59 and 60 of the Formula I.

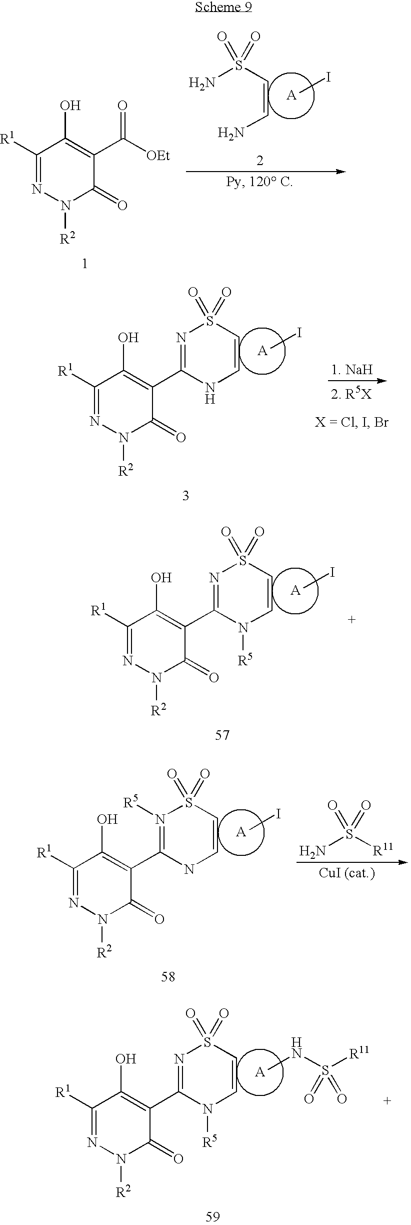

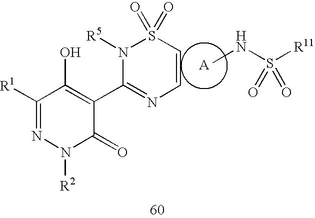

In this general procedure, compound 1 was condensed with compound 2 in pyridine solvent at 120° C. to form compound 3 which can be further alkylated to form compound 57 and/or compound 58. Treatment of compound 57 and/or 58 with sulfonamide in the presence of the catalytic amount of CuI can give the desired product of 59 and/or 60.

### EXAMPLE 9-1

Scheme 9a describes the synthesis of compound 59a of Formula I.

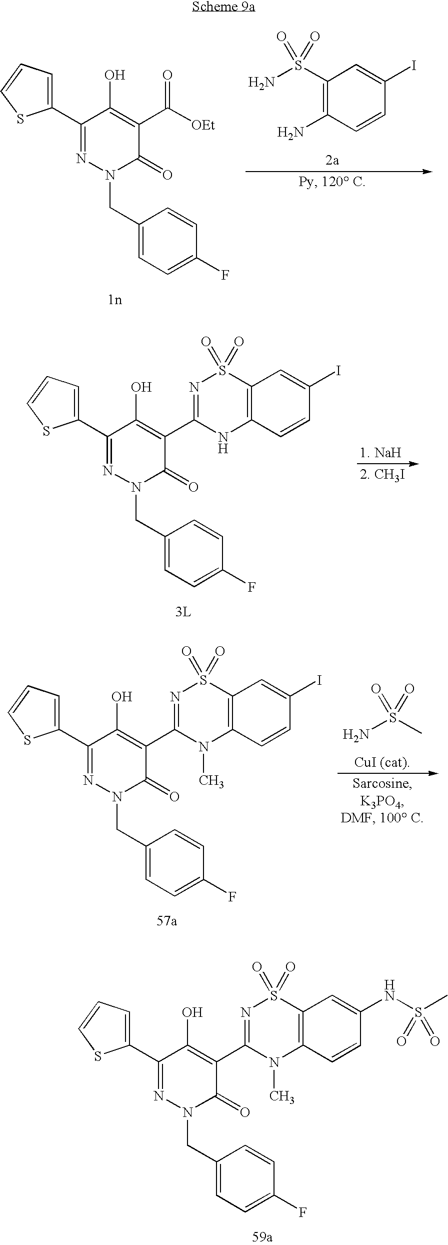

### 2-(4-Fluoro-benzyl)-5-hydroxy-4-(7-iodo-1,1-dioxo-1,4-dihydro-1λ⁶-benzo[1,2,4]thiadiazin-3-yl)-6-thiophen-2-yl-2H-pyridazin-3-one (3L)

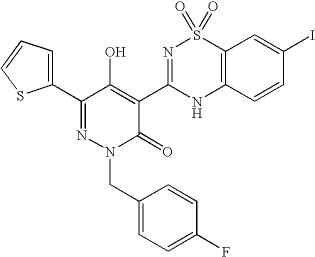

Compound 1n (782.9 mg), compound 2a (685.3 mg) and pyridine (6 mL) were mixed together. The mixture was heated at 120° C. under N₂ with stirring for 22 hours. The reaction mixture was concentrated under reduced pressure at 70° C. to remove the pyridine solvent. The residue was purified by flash chromatography on silica gel (MeOH in DCM) to give a solid as desired product (3L) with 90% HPLC purity. MeOH (4 mL) was added into this solid, sonicated and centrifuged. The top clear solution was removed. The remaining solid was dried over high vacuum overnight to give the pure desired product (3L) (109.4 mg) in 12.3% isolated yield. LC-MS (ES⁺): m/e=609.2 [M+1]⁺ (exact ms: 607.95).

### 2-(4-Fluoro-benzyl)-5-hydroxy-4-(7-iodo-4-methyl-1,1-dioxo-1,4-dihydro-1λ⁶-benzo[1,2,4]thiadiazin-3-yl)-6-thiophen-2-yl-2H-pyridazin-3-one (57a)

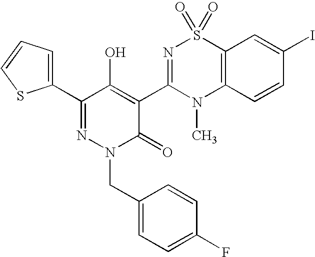

Compound 3L (45.7 mg) was dissolved in 750 mL of DMF. NaH (60% in mineral oil) (9 mg) was added and the resulted mixture was stirred at 45° C. for 1 hour. MeI (96 μL) was added and the reaction mixture was stirred at 45° C. overnight. LC-MS spectrum confirmed the completion of the reaction. EtOAc (6 mL) and 2 mL of H₂O was added and the product was extracted into EtOAc layer which was further washed with saturated NH₄Cl in water by extraction. The organic layer was concentrated under reduced pressure to give the crude product. The residue was purified by flash chromatography on silica gel (MeOH in DCM) to give the desired product (57a, 29.7 mg) as a yellow solid in 63.6% isolated yield. LC-MS (ES⁺): m/e=623.3 [M+1]⁺ (exact ms: 622). Multiple batches of the above same reaction were performed and the isolated yield was ranging from 46% to 64% depending on the reaction scale and the method of the purification was used. ¹H NMR (DMSO-d₆): δ 8.11 (s, br, 1H), 8.09 (d, 1H, J=2 Hz), 7.85 (d, 1H, J₁=3.6 Hz, J₂=0.8 Hz), 7.62 (d, 1H, J=4.8 Hz), 7.45 (d, 1H, J=9.2 Hz), 7.39 (dd, 2H, J=9.2 Hz. J₂=5.6 Hz), 7.17 (d, 1H, J=9.2 Hz), 7.15 (d, 1H, J=8.8 Hz), 7.12 (dd, 1H, J=5.2 Hz, J₂=4 Hz), 5.28 (d, 1H, J=14.4 Hz), 5.16 (d, 1H, J=14 Hz), 3.50 (s, 3H). A proton NMR NOE study shows that the methyl group has NOE interaction with the proton on the phenyl ring that is at the meto-position of iodo. This NOE experiment confirmed the position of the methyl group as that shown in the structure in Scheme 9a.

### N-{3-[2-(4-Fluoro-benzyl)-5-hydroxy-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-methanesulfonamide (59a)

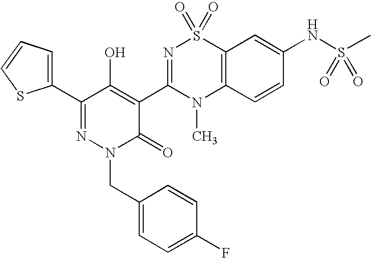

Compound 57a (35.3 mg), methylsulfonamide (11 mg), sarcosine (2 mg), CuI (2.2 mg), K₃PO₄ (36 mg) and DMF (650 μL) were mixed. The mixture was degassed and stirred at 100° C. under N₂ for 24 hours. The reaction was cooled and the mixture was filtered through a small plug of the celite under reduced pressure and washed with 10% MeOH in DCM. The filtrate was concentrated and the residue was purified by HPLC (CH₃CN/H₂O) to give the desired product (59a) (18.75 mg) as an off-white solid. LC-MS (ES): m/e=590.1 [M+1]⁺ (exact ms: 589.06). ¹H NMR (DMSO-d₆, ppm): δ 10.19 (s, br, 1H), 7.85 (dd, 1H, J=3.6 Hz, J₂=1.2 Hz), 7.57-7.67 (m, 4H), 7.35-7.41 (m, 2H), 7.09-7.19 (m, 3H), 5.27 (d, 1H, J=14.4 Hz), 5.15 (d, 1H, J=14.4 Hz), 3.50 (s, 3H), 3.06 (s, 3H).

Method 10: Scheme 10 describes a general method that was used to prepare compound 62 and 63 of the Formula I.

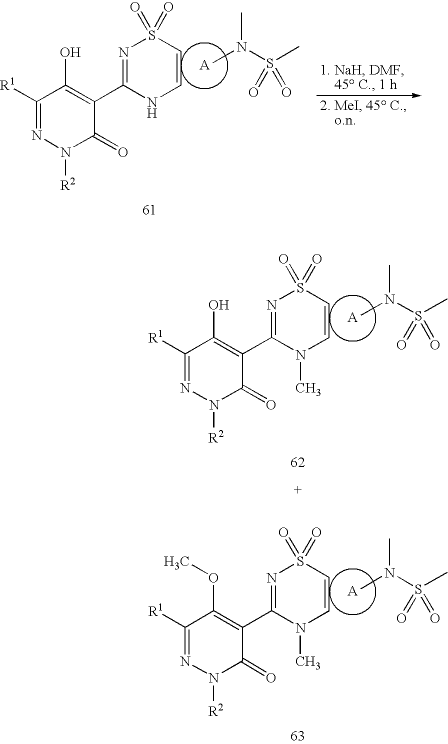

In this general method, compound 61 was alkylated at both N-position and O-position on the ring by using NaH as the base and MeI as the alkylating agent to form compound 62 and 63.

### EXAMPLE 10-1

Scheme 10a describes the synthesis of the compound 62a and 63a of Formula I.

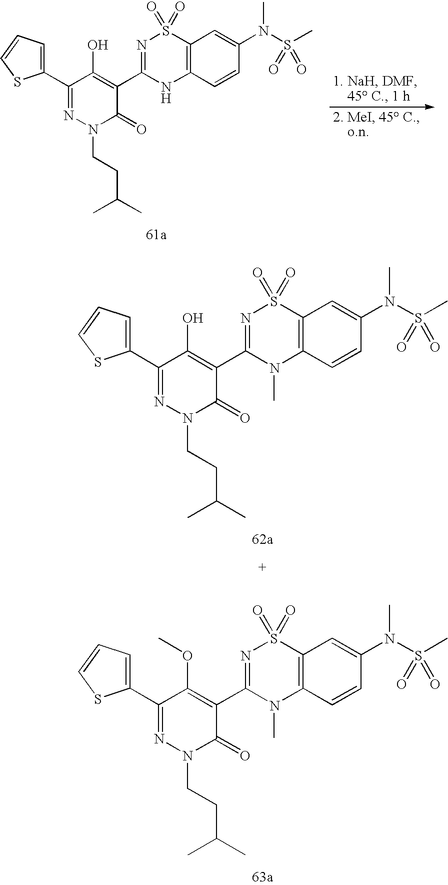

In this specific synthesis, compound 61a (150 mg, 0.27 mmol) was dissolved in 6 mL of DMF with help of the slight heating. NaH (60% mineral oil) (32.4 mg, 0.81 mmol) was added and the mixture was stirred at 45° C. for 1 hour. MeI (86 uL, 5 equivalents) was added and the reaction mixture was stirred at 45° C. overnight. LC-MS result showed both mon- and bis-alkylation products. After extraction work up [EtOAc (20 mL×2)/H₂O (3 mL, Brine 4 mL)], the combined organic layer was dried over Na₂SO₄, filtered and concentrated. The residue was purified by reverse-phase HPLC (ACN/H₂O with 0.05% TFA) to give the compound 62a (73.4 mg) in 48% yield and compound 63a (31.7 mg) in 20.3% yield. The analytical data for both compounds are listed below.

For compound 62a: LC-MS (ES⁺): m/e=566.4 [M+1]⁺ (exact ms: 565.11). ¹H NMR (DMSO-d₆): δ 7.90 (d, 1H, J=2.8 Hz), 7.85 (dd, 1H, J1=3.6 Hz, J2=1.2 Hz), 7.8 (dd, 1H, 9.2 Hz, J2=2.8 Hz), 7.68 (d, 1H, J=9.2 Hz), 7.62 (dd, 1H, J1=5.2 Hz, J2=1.2 Hz), 7.13 (d, 1H, J1=5.2 Hz, J2=3.6 Hz), 4.10-4.18 (m, 1H), 3.99-4.07 (m, 1H), 3.53 (s, 3H), 3.33 (s, 3H), 3.02 (s, 3H), 1.56-1.69 (m, 3H), 0.92-0.95 (m, 6H). The NMR NOE spectra confirmed the alkylation occurred at N-position on the ring.

For compound 63a: LC-MS (ES⁺): m/e=580.2 [M+1]⁺ (exact ms: 579.13). ¹H NMR (DMSO-d₆): δ 7.94 (d, 1H, J=2.8 Hz), 7.87 (dd, 1H, J1=8.8 Hz, J2=2.4 Hz), 7.80 (d, 1H, J=9.2 Hz), 7.75 (dd, 1H, J1=3.6 Hz, J2=1.2 Hz), 7.69 (dd, 1H, J1=5.2 Hz, J2=1.2 Hz), 7.16 (dd, 1H, J1=4.8 Hz, J2=3.6 Hz), 4.085 (s, 3H), 4.03-4.17 (m, 2H), 3.74 (s, 3H), 3.34 (s, 3H), 3.03 (s, 3H), 1.54-1.69 (m, 3H), 0.91-0.93 (m, 6H).

Method 11: Scheme 11 describes another method that was used to prepare compound 65 of the Formula I where R¹⁰ is not H.

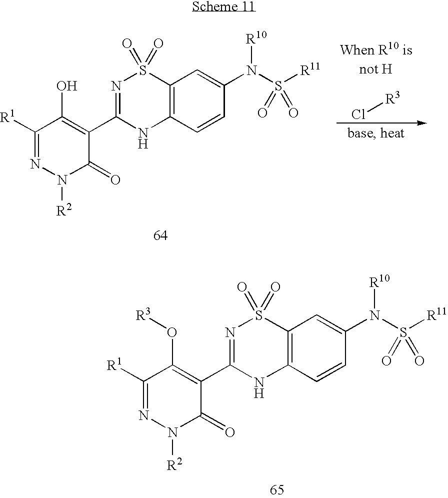

In this general procedure, the enol group of the compound 64 (when R¹⁰ is not H) can be alkylated in the presence of base in the heating condition to form compound 65.

### EXAMPLE 11-1

Scheme 11a describes the synthesis of compound 65a of Formula I.

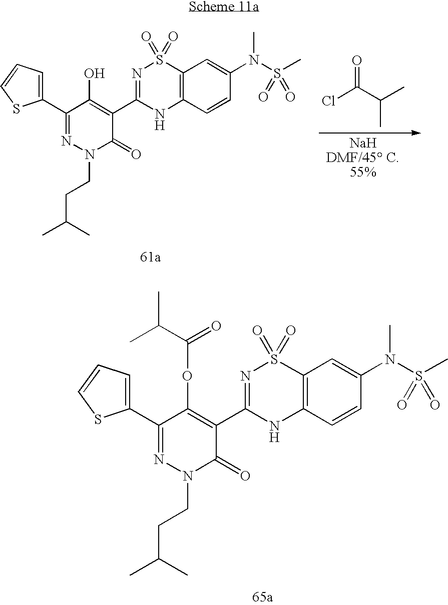

N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-methyl-methanesulfonamide (61a) (30 mg, 0.054 mmol) was dissolved in anhydrous DMF (2 mL) under N₂ atmosphere, NaH (Aldrich) (60% in mineral oil, 8.7 mg, 0.217 mmol) was added. The mixture was stirred at r.t. for 5 minutes and then at 45° C. for 15 minutes, isobutyryl chloride (Aldrich) (0.29 mL, 2.7 mmol) was added and the resulting mixture was stirred at 45° C. for 1 hour. LC-MS showed the desired product was the major peak and small amount of starting material was left.

The mixture was quenched with MeOH and concentrated to give crude product. The crude compound was purified by prep-TLC plate with 5% MeOH/DCM as solvent to give desired product, isobutyric acid 5-[7-(methanesulfonyl-methyl-amino)-1,1-dioxo-1,4-dihydro-1λ⁶-benzo[1,2,4]thiadiazin-3-yl]-1-(3-methyl-butyl)-6-oxo-3-thiophen-2-yl-1,6-dihydro-pyridazin-4-yl ester (65a) (18 mg, 55%) as a yellow solid. ¹H NMR (400 MHz, DMSO-d₆): δ 12.80 (s, 1H), 7.87 (d, 1H, J=4.0 Hz), 7.75 (m, 2H), 7.56 (dd, 1H, J=3.6, 0.8 Hz), 7.43 (d, 1H, J=8.8 Hz), 7.18 (dd, 1H, J=5.2, 3.6 Hz), 4.22 (t, 2H, J=6.8 Hz), 3.30 (s, 3H), 3.01 (s, 3H), 2.90 (m, 1H), 1.70 (m, 3H), 1.10 (d, 6H, J=7.2 Hz), 0.95 (d, 6H, J=6.0 Hz); LC-MS (ESI⁺): m/e=622.30 [M+1]⁺ (exact ms: 621.14).

### EXAMPLE 11-2

Scheme 11b describes the synthesis of compound 65b of Formula I.

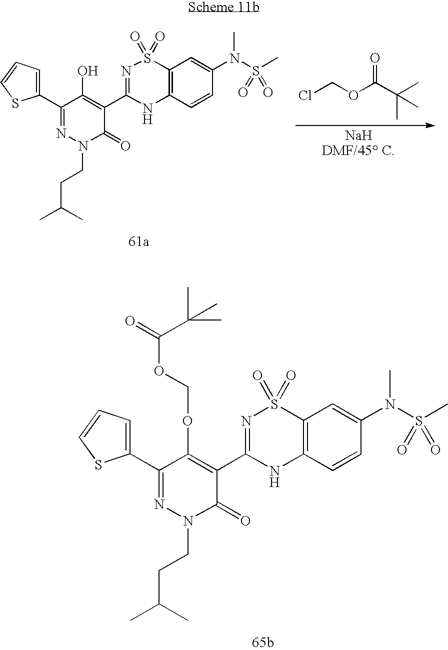

The synthetic procedure was the same as that described in Scheme 11a except the reagent was different and the analytical data for the final product (65b) is listed below. ¹H NMR (400 MHz, DMSO-d₆): δ 7.92 (d, 1H, J=2.0 Hz), 7.75 (dd, 1H, J=8.4, 2.4 Hz), 7.69 (dd, 1H, J=5.2, 1.2 Hz), 7.64 (dd, 1H, J=3.6, 1.2 Hz), 7.44 (d, 1H, J=8.8 Hz), 7.13 (dd, 1H, J=5.2, 4.0 Hz), 5.75 (s, 2H), 4.16 (t, 2H, J=6.8 Hz), 3.30 (s, 3H), 3.01 (s, 3H), 1.67 (m, 3H), 0.96 (s, 9H), 0.94 (d, 9H, J=6.4 Hz); LC-MS (ESI): m/e=667.10 [M+1]⁺ (exact ms: 665.16).

Method 12: Scheme 12 describes a general method that was used to make the compound 66 of Formula I.

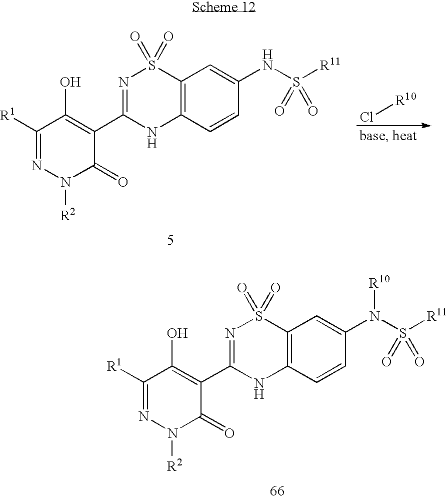

In this general procedure, the sulfonamide nitrogen of compound 5 can be alkylated in the presence of a base under heating condition to give compound 66.

### EXAMPLE 12-1

Scheme 12a descries the synthesis of compound 66a of the Formula I.

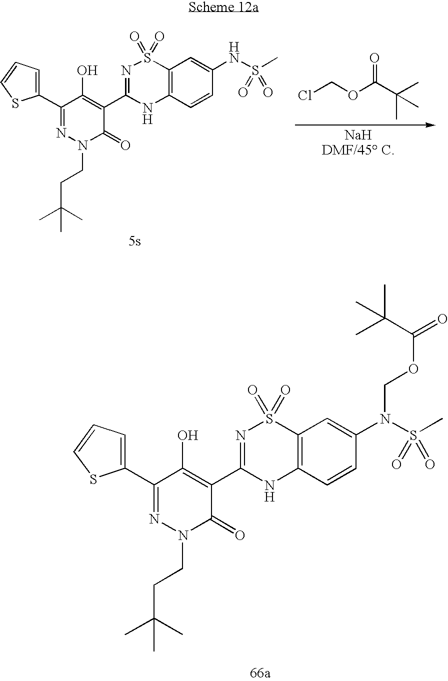

### 2,2-Dimethyl-prop ionic acid ({3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-amino)-methyl ester (66a)

N-{3-[2-(3,3-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide (5s) (60 mg, 0.11 mmol) was dissolved in anhydrous DMF (3 mL) under N₂ atmosphere, NaH (Aldrich) (60% in mineral oil, 17.4 mg, 0.44 mmol) was added. The mixture was stirred at 45° C. for 30 min, chloromethyl pivalate (Aldrich) (0.063 mL, 0.44 mmol) was added and the resulting mixture was stirred at 45° C. for 6 hours. LC-MS showed the desired product was formed and some starting material was left. More chloromethyl pivalate (Aldrich) (0.08 mL, 0.55 mmol) was added and the mixture was stirred for 14 hours. The reaction was quenched with H₂O and concentrated under reduced pressure to give crude product. The crude compound was purified by prep-TLC plate with 7% MeOH/DCM as solvent to give desired product, 2,2-Dimethyl-propionic acid ({3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-amino)-methyl ester (66a) (43 mg, 59%) as a yellow solid. ¹H NMR (400 MHz, DMSO-d₆): δ 7.87 (dd, 1H, J=3.6, 1.2 Hz), 7.76 (d, 1H, J=2.4 Hz), 7.62 (dd, 1H, J=8.4, 2.4 Hz), 7.54 (dd, 1H, J=4.8, 0.8 Hz), 7.50 (d, 1H, J=8.8 Hz), 7.09 (dd, 1H, J=5.2, 3.6 Hz), 5.60 (s, 2H), 4.06 (m, 2H), 3.19 (s, 3H), 1.64 (m, 2H), 1.19 (s, 9H), 0.97 (s, 9H); LC-MS (ESI⁺): m/e=666.4 [M+1]⁺ (exact ms: 665.16).

The following compounds were prepared in an analogous manner to the procedure shown in Scheme 12a.

### 2,2-Dimethyl-propionic acid ({3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-amino)-methyl ester

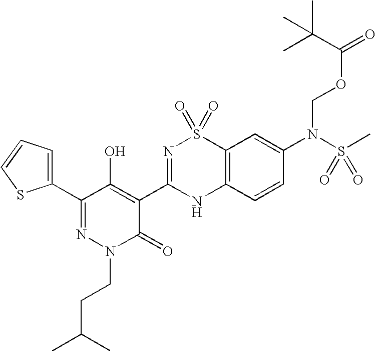

¹H NMR (400 MHz, DMSO-d₆): δ 7.88 (dd, 1H, J=3.6, 1.2 Hz), 7.76 (d, 1H, J=2.4 Hz), 7.62 (dd, 1H, J=8.4, 2.0 Hz), 7.53 (dd, 1H, J=5.2, 1.2 Hz), 7.48 (d, 1H, J=8.8 Hz), 7.09 (dd, 1H, J=5.2, 3.6 Hz), 5.60 (s, 2H), 4.05 (t, 2H, J=6.4 Hz), 3.19 (s, 3H), 1.62 (m, 3H), 1.19 (s, 9H), 0.93 (d, 9H, J=6.8 Hz); LC-MS (ESI⁺): m/e=652.1 [M+1]⁺ (exact ms: 651.15).

### EXAMPLE 12-2

Scheme 12b describes the synthesis of compound 66b of Formula I.

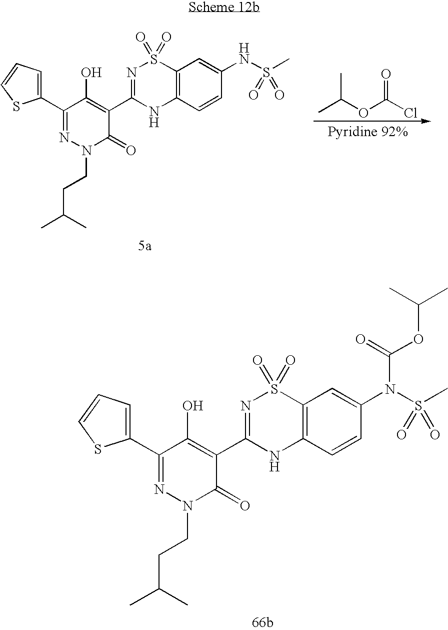

### N-{3-[5-Hydro-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-isopopyl-carbamate-methanesulfonamide (66b)

N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide (5a) (13 mg, 0.024 mmol) was dissolved in anhydrous pyridine (1 mL) under N₂ atmosphere, isopropyl chloroformate (Aldrich) (1.0 M in toluene, 0.12 mL, 0.12 mmol) was added slowly. The resulting mixture was stirred at rt for 1.5 hours, LC-MS showed completion of the reaction. The reaction was quenched by MeOH (1 mL) and then concentrated under reduced pressure to give crude product. The crude product was purified by prep-TLC plate with 10% MeOH/DCM as solvent to give desired product, N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-isopopyl-carbamate-methanesulfonamide (66b) (13.5 mg, 92%) as a yellow solid. ¹H NMR (400 MHz, DMSO-d₆): δ 7.88 (dd, 1H, J=3.6, 1.2 Hz), 7.80 (d, 1H, J=2.4 Hz), 7.55 (dd, 1H, J=8.8, 2.4 Hz), 7.53 (m, 1H), 7.43 (d, 1H, J=8.8 Hz), 7.09 (dd, 1H, J=5.2, 3.6 Hz), 4.95 (m, 1H), 4.05 (t, 2H, J=6.4 Hz), 3.61 (s, 3H), 1.62 (m, 3H), 1.20 (d, 6H, J=6.4 Hz), 0.94 (d, 6H, J=6.4 Hz); LC-MS (ESI): m/e=624.3 [M+1]⁺ (exact ms: 623.12).

### EXAMPLE 12-3

Scheme 12c describes the synthesis of compound 66c of Formula I.

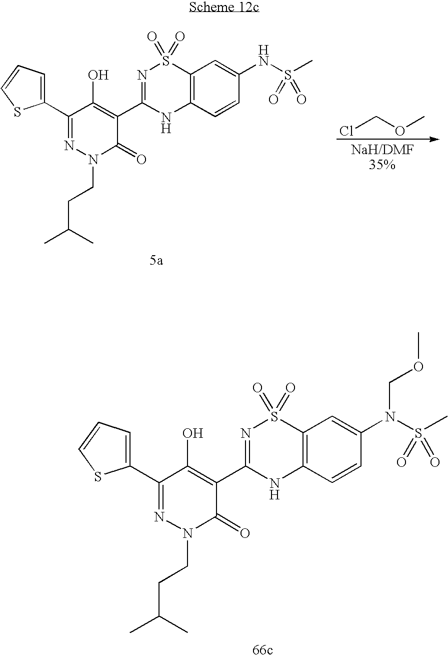

### N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-methoxymethyl-methanesulfonamide (66c)

N-{3-[5-Hydroxy-2-(3-methyl-butyl)-3-oxo-6-thiophen-2-yl-2,3-dihydro-pyridazin-4-yl]-1,1-dioxo-1,4-dihydro-1l6-benzo[1,2,4]thiadiazin-7-yl}-methanesulfonamide (5a) (30 mg, 0.056 mmol) was dissolved in anhydrous DMF (1.5 mL) under N₂ atmosphere, NaH (Aldrich) (60% in mineral oil, 8.9 mg, 0.223 mmol) was added. The mixture was stirred at room temperature for 15 min, chloromethyl methyl ether (Aldrich) (0.1 mL, 1.2 mmol) was added and the resulting mixture was stirred for 3 hours. LC-MS showed the desired product was formed and some starting material was left. The reaction was quenched with H₂O and concentrated under reduced pressure to give crude product. The crude compound was purified by prep-HPLC to give desired product, N-{3-[5-Hydroxy-2-(3-methyl-butyl)-3-oxo-6-thiophen-2-yl-2,3-dihydro-pyridazin-4-yl]-1,1-dioxo-1,4-dihydro-1l6-benzo[1,2,4]thiadiazin-7-yl}-N-methoxymethyl-methanesulfonamide (66c) (11.4 mg, 35%) as a yellow solid. ¹H NMR (400 MHz, DMSO-d₆): δ 7.89 (dd, 1H, J=3.6, 1.2 Hz), 7.85 (d, 1H, J=2.4 Hz), 7.70 (dd, 1H, J=5.2, 2.4 Hz), 7.59 (dd, 1H, J=4.8, 1.2 Hz), 7.55 (d, 1H, J=8.8 Hz), 7.11 (dd, 1H, J=5.2, 3.6 Hz), 4.97 (s, 2H), 4.10 (t, 2H, J=6.8 Hz), 3.33 (s, 3H), 3.10 (s, 3H), 1.64 (m, 3H), 0.94 (d, 6H, J=6.4 Hz); LC-MS (ESI): m/e=582.5 [M+1]⁺ (exact ms: 581.11).

The following compound was made in an analogous manner to the procedure described in Scheme 12c.

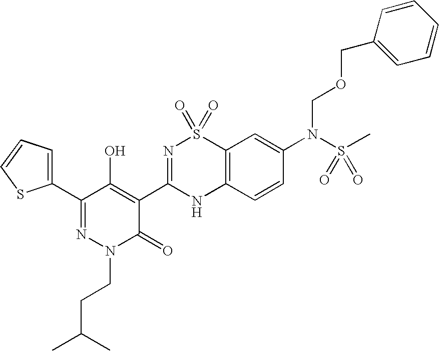

### N-Benzyloxymethyl-N-{3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonamide

¹H NMR (400 MHz, DMSO-d₆): δ 7.94 (d, 1H, J=2.4 Hz), 7.89 (dd, 1H, J=4.0, 1.2 Hz), 7.74 (dd, 1H, J=8.4, 2.4 Hz), 7.63 (dd, 1H, J=4.8, 0.8 Hz), 7.60 (d, 1H, J=8.8 Hz), 7.32 (m, 5H), 7.14 (dd, 1H, J=5.2, 4.0 Hz), 5.11 (s, 2H), 4.61 (s, 2H), 4.13 (t, 2H, J=6.8 Hz), 3.13 (s, 3H), 1.65 (m, 3H), 0.94 (d, 6H, J=6.0 Hz); LC-MS (ESI): m/e=658.3 [M+1]⁺ (exact ms: 657.14).

### EXAMPLE 12-4

Scheme 12d describes the synthesis of compound 66d of Formula I.

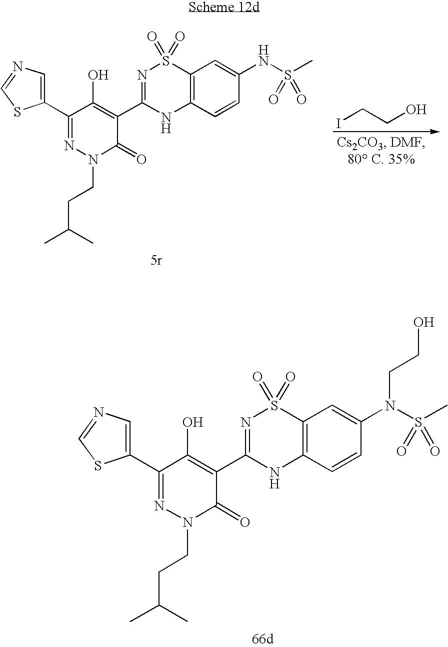

### N-(2-Hydroxy-ethyl)-N-{3-[5-hydroxy-2-(3-methyl-butyl)-3-oxo-6-thiazol-5-yl-2,3-dihydro-pyridazin-4-yl]-1,1-dioxo-1,4-dihydro-1l6-benzo[1,2,4]thiadiazin-7-yl}-methanesulfonamide (66d)

N-{3-[5-Hydroxy-2-(3-methyl-butyl)-3-oxo-6-thiazol-5-yl-2,3-dihydro-pyridazin-4-yl]-1,1-dioxo-1,4-dihydro-1l6-benzo[1,2,4]thiadiazin-7-yl}-methanesulfonamide (5r) (59.8 mg, 0.11 mmol), 2-Iodo-ethanol (15 uL, 0.18 mmol), Cs₂CO₃ (108 mg, 0.33 mmol) and DMF (2 mL) were mixed and the mixture was stirred at 80° C. overnight. The reaction mixture was cooled down and filtered through a small plug of cotton and washed with 1 mL of DMF. The filtrate was directly loaded on reverse-phase HPLC purification (5-95% CH₃CN in H₂O with 0.05% TFA) to give 18.75 mg of pure desired product (66d) in 29.3% isolated yield.

¹H NMR (400 MHz, DMSO-d₆): δ 9.2 (br, <1H), 8.7 (br, <1H), 7.81 (d, 1H, J=2 Hz), 7.64 (dd, 1H, J₁=8.6 Hz, J₂=2.2 Hz), 7.47 (d, 1H, J=8.8 Hz), 4.07 (t, 2H, J=5.6 Hz), 3.70 (t, 2H, J=5.8 Hz), 3.41 (t, 2H, J=6 Hz), 3.04 (s, 3H), 1.56-1.67 (m, 3H), 0.94 (d, 6H, J=6.4 Hz). LC-MS (ESI⁺): m/e=583.2 [M+1]⁺ (exact ms: 582.10).

Method 13: Scheme 13 describes another general procedure that was used to prepare the compound 66 of the Formula I where R¹⁰ is not H.

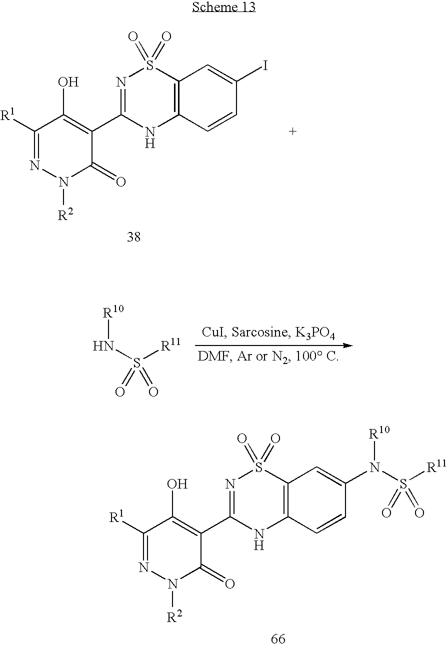

In this general procedure, the iodo group of compound 38 can be directly replaced with N-alklated sulfonamide in the presence of the catalytic amount of CuI, sarcosine and K₃PO₄ under argon or nitrogen atmosphere in the heating condition to give compound 66.

### EXAMPLE 13-1

Scheme 13a describes the synthesis of compound 61a of the Formula I.

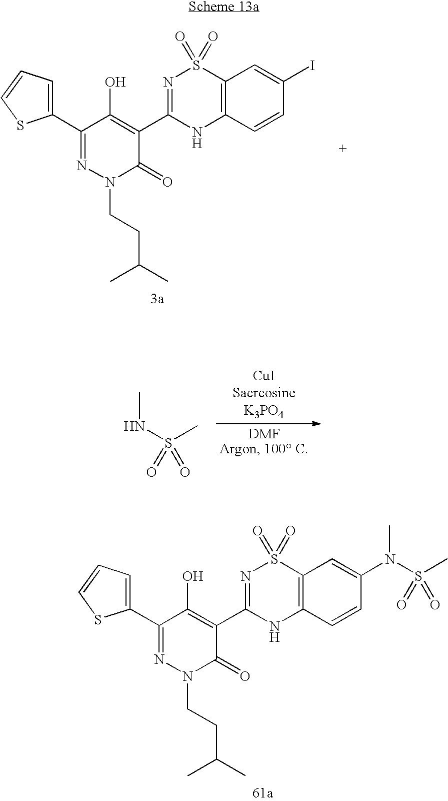

5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (0.2 g, 0.351 mmol), potassium triphosphate (0.186 g, 0.877 mmol), sarcosine (0.008 g, 0.070 mmol), and copper (I) iodide (0.003 g, 0.0175 mmol) were combined. Anhydrous N,N-dimethylformamide (0.7 mL) was added followed by N-methyl-methanesulfonamide (0.040 g, 0.421 mmol). The solution was degassed while stirring under vacuum and the flask charged with argon. The mixture was stirred at 100° C. for 32 h. TLC indicated mostly starting materials present. Additional potassium triphosphate (0.698 g, 3.29 mmol), sarcosine (0.028 g, 0.263 mmol), copper (I) iodide (0.013 g, 0.066 mmol) and N-methyl-methanesulfonamide (0.150 g, 1.58 mmol) were added. The solution was degassed while stirring under vacuum and the flask charged with argon. The mixture was stirred at 100° C. for 16 h. TLC indicated complete consumption of the starting materials. Upon cooling, the mixture was diluted with ethyl acetate (20 mL), washed with 1 M aqueous hydrochloric acid (2×10 mL), dried over magnesium sulfate and concentrated in vacuo. Purification by flash column chromatography (2% methanol in methylene chloride, Merck silica gel 60, 40-63 μm) afforded the desired product, N-{3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-methyl-methanesulfonamide (61a) (0.023 g, 0.042 mmol, 11.9% yield), as a pale yellow powder. ¹H NMR (400 MHz, DMSO-d₆) δ: 0.96 (6H, d, J=6.2 Hz), 1.60-1.70 (3H, m), 3.01 (3H, s), 3.31 (3H, s), 4.15 (2H, t, J=7.1 Hz), 7.16 (1H, dd, J₁=5.5 Hz, J₂=3.9 Hz), 7.62-7.67 (2H, m), 7.73 (1H, dd, J₁=8.7 Hz, J₂=2.4 Hz), 7.87 (1H, d, J=2.4 Hz), 7.90 (1H, d, J=2.3 Hz). LC-MS (ESI): (exact mass: 551.10) m/z=552.24 [M+H⁺] (100%).

### EXAMPLE 13-2

Scheme 13b describes the synthesis of compound 61b of the Formula I.

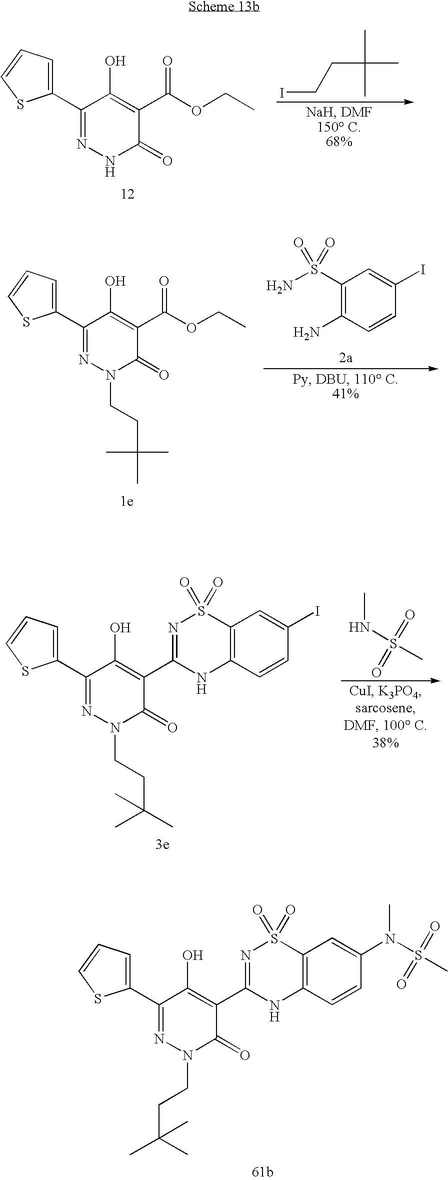

The synthesis of compound 1e was achieved by the alkylation from compound 12 using tert-butylethyl iodine which is similar to that described in Scheme 1f. The compound 3e and 61b were made using the same method as that described in Scheme 13a.

### N-{3-[2-(3,3-Dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-methyl-methanesulfonamide (61b)

¹H NMR (400 MHz, DMSO-d₆) δ: 0.98 (s, 9H), 1.67 (t, 2H, J=8.1 Hz), 3.01 (s, 3H), 3.30 (s, 3H), 4.13 (t, 2H, J=7.8 Hz), 7.13-7.15 (m, 1H), 7.59 (d, 1H, J=9.5 Hz), 7.63 (d, 1H, J=4.6 Hz), 7.71 (dd, 1H, J₁=8.7 Hz, J₂=2.3 Hz), 7.84 (d, 1H, J=2.2 Hz), 7.90 (d, 1H, J=3.9 Hz). LC-MS (ESI⁺): m/e 566.3 [M+1]⁺ (exact ms: 565.11).

The following compounds were prepared in an analogous manner to the procedure described in Scheme 13a.

### N-{3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-N-isopropyl-methanesulfonamide

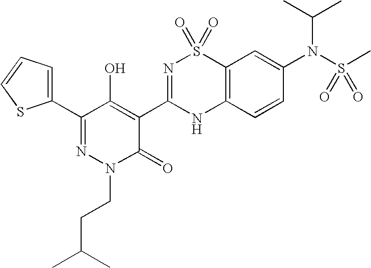

¹H NMR (400 MHz, DMSO-d₆): δ 7.90 (m, 1H), 7.15 (m, 1H), 7.66 (d, 1H, J=5.2 Hz), 7.63 (m, 2H), 7.16 (dd, 1H, J=4.8, 3.6 Hz), 4.36 (m, 1H), 4.16 (t, 2H, J=6.8 Hz), 3.13 (s, 3H), 1.66 (m, 3H), 1.11 (d, 6H, J=6.4 Hz), 0.95 (d, 9H, J=6.4 Hz); LC-MS (ESI): m/e=580.40 [M+1]⁺ (exact ms: 579.13).

### N-{3-[5-Hydroxy-2-(3-methyl-butyl)-3-oxo-6-phenyl-2,3-dihydro-pyridazin-4-yl]-1,1-dioxo-1,4-dihydro-1l6-benzo[1,2,4]thiadiazin-7-yl}-N-methyl-methanesulfonamide

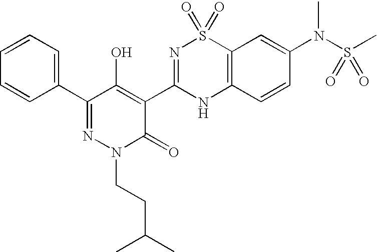

¹H NMR (400 MHz, CDCl₃) δ: 1.03 (6H, d, J=6.2 Hz), 1.68-1.81 (3H, m), 2.90 (3H, s), 3.40 (3H, s), 4.33 (2H, t, J=7.5 Hz), 7.36 (1H, d, J=8.7 Hz), 7.48-7.50 (3H, m), 7.79-7.84 (4H, m), LC-MS (ESI⁺): m/e=546.4 [M+H]⁺ (exact ms: 545.14).

### EXAMPLE 13-3

Scheme 13c describes the synthesis of compound 61c of Formula I.

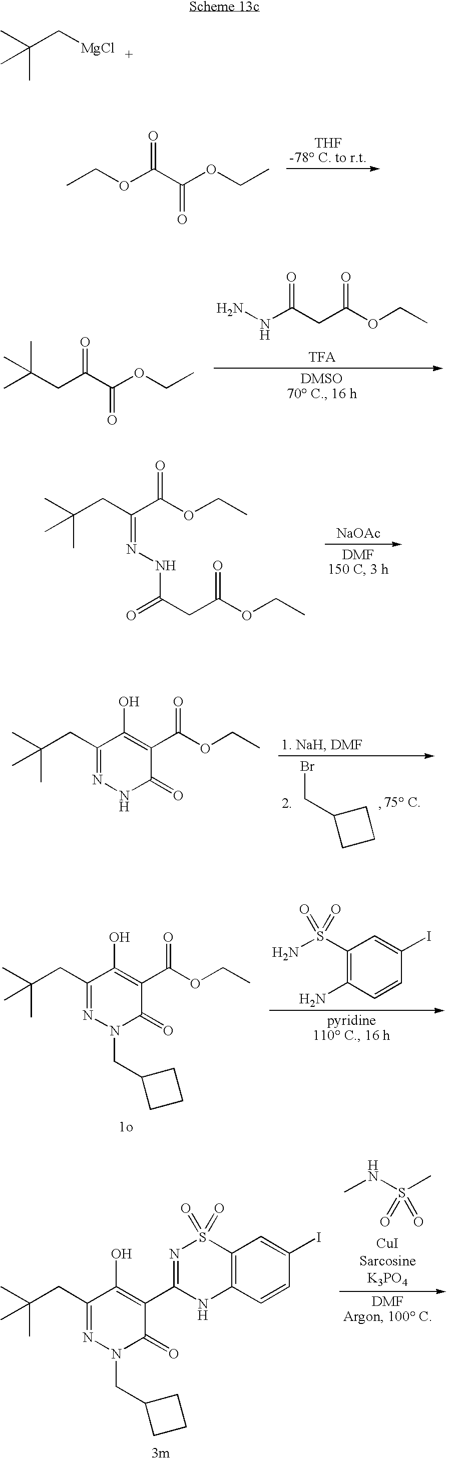

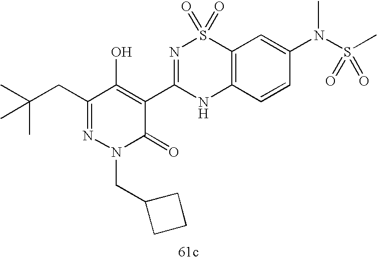

### 2-[(2-Ethoxycarbonyl-acetyl)-hydrazono]-4,4-dimethyl-pentanoic acid ethyl ester

Oxalic acid diethyl ester (6.9 g, 47.17 mmol) was dissolved in anhydrous THF (200 mL). The solution was chilled to −78° C. under N₂. While stirring, a 1 M solution of 2,2-dimethylpropylmagnesium chloride (47.17 mL, 47.17 mmol) was transferred via canula over a period of 10 min. The reaction mixture continued to stir at −78° C. for 1 h. The ice bath was removed and the reaction mixture warmed to room temperature over 30 min. and continued to stir for 2 h. The mixture was poured into 1M HCl (200 mL) and the product was extracted into EtOAc (200 mL). The organic phase was washed with 1M HCl (100 mL), brine (100 mL), dried over MgSO₄ and concentrated to afford 4,4-dimethyl-2-oxo-pentanoic acid ethyl ester as a golden oil.

The above oil was dissolved in DMSO (236 mL) and hydrazinocarbonyl-acetic acid ethyl ester (6.9 g, 47.2 mmol) was added followed by TFA (0.9 mL). The mixture was heated at 70° C. while stirring for 16 h. Upon cooling to room temperature the mixture was diluted with EtOAc (500 mL) and washed with 1 M HCl (2×250 mL), water (2×250 mL), brine (250 mL), dried over MgSO₄ and concentrated to a golden oil. Purification by flash column chromatography (20% EtOAc/Hexanes, Merck silica gel 60, 40-63 μm) afforded the desired product, 2-[(2-ethoxycarbonyl-acetyl)-hydrazono]-4,4-dimethyl-pentanoic acid ethyl ester (1.9 g, 6.33 mmol, 13.4% yield over two steps) as a yellow waxy solid. ¹H NMR (400 MHz, CDCl₃) δ: 0.99 (s, 9H), 1.27 (t, 3H, J=6.9 Hz), 1.34 (t, 3H, J=7.1 Hz), 2.55 (s, 2H), 3.78 (s, 2H), 4.20 (quartet, 2H, J=7.3 Hz), 4.27 (quartet, 2H, J=7.0 Hz), 9.17 (s, 1H). LC-MS (ESI): m/e=301.2 [M+H⁺] (100%) (exact mass: 300.17).

### 6-(2,2-Dimethyl-propyl)-5-hydroxy-3-oxo-2,3-dihydro-pyridazine-4-carboxylic acid ethyl ester

2-[(2-Ethoxycarbonyl-acetyl)-hydrazono]-4,4-dimethyl-pentanoic acid ethyl ester (1.3 g, 4.33 mmol) was dissolved in DMF (10 mL) and NaOAc (0.711 g, 8.67 mmol) was added. The flask was evacuated and filled with N₂. The mixture was heated at 150° C. for 3 h. Upon cooling to 25° C., 1 M HCl (30 mL) was added. The product was extracted into EtOAc (3×10 mL). The combined organic layers were washed with brine (2×5 mL), dried over MgSO₄ and concentrated in vacou to an orange oil. Purification by flash column chromatography (50% EtOAc/Hexanes, Merck silica gel 60, 40-63 μm) afforded the desired product, 6-(2,2-dimethyl-propyl)-5-hydroxy-3-oxo-2,3-dihydro-pyridazine-4-carboxylic acid ethyl ester (0.162 g, 0.637 mmol, 15% yield) as a beige powder. ¹H NMR (400 MHz, DMSO-d₆) δ: 0.91 (s, 9H), 1.27 (t, 3H, J=7.4 Hz), 2.48 (s, 2H), 4.28 (quartet, 2H, J=7.0 Hz).

### 2-Cyclobutylmethyl-6-(2,2-dimethyl-propyl)-5-hydroxy-3-oxo-2,3-dihydro-pyridazine-4-carboxylic acid ethyl ester (1o)

6-(2,2-Dimethyl-propyl)-5-hydroxy-3-oxo-2,3-dihydro-pyridazine-4-carboxylic acid ethyl ester (0.162 g, 0.637 mmol) was dissolved in anhydrous DMF (2 mL). A 60% suspension of NaH in mineral oil (0.059 g, 1.47 mmol) was added. The mixture was stirred at 25° C. for 10 min. with occasional venting. Bromomethyl-cyclobutane (0.075 mL, 0.669 mmol) was added and the mixture stirred at 75° C. for 4 hours. Upon cooling to 25° C., the mixture was diluted with 1 M HCl (25 mL) and the product extracted into EtOAc (30 mL). The organic phase was further washed with 1 M HCl (20 mL), brine (20 mL), dried over MgSO₄ and concentrated in vacuo. Purification of the residue by flash column chromatography (20% EtOAc/Hexanes) afforded the desired product, 2-Cyclobutylmethyl-6-(2,2-dimethyl-propyl)-5-hydroxy-3-oxo-2,3-dihydro-pyridazine-4-carboxylic acid ethyl ester (1o) (0.079 g, 0.245 mmol, 38% yield), as an oil. ¹H NMR (400 MHz, DMSO-d₆) δ: 0.91 (s, 9H), 1.26 (t, 3H, J=7.1 Hz), 1.70-1.86 (m, 4H), 1.90-1.98 (m, 2H), 2.49 (s, 2H), 2.71 (septet, 1H, J=7.6 Hz), 3.99 (d, 2H, J=6.8 Hz), 4.25 (quartet, 2H, J=7.0 Hz). LC-MS (ESI⁺): m/e=323.39 [M+H⁺] (100%) (exact mass: 322.19).

### 2-Cyclobutylmethyl-6-(2,2-dimethyl-propyl)-5-hydroxy-4-(7-iodo-1,1-dioxo-1,4-dihydro-1λ⁶-benzo[1,2,4]thiadiazin-3-yl)-2H-pyridazin-3-one (3m)

2-Cyclobutylmethyl-6-(2,2-dimethyl-propyl)-5-hydroxy)-3-oxo-2,3-dihydro-pyridazine-4-carboxylic acid ethyl ester (1o) (0.071 g, 0.22 mmol) and 2-amino-5-iodo-benzenesulfonamide (0.069 g, 0.231 mmol) were combined and dissolved in anhydrous pyridine (1.2 mL). The flask was degassed and backfilled with N₂. The mixture stirred at 110° C. for 16 h followed by 120° C. for 24 h. Upon cooling to 25° C., the mixture was concentrated in vacuo to a brown oil. The oil was dissolved in EtOAc (20 mL), washed with 1M HCl (10 mL), brine (10 mL), dried over MgSO₄ and concentrated to afford the crude product, 2-cyclobutylmethyl-6-(2,2-dimethyl-propyl)-5-hydroxy-4-(7-iodo-1,1-dioxo-1,4-dihydro-1λ⁶-benzo[1,2,4]thiadiazin-3-yl)-2H-pyridazin-3-one (3m), as a brown oil which was used directly in the next step without further purification or characterization. LC-MS (ESI): m/e=557.26 [M+H⁺] (100%) (exact mass: 556.06).

### N-{3-[2-Cyclobutylmethyl-6-(2,2-dimethyl-propyl)-5-hydroxy-3-oxo-2,3-dihydro-pyridazin-4-yl]-1,1-dioxo-1,4-dihydro-1λ⁶-benzo[1,2,4]thiadiazin-7-yl}-N-methyl-methanesulfonamide (61c)

The residue from above, 2-cyclobutylmethyl-6-(2,2-dimethyl-propyl)-5-hydroxy-4-(7-iodo-1,1-dioxo-1,4-dihydro-1λ⁶-benzo[1,2,4]thiadiazin-3-yl)-2H-pyridazin-3-one (3m), was combined with potassium triphosphate (0.305 g, 1.44 mmol), sarcosine (0.015 g, 0.172 mmol), and copper (I) iodide (0.014 g, 0.07 mmol). Anhydrous DMF (3 mL) was added followed by N-methyl-methanesulfonamide (0.471 g, 4.31 mmol). The solution was degassed while stirring under vacuum and the flask backfilled with N₂. The mixture stirred at 100° C. for 16 h. Upon cooling 25° C., the mixture was diluted with EtOAc (100 mL) and shaken with 1M HCl (100 mL). The resulting suspension was filtered through paper to remove the solids. The organic filtrate was washed with 1M HCl (2×50 mL), brine (50 mL), dried over MgSO₄ and concentrated in vacuo. Purification by reverse phase HPLC (50%-100% acetonitrile in water, 20 min), followed by trituration from a minimal amount of 3:1 hexanes in EtOAc followed by filtration afforded the desired product, N-{3-[2-cyclobutylmethyl-6-(2,2-dimethyl-propyl)-5-hydroxy-3-oxo-2,3-dihydro-pyridazin-4-yl]-1,1-dioxo-1,4-dihydro-1λ⁶-benzo[1,2,4]thiadiazin-7-yl}-N-methyl-methanesulfonamide (61c) (0.0168 g, 0.031 mmol, 14% yield over two steps) as a beige powder. ¹H NMR (400 MHz, DMSO-d₆) δ: 0.97 (s, 9H), 1.80-1.90 (m, 4H), 1.95-2.04 (m, 2H), 2.59 (s, 2H), 2.80 (quintet, 1H, J=7.4 Hz), 3.02 (s, 3H), 3.31 (s, 3H), 4.15 (d, 2H, J=7.3 Hz), 7.70 (d, 1H, J=9.1 Hz), 7.77 (dd, 1H, J1=8.5 Hz, J2=2.3 Hz), 7.90 (d, 1H, J=2.4 Hz), 13.94 (bs, 1H). LC-MS (ESI⁺): m/e=538.44 [M+H⁺] (100%) (exact mass: 537.17).

### EXAMPLE 13-4

Scheme 13d describes the synthesis of compound 66a of the Formula I.

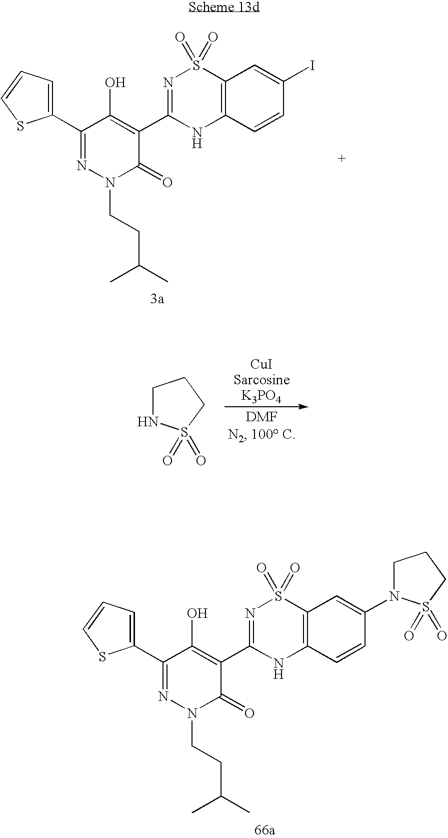

5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (0.25 g, 0.438 mmol), potassium triphosphate (0.465 g, 2.19 mmol), sarcosine (0.023 g, 0.263 mmol), and copper (I) iodide (0.021 g, 0.110 mmol) were combined. Anhydrous N,N-dimethylformamide (3 mL) was added followed by isothiazolidine 1,1-dioxide (0.531 g, 4.38 mmol, prepared according to the procedure from Org. Lett; 5; 22; 2003; 4175-4178). The solution was degassed while stirring under vacuum and the flask charged with nitrogen. The mixture stirred at 100° C. for 16 h. LC-MS indicated the major product to be the amino acid intermediate. Additional isothiazolidine 1,1-dioxide (0.531 g, 4.38 mmol) was added. The solution was degassed while stirring under vacuum and the flask charged with nitrogen. The mixture stirred at 100° C. for 16 h. LC-MS indicated complete reaction at this point.

Upon cooling, the mixture was diluted with ethyl acetate (80 mL), washed with 1M aqueous hydrochloric acid (2×10 mL), saturated aqueous ammonium chloride (10 mL), dried over magnesium sulfate filtered. Methyl alcohol (100 mL) was added and the desired product crystallized over a period of 2 h. Collection by filtration followed by rinsing with methyl alcohol (2×10 mL) followed by drying in vacuo for 3 h afforded the desired product, 4-[7-(1,1-dioxo-1λ⁶-isothiazolidin-2-yl)-1,1-dioxo-1,4-dihydro-1λ⁶-benzo[1,2,4]thiadiazin-3-yl]-5-hydroxy-2-(3-methyl-butyl)-6-thiophen-2-yl-2H-pyridazin-3-one (66a) (0.0693, 0.123 mmol, 28% yield), as an orange powder. ¹H NMR (400 MHz, DMSO-d₆) δ: 0.96 (6H, d, J=6.2 Hz), 1.59-1.71 (3H, m), 2.44 (2H, quintet, J=7.1 Hz), 3.59 (2H, t, J=6.9 Hz), 3.85 (2H, t, J=6.7 Hz), 4.17 (2H, t, J=6.7 Hz), 7.17 (1H, dd, J=5.6 Hz, J₂=3.9 Hz), 7.54-7.58 (2H, m), 7.68-7.71 (2H, m), 7.91 (1H, d, J=4.0 Hz), 13.94 (1H, s). LC-MS (ESI): (exact mass: 563.10): m/e=564.66 [M+H]⁺ (100%).

### EXAMPLE 13-5

Scheme 13e describes the synthesis of compound 61d of the Formula I.

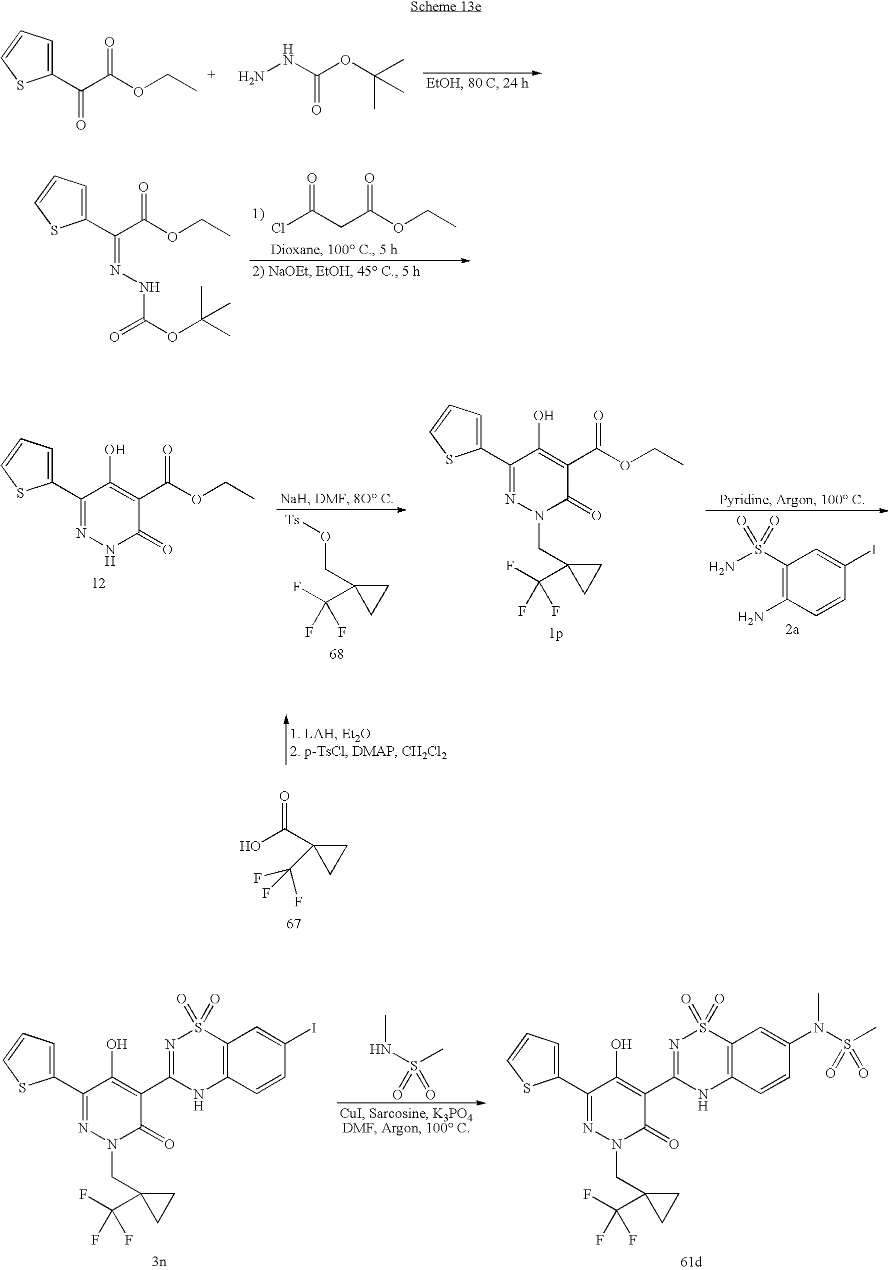

### Toluene-4-sulfonic acid 1-trifluoromethyl-cyclopropylmethyl ester (68)

1-Trifluoromethyl-cyclopropanecarboxylic acid (2 g, 12.98 mmol) was dissolved in anhydrous diethyl ether (25 mL). The solution was chilled to 0° C. A 2M solution of lithium aluminum hydride in tetrahydrofuran (7.5 mL, 15 mmol) was added dropwise to the stirring solution. The mixture stirred at 0° C. for 30 min and at room temperature for 4 h. The excess lithium aluminum hydride was quenched by the careful addition of water (20 mL). The mixture was diluted with diethyl ether (100 mL), washed with 0.5M aqueous hydrochloric acid (2×25 mL), saturated aqueous sodium chloride (15 mL), dried over magnesium sulfate filtered and concentrated under partial vacuum (just enough to remove diethyl ether) to a clear oil. The oil was dissolved in methylene chloride (32 mL). 4-Methyl-benzenesulfonyl chloride (2.72 g, 14.28 mmol), triethylamine (2.37 mL, 16.87 mmol) and dimethyl-pyridin-4-yl-amine (0.16 g, 1.3 mmol) were added and the mixture stirred at 45° C. for 16 h. The mixture was diluted with methylene chloride (150 mL), washed with 1M aqueous hydrochloric acid (2×25 mL), saturated aqueous sodium bicarbonate solution (25 mL), and passed through a plug of silica gel. The filtrate was concentrated in vacuo to afford the desired product, toluene-4-sulfonic acid 1-trifluoromethyl-cyclopropylmethyl ester (68) (2.32 g, 7.89 mmol, 60.8%), as a golden oil. ¹H NMR (400 MHz, DMSO-d) δ: 0.92-0.96 (2H, m), 1.04-1.07 (2H, m), 2.42 (3H, s), 4.17 (2H, s), 7.47 (2H, d, J=7.8 Hz), 7.77 (2H, d, J=8.6 Hz).

### 5-Hydroxy-3-oxo-6-thiophen-2-yl-2-(1-trifluoromethyl-cyclopropylmethyl)-2,3-dihydro-pyridazine-4-carboxylic acid ethyl ester (1p)

5-hydroxy-3-oxo-6-thiophen-2-yl-2,3-dihydro-pyridazine-4-carboxylic acid ethyl ester (12) (0.5 g, 1.88 mmol) (see Scheme 1h) was suspended in anhydrous N,N-dimethylformamide (9.4 mL). A 60% suspension of sodium hydride in mineral oil (0.166 g, 4.14 mmol) was added. The mixture stirred in a sealed vial for 10 minutes with occasional venting. Toluene-4-sulfonic acid 1-trifluoromethyl-cyclopropylmethyl ester (68) (0.609 g, 2.07 mmol) was added and the mixture stirred at 80° C. for 7 hours. Upon cooling, the mixture diluted with ethyl acetate (300 mL), washed with 1M aqueous hydrochloric acid (2×100 mL), water (50 mL), saturated aqueous sodium chloride (50 mL), dried over magnesium sulfate filtered and concentrated in vacuo. Purification by flash column chromatography (2% methanol in methylene chloride, Merck silica gel 60, 40-63 μm) afforded the desired product, 5-hydroxy-3-oxo-6-thiophen-2-yl-2-(1-trifluoromethyl-cyclopropylmethyl)-2,3-dihydro-pyridazine-4-carboxylic acid ethyl ester (1p) (0.252 g, 0.649 mmol, 35% yield), as a yellow, waxy solid. ¹H NMR (400 MHz, CDCl₃) δ: 1.07-1.14 (4H, m), 1.49 (3H, t, J=7.0 Hz), 4.43 (2H, s), 4.54 (2H, quartet, J=7.3 Hz), 7.11 (1H, dd, J=5.3 Hz, J₂=3.5 Hz), 7.41 (1H, d, J=5.5 Hz), 7.91 (1H, d, J=3.8 Hz).

### 5-Hydroxy-4-(7-iodo-1,1-dioxo-1,4-dihydro-1λ6-benzo[1,2,4]thiadiazin-3-yl)-6-thiophen-2-yl-2-(1-trifluoromethyl-cyclopropylmethyl)-2H-pyridazin-3-one (3n)

5-Hydroxy-3-oxo-6-thiophen-2-yl-2-(1-trifluoromethyl-cyclopropylmethyl)-2,3-dihydro-pyridazine-4-carboxylic acid ethyl ester (1p) (0.2 g, 0.515 mmol) and 2-amino-5-iodo-benzenesulfonamide (2a) (0.168 g, 0.566 mmol) were combined and anhydrous pyridine (3.25 mL) was added. The solution was degassed while stirring under vacuum and the flask charged with argon. The mixture stirred at 110° C. for 16 h. Upon cooling, the solution was concentrated in vacuo to a thick brown oil. Addition of methyl alcohol (5 mL) caused the desired product to precipitate. Collection by filtration and rinsing with methyl alcohol (5 mL) followed by drying in vacuo for 16 h afforded the desired product, 5-hydroxy-4-(7-iodo-1,1-dioxo-1,4-dihydro-1λ⁶-benzo[1,2,4]thiadiazin-3-yl)-6-thiophen-2-yl-2-(1-trifluoromethyl-cyclopropylmethyl)-2H-pyridazin-3-one (3n) (0.16 g, 0.257 mmol, 50% yield), as a beige/yellow powder. LC-MS (ESI): (exact mass: 621.95): m/e=623.08 [M+H⁺] (100%).

### N-{3-[5-hydroxy-3-oxo-6-thiophen-2-yl-2-(1-trifluoromethyl-cyclopropylmethyl)-2,3-dihydro-pyridazin-4-yl]-1,1-dioxo-1,4-dihydro-1λ⁶-benzo[1,2,4]thiadiazin-7-yl}-N-methyl-methanesulfonamide (61d)

5-Hydroxy-4-(7-iodo-1,1-dioxo-1,4-dihydro-1λ⁶-benzo[1,2,4]thiadiazin-3-yl)-6-thiophen-2-yl-2-(1-trifluoromethyl-cyclopropylmethyl)-2H-pyridazin-3-one (3n) (0.06 g, 0.096 mmol), potassium triphosphate (0.102 g, 0.482 mmol), sarcosine (0.005 g, 0.058 mmol), and copper (1) iodide (0.005 g, 0.024 mmol) were combined. Anhydrous N,N-dimethylformamide (1 mL) was added followed by N-methyl-methanesulfonamide (0.105 g, 0.964 mmol). The solution was degassed while stirring under vacuum and the flask charged with argon. The mixture stirred at 100° C. for 4 h. LC-MS indicated complete coupling at this point. Upon cooling, the mixture was diluted with ethyl acetate (50 mL), washed with 1M aqueous hydrochloric acid (2×15 mL), saturated aqueous sodium chloride (15 mL), dried over magnesium sulfate and concentrated in vacuo. Purification by reverse phase HPLC (50%-100% acetonitrile in water, 20 min), followed by trituration from a minimal amount of methanol followed by filtration afforded the desired product, N-{3-[5-hydroxy-3-oxo-6-thiophen-2-yl-2-(1-trifluoromethyl-cyclopropylmethyl)-2,3-dihydro-pyridazin-4-yl]-1,1-dioxo-1,4-dihydro-1λ⁶-benzo[1,2,4]thiadiazin-7-yl}-N-methyl-methanesulfonamide (61d) (0.0225 g, 0.037 mmol, 39% yield), as a yellow powder.

¹H NMR (400 MHz, DMSO-d₆) δ: 1.04-1.09 (2H, m), 1.15-1.19 (2H, m), 3.00 (3H, s), 3.30 (3H, s), 4.38 (2H, s), 7.14 (1H, dd, J₁=5.3 Hz, J₂=3.6 Hz), 7.54 (1H, d, J=8.5 Hz), 7.63 (1H, d, J=3.7 Hz), 7.69 (1H, dd, J=9.2 Hz, J₂=2.4 Hz), 7.82 (1H, d, J=2.3 Hz), 7.91 (1H, d, J=3.9 Hz). LC-MS (ESI⁺): (exact mass: 603.05) m/e=604.35 [M+H]⁺ (100%).

Method 14-1: Schemes 14a describes the synthesis of compound 72 of Formula I.

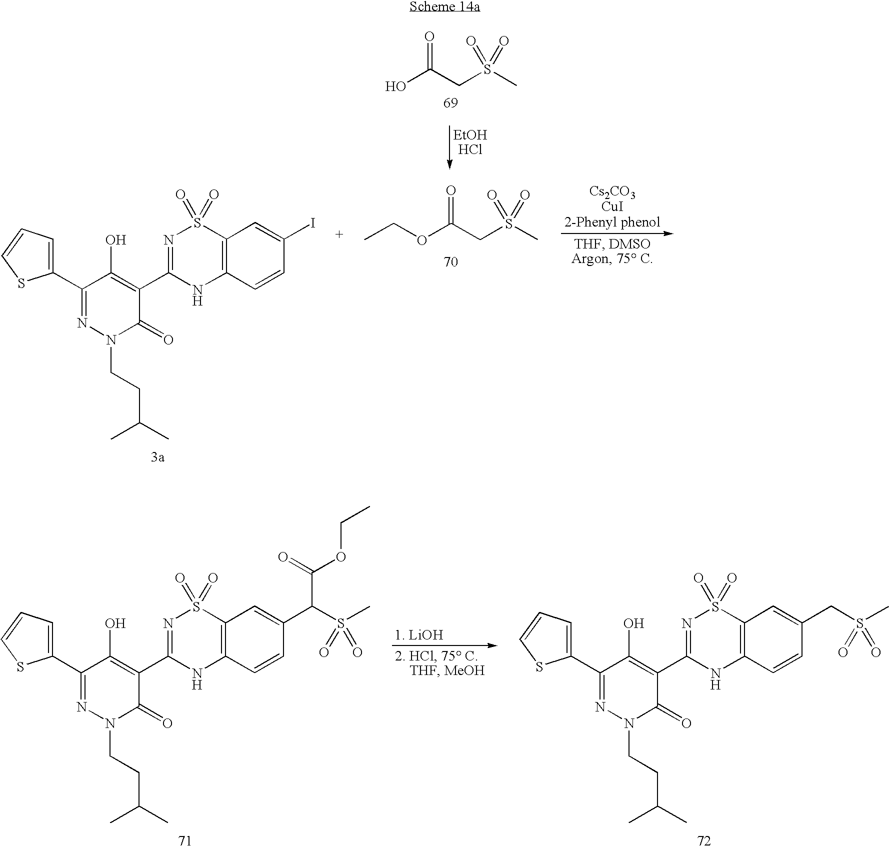

Methanesulfonyl-acetic acid (69) (5.3 g, 38.4 mmol) was suspended in ethyl alcohol (50 mL). A 4M solution of HCl in dioxane (8 mL) was added. The mixture stirred at reflux for 0.5 h, everything had dissolved at this point. The mixture continued to stir at reflux for a total of 16 h. Upon cooling, the solution was concentrated in vacuo to approximately 20 mL. The oil was diluted with ethyl acetate (300 mL), washed with water (100 mL), brine (100 ml), dried over magnesium sulfate and concentrated in vacuo to a golden oil. Purification by flash column chromatography (30% ethyl acetate in hexanes, Merck silica gel 60, 40-63 μm) afforded the desired product, methanesulfonyl-acetic acid ethyl ester (70) (5.67 g, 34.12 mmol, 89% yield), as a clear oil upon concentrating. ¹H NMR (400 MHz, DMSO-d₆) δ: 1.22 (3H, t, J=7.0 Hz), 3.13 (3H, s), 4.17 (2H, quartet, J=7.0 Hz), 4.38 (2H, s). 5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (0.4 g, 0.702 mmol), cesium carbonate (0.913 g, 2.81 mmol), copper (I) iodide (0.2 g, 1.05 mmol) and 2-phenyl phenol (0.048 g, 0.28 mmol) were combined. Anhydrous tetrahydrofuran (4 mL) and dimethylsulfoxide (1 mL) were added followed by methanesulfonyl-acetic acid ethyl ester (0.351 g, 2.11 mmol). The solution was degassed while stirring under vacuum and the flask charged with argon. The mixture stirred at 75° C. for 24 h. LC-MS indicated some product with mostly starting materials present. Additional methanesulfonyl-acetic acid ethyl ester (0.351 g, 2.11 mmol), copper (I) iodide (0.2 g, 1.05 mmol), cesium carbonate (0.46 g, 1.41 mmol) and tetrahydrofuran (3 mL) were added. The solution was degassed while stirring under vacuum and the flask charged with argon. The mixture stirred at 75° C. for 16 h.

Upon cooling, the mixture was diluted with ethyl acetate (250 mL) and washed with 1M aqueous hydrochloric acid (150 mL). The organic phase was passed through a plug of celite. The filtrate was washed with 1 M aqueous hydrochloric acid (100 mL), brine (100 mL), dried over magnesium sulfate and concentrated in vacuo to a brown oil. Addition of methanol (4 mL) caused the desired product to precipitate. Collection by filtration followed by drying in vacuo afforded the desired product, {3-[5-hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-acetic acid ethyl ester (71) (0.156 g, 0.256 mmol, 37% yield), as an orange powder. LC-MS (ESI): (exact mass: 608.11) m/z=609.23 [M+H⁺] (100%). {3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-acetic acid ethyl ester (71) (0.1 g, 0.164 mmol) was dissolved in tetrahydrofuran (1 mL) and methanol (1 mL) was added. A 2M aqueous solution of lithium hydroxide (1 mL) was added and the mixture stirred at room temperature for 30 min. LC-MS indicated complete hydrolysis to the carboxylic acid intermediate, {3-[5-Hydroxy-2-(3-methyl-butyl)-3-oxo-6-thiophen-2-yl-2,3-dihydro-pyridazin-4-yl]-1,1-dioxo-1,4-dihydro-1λ⁶-benzo[1,2,4]thiadiazin-7-yl}-methanesulfonyl-acetic acid, LC-MS (ESI): (exact mass: 580.08) m/z=581.10 [M+H⁺] (100%). A solution of 6M aqueous hydrochloric acid (0.4 mL) and tetrahydrofuran (4 mL) were added directly to the reaction mixture. Slight precipitation was observed. The mixture stirred at 75° C. for 4 h. LC-MS indicated complete de-carboxylation at this point. The mixture was diluted with ethyl acetate (100 mL), washed with brine (50 mL), dried over magnesium sulfate and concentrated in vacuo to a brown oil. The oil was dissolved in N,N-dimethylformamide (4 mL) and methyl alcohol (10 mL) was added. A precipitate formed and was removed by filtration. LC-MS indicated the precipitate to be a side product. The filtrate was concentrated in vacuo. Purification by reverse phase HPLC (75%-100% acetonitrile in water, 20 min), followed by trituration from a minimal amount of methanol followed by filtration afforded the desired product, 5-hydroxy-4-(7-methanesulfonylmethyl-1,1-dioxo-1,4-dihydro-1λ⁶-benzo[1,2,4]thiadiazin-3-yl)-2-(3-methyl-butyl)-6-thiophen-2-yl-2H-pyridazin-3-one (72) (0.0059 g, 0.01 mmol, 6.7% yield), as a beige powder. ¹H NMR (400 MHz, DMSO-d₆) δ: 0.95 (6H, d, J=6.2 Hz), 1.58-1.69 (3H, m), 2.94 (3H, s), 4.14 (2H, t, J=6.6 Hz), 4.66 (2H, s), 7.15 (1H, t, J=4.2 Hz), 7.59 (1H, d, J=7.7 Hz), 7.65 (1H, d, J=4.7 Hz), 7.70 (1H, dd, J₁=8.6 Hz, J₂=1.8 Hz), 7.87-7.95 (2H, m). LC-MS (ESI): (exact mass: 536.09) m/z=537.0 [M+H]⁺ (100%).

Method 14-2: Scheme 14b describes the synthesis of compound 73 and 74 of Formula I.

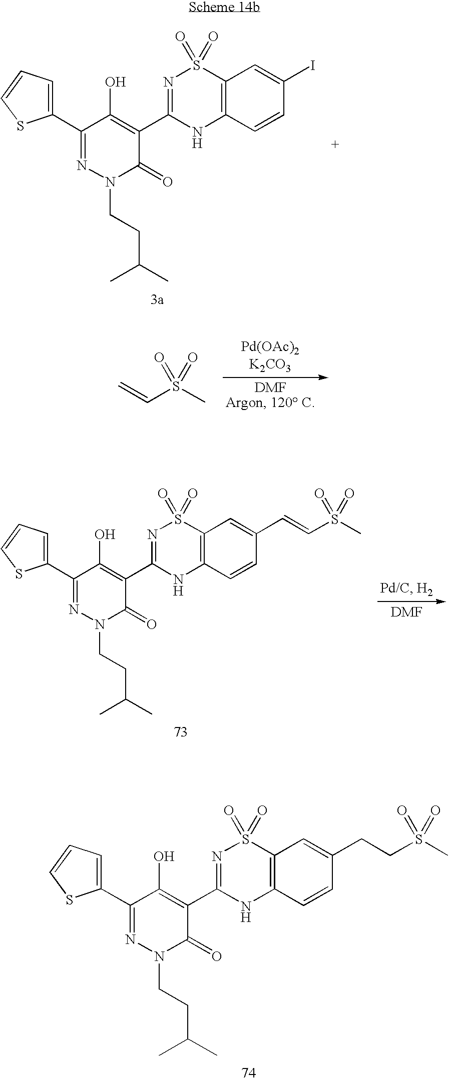

5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (0.15 g, 0.263 mmol), palladium(II) acetate (0.012 g, 0.053 mmol) and potassium carbonate (0.091 g, 0.658 mmol) were combined. Anhydrous N,N-dimethylformamide (3 mL) was added followed by methanesulfonyl-ethene (0.42 g, 3.94 mmol). The mixture was degassed while stirring under vacuum and the flask charged with argon. The mixture stirred at 120° C. for 60 min. TLC indicated complete reaction at this point. Upon cooling, the mixture was diluted with ethyl acetate (50 mL), washed with 1M aqueous hydrochloric acid (2×15 mL), saturated aqueous sodium chloride (5 mL), dried over magnesium sulfate filtered and concentrated in vacuo to a brown solid. Trituration with methyl alcohol (5 mL) followed by collection by filtration and rinsing with methyl alcohol (5 mL), N,N-dimethylformamide (2 mL) and methyl alcohol (5 mL) successively, followed by drying in vacuo for 24 h afforded the desired product, 5-hydroxy-4-[7-(2-methanesulfonyl-vinyl)-1,1-dioxo-1,4-dihydro-1λ⁶-benzo[1,2,4]thiadiazin-3-yl]-2-(3-methyl-butyl)-6-thiophen-2-yl-2H-pyridazin-3-one (73) (0.056 g, 0.102 mmol, 39% yield), as a beige powder. ¹H NMR (400 MHz, DMSO-d₆) δ: 0.95 (6H, d, J=6.3 Hz), 1.58-1.69 (3H, m), 3.10 (3H, s), 4.15 (2H, t, J=6.7 Hz), 7.16 (1H, dd, J₁=5.5 Hz, J₂=3.9 Hz), 7.58-7.71 (4H, m), 7.90 (1H, d, J=3.9 Hz), 8.05 (1H, dd, J₁=8.6 Hz, J₂=2.2 Hz), 8.29 (1H, d, J=1.6 Hz). LC-MS (ESI): (exact mass: 548.09) m/z=549.38 [M+H⁺] (100%). 5-Hydroxy-4-[7-(2-methanesulfonyl-vinyl)-1,1-dioxo-1,4-dihydro-1λ⁶-benzo[1,2,4]thiadiazin-3-yl]-2-(3-methyl-butyl)-6-thiophen-2-yl-2H-pyridazin-3-one (73) (0.05 g, 0.091 mmol) was dissolved in N,N-dimethylformamide (3.5 mL) with gentle warming (˜65° C.). Upon cooling, 10%, wet, palladium on carbon (0.05 g) was added. The mixture was degassed while stirring under vacuum and the flask charged with hydrogen gas. Additionally, hydrogen gas was bubbled through the reaction mixture while stirring for 30 min. LC-MS indicated complete hydrogenation at this point. The mixture was filtered through celite and the filtrate was diluted with methyl alcohol (15 mL). The desired product crystallized over a period of 2 h. Collection by filtration followed by rinsing with methyl alcohol (2×5 mL) followed by drying in vacuo for 2 h afforded the desired product, 5-hydroxy-4-[7-(2-methanesulfonyl-ethyl)-1,1-dioxo-1,4-dihydro-1λ⁶-benzo[1,2,4]thiadiazin-3-yl]-2-(3-methyl-butyl)-6-thiophen-2-yl-2H-pyridazin-3-one (74) (0.0364 g, 0.066 mmol, 73% yield), as a greenish, fluffy solid. ¹H NMR (400 MHz, DMSO-d₆) δ: 0.96 (6H, d, J=6.2 Hz), 1.59-1.71 (3H, m), 3.02 (3H, s), 3.13-3.17 (2H, m), 3.49-3.53 (2H, m), 4.18 (2H, t, J=6.9 Hz), 7.17 (1H, dd, J₁=5.6 Hz, J₂=4.0 Hz), 7.59 (1H, d, J=8.5 Hz), 7.68-7.71 (2H, m), 7.88 (1H, d, J=1.6 Hz), 7.91 (1H, d, J=4.0 Hz), 13.86 (1H, s). LC-MS (ESI): (exact mass: 548.09) m/z=549.38 [M+H⁺] (100%).

Method 14-3: Scheme 14c describes the synthesis of compound 78 of Formula I.

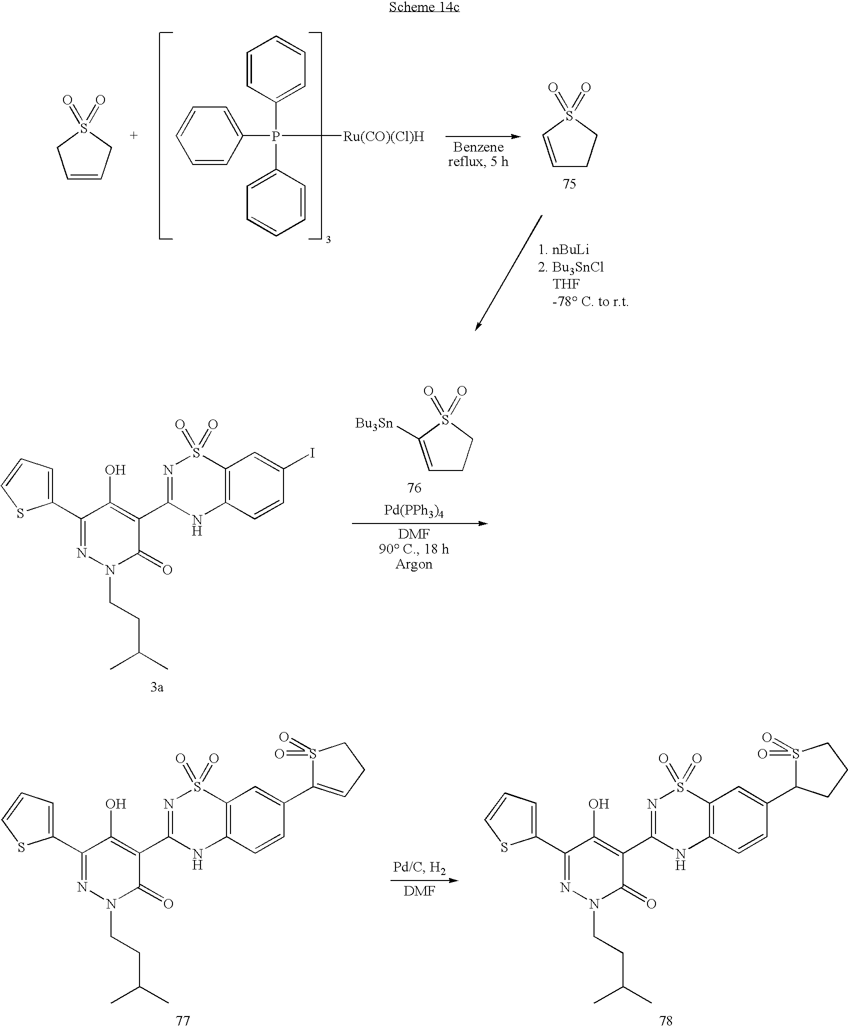

### 2,3-Dihydro-thiophene 1,1-dioxide (75)

2,5-Dihydro-thiophene 1,1-dioxide (10 g, 84.6 mmol) was dissolved in anhydrous benzene (60 mL). Carbonylchlorohydridotris(triphenylphosphine)ruthenium(II) (1.61 g, 1.69 mmol) was added and the mixture was heated to reflux while stirring for 5 h. Upon cooling to 25° C., the solution was decanted away from the black solid. The solution was concentrated in vacuo to give brown oil. Purification of the residue by flash column chromatography (50-70% EtOAc/Hexanes) afforded the desired product, 2,3-dihydro-thiophene 1,1-dioxide (75) (3.51 g, 29.6 mmol, 35% yield) as a yellow/brown oil that solidified when placed under vacuum. ¹H NMR (400 MHz, CDCl₃) δ: 2.91-2.96 (m, 2H), 3.21 (t, 2H, J=6.6 Hz), 6.62-6.65 (m, 1H), 6.72-6.76 (m, 1H).

### Tributyl-(1,1-dioxo-4,5-dihydro-1H-1λ⁶-thiophen-2-yl)-stannane (76)

2,3-Dihydro-thiophene 1,1-dioxide (75) (1.21 g, 10.2 mmol) was dissolved in THF (60 mL). The mixture was cooled to −78° C. and a 1.6M solution of n-butyl-lithium (6.72 mL, 10.7 mmol) was added dropwise over a period of 5 min. The mixture continued to stir at −78° C. for 30 min. Tri-butyl-tin-chloride (3.04 mL, 11.2 mmol) was added dropwise over a period of 5 min. The mixture stirred at −78° C. for 45 min and was then warmed to 25° C. over 45 min. The solution was concentrated in vacuo, dissolved in CHCl₃, poured into water (100 mL) and extracted into a 1:1 mixture of EtOAc and hexanes. The organic layer was dried over Na₂SO₄ and concentrated in vacuo to a brown/orange oil. Purification of the residue by flash column chromatography (20-30% EtOAc/Hexanes) afforded the desired product, tributyl-(1,1-dioxo-4,5-dihydro-1H-1λ⁶-thiophen-2-yl)-stannane (76) (1.13 g, 2.78 mmol, 27% yield) as a clear oil. ¹H NMR (400 MHz, CDCl₃) δ: 0.92 (t, 9H, J=7.5 Hz), 1.17-1.21 (m, 6H), 1.29-1.40 (m, 6H), 1.53-1.61 (m, 6H), 2.95-3.01 (m, 2H), 3.11-3.15 (m, 2H), 6.57 (t, 1H, J=3.1 Hz).

### 4-[7-(1,1-Dioxo-4,5-dihydro-1H-1λ⁶-thiophen-2-yl)-1,1-dioxo-1,4-dihydro-1λ⁶-benzo[1,2,4]thiadiazin-3-yl]-5-hydroxy-2-(3-methyl-butyl)-6-thiophen-2-yl-2H-pyridazin-3-one (77)

5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (0.214 g, 0.375 mmol) was suspended in anhydrous DMF (3.75 mL). Tributyl-(1,1-dioxo-4,5-dihydro-1H-1λ⁶-thiophen-2-yl)-stannane (76) (0.168 g, 0.413 mmol) was added followed by tetrakis(triphenylphosphine)palladium(0) (0.043 g, 0.0375 mmol). The solution was degassed while stirring under vacuum and then backfilled with argon. The mixture stirred at 90° C. for 18 h. Upon cooling 25° C., the mixture was diluted with EtOAc (100 mL) and shaken with 1 M HCl (3×50 mL), brine (50 mL), dried over MgSO₄ and concentrated to a brown/orange solid. Purification of the residue twice by flash column chromatography (column 1: 100% EtOAc, column 2: 0-30% EtOAc in CH₂Cl₂) afforded the desired product, 4-[7-(1,1-dioxo-4,5-dihydro-1H-1λ⁶-thiophen-2-yl)-1,1-dioxo-1,4-dihydro-1λ⁶-benzo[1,2,4]thiadiazin-3-yl]-5-hydroxy-2-(3-methyl-butyl)-6-thiophen-2-yl-2H-pyridazin-3-one (77) (0.118 g, 0.210 mmol, 56% yield) as a brown oil. LC-MS (ESI): m/e=561.3 [M+H⁺] (100%) (exact mass: 560.09).

### 4-[7-(1,1-Dioxo-tetrahydro-1λ⁶-thiophen-2-yl)-1,1-dioxo-1,4-dihydro-1λ⁶-benzo[1,2,4]thiadiazin-3-yl]-5-hydroxy-2-(3-methyl-butyl)-6-thiophen-2-yl-2H-pyridazin-3-one (78)

4-[7-(1,1-Dioxo-4,5-dihydro-1H-1λ⁶-thiophen-2-yl)-1,1-dioxo-1,4-dihydro-1λ6-benzo[1,2,4]thiadiazin-3-yl]-5-hydroxy-2-(3-methyl-butyl)-6-thiophen-2-yl-2H-pyridazin-3-one (77) (0.118 g, 0.21 mmol) was dissolved in DMF (3 mL). Palladium on carbon (0.15 g, 5% dry) was added. The solution was degassed while stirring under vacuum and then backfilled with hydrogen. Additional Palladium on carbon (100 mg) was added after stirring for 4 h. The solution was degassed while stirring under vacuum and then backfilled with hydrogen. The mixture continued to stir at 25° C. for 16 h. The mixture was filtered through celite and concentrated in vacuo to an orange oil. Purification by reverse phase HPLC (50-100% acetonitrile in water, 20 min), followed by trituration from a minimal amount of 1:1 MeOH and water followed by filtration afforded the desired product, 4-[7-(1,1-dioxo-tetrahydro-1λ⁶-thiophen-2-yl)-1,1-dioxo-1,4-dihydro-1λ⁶-benzo[1,2,4]thiadiazin-3-yl]-5-hydroxy-2-(3-methyl-butyl)-6-thiophen-2-yl-2H-pyridazin-3-one (78) (0.0055 g, 0.01 mmol, 5% yield) as a pale yellow powder. ¹H NMR (400 MHz, CDCl₃) δ: 1.03 (d, 6H, J=6.9 Hz), 1.70 (quintet, 1H, J=6.4 Hz), 1.78 (quartet, 2H, J=7.0 Hz), 2.23-2.35 (m, 1H), 2.40-2.54 (m, 2H), 2.60-2.67 (m, 1H), 3.18-3.25 (m, 1H), 3.33-3.39 (m, 1H), 4.25 (dd, 1H, J=11.8 Hz, J₂=7.0 Hz), 4.30 (t, 2H, J=7.4 Hz), 7.14 (t, 1H, J=4.3 Hz), 7.37 (d, 1H, J=8.5 Hz), 7.46 (d, 1H, J=4.5 Hz), 7.73 (dd, 1H, J=8.6 Hz, J₂=1.5 Hz), 7.95 (s, 1H), 7.99 (d, 1H, J=3.8 Hz). LC-MS (ESI): m/e=563.5 [M+H⁺] (100%) (exact mass: 562.10).

Method 15: Scheme 15 describes the synthesis of compound 66b of the Formula I.

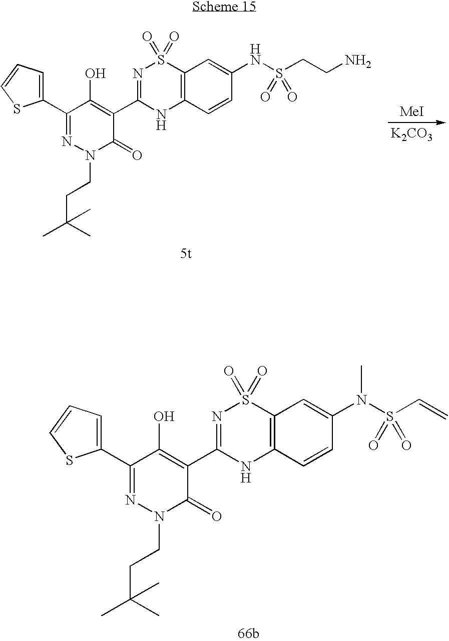

N-{3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-2-amino-ethanesulfonamide (5t) (80 mg, 0.14 mmol), MeI (0.4 mmol) and K₂CO₃ (38 mg, 0.28 mmol) were added into 1 mL of DMF, stirred at r.t for 24 hours. The LC-MS result indicated completion of the reaction. The reaction mixture was concentrated under reduced pressure. The residue was purified by HPLC to give pure desired product (66b) (9.12 mg, 12%). ¹H NMR (400 MHz, CDCl₃): δ 7.89 (d, 1H, J=4.0 Hz), 7.77 (m, 1H), 7.66 (m, 2H), 7.54 (d, 1H, J=8.8 Hz), 7.13 (dd, 1H, J=8.8, 4.0 Hz), 6.90 (d, 1H, J=16.4 Hz), 6.18 (d, 1H, J=10.4 Hz), 6.05 (d, 1H, J=16.4 Hz), 4.10 (m, 2H), 3.25 (s, 3H), 1.67 (m, 2H), 0.97 (s, 9H); LC-MS (ESI): m/e=578.3 [M+1]⁺ (exact ms:577.11).

Schemes 16-26 describe the synthetic routes and procedures for the indicated intermediates.

Scheme 16 describes the synthesis of the intermediate 2a.

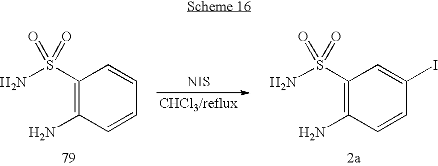

### 2-Amino-5-iodo-benzenesulfonamide (2a)

2-Amino-benzenesulfonamide (Aldrich) (79) (5.15 g, 29.3 mmol) was dissolved in chloroform (87 mL), and N-iodosuccinimide (Aldrich) (7.29 g, 30.77 mmol) was added under N₂ atmosphere. The mixture was refluxed for 24 hours, cooled to rt, and filtered through a center funnel. The solid was further washed with chloroform and 10% MeOH/CHCl₃ (3-8 times) to give desired product (2a) (6.78 g, 78%) as a brown crystalline solid. ¹H NMR (400 MHz, DMSO-d₆): 7.73 (d, 1H, J=2.0 Hz), 7.45 (dd, 1H, J=8.8, 2.4 Hz), 7.31 (s, 2H), 6.62 (d, 1H, J=8.8 Hz), 5.98 (s, 2H).

Scheme 17 describes the general synthesis of sulfonamide intermediate 4.

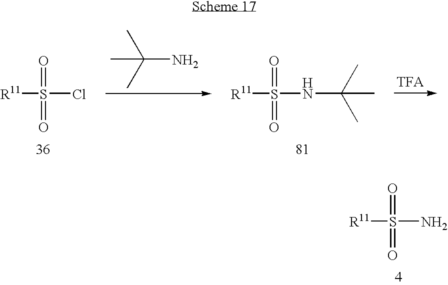

In a general procedure, sulfonylchloride (36) was treated with tert-butyl amine to form intermediate 81. The tert-butyl protecting group of compound 81 was removed by treatment with TFA to give sulfonamide 4.

### EXAMPLE 17-1

Scheme 17a describes the synthesis of compound 4b.

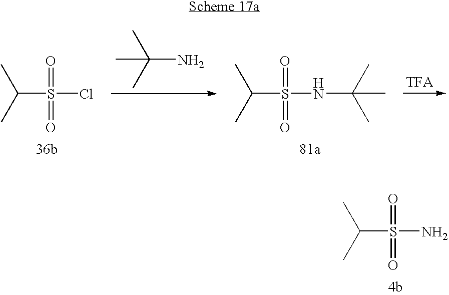

In this specific example, tert-butyl amine (15.75 mL, 150 mmol) was dissolved in anhydrous THF (85 mL) and cooled to −20° C. under N₂ atmosphere, isopropylsulfonyl chloride (36b) (8.64 mL, 75 mmol) was added in a period of 15 min. The reaction mixture was stirred from 0° C. to rt for 24 hours, filtered though a centered funnel and the filtrate was concentrated under reduced pressure. The residue was dissolved in DCM (200 mL), washed with 1.0M aqueous HCl, H₂O and brine, dried over Na₂SO₄. The solvent was removed under reduced pressure to give desired product 81a as a white solid. Compound 81a was dissolved in TFA (40 mL), and stirred for 24 hours under N₂ atmosphere. TFA was removed under reduced pressure and the residue was recystallized from benzene-ethanol-hexanes to give desired propane-2-sulfonic acid amide (4b) (4.4 g, 48%) as a white crystalline. ¹H NMR (400 MHz, DMSO-d₆) δ: 6.60 (s, 2H), 3.00 (m, 1H), 1.21 (d, 6H, J=6.4 Hz).

### EXAMPLE 17-2

Scheme 17b describes the synthesis of compound 4c.

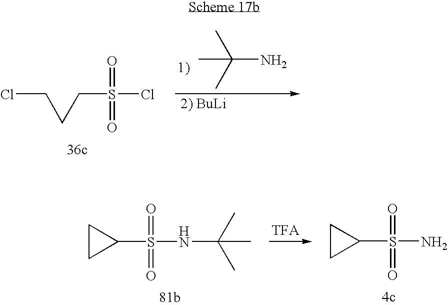

In this specific example, the intermediate 4c was prepared based on the procedure described in J. Li et. al., Synlett, 725-728 (2006).

Cyclopropanesulfonic acid amide (4c). ¹H NMR (400 MHz, DMSO-d₆) δ: 6.74 (s, 2H), 2.48 (m, 1H), 0.89 (m, 4H).

Scheme 18 describes a general procedure for synthesis of 2-Amino-5-alkanesulfonylamino-benzenesulfonamide 84.

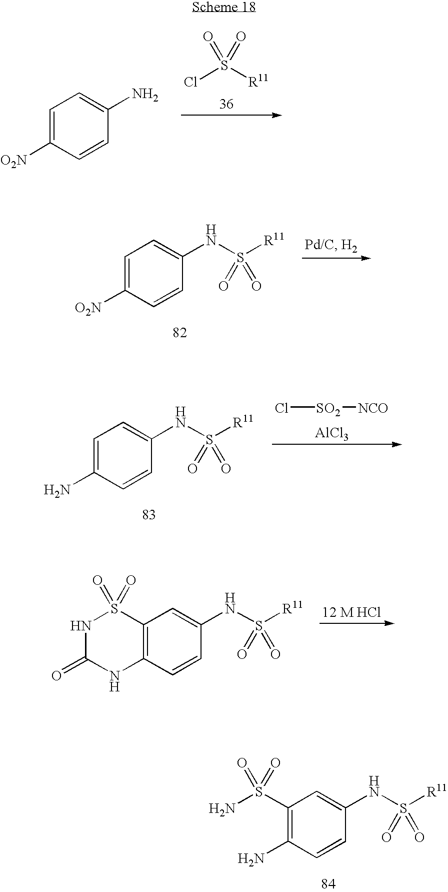

In a general procedure, 4-nitro-aniline can react with alkanesulfonyl chloride (36) under basic conditions to form sulfonamide intermediate 82 which can be reduced to the compound 83. Compound 83 can be treated with chloro-sulfonyl-isocyanate in the presence of AlCl₃ followed by treating with HCl to give the desired intermediate 84.

### EXAMPLE 18-1

Scheme 18a describes the synthesis of 2-Amino-5-methanesulfonylamino-benzenesulfonamide 16a.

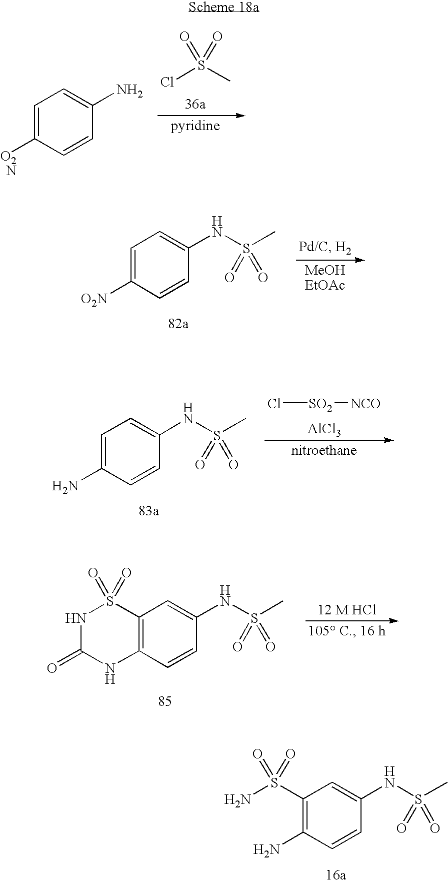

### N-(4-Nitro-phenyl)-methanesulfonamide (82a)

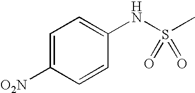

4-Nitro-phenylamine (25 g, 181 mmol) was dissolved in pyridine (450 mL). Methanesulfonyl chloride (36a) (14.0 mL, 181 mmol) was added dropwise while stirring. The mixture stirred for 24 hours at room temperature. The solution was concentrated in vacuo to a volume of 50 mL. The mixture was diluted with ethyl acetate (400 mL), washed with 1M aqueous hydrochloric acid (5×200 mL). The combined aqueous layers were back extracted with ethyl acetate (200 mL). The combined organic layers were dried over magnesium sulfate and concentrated in vacuo to a volume of ˜250 mL. The product precipitated and was collected by vacuum filtration. The filtrate was concentrated in vacuo to about half volume and additional product precipitated. The solid was collected by vacuum filtration. The solids were combined to afford the desired product 82a, N-(4-nitro-phenyl)-methanesulfonamide (25 g, 64% yield). ¹H NMR (400 MHz, DMSO-d₆) δ: 3.17 (s, 3H), 7.35 (d, 2H, J=9.4 Hz), 8.20 (d, 2H, J=9.1 Hz), 10.69 (s, 1H).

### N-(4-Amino-phenyl)-methanesulfonamide (83a)

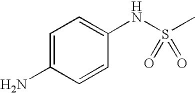

N-(4-Nitro-phenyl)-methanesulfonamide (82a) (25 g, 115.62 mmol) was dissolved in N,N-dimethylformamide (15 mL) with gentle heating to −50° C. via heat gun. Ethyl acetate (100 mL) and methyl alcohol (100 mL) were added followed by 10% palladium on carbon (4 g). The mixture was degassed while stirring and the flask was charged with hydrogen gas via balloon. The mixture stirred at room temperature for 4.5 hours. The mixture was filtered through celite (rinsed with ethyl acetate) and concentrated in vacuo to a yellow green solution with a volume of ˜10 mL. Methylene chloride (−50 mL) was added and a solid began to precipitate. The mixture stirred at room temperature for 30 minutes. The solid was collected by vacuum filtration and dried in vacuo to afford the desired product, N-(4-amino-phenyl)-methanesulfonamide 83a (15.32 g, 71% yield) as a beige powder. ¹H NMR (400 MHz, DMSO-d₆) δ: 2.79 (s, 3H), 5.00 (s, 2H), 6.49 (d, 2H, J=8.5 Hz), 6.87 (d, 2H, J=8.6 Hz), 8.87 (s, 1H).

### N-(1,1,3-Trioxo-1,2,3,4-tetrahydro-1λ⁶-benzo[1,2,4]thiadiazin-7-yl)-methanesulfonamide (85)

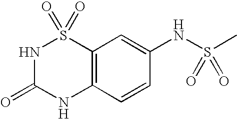

Chloro-sulfonyl-isocyanate (1.7 mL, 19.6 mmol) was dissolved in nitroethane (10 mL) and chilled to −40° C. under nitrogen. N-(4-Amino-phenyl)-methanesulfonamide (83a) (3 g, 16.1 mmol) was added dropwise as a pre-dissolved solution in nitroethane (25 mL). The mixture stirred at 40° C. for 15 minutes. Aluminum chloride (8 g, 60 mmol) was added and the mixture was heated at 110° C. for 30 minutes while stirring. The mixture was poured over ice (˜150 g). Upon melting, the product was extracted into ethyl acetate (5×250 mL). The combined organic phase was dried over magnesium sulfate and concentrated in vacuo to afford the desired product, N-(1,1,3-trioxo-1,2,3,4-tetrahydro-1λ⁶-benzo[1,2,4]thiadiazin-7-yl)-methanesulfonamide (85) (3.63 g, 77% yield) as a beige solid. ¹H NMR (400 MHz, DMSO-d₆) δ: 3.00 (s, 3H), 7.22 (d, 1H, J=8.5 Hz), 7.46 (dd, 1H, J₁=8.8 Hz, J₂=2.7 Hz), 7.51 (d, 1H, J=2.4 Hz), 9.92 (s, 1H), 11.20 (s, 1H).

### 2-Amino-5-methanesulfonylamino-benzenesulfonamide (16a)

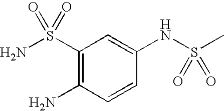

N-(1,1,3-Trioxo-1,2,3,4-tetrahydro-1λ⁶-benzo[1,2,4]thiadiazin-7-yl)-methanesulfonamide (85) (1 g, 3.4 mmol) was suspended in 12M aqueous hydrochloric acid (60 mL). The mixture was stirred at 105° C. for 16 hours. All solids were dissolved at this point. The mixture was diluted with water (250 mL). The solution was concentrated in vacuo to an orange solid. The solid was dissolved in water (20 mL) and concentrated in vacuo to an orange solid. The solid was dissolved in water (5 mL) and the product was extracted into ethyl acetate (6×20 mL). The combined organic phase was dried over magnesium sulfate and concentrated in vacuo to an orange solid. Purification by flash column chromatography (75% ethyl acetate in hexanes, Merck silica gel 60, 40-63 μm) afforded the desired product, 2-amino-5-methanesulfonylamino-benzenesulfonamide (16a) (0.41 g, 45% yield), as a beige solid upon concentrating in vacuo. ¹H NMR (400 MHz, DMSO-d₆) δ: 2.86 (s, 3H), 5.77 (s, 2H), 6.76 (d, 1H, J=8.6 Hz), 7.11 (dd, 1H, J₁=8.6 Hz, J₂=2.4 Hz), 7.25 (bs, 2H), 7.43 (d, 1H, J=3.1 Hz), 9.16 (s, 1H).

Scheme 19 describes the synthesis of (7-methanesulfonylamino-1,1-dioxo-1,4-dihydro-1λ⁶-benzo[1,2,4]thiadiazin-3-yl)-acetic acid (40).

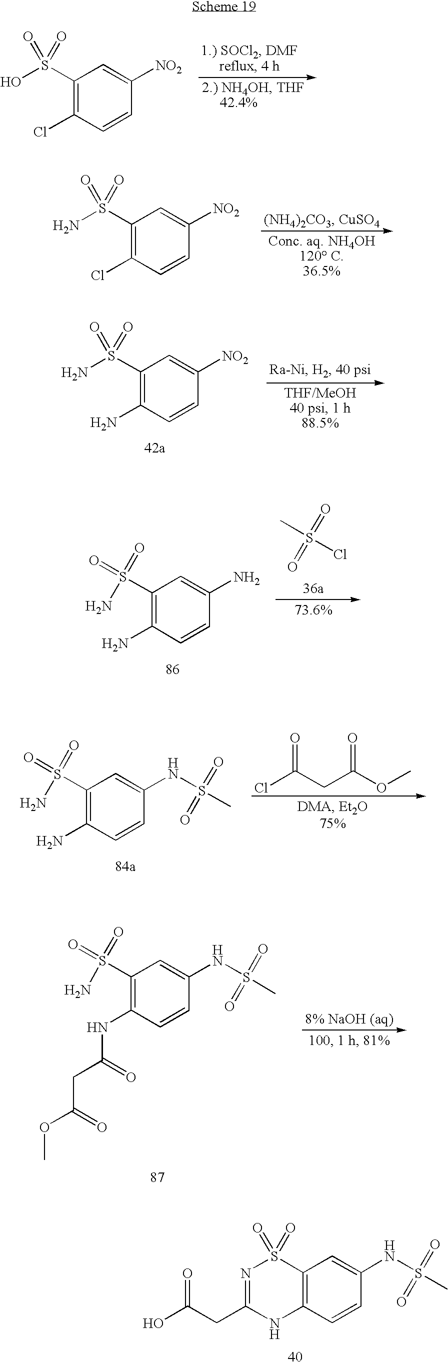

### 2-Chloro-5-nitrobenzenesulfonamide

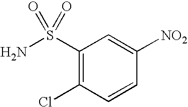

### 2-amino-5-methanesulfonylamino-benzenesulfonamide (84a)

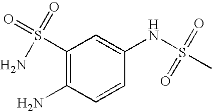

A mixture of 2,5-diamino-benzenesulfonamide (86) (5.71 g, 30.5 mmol) was dissolved in dichloromethane (140 ml, 0.21M), followed by addition of pyridine (9.65 g, 122 mmol). The mixture was cooled to 0° C. and methanesulfonyl chloride (36a) (3.84 g, 33.5 mmol) was dissolved in dichloromethane (70 ml, 0.43M) and added over a period of 1 hour. The reaction mixture was then allowed to warm up and was stirred for 17 h at 25° C. The resulting mixture was concentrated under reduced pressure, resuspended in ethyl acetate (400 ml) and washed with 1N HCl (400 ml). Precipitate formed upon agitation, and was filtered off. Aqueous phase was extracted once more with ethyl acetate (400 ml), organic layers combined and concentrated under reduced pressure to near dryness (50 ml), and filtered, isolating additional desired product. Two filter cakes were combined, dried under high vacuum to afford the desired product (84a) (5.96 g, 73.6%) as a pale pink solid. ¹H NMR (400 MHz, CD₃OD) δ: 2.93 (s, 3H), 6.93 (d, 1H, J=9.2 Hz), 7.25 (dd, 1H, J₁=8.5 Hz, J₂=3.3 Hz), 7.64 (d, 1H, J=2.2 Hz), LC-MS (ESI): m/e=266.3 [M+1]⁺ (exact ms: 265.02).

### N-(4-methanesulfonylamino-2-sulfamoyl-phenyl)-malonamic acid methyl ester (87)

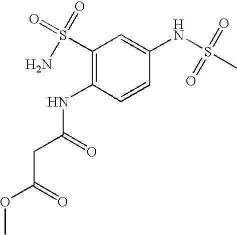

2-amino-5-methanesulfonylamino-benzenesulfonamide (84a) (3.7 g, 13.94 mmol) was dissolved in N,N-dimethylacetamide (11.1 ml, 1.25M) and diethyl ether (11.1 ml, 1.25M) and cooled to 0° C. Chlorocarbonyl-acetic acid methyl ester was then added dropwise over a period of 10 minutes. Upon addition, mixture was allowed to warm up to 25° C. and stirring continued for 1 hour. The mixture was dissolved in ethyl acetate (200 ml) and washed with water (200 ml). The aqueous phase was washed once again with ethyl acetate (200 ml), organic layers combined and concentrated under reduced pressure to near dryness (50 ml). To the stirring mixture of product and ethyl acetate, hexanes were added (50 ml) in a dropwise manner, and stirring continued for 4 hours. The resulting mixture was then filtered, dried under high vacuum to afford the desired product (87) (3.96 g, 75%) as a white solid. ¹H NMR (400 MHz, CD₃OD) δ: 1.32 (t, 3H, J=7.0 Hz), 3.01 (s, 3H), 3.60 (s, 2H), 4.25 (quartet, 2H, J=7.0 Hz), 7.44 (dd, 1H, J₁=2.3 Hz, J₂=2.2 Hz), 7.87 (d, 1H, J=2.3 Hz), 7.92 (d, 1H, J=9.6 Hz), LC-MS (ESI⁺): m/e=363.1 [M+1]⁺ (exact ms: 365.04).

### (7-Methanesulfonylamino-1,1-dioxo-1,4-dihydro-1λ⁶-benzo[1,2,4]thiadiazin-3-yl)-acetic acid (40)

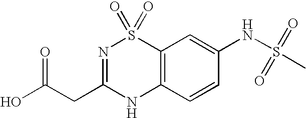

N-(4-methanesulfonylamino-2-sulfamoyl-phenyl)-malonamic acid methyl ester (87) was dissolved in 8 wt % NaOH in water (2.1 g, 52.5 mmol) and heated to 100° C. for 1.5 hours. Upon complete conversion, the mixture was cooled and the pH adjusted to 2-3 by addition of 12N HCl at which point product precipitates out. Solids were filtered and dried under vacuum to afford desired product (40) (0.71 g, 81%). ¹H NMR (400 MHz, DMSO-d₆) δ: 2.20 (s, 3H), 2.89 (s, 2H), 7.16 (d, 1H, J=9.2 Hz), 7.31 (dd, 1H, J₁=8.6 Hz, J₂=2.4 Hz), 7.40 (d, 1H, J=2.2 Hz), LC-MS (ESI): m/e=334.0 [M+1]⁺ (exact ms: 333.01).

Scheme 20 describes the synthesis of Synthesis of (4-Amino-3-sulfamoyl-phenyl)-carbamic acid tert-butyl ester (89).

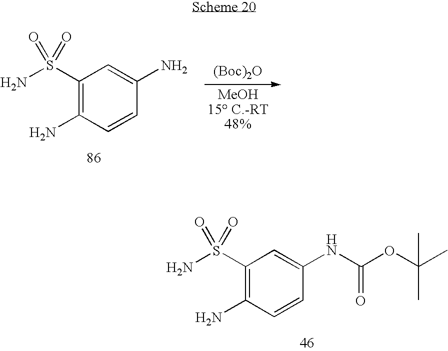

To a solution of 2,5-diaminobenzenesulfonamide (86) (7.22 g, 38.56 mmol) in methanol (50 mL) at 15° C. was added to Boc anhydride (8.92 g, 40.88 mmol) in methanol (20 mL). The solution was then warmed to RT and mixed for 4 h then the reaction was quenched by addition of N,N-dimethylenediamine (0.63 mL). The solvent was removed under reduced pressure, the solid was redissolved in ethyl acetate and filtered through a fritted funnel (medium) and the ethyl acetate was removed under reduced pressure. Product isolation was achieved by recrystallization with ethyl acetate/heptane (1/1, 4 volumes) to give tert-butyl 4-amino-3-(aminosulfonyl)phenyl carbamate (46) (5.32 g, 48%).

Scheme 21 describes the synthesis of toluene-4-sulfonic acid 1-trifluoromethyl-cyclobutylmethyl ester (90).

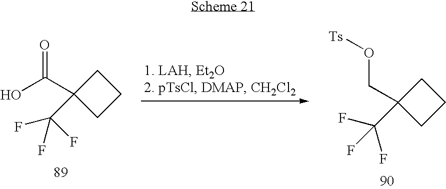

### Toluene-4-sulfonic acid 1-trifluoromethyl-cyclobutylmethyl ester (90)

1-Trifluoromethyl-cyclobutanecarboxylic acid (89) (1.5 g, 8.93 mmol) was dissolved in anhydrous Et₂O (15 mL). The solution was chilled to 0° C. A 2M solution of LAH in THF (5 mL, 10 mmol) was added dropwise to the stirring solution. The mixture stirred at 0° C. for 30 min and at room temperature for 3 h. The excess LAH was quenched by the careful addition of water (20 mL). The mixture was diluted with Et₂O (20 mL) and washed with 1M HCl (25 mL). The aqueous layer was back extracted with Et₂O (2×15 mL). The combined organic extracts were dried over MgSO₄ and filtered. Triethylamine (1.63 mL, 11.6 mmol), 4-methyl-benzenesulfonyl chloride (1.87 g, 9.82 mmol) and dimethyl-pyridin-4-yl-amine (0.893 g) were added to the ether solution. The mixture stirred at room temperature for 16 h. Only starting materials were observed. The mixture was concentrated under partial vacuum to afford a thick oil. The oil was dissolved in CH₂Cl₂ (50 mL) and stirred at 45° C. for 16 h. The mixture was diluted with CH₂Cl₂ (50 mL), washed with 1M HCl (25 mL), aqueous saturated NaHCO₃ (25 mL), brine (25 mL) and passed through a plug of silica gel. The filtrate was concentrated in vacuo to afford the desired product, toluene-4-sulfonic acid 1-trifluoromethyl-cyclobutylmethyl ester (90) (1.86 g, 6 mmol, 68%), as a clear oil. ¹H NMR (400 MHz, DMSO-d₆) δ: 1.81-1.97 (m, 4H), 2.11-2.22 (m, 2H), 2.42 (s, 3H), 4.26 (s, 2H), 7.48 (d, 2H, J=8.4 Hz), 7.80 (d, 2H, J=7.7 Hz).

Scheme 22 describes the synthesis of toluene-4-sulfonic acid 2-cyclobutyl-ethyl ester (92).

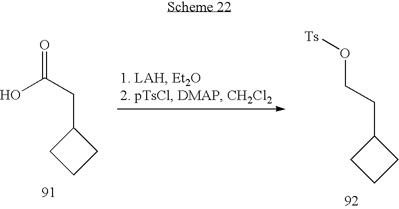

Cyclobutyl-acetic acid (91) (1 g, 8.76 mmol) was dissolved in Et₂O (15 mL). A 2M solution of LAH in THF (5 mL, 10 mmol) was added dropwise to the stirring solution. The mixture stirred at 0° C. for 30 min and at room temperature for 4 h. The excess LAH was quenched by the careful addition of water (20 mL). The mixture was diluted with Et₂O (100 mL), washed with 0.5M HCl (2×25 mL), brine (15 mL), dried over MgSO₄ filtered and concentrated under partial vacuum (just enough to remove diethyl ether) to a clear oil. The oil was dissolved in CH₂Cl₂ (20 mL). 4-Methyl-benzenesulfonyl chloride (1.84 g, 9.64 mmol), Et₃N (1.6 mL, 11.39 mmol) and dimethyl-pyridin-4-yl-amine (0.108 g, 0.88 mmol) were added and the mixture stirred at room temperature for 16 h. The mixture was diluted with CH₂Cl₂ (50 mL) followed by 1M HCl (25 mL). The mixture was passed through a plug of silica gel. The filtrate was concentrated in vacuo to afford the desired product, toluene-4-sulfonic acid 2-cyclobutyl-ethyl ester (92) (1.243 g, 4.89 mmol, 55.7%), as a clear oil. ¹H NMR (400 MHz, DMSO-d₆) δ: 1.46-1.56 (m, 2H), 1.65 (quartet, 2H, J=6.8 Hz), 1.69-1.80 (m, 2H), 1.84-1.91 (m, 2H), 2.22 (septet, 1H, J=7.8 Hz), 2.41 (s, 3H), 3.92 (t, 2H, J=6.3 Hz), 7.46 (d, 2H, J=8.1 Hz), 7.75 (d, 2H, J=7.7 Hz).

Scheme 23 describes the synthesis of toluene-4-sulfonic acid 2-cyclopentyl-ethyl ester (94).

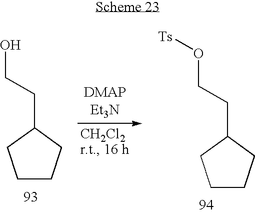

### Toluene-4-sulfonic acid 2-cyclopentyl-ethyl ester (94)

2-Cyclopentyl-ethanol (93) (2 g, 17.5 mmol) was dissolved in CH₂Cl₂ (90 mL). 4-Methyl-benzenesulfonyl chloride (3.66 g, 19.2 mmol), Et₃N (3.19 mL, 22.75 mmol) and dimethyl-pyridin-4-yl-amine (0.214 g, 1.75 mmol) were added and the mixture stirred at room temperature for 16 h. The mixture was washed with 1M HCl (3×25 mL). The organic phase was passed through a plug of silica gel. The filtrate was concentrated to afford the desired product, toluene-4-sulfonic acid 2-cyclopentyl-ethyl ester (94) (4.61 g, 17.18 mmol, 98.2% yield), as a yellow oil. ¹H NMR (400 MHz, DMSO-d₆) δ: 0.93-1.01 (m, 2H), 1.36-1.62 (m, 8H), 1.72 (septet, 1H, J=7.6 Hz), 2.41 (s, 3H), 4.00 (t, 2H, J=6.2 Hz), 7.46 (d, 2H, J=8.0 Hz), 7.76 (d, 2H, J=8.1 Hz).

Scheme 24 describes the synthesis of toluene-4-sulfonic acid 3-methyl-pentyl ester (96).

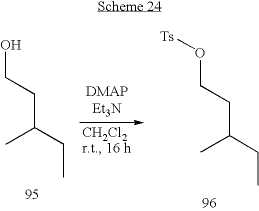

### Toluene-4-sulfonic acid 3-methyl-pentyl ester

3-Methyl-pentan-1-ol (95) (2 g, 19.57 mmol) was dissolved in CH₂Cl₂ (50 mL). 4-Methyl-benzenesulfonyl chloride (4.1 g, 21.53 mmol), Et₃N (3.6 mL, 25.44 mmol) and dimethyl-pyridin-4-yl-amine (0.244 g, 2 mmol) were added and the mixture stirred at room temperature for 16 h. The mixture was diluted with CH₂Cl₂ (50 mL) and washed with 1M HCl (3×50 mL). The organic phase was passed through a plug of silica gel. The filtrate was concentrated to afford the desired product, toluene-4-sulfonic acid 3-methyl-pentyl ester (96) (3.52 g, 13.73 mmol, 70.2% yield), as a clear/yellow oil. ¹H NMR (400 MHz, DMSO-d₆) δ: 0.73-0.84 (m, 6H), 0.98-1.10 (m, 1H), 1.15-1.25 (m, 1H), 1.30-1.39 (m, 2H), 1.52-1.62 (m, 1H), 2.41 (s, 3H), 3.98-4.07 (m, 2H), 7.45 (d, 2H, J=8.6 Hz), 7.76 (d, 2H, J=8.9 Hz).

Scheme 25 describes the synthesis of 5-hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a).

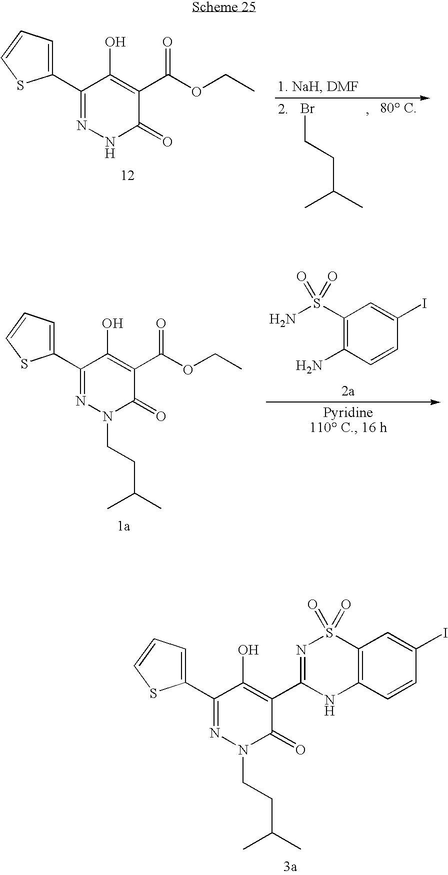

### 5-Hydroxy-2-(3-methyl-butyl)-3-oxo-6-thiophen-2-yl-2,3-dihydro-pyridazine-4-carboxylic acid ethyl ester (1a)

5-Hydroxy-3-oxo-6-thiophen-2-yl-2,3-dihydro-pyridazine-4-carboxylic acid ethyl ester (12) (5 g, 18.78 mmol) was suspended in anhydrous DMF (60 mL). A 60% suspension of NaH in mineral oil (1.73 g, 43.19 mmol) was added. The mixture was stirred at 25° C. for 10 min. with occasional venting. 1-Bromo-3-methyl-butane (2.36 mL, 19.7 mmol) was added and the mixture stirred at 75° C. for 16 hours. Upon cooling to 25° C., the mixture was diluted with 1 M HCl (250 mL) and the product extracted into EtOAc (300 mL). The organic phase was further washed with 1 M HCl (2×200 mL), brine (200 mL), dried over MgSO₄ and concentrated in vacuo. Purification of the residue by flash column chromatography (100% CH₂Cl₂) afforded the desired product, 5-hydroxy-2-(3-methyl-butyl)-3-oxo-6-thiophen-2-yl-2,3-dihydro-pyridazine-4-carboxylic acid ethyl ester (1a) (6.2 g, 18.45 mmol, 98% yield), as a yellow waxy solid. ¹H NMR (400 MHz, CDCl₃): δ=0.99 (d, 6H, J=6.2 Hz), 1.50 (t, 3H, J=7.1 Hz), 1.64-1.76 (m, 3H), 4.22 (t, 2H, J=7.1 Hz), 4.53 (q, 2H, J=7.0 Hz), 7.10 (dd, 1H, J=4.9, 3.6 Hz), 7.38 (d, 1H, J=4.0 Hz), 7.88 (d, 1H, J=4.6 Hz), 13.87 (s, 1H). ¹³C NMR (100 MHz, CDCl₃): δ=14.3, 22.6, 25.9, 37.3, 50.8, 63.1, 103.2, 127.3, 127.6, 128.2, 134.8, 135.8, 156.5, 163.7, 171.4. LC-MS (ESI⁺): m/e=337.2 [M+H⁺] (100%) (exact mass: 336.11).

### 5-Hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one

5-Hydroxy-2-(3-methyl-butyl)-3-oxo-6-thiophen-2-yl-2,3-dihydro-pyridazine-4-carboxylic acid ethyl ester (1a) (5 g, 14.9 mmol) and 2-amino-5-iodo-benzenesulfonamide (2a) (4.66 g, 15.6 mmol) were combined in anhydrous pyridine (75 mL). The flask was degassed and backfilled with argon. The mixture stirred at 110° C. for 16 h. Upon cooling to room temperature, the mixture was concentrated in vacuo to a volume of approximately 10 mL. MeOH (75 mL) was added. The product slowly precipitated while stirring over 4 h. The solid was collected by filtration, rinsed with MeOH (2×20 mL) and dried under vacuum to afford the desired product, 5-hydroxy-4-(7-iodo-1,1-dioxo-1,4-dihydro-1λ⁶-benzo[1,2,4]thiadiazin-3-yl)-2-(3-methyl-butyl)-6-thiophen-2-yl-2H-pyridazin-3-one (3a) (3.36 g, 5.89 mmol, 40% yield), as a beige powder. ¹H NMR (400 MHz, DMSO-d₆) δ: 0.95 (d, 6H, J=6.2 Hz), 1.57-1.71 (m, 3H), 4.15 (t, 2H, J=6.5 Hz), 7.16 (t, 1H, J=4.3 Hz), 7.40 (d, 1H, J=8.6 Hz), 7.67 (d, 1H, J=5.4 Hz), 7.90 (d, 1H, J=3.8 Hz), 8.02 (dd, 1H, J₁=8.6 Hz, J₂=1.6 Hz), 8.11 (d, 1H, J=2.3 Hz), 13.93 (s, 1H). LC-MS (ESI): m/e=571.20 [M+H⁺] (100%) (exact mass: 569.99).

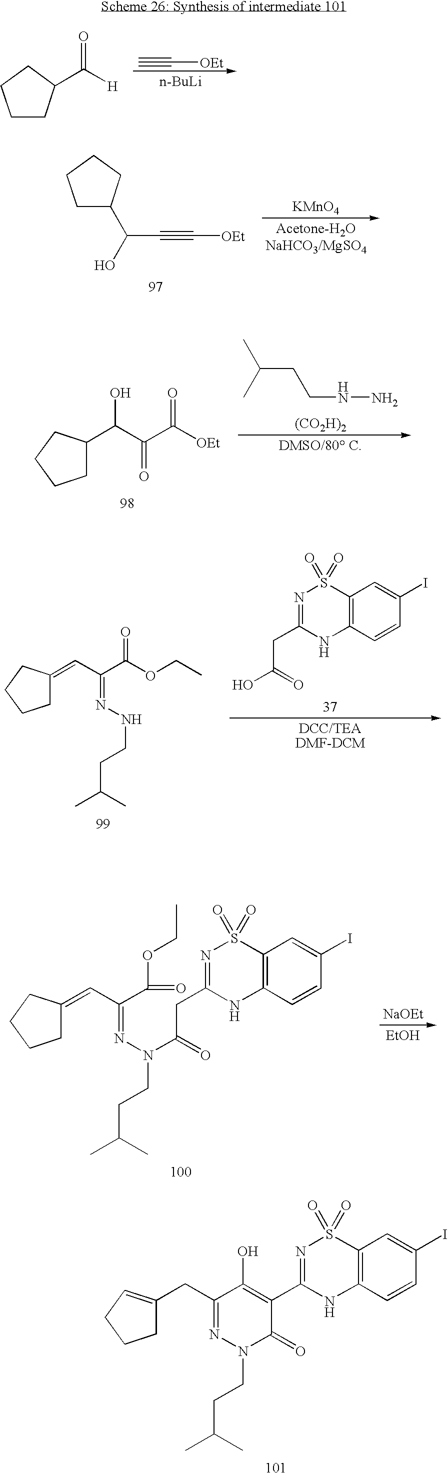

In a typical synthetic route, α-keto-ester 98 was made from cyclopantanone. See J. H. Tatlock, J. Org. Chem. 60, 6221-6223 (1995).

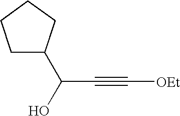

### 1-Cyclopentyl-3-ethoxy-prop-2-yn-1-ol (97)

To a solution of ethyl ethynyl ether (5 g, mmol, 50 wt % solution in hexanes, 71.4 mmol) in anhydrous THF at −78° C. under N₂, n-BuLi (2.5 M in Hexanes, 28.6 mL, 71.4 mmol) was added slowly over 20 min. The reaction mixture was stirred at −78° C. for 2 h. A solution of cyclopentanecarbaldehyde (5.83 g, 59.5 mmol) in dry THF (50 mL) was added slowly over 15 min. The resulting mixture was stirred for 2 h until TLC showed completion of the reaction. The mixture was poured into NH₄Cl aqueous solution, extracted with EtOAc. The combined organic layers were washed brine and dried over Na₂SO₄. The solvents were removed under reduced pressure and the residue was purified by flash chromatography on silica gel to give the desired product 1-cyclopentyl-3-ethoxy-prop-2-yn-1-ol (97) (3.80 g, 38%). ¹H NMR (400 MHz, CDCl₃): δ 4.29 (m, 1H), 4.10 (m, 2H), 2.16 (m, 1H), 1.79 (m, 2H), 1.65 (m, 2H), 1.61 (m, 2H), 1.47 (m, 2H), 1.41 (t, 3H, J=7.2 Hz).

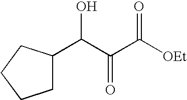

### 3-Cyclopentyl-3-hydroxy-2-oxo-propionic acid ethyl ester (98)

To a solution of 1-cyclopentyl-3-ethoxy-prop-2-yn-1-ol (97) (1.66 g, 10 mmol) in acetone (100 mL), a solution of NaHCO₃ (504 mg, 6 mmol) and MgSO₄ (2.40 g, 20 mmol) in H₂O (100 mL) was added, followed by KMnO₄ (3.95 g, 25 mmol). The reaction mixture was stirred at rt for 15 min, and then poured into H₂O, extracted with EtOAc (3×). The combined organic layers were washed with H₂O (3×) and brine until colorless, and dried over Na₂SO₄. The solvents were removed under reduced pressure and the residue was purified by flash chromatography on silica gel to give the desired product 3-cyclopentyl-3-hydroxy-2-oxo-propionic acid ethyl ester (98) (1.0 g, 54%). The crude product was used directly to next step without purification.

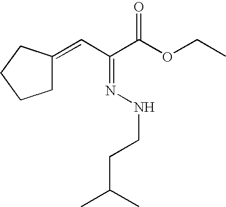

### 3-Cyclopentylidene-2-[(3-methyl-butyl)-hydrazono]-propionic acid ethyl ester (99)

To a solution of 3-cyclopentyl-3-hydroxy-2-oxo-propionic acid ethyl ester (98) (1.0 g, 5.37 mmol) in absolute ethanol (10 mL), (3-Methyl-butyl)-hydrazine oxalate (1.03 g, 5.37 mmol) and NaOAc (528 mg, 6.44 mmol) were added. The mixture was stirred at 80° C. under N₂ atmosphere for 2 h. The reaction mixture was diluted with EtOAc, washed with H₂O and brine, dried over Na₂SO₄. The solvents were removed under reduced pressure and the residue was purified by flash chromatography on silica gel to give the desired product 3-cyclopentylidene-2-[(3-methyl-butyl)-hydrazono]-propionic acid ethyl ester (99) (300 mg, 21%). ¹H NMR (400 MHz, CDCl₃): δ 5.81 (m, 1H), 4.28 (q, 2H, J=7.2 Hz), 3.51 (t, 2H, J=7.6 Hz), 2.79 (m, 1H), 2.48 (m, 2H), 2.07 (m, 2H), 1.78-1.60 (m, 5H), 1.50 (m, 2H), 1.35 (t, 3H, J=7.2 Hz), 0.94 (d, 6H, J=6.8 Hz); LC-MS (ESI): m/e 267.4 [M+H]⁺ (exact ms: 266.20).

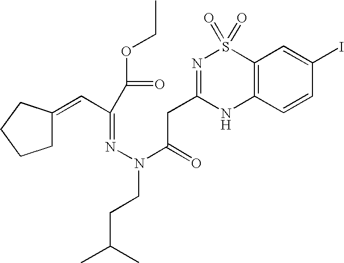

### 3-Cyclopentylidene-2-[[2-(7-iodo-1,1-dioxo-1,4-dihydro-1l6-benzo[1,2,4]thiadiazin-3-yl)-acetyl]-(3-methyl-butyl)-hydrazono]-propionic acid ethyl ester (100)

To a solution of 3-cyclopentylidene-2-[(3-methyl-butyl)-hydrazono]-propionic acid ethyl ester (99) (290 mg, 1.09 mmol), and (7-Iodo-1,1-dioxo-1,4-dihydro-1,6-benzo[1,2,4]thiadiazin-3-yl)-acetic acid (37) (440 mg, 1.20 mmol) in 1 mL of anhydrous DMF and 3 mL of anhydrous methylene chloride, 1,3-dicyclohexyl-carbodiimide (DCC) (1.0 M in DCM, 1.3 mL, 1.3 mmol) was added and the resulting mixture was stirred at room temperature for 1 h. Triethylamine (TEA) (0.3 mL, 2.18 mmol) was added to the reaction mixture and stirred at rt for 1.5 h. The solid was filtered off through a centered funnel and the filtrate was concentrated under reduced pressure to give desired amide product, 3-cyclopentylidene-2-[[2-(7-iodo-1,1-dioxo-1,4-dihydro-1l6-benzo[1,2,4]thiadiazin-3-yl)-acetyl]-(3-methyl-butyl)-hydrazono]-propionic acid ethyl ester (100), LC-MS (ESI⁺): m/e 615.3 [M+1]⁺, (exact ms: 614.11). The crude material was used directly to next step without purification.

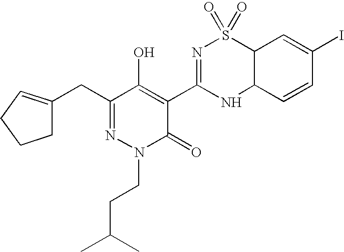

### 6-Cyclopent-1-enylmethyl-5-hydroxy-4-(7-iodo-1,1-dioxo-1,4-dihydro-1l6-benzo[1,2,4]thiadiazin-3-yl)-2-(3-methyl-butyl)-2H-pyridazin-3-one (101)

The above obtained crude product 3-cyclopentylidene-2-[[2-(7-iodo-1,1-dioxo-1,4-dihydro-1l6-benzo[1,2,4]thiadiazin-3-yl)-acetyl]-(3-methyl-butyl)-hydrazono]-propionic acid ethyl ester (100) was dissolved in 4 mL of EtOH, NaOEt (0.81 mL, 2.18 mmol) was added. The mixture was stirred from room temperature to 50° C. for 40 min. LC-MS showed completion of the reaction. The mixture was cooled to room temperature, 5% HCl aqueous solution was added to adjust PH value to 6, extracted with EtOAc. The combined organic layers were washed brine and dried over Na₂SO₄. The solvents were removed under reduced pressure and the residue was purified by flash chromatography on silica gel to give the desired product, 6-cyclopent-1-enylmethyl-5-hydroxy-4-(7-iodo-1,1-dioxo-1,4-dihydro-1l6-benzo[1,2,4]thiadiazin-3-yl)-2-(3-methyl-butyl)-2H-pyridazin-3-one (101), (80 mg, 13% two steps). ¹H NMR (400 MHz, CDCl₃): δ 8.27 (d, 1H, J=2.0 Hz), 7.93 (dd, 1H, J=8.8, 2.0 Hz), 7.06 (d, 1H, J=8.8 Hz), 5.40 (m, 1H), 4.22 (t, 2H, J=6.8 Hz), 3.52 (s, 2H), 2.33 (m, 4H), 1.92 (m, 2H), 1.71 (m, 3H), 1.00 (d, 6H, J=6.8 Hz); LC-MS (ESI): m/e 569.4 [M+H]⁺ (exact ms: 568.06).

### Biological Testing

The ability of compounds of Formula I to inhibit HCV replication can be demonstrated in the following in vitro assays.

### NS5B Polymerase Inhibition Assay (IC₅₀)

Compounds were tested for HCV polymerase inhibition. Assays were performed in a 96-well streptavidin-coated FlashPlate using 50 nM enzyme, 0.5 μCi of [α-³³P]GTP, 0.6 μM GTP, and 50 nM 5′biotinylated oligo (rG₁₃)/poly rC in 20 mM Tris-HCl, pH 7.5, 5 mM MgCl₂, 20 mM NaCl, 5 mM dithiothreitol, and 0.1 g/L BSA. The reaction was stopped by aspiration after 2 h at room temperature and the plate was washed several times. After washing, incorporated radioactivity was counted using a Microbeta scintillation counter.

The compounds of Formula I listed individually in Table I exhibited surprisingly potent NS5B polymerase inhibition, wherein the activity (IC₅₀) is defined as follows:

TABLE 1

| Structure | Name | IC₅₀ (avg) |
|---|---|---|
| 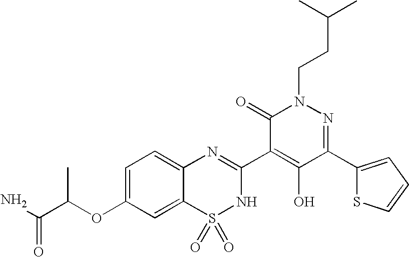 | 2-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yloxy}-propionamide | +++ |
| 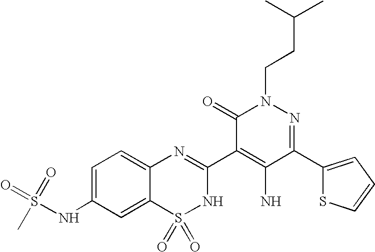 | N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 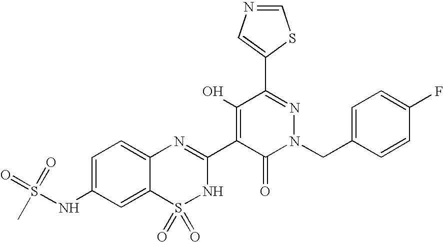 | N-{3-[2-(4-Fluoro-benzyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide | +++ |
| 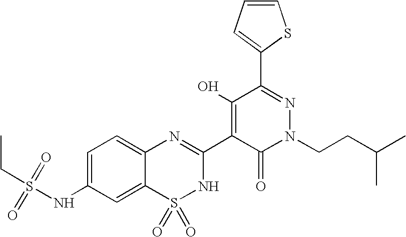 | Ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 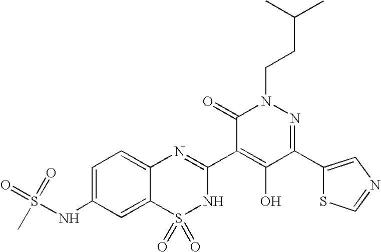 | N-{3-[5-Hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide | +++ |
| 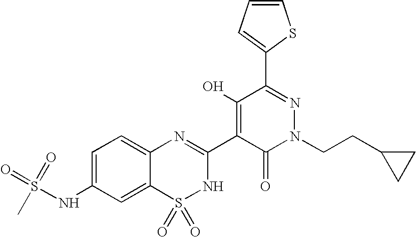 | N-{3-[2-(2-Cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 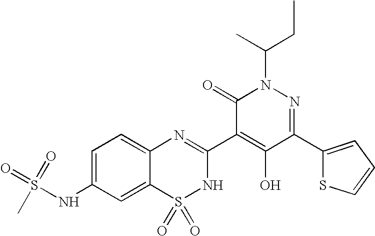 | N-[3-(2-sec-Butyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | ++ |
| 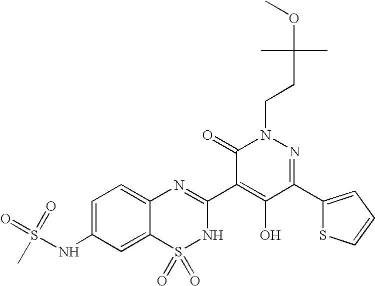 | N-{3-[5-Hydroxy-2-(3-methoxy-3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 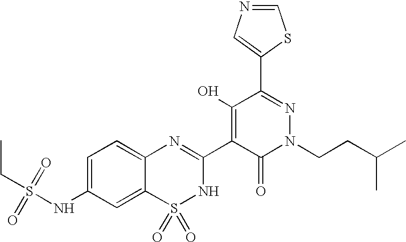 | Ethanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide | +++ |
| 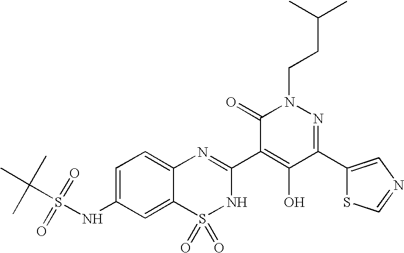 | 2-Methyl-propane-2-sulfonic acid{3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide | +++ |
| 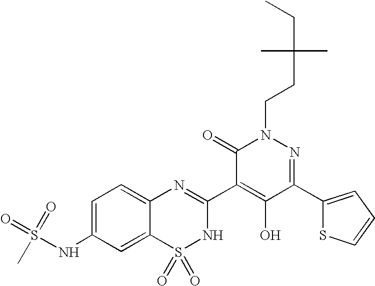 | N-{3-[2-(3,3-Dimethyl-pentyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 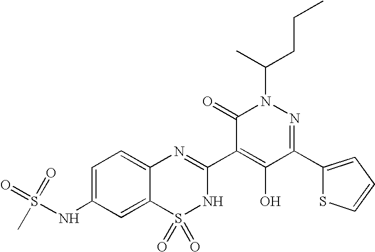 | N-{3-[5-Hydroxy-2-(1-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | ++ |
| 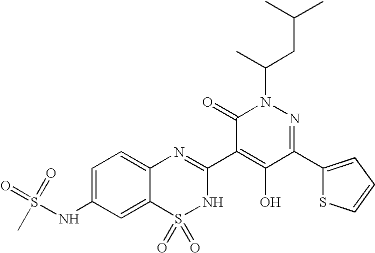 | N-{3-[2-(1,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | ++ |
| 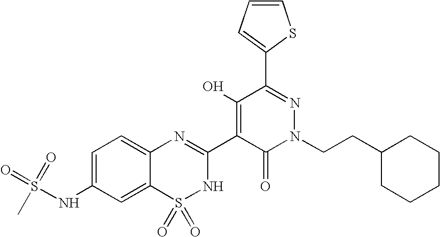 | N-{3-[2-(2-Cyclohexyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | ++ |
| 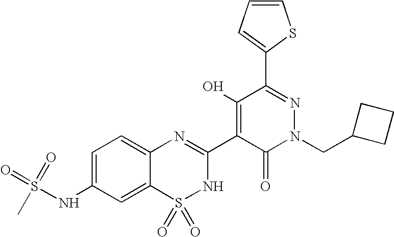 | N-[3-(2-Cyclobutylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 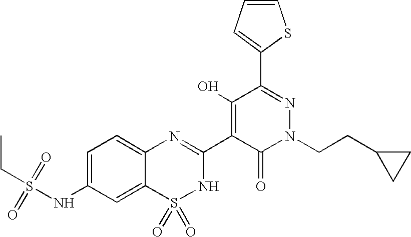 | Ethanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 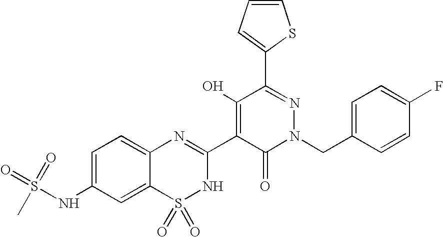 | N-{3-[2-(4-Fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 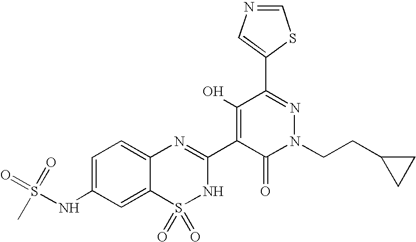 | N-{3-[2-(2-Cyclopropyl-ethyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide | +++ |
| 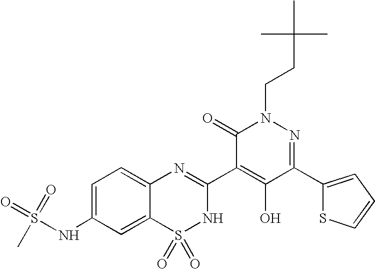 | N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 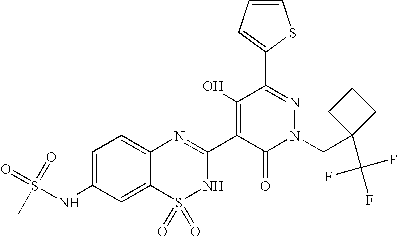 | N-{3-[5-Hydroxy-3-oxo-6-thiophen-2-yl-2-(1-trifluoromethyl-cyclobutylmethyl)-2,3-dihydro-pyridazin-4-yl]-1,1-dioxo-1,2-dihydro-1λ⁶-benzo[1,2,4]thiadiazin-7-yl}-methanesulfonamide | +++ |
| 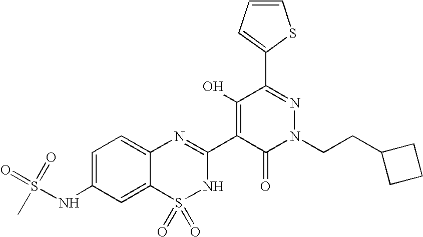 | N-{3-[2-(2-Cyclobutyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 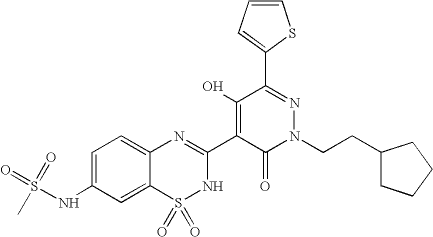 | N-{3-[2-(2-Cyclopentyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 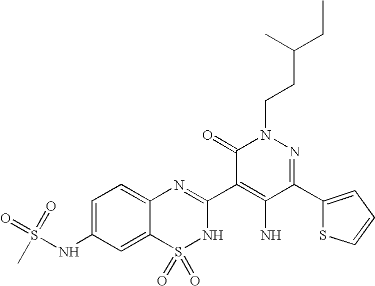 | N-{3-[5-Hydroxy-2-(3-methyl-pentyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 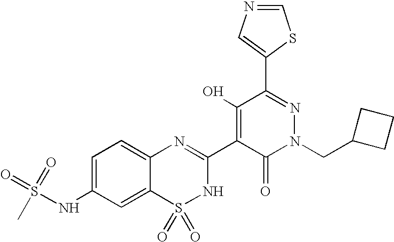 | N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-methanesulfonamide | +++ |
| 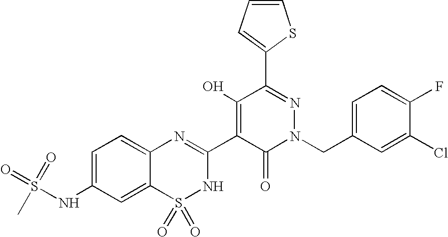 | N-{3-[2-(3-Chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 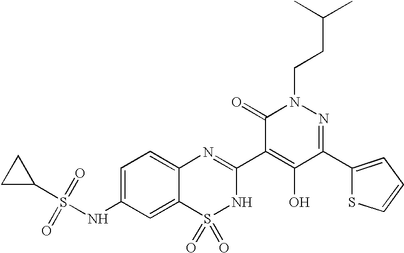 | Cyclopropanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 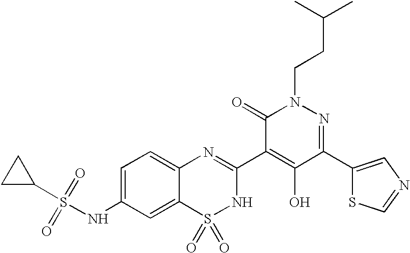 | Cyclopropanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide | +++ |
| 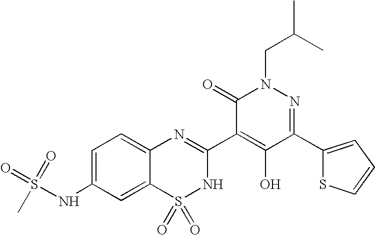 | N-[3-(5-Hydroxy-2-isobutyl-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 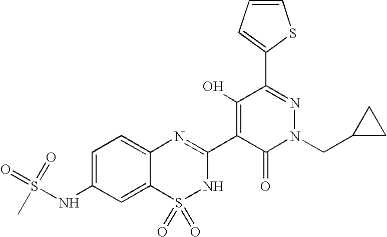 | N-[3-(2-Cyclopropylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 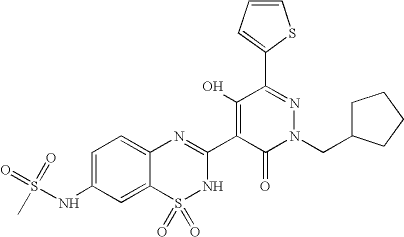 | N-[3-(2-Cyclopentylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 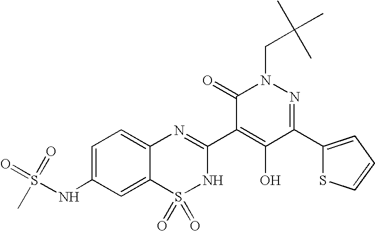 | N-{3-[2-(2,2-Dimethyl-propyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 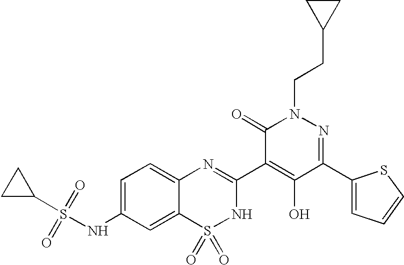 | Cyclopropanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 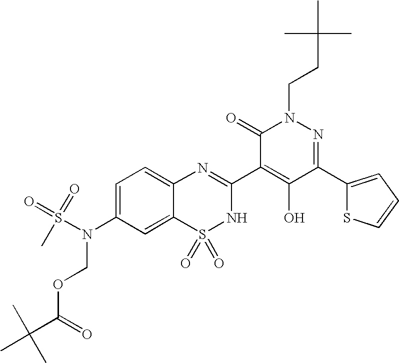 | 2,2-Dimethyl-propionic acid ({3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methylester | ++ |
| 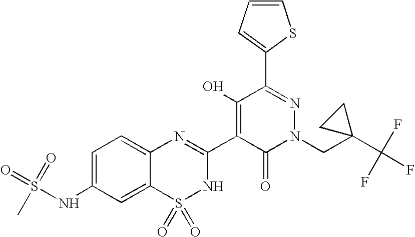 | N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-methanesulfonamide | +++ |
| 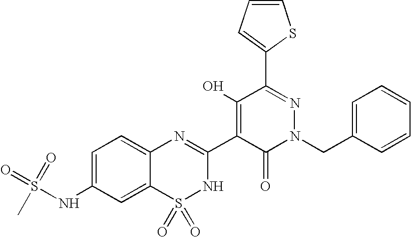 | N-[3-(2-Benzyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 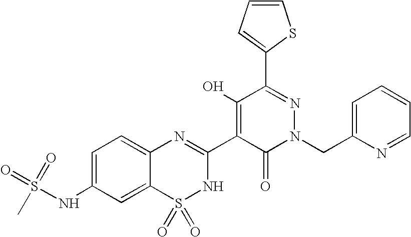 | N-[3-(5-Hydroxy-3-oxo-2-pyridin-2-ylmethyl-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide | +++ |
| 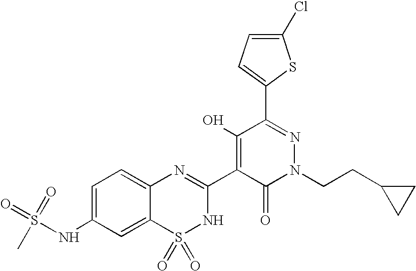 | N-{3-[6-(5-Chloro-thiophen-2-yl)-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl)-methanesulfonamide | ++ |
| 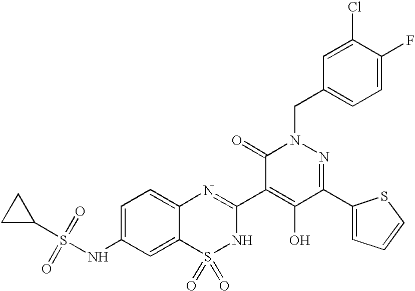 | Cyclopropanesulfonic acid {3-[2-(3-chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | ++ |
| 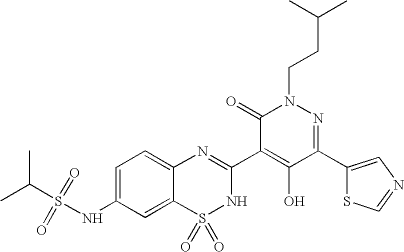 | Propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide | +++ |
| 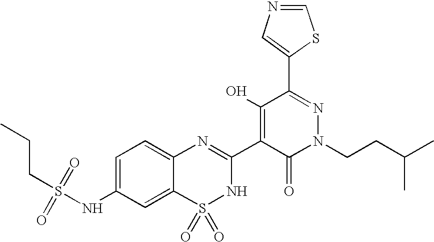 | Propane-1-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide | +++ |
| 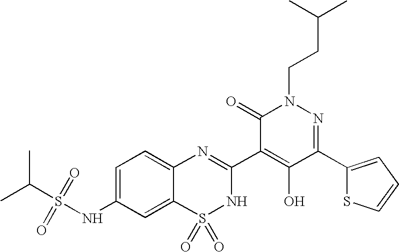 | Propane-2-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 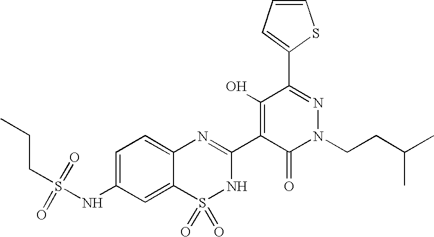 | Propane-1-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 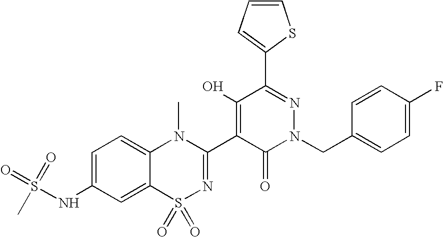 | N-{3-[2-(4-Fluoro-benzyl)-5-hydroxy-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-methanesulfonamide | + |
| 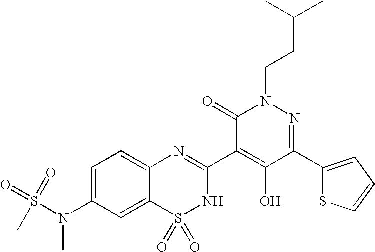 | N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide | +++ |
| 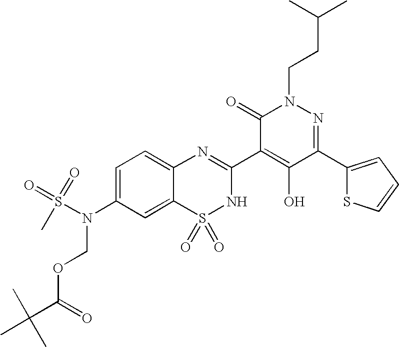 | 2,2-Dimethyl-propionic acid ({3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methylester | ++ |
| 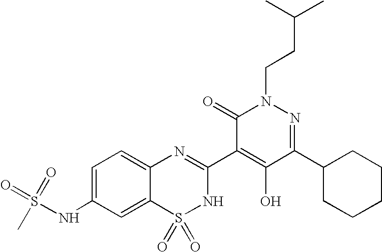 | N-{3-[6-Cyclohexyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 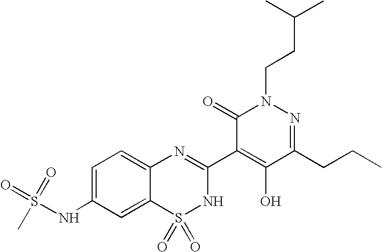 | N-{3-[5-Hydroxy-2-(3-methyl-butyl)-3-oxo-6-propyl-2,3-dihydro-pyridazin-4-yl]-1,1-dioxo-1,2-dihydro-1λ⁶-benzo[1,2,4]thiadiazin-7-yl}-methanesulfonamide | +++ |
| 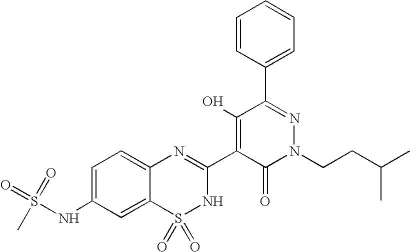 | N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-methanesulfonamide | +++ |
| 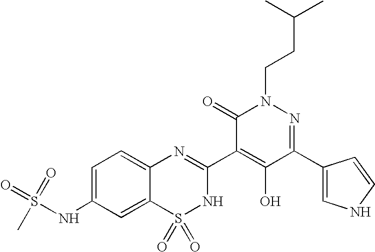 | N-{3-[5-Hydroxy-2-(3-methyl-butyl)-3-oxo-6-(1H-pyrrol-3-yl)-2,3-dihydro-pyridazin-4-yl]-1,1-dioxo-1,2-dihydro-1λ⁶-benzo[1,2,4]thiadiazin-7-yl}-methanesulfonamide | +++ |
| 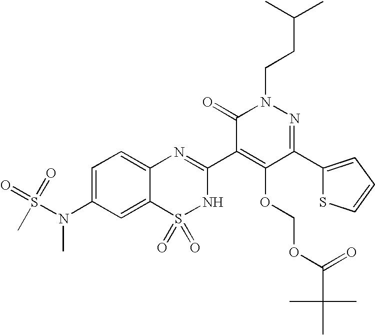 | 2,2-Dimethyl-propionic acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yloxymethyl ester | + |
| 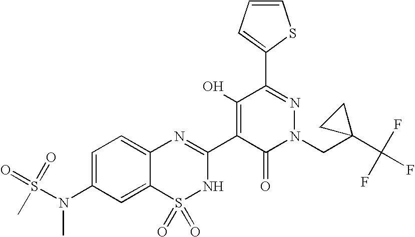 | N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-N-methyl-methanesulfonamide | +++ |
| 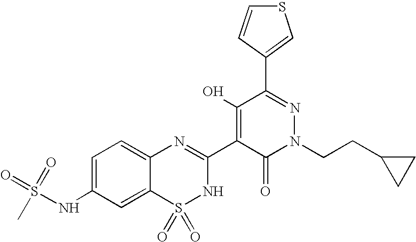 | N-{3-[2-(2-Cyclopropyl-ethyl)-5-hydroxy-3-oxo-6-thiophen-3-yl-2,3-dihydro-pyridazin-4-yl]-1,1-dioxo-1,2-dihydro-1λ⁶-benzo[1,2,4]thiadiazin-7-yl}-methanesulfonamide | +++ |
| 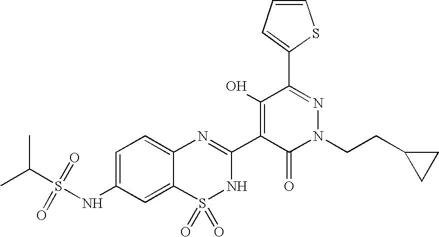 | Propane-2-sulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 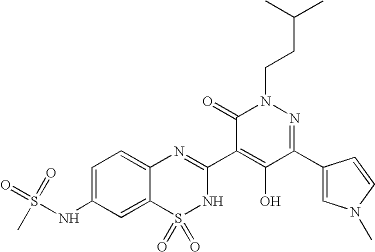 | N-{3-[5-Hydroxy-2-(3-methyl-butyl)-6-(1-methyl-1H-pyrrol-3-yl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 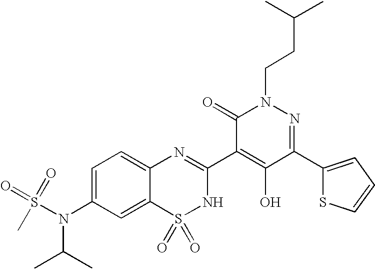 | N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-isopropyl-methanesulfonamide | + |
| 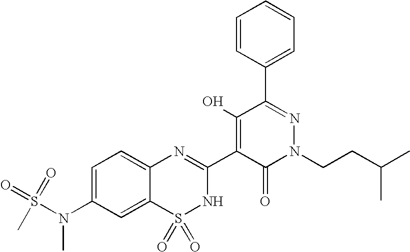 | N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-N-methyl-methanesulfonamide | + |
| 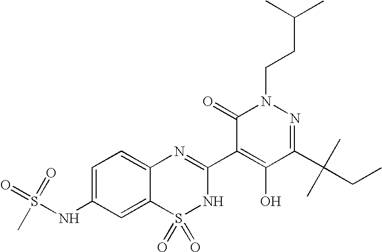 | N-{3-[6-(1,1-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 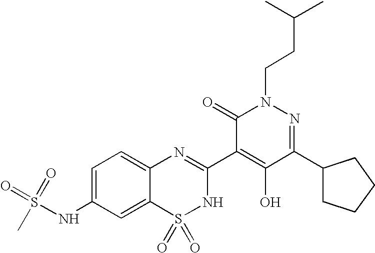 | N-{3-[6-Cyclopentyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 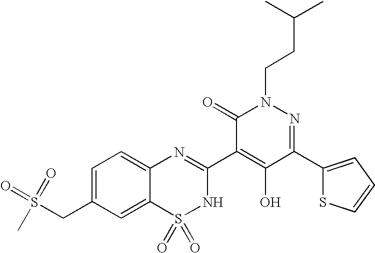 | 5-Hydroxy-4-(7-methanesulfonylmethyl-1,1-dioxo-1,2-dihydro-1λ⁶-benzo[1,2,4]thiadiazin-3-yl)-2-(3-methyl-butyl)-6-thiophen-2-yl-2H-pyridazin-3-one | ++ |
| 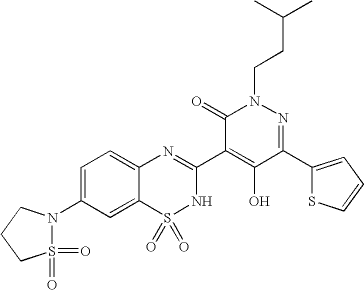 | 4-[7-(1,1-Dioxo-1l6-isothiazolidin-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one | ++ |
| 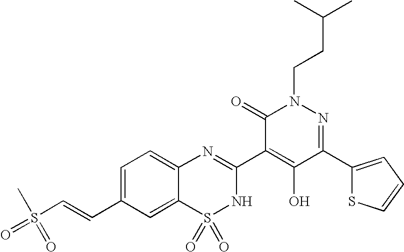 | 5-Hydroxy-4-[7-(2-methanesulfonyl-vinyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one | ++ |
| 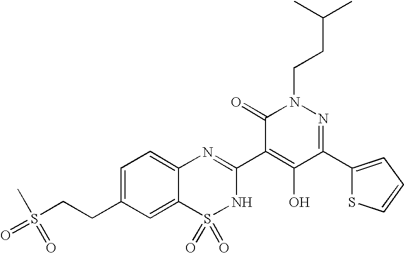 | 5-Hydroxy-4-[7-(2-methanesulfonyl-ethyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one | ++ |
| 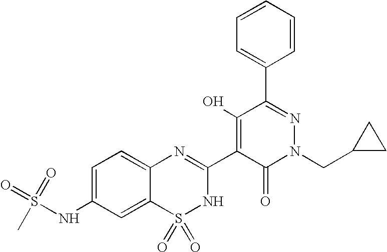 | N-[3-(2-Cyclopropylmethyl-5-hydroxy-3-oxo-6-phenyl-2,3-dihydro-pyridazin-4-yl)-1,1-dioxo-1,2-dihydro-1l6-benzo[1,2,4]thiadiazin-7-yl]-methanesulfonamide | ++ |
| 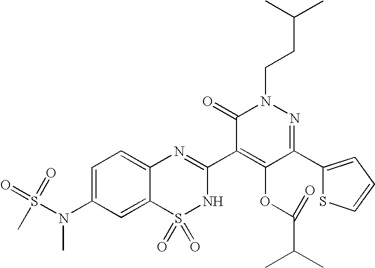 | Isobutyric acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yl ester | + |
| 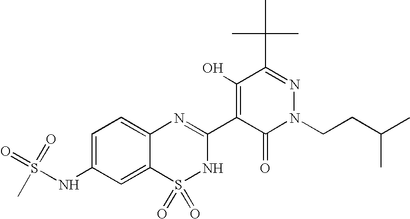 | N-{3-[6-tert-Butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 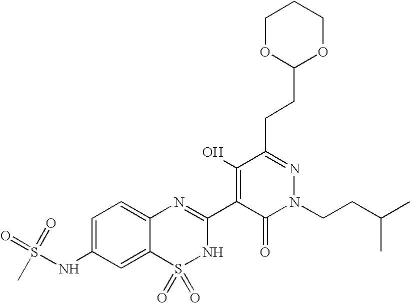 | N-{3-[6-(2-[1,3]Dioxan-2-yl-ethyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 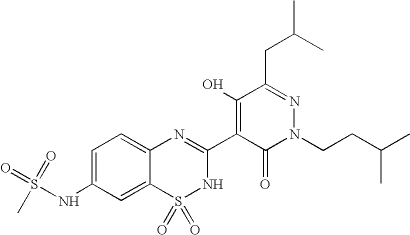 | N-{3-[5-Hydroxy-6-isobutyl-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 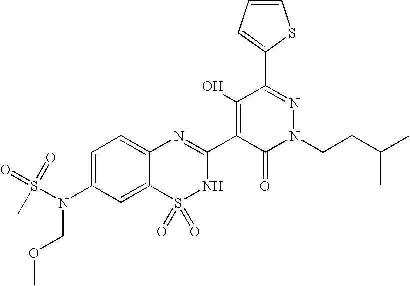 | N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methoxymethyl-methanesulfonamide | ++ |
| 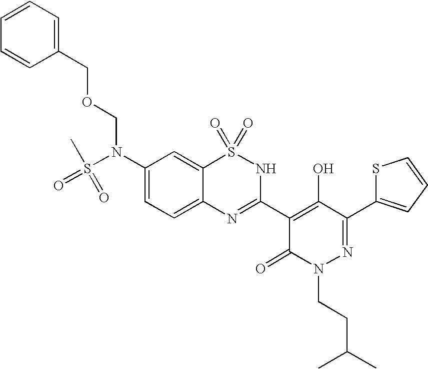 | N-Benzyloxymethyl-N-{3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | + |
| 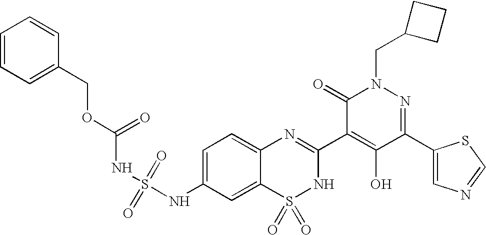 | N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-3-carbamic-acid-benzyl-ester-sulfamide | +++ |
| 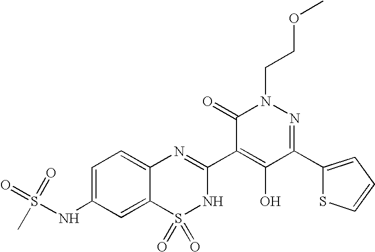 | N-{3-[5-Hydroxy-2-(2-methoxy-ethyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide | +++ |
| 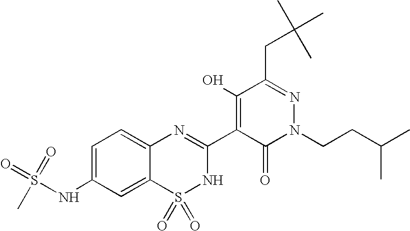 | N-{3-[6-(2,2-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 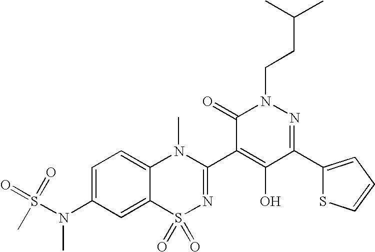 | N-{3-[5-Hydroxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide | + |
| 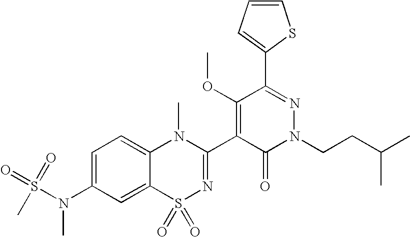 | N-{3-[5-Methoxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide | notdetermined |
| 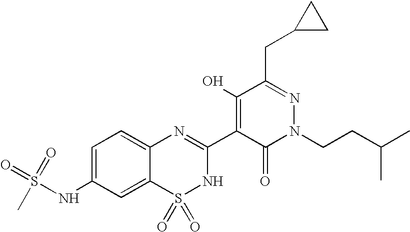 | N-{3-[6-Cyclopropylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 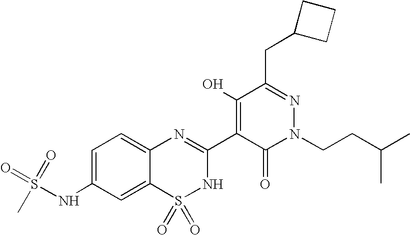 | N-{3-[6-Cyclobutylmethyl-5-hydroxy-2-(3-methyl-butyl)-3-oxo-2,3-dihydro-pyridazin-4-yl]-1,1-dioxo-1,2-dihydro-1λ⁶-bnezo[1,2,4]thiadiazin-7-yl}-methanesulfonamide | ++ |
| 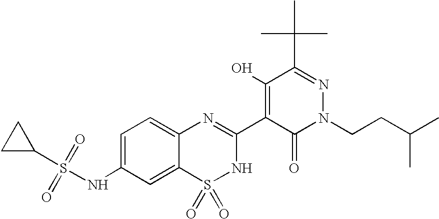 | Cyclopropanesulfonic acid {3-[6-tert-butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-amide | + |
| 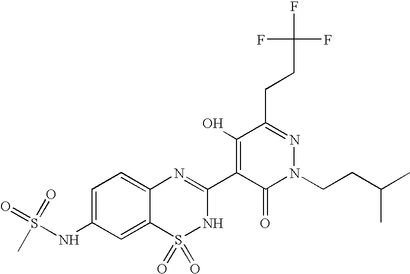 | N-{3-[5-Hydroxy-2-(3-methyl-butyl)-3-oxo-6-(3,3,3-trifluoro-propyl)-2,3-dihydro-pyridazin-4-yl]-1,1-dioxo-1,2-dihydro-1λ⁶-benzo[1,2,4]thiadiazin-7-yl}-methanesulfonamide | ++ |
| 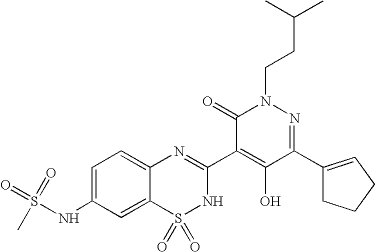 | N-{3-[6-Cyclopent-1-enyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 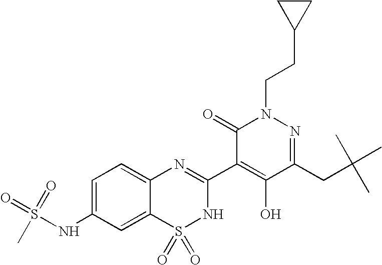 | N-{3-[2-(2-Cyclopropyl-ethyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 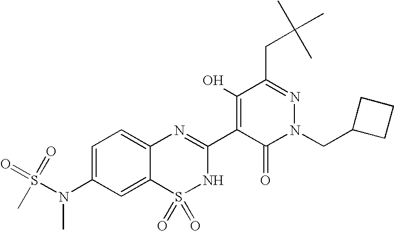 | N-{3-[2-Cyclobutylmethyl-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-N-methyl-methanesulfonamide | + |
| 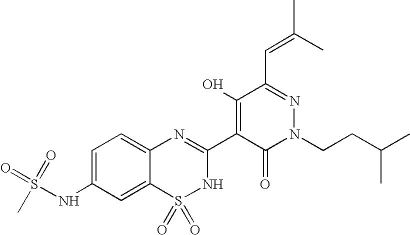 | N-{3-[5-Hydroxy-2-(3-methyl-butyl)-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 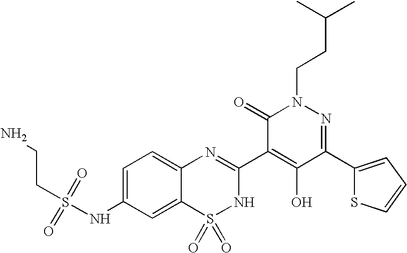 | 2-Amino-ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 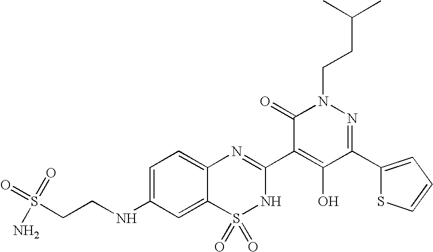 | 2-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-ylamino}-ethanesulfonic acid amide | ++ |
| 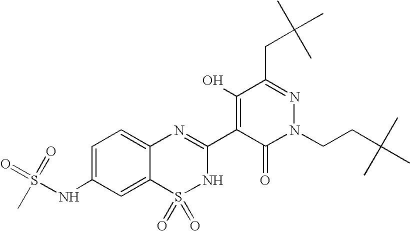 | N-{3-[2-(3,3-Dimethyl-butyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 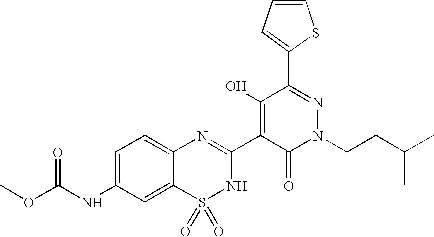 | {3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid methyl ester | ++ |
| 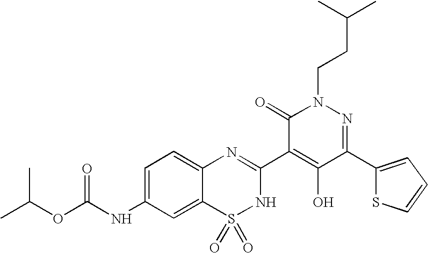 | {3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid isopropyl ester | + |
| 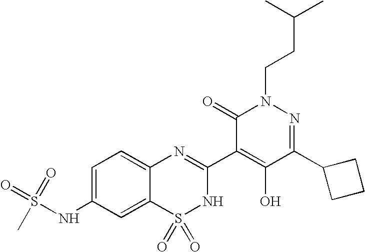 | N-{3-[6-Cyclobutyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 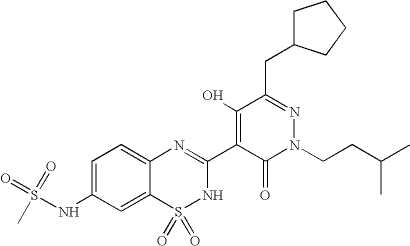 | N-{3-[6-Cyclopentylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 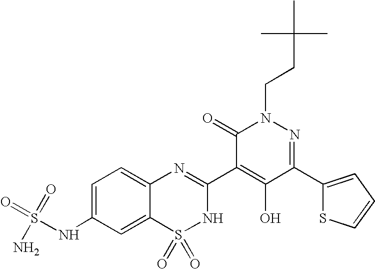 | N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-sulfamide | +++ |
| 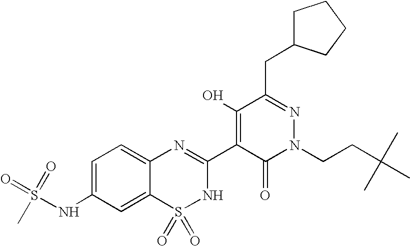 | N-{3-[6-Cyclopentylmethyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 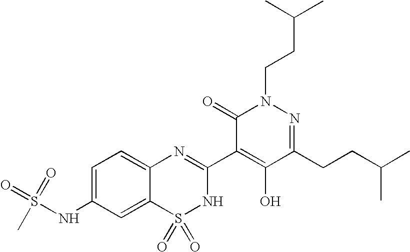 | N-{3-[5-Hydroxy-2,6-bis-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 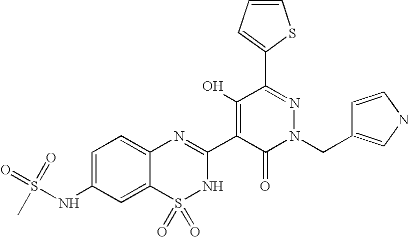 | N-[3-(5-Hydroxy-3-oxo-6-thiophen-2-yl-2-thiophen-3-ylmethyl-2,3-dihydro-pyridazin-4-yl)-1,1-dioxo-1,2-dihydro-1λ⁶-benzo[1,2,4]thiadiazin-7-yl]-methanesulfonamide | +++ |
| 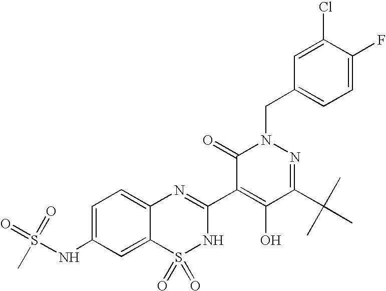 | N-{3-[6-tert-Butyl-2-(3-chloro-4-fluoro-benzyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 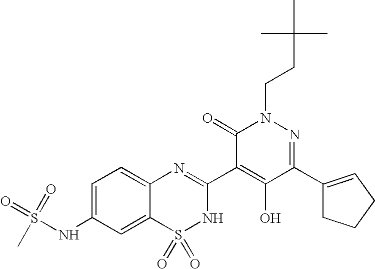 | N-{3-[6-Cyclopent-1-enyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 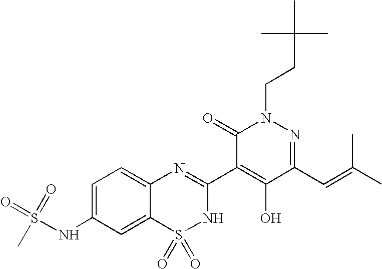 | N-{3-[2-(3,3-Dimethyl-butyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 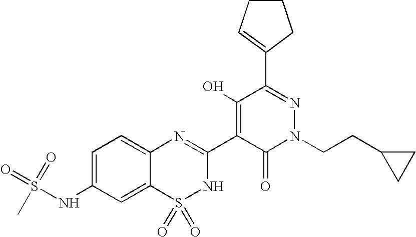 | N-{3-[6-Cyclopent-1-enyl-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 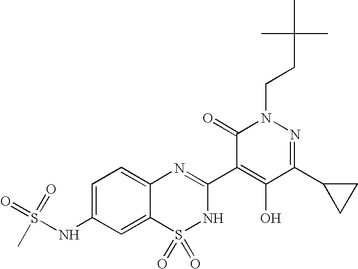 | N-{3-[6-Cyclopropyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 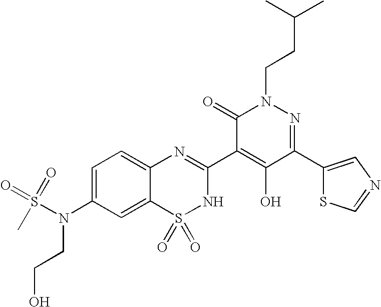 | N-(2-Hydroxy-ethyl)-N-{3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide | ++ |
| 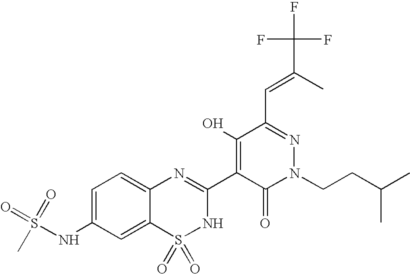 | N-{3-[5-Hydroxy-2-(3-methyl-butyl)-3-oxo-6-(3,3,3-trifluoro-2-methyl-propenyl)-2,3-dihydro-pyridazin-4-yl]-1,1-dioxo-1,2-dihydro-1λ⁶-benzo[1,2,4]thiadiazin-7-yl}-methanesulfonamide | ++ |
| 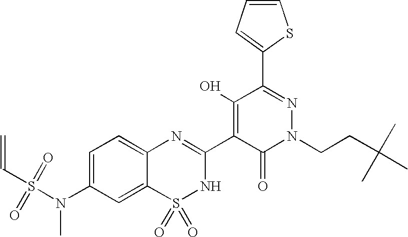 | Ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methyl-amide | + |
| 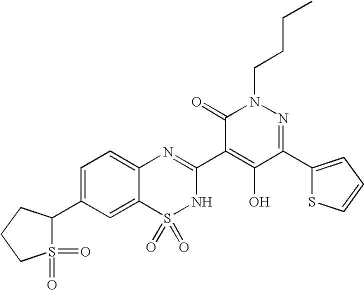 | 4-[7-(1,1-Dioxo-tetrahydro-1l6-thiophen-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one | ++ |
| 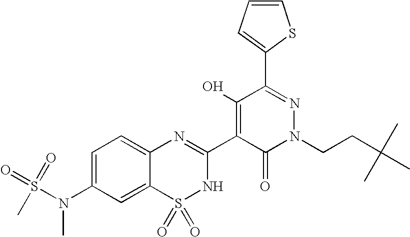 | N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide | ++ |
| 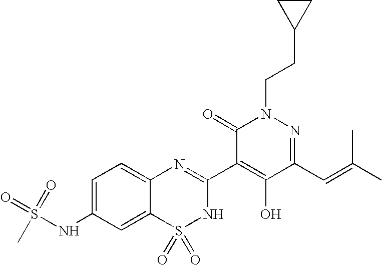 | N-{3-[2-(2-Cyclopropyl-ethyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide | +++ |
| 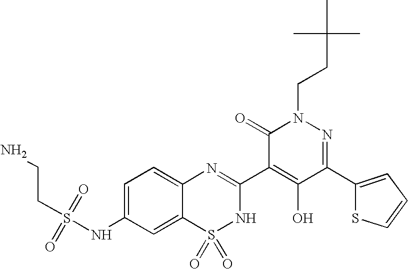 | 2-Amino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide | +++ |
| 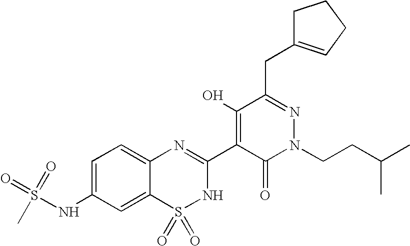 | N-{3-[6-Cyclopent-1-enylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide | ++ |
| 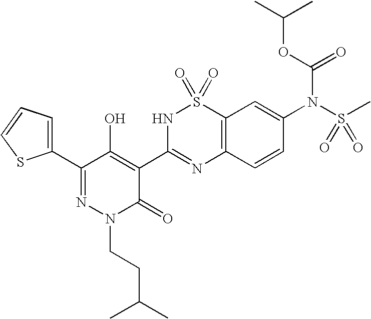 | N-({3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methanesulfonyl)-isopropylcarbamate | ++ |
| 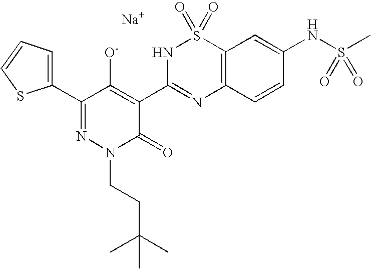 | N-{3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide, sodium salt | +++ |
| 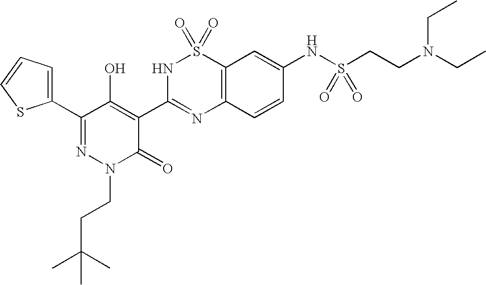 | 2-Diethylamino-ethanesulfonicacid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide | ++ |

10 μM to 1 μM: “+”
≦1 μM to 0.1 μM: “++”
≦0.1 μM: “+++”

The measured mass (LC-MS) for each tested compound in Formula I corresponded to the predicted mass.

### HCV Replicon Assay (Replicon EC₅₀ (μM))

Cell Line:
Human hepatocyte Huh7 cells containing the HCV luciferase reporter replicon were obtained from Ralf Bartenschlager at the University of Mainz, Germany. These cells are maintained under Neomycin selection and passaged when 80-90% confluent.
This cell line contains an autonomously replicating RNA element (replicon) incorporating the non-structural HCV elements necessary for replication, and upon which the survival of the replicon in the cell depends. The replicon also encodes the synthesis of firefly luciferase. Inhibition of any of the critical HCV functions by a compound leads to loss of the replicon and subsequent loss of luciferase. The amount of luciferase remaining following a standard incubation with a compound is a measure of the anti-HCV activity of the compound.

The assay is conducted by preparing sufficient 96-well plates containing these cells, wherein cells are seeded at 6000 cells/well in a 96-well plate, in 200 μl of final media volume. Cells are then incubated for 24 hours at 37° C., 5% CO₂, and 95% humidity before compound is added.

Six point half-log concentration response assays are conducted to determine potency/EC₅₀ of Formula I compounds to inhibit HCV replicon replication. The final percent DMSO acceptable in this assay system is 0.6%. Compounds are diluted in media in an appropriate format and 5 μl of each drug dilution is added to each well. Cells are then incubated with compounds for 3 days at 37° C., 5% CO₂, and 95% humidity.

Following this incubation period, plates are washed twice with phosphate buffered saline to remove media, and cells are lysed by the addition of 25 μl of 1× Passive Lysis Buffer (Promega Cat#E1941) to each well, and gently shaken at room temperature for 20 minutes. The luciferase activity in each well is then determined, and the inhibition calculated by reference to appropriate controls.

### HCV Cell Cytotoxicity Assay (Replicon CC₅₀ (μM))

Cell toxicity assays for CC₅₀ determination are conducted on Huh7 cells in a protocol parallel to the HCV replicon assay described above. However, rather than measuring the luciferase activity, cell number is measured using XTT (Sigma) and detected with a fluorometer at 450 nM/650 nM using Genios/Tecan (Xfluor). For these studies, cells are incubated in 96 well microtiter plates for 3 days with several concentrations of each compound, wherein the concentrations encompassed the range previously found to be effective in inhibiting HCV replication. At the end of that time, the cells are treated with an XTT solution and toxicity measured and CC₅₀ determinations automatically recorded.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention.

What is claimed is:

1. A compound of Formula I 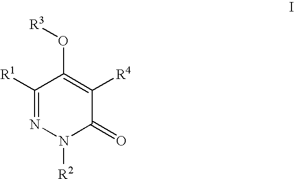
wherein
R¹ is hydrogen, cyano, halo, C₁-C₆ alkyl, C₁-C₆ haloalkyl, C₁-C₆ hydroxyalkyl, C₁-C₆ alkoxy, C₁-C₆ alkenyl, C₁-C₆ alkynyl, —CO₂R⁷, —C(O)NR⁷R⁸, C₃-C₈ cycloalkyl, aryl, or heterocyclyl having 1, 2, or 3 N, O, or S atoms, wherein R⁷ and R⁸ are independently hydrogen, C₁-C₆ alkyl, aryl, or heterocyclyl,
R² is hydrogen, C₁-C₆ alkyl, C₁-C₆ alkenyl, C₁-C₆ alkynyl, C₁-C₆ haloalkyl, C₁-C₆ hydroxyalkyl, C₁-C₆ alkoxy, C₃-C₈ cycloalkyl, aryl, or heterocyclyl having 1, 2, or 3 N, O, or S atoms, —C₁-C₆ alkylene(C₃-C₈ cycloalkyl), —C₁-C₆ alkylene(aryl), or —C₁-C₆ alkylene (heterocyclyl),
R³ is hydrogen, C₁-C₆ alkyl, or —(C₁-C₆ alkylene)ₙ-(O)ₜC(O)R⁹, wherein n and t are independently 0 or 1, wherein R⁹ is C₁-C₆ alkyl, aryl, or heterocyclyl,
R⁴ is selected from 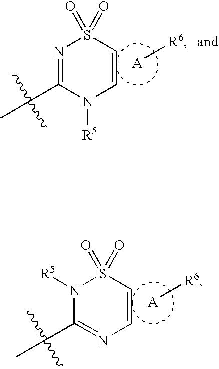
wherein R⁵ is hydrogen or C₁-C₆ alkyl, and
Ring A is a 6-membered aryl or heterocyclyl, substituted by 1-3
R⁶ moieties, wherein R⁶ is —NR¹⁰CO₂R¹¹, —NR¹⁰SO₂R¹¹, —NR¹⁰SO₂NR¹²R¹³, —NR(C₁-C₆ alkylene)SO₂NR¹²R¹³, —(C₁-C₆ alkylene)-SO₂R¹¹, —(C₁-C₆ alkylene)-CHR¹⁴SO₂R¹¹ or —(C₁-C₆ alkenyl)-SO₂R¹¹, wherein R¹⁰ is hydrogen, C₁-C₆ alkyl, —CO₂R¹¹, or —(C₁-C₆ alkylene)-OC(O)(C₁-C₆ alkyl), R¹¹ is C₁-C₆ alkyl, C₁-C₆ alkenyl, —(C₁-C₆ alkylene)NH₂, aryl, C₃-C₈ cycloalkyl, or heterocyclyl, R¹² and R¹³ are independently hydrogen, C₁-C₆ alkyl, or —CO₂R⁹, R¹⁴ is H or C₁-C₆ alkyl, or R¹⁰ and R¹¹ or R¹¹ and R¹⁴ combine with the hetero atom(s) to which they are attached to form a 5- or 6-membered heterocyclyl ring, and
wherein the above alkyl, alkylene, alkenyl, alkynyl, aryl, cycloalkyl, or heterocyclyl moieties are each optionally and independently substituted by 1-3 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents,
or a pharmaceutically acceptable salt, hydrate, solvate, tautomer or stereoisomer thereof.

2. The compound of claim 1, wherein R¹ is hydrogen, cyano, halo, C₁-C₆ alkyl, C₁-C₆ haloalkyl, C₁-C₆ hydroxyalkyl, C₁-C₆ alkoxy, C₁-C₆ alkenyl, C₁-C₆ alkynyl, —CO₂R⁷, —C(O)NR⁷R⁸, aryl, or heterocyclyl having 1, 2, or 3 N, O, or S atoms, wherein R⁷ and R⁸ are independently hydrogen, C₁-C₆ alkyl, aryl or heterocyclyl having 1 or 2 N, O, or S atoms.

3. The compound of claim 2, wherein R¹ is heterocyclyl having 1 N, O, or S atom.

4. The compound of claim 2, wherein R¹ is selected from

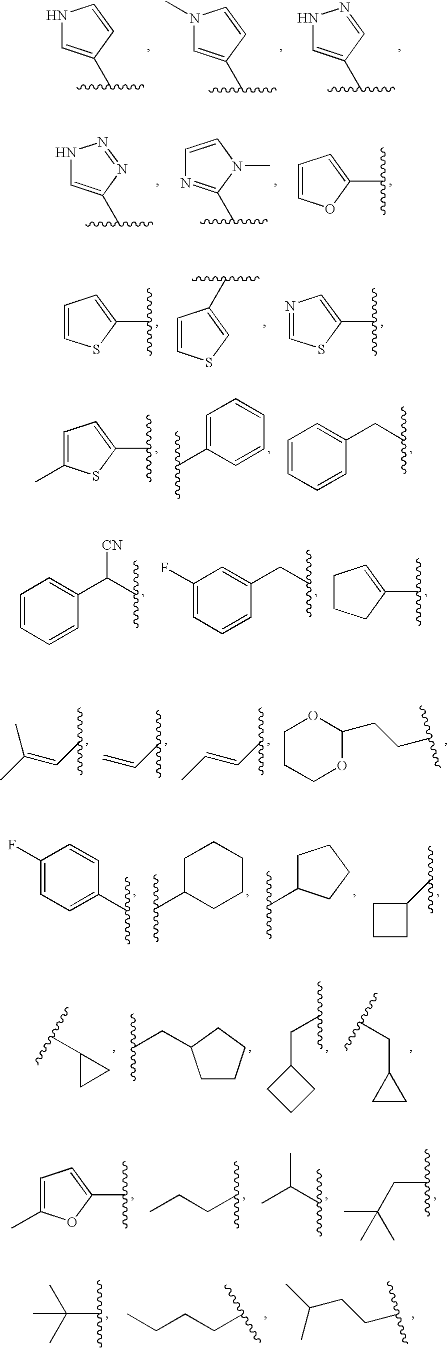

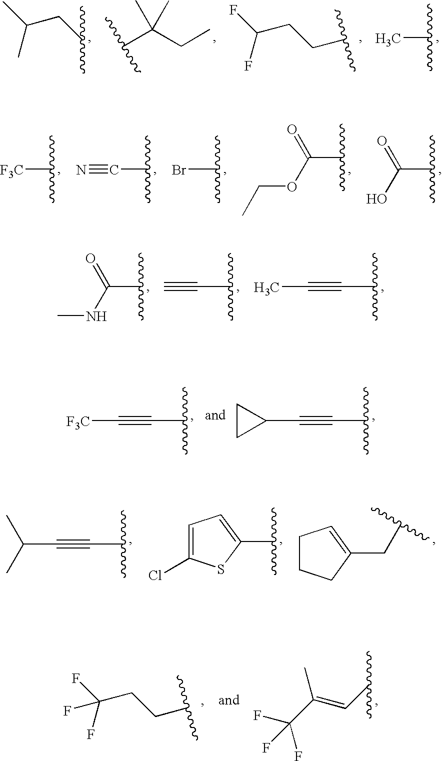

5. The compound of claim 4, wherein R¹ is selected from

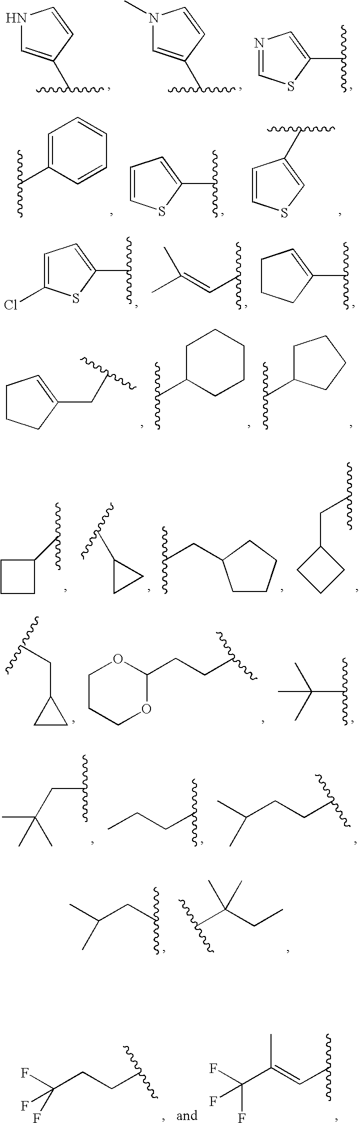

6. The compound of claim 1, wherein R² is selected from C₁-C₆ alkyl, alkenyl, alkynyl, C₁-C₆ haloalkyl, C₃-C₈ cycloalkyl, aryl, and heterocyclyl having 1, 2, or 3 N, O, or S atoms.

7. The compound of claim 6, wherein R² is selected from C₁-C₆ alkyl, C₃-C₈ cycloalkyl, aryl, and heterocyclyl having 1, 2, or 3 N, O, or S atoms.

8. The compound of claim 6, wherein R² is selected from 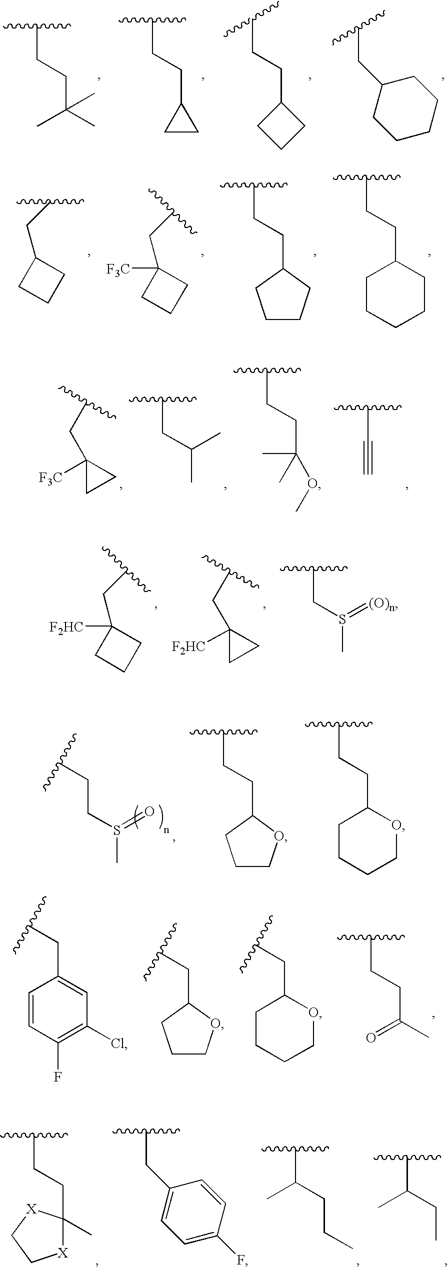 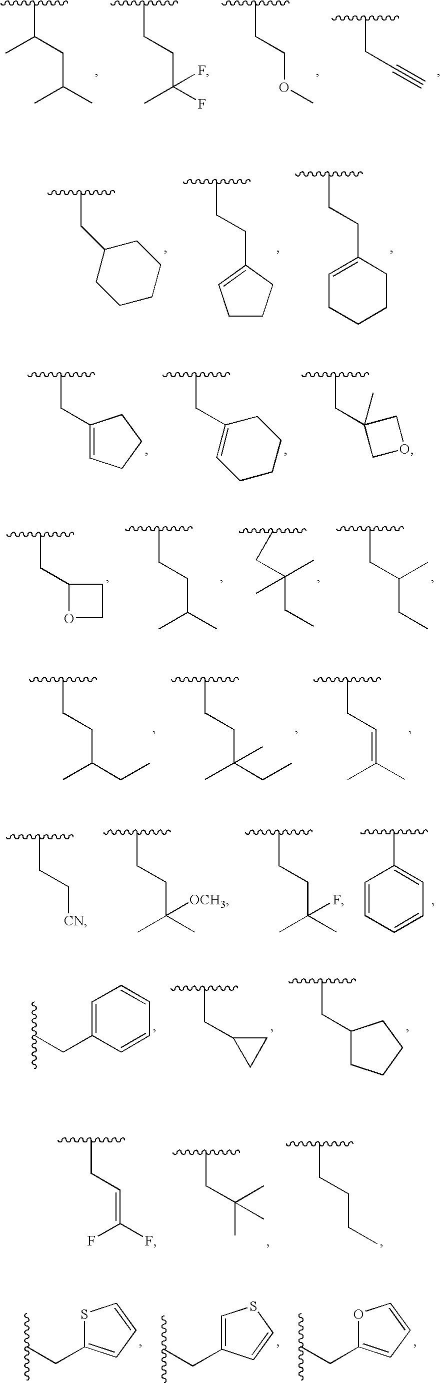 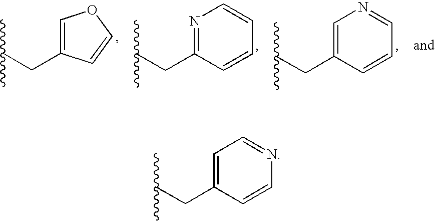
wherein X is O or Sand n=0, 1, or 2.

9. The compound of claim 8, wherein R² is selected from

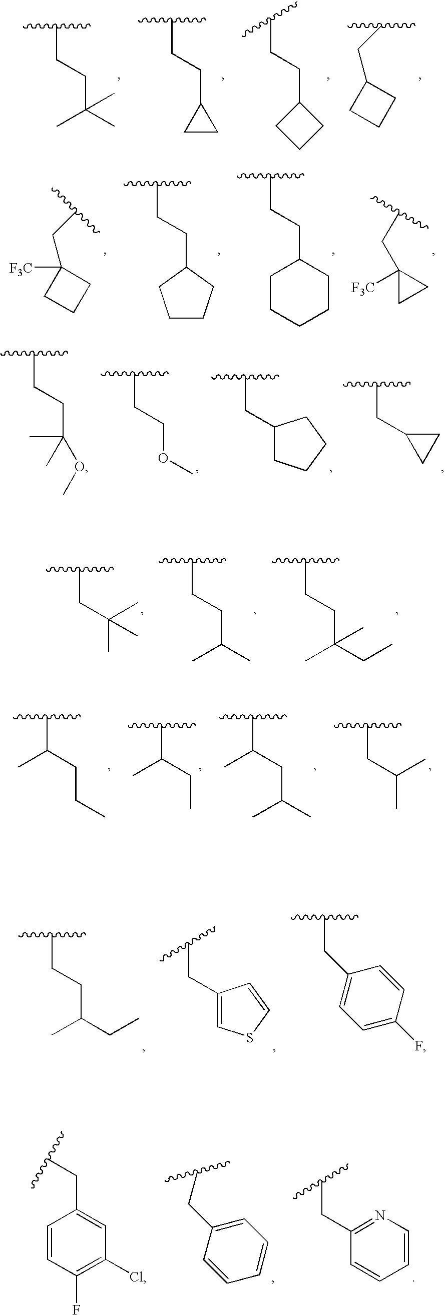

10. The compound of claim 1, wherein R³ is hydrogen, methyl, or —(CH₂)ₙ—(O)ₜC(O)R⁹, wherein n and t are independently 0 or 1.

11. The compound of claim 1, wherein R⁵ is hydrogen or methyl.

12. The compound of claim 1, wherein Ring A is selected from

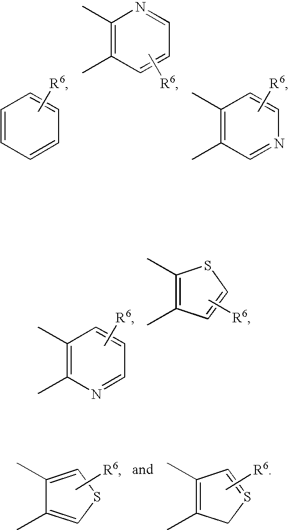

13. The compound of claim 12, wherein Ring A is 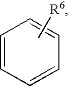
wherein R⁶ is —NR¹⁰SO₂R¹¹ or —NR¹⁰SO₂NR¹²R¹³, wherein R¹⁰ is hydrogen or C₁-C₆ alkyl, R¹¹ is C₁-C₆ alkyl, and R¹² and R¹³ are independently hydrogen or C₁-C₆ alkyl.

14. A compound selected from
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(4-Fluoro-benzyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
Ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-sec-Butyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[5-Hydroxy-2-(3-methoxy-3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Ethanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
2-Methyl-propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
N-{3-[2-(3,3-Dimethyl-pentyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(1-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(1,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclohexyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
Ethanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[2-(4-Fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-5-hydroxy-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-3-oxo-6-thiophen-2-yl-2-(1-trifluoromethyl-cyclobutylmethyl)-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclobutyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopentyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-pentyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-methanesulfonamide;
N-{3-[2-(3-Chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Cyclopropanesulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
N-[3-(5-Hydroxy-2-isobutyl-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(2-Cyclopropylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(2-Cyclopentylmethyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[2-(2,2-Dimethyl-propyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
2,2-Dimethyl-propionic acid ({3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methyl ester;
N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-methanesulfonamide;
N-[3-(2-Benzyl-5-hydroxy-3-oxo-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-[3-(5-Hydroxy-3-oxo-2-pyridin-2-ylmethyl-6-thiophen-2-yl-2,3-dihydro-pyridazin-4-yl)-1,1-dioxo-1,2-dihydro-1λ⁶-benzo[1,2,4]thiadiazin-7-yl]-methanesulfonamide;
N-{3-[6-(5-Chloro-thiophen-2-yl)-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[2-(3-chloro-4-fluoro-benzyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Propane-2-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
Propane-1-sulfonic acid {3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-amide;
Propane-2-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
Propane-1-sulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[2-(4-Fluoro-benzyl)-5-hydroxy-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide;
2,2-Dimethyl-propionic acid ({3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonyl-amino)-methyl ester;
N-{3-[6-Cyclohexyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-propyl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-(1H-pyrrol-3-yl)-2,3-dihydro-pyridazin-4-yl]-1,1-dioxo-1,2-dihydro-1λ⁶-benzo[1,2,4]thiadiazin-7-yl}-methanesulfonamide;
2,2-Dimethyl-propionic acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yloxymethyl ester;
N-{3-[5-Hydroxy-3-oxo-6-thiophen-2-yl-2-(1-trifluoromethyl-cyclopropylmethyl)-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[2-(2-Cyclopropyl-ethyl)-5-hydroxy-3-oxo-6-thiophen-3-yl-2,3-dihydro-pyridazin-4-yl]-1,1-dioxo-1,2-dihydro-1λ⁶-benzo[1,2,4]thiadiazin-7-yl}-methanesulfonamide;
Propane-2-sulfonic acid {3-[2-(2-cyclopropyl-ethyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-6-(1-methyl-1H-pyrrol-3-yl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-isopropyl-methanesulfonamide;
N-{3-[5-Hydroxy-2-(3-methyl-butyl)-3-oxo-6-phenyl-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[6-(1,1-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopentyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
5-Hydroxy-4-(7-methanesulfonylmethyl-1,1-dioxo-1,2-dihydro-1λ⁶-benzo[1,2,4]thiadiazin-3-yl)-2-(3-methyl-butyl)-6-thiophen-2-yl-2H-pyridazin-3-one;
4-[7-(1,1-Dioxo-1l6-isothiazolidin-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one;
5-Hydroxy-4-[7-(2-methanesulfonyl-vinyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one;
5-Hydroxy-4-[7-(2-methanesulfonyl-ethyl)-1,1-dioxo-1,2-dihydro-1λ⁶-benzo[1,2,4]thiadiazin-3-yl]-2-(3-methyl-butyl)-6-thiophen-2-yl-2H-pyridazin-3-one;
N-[3-(2-Cyclopropylmethyl-5-hydroxy-3-oxo-6-phenyl-2,3-dihydro-pyridazin-4-yl)-1,1-dioxo-1,2-dihydro-1l6-benzo[1,2,4]thiadiazin-7-yl]-methanesulfonamide;
Isobutyric acid 5-[7-(methanesulfonyl-methyl-amino)-1,1-dioxo-1,2-dihydro-1λ⁶-benzo[1,2,4]thiadiazin-3-yl]-1-(3-methyl-butyl)-6-oxo-3-thiophen-2-yl-1,6-dihydro-pyridazin-4-yl ester;
N-{3-[6-tert-Butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-(2-[1,3]Dioxan-2-yl-ethyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-6-isobutyl-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methoxymethyl-methanesulfonamide;
N-Benzyloxymethyl-N-{3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-[3-(2-Cyclobutylmethyl-5-hydroxy-3-oxo-6-thiazol-5-yl-2,3-dihydro-pyridazin-4-yl)-1,1-dioxo-1,2-dihydro-1λ⁶-benzo[1,2,4]thiadiazin-7-yl]-3-carbamic-acid-benzyl-ester-sulfamide;
N-{3-[5-Hydroxy-2-(2-methoxy-ethyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide;
N-{3-[6-(2,2-Dimethyl-propyl)-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide;
N-{3-[5-Methoxy-2-(3-methyl-butyl)-3-oxo-6-thiophen-2-yl-2,3-dihydro-pyridazin-4-yl]-4-methyl-1,1-dioxo-1,4-dihydro-1λ⁶-benzo[1,2,4]thiadiazin-7-yl}-N-methyl-methanesulfonamide;
N-{3-[6-Cyclopropylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclobutylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
Cyclopropanesulfonic acid {3-[6-tert-butyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-amide;
N-{3-[5-Hydroxy-2-(3-methyl-butyl)-3-oxo-6-(3,3,3-trifluoro-propyl)-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(2-Cyclopropyl-ethyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-Cyclobutylmethyl-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[5-Hydroxy-2-(3-methyl-butyl)-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
2-Amino-ethanesulfonic acid {3-[5-hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
2-{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-ylamino}-ethanesulfonic acid amide;
N-{3-[2-(3,3-Dimethyl-butyl)-6-(2,2-dimethyl-propyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid methyl ester;
{3-[5-Hydroxy-2-(3-methyl-butyl)-3-oxo-6-thiophen-2-yl-2,3-dihydro-pyridazin-4-yl]-1,1-dioxo-1,2-dihydro-1λ⁶-benzo[1,2,4]thiadiazin-7-yl}-carbamic acid isopropyl ester;
N-{3-[6-Cyclobutyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopentylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-sulfamide;
N-(3-[6-Cyclopentylmethyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2,6-bis-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-[3-(5-Hydroxy-3-oxo-6-thiophen-2-yl-2-thiophen-3-ylmethyl-2,3-dihydro-pyridazin-4-yl)-1,1-dioxo-1,2-dihydro-1λ⁶-benzo[1,2,4]thiadiazin-7-yl]-methanesulfonamide;
N-{3-[6-tert-Butyl-2-(3-chloro-4-fluoro-benzyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopent-1-enyl-2-(2-cyclopropyl-ethyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[6-Cyclopropyl-2-(3,3-dimethyl-butyl)-5-hydroxy-3-oxo-2,3-dihydro-pyridazin-4-yl]-1,1-dioxo-1,2-dihydro-1λ⁶-benzo[1,2,4]thiadiazin-7-yl}-methanesulfonamide;
N-(2-Hydroxy-ethyl)-N-{3-[5-hydroxy-2-(3-methyl-butyl)-3-oxo-6-thiazol-5-yl-2,3-dihydro-pyridazin-4-yl]-1,1-dioxo-1,2-dihydro-1λ⁶-benzo[1,2,4]thiadiazin-7-yl}-methanesulfonamide;
N-{3-[5-Hydroxy-2-(3-methyl-butyl)-3-oxo-6-(3,3,3-trifluoro-2-methyl-propenyl)-2,3-dihydro-pyridazin-4-yl]-1,1-dioxo-1,2-dihydro-1λ⁶-benzo[1,2,4]thiadiazin-7-yl}-methanesulfonamide;
Ethenesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methyl-amide;
4-[7-(1,1-Dioxo-tetrahydro-1l6-thiophen-2-yl)-1,1-dioxo-1,2-dihydro-1λ⁶-benzo[1,2,4]thiadiazin-3-yl]-5-hydroxy-2-(3-methyl-butyl)-6-thiophen-2-yl-2H-pyridazin-3-one;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-N-methyl-methanesulfonamide;
N-{3-[2-(2-Cyclopropyl-ethyl)-5-hydroxy-6-(2-methyl-propenyl)-3-oxo-2,3-dihydro-pyridazin-4-yl]-1,1-dioxo-1,2-dihydro-1λ⁶-benzo[1,2,4]thiadiazin-7-yl}-methanesulfonamide;
2-Amino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-amide;
N-{3-[6-Cyclopent-1-enylmethyl-5-hydroxy-2-(3-methyl-butyl)-3-oxo-2,3-dihydro-pyridazin-4-yl]-1,1-dioxo-1,2-dihydro-1λ⁶-benzo[1,2,4]thiadiazin-7-yl}-methanesulfonamide;
N-{3-[2-(3,3-Dimethyl-butyl)-5-hydroxy-3-oxo-6-thiophen-2-yl-2,3-dihydro-pyridazin-4-yl]-1,1-dioxo-1,2-dihydro-1λ⁶-benzo[1,2,4]thiadiazin-7-yl}-methanesulfonamide, sodium salt; and
2-Diethylamino-ethanesulfonic acid {3-[2-(3,3-dimethyl-butyl)-5-hydroxy-3-oxo-6-thiophen-2-yl-2,3-dihydro-pyridazin-4-yl]-1,1-dioxo-1,4-dihydro-1λ⁶-benzo[1,2,4]thiadiazin-7-yl}-amide.

15. A pharmaceutically acceptable composition comprising a compound of claim 1 and a pharmaceutically acceptable carrier.

16. A method of inhibiting hepatitis C virus replication comprising exposing hepatitis C virus to a therapeutically effective concentration of a compound of claim 1.

17. A method for treating or preventing hepatitis C virus infection in a mammal in need thereof, comprising administering to the mammal a therapeutically or prophylactically effective amount of a compound of claim 1.

18. The method of claim 17 wherein the mammal is a human.

19. The method of claim 18 further comprising administering an additional therapeutic agent to the mammal.

20. The method of claim 19 wherein the additional therapeutic agent is selected from the group consisting of an antibiotic, an antiemetic agent, an antidepressant, an antifungal agent, an anti-inflammatory agent, an antiviral agent, an anticancer agent, an immunomodulatory agent, an α-interferon, a β-interferon, a ribavirin, an alkylating agent, a hormone, a cytokine and a toll receptor-like modulator.

21. The method of claim 20 wherein the additional therapeutic agent is a toll receptor-like modulator.
